(19) United States
(12) Patent Application Publication
(10) Pub. No.: US 2017/0121315 A1
(43) Pub. Date: May 4, 2017

(54) HETEROBICYCLICALLY SUBSTITUTED 4-OXOBUTANE ACID DERIVATIVES AND USE THEREOF

(71) Applicant: BAYER PHARMA AKTIENGESELLSCHAFT, Berlin (DE)

(72) Inventors: Hartmut BECK, Wuppertal (DE); Volkhart Min-Jian LI, Velbert (DE); Yolanda CANCHO GRANDE, Leverkusen (DE); Andreas TIMMERMAN, Düsseldorf (DE); Dirk BROHM, Mettmann (DE); Hannah JÖRIßEN, Heiligenhaus (DE); Pamela BOGNER, München (DE); Michael GERISCH, Wuppertal (DE); Dieter LANG, Velbert (DE)

(21) Appl. No.: 15/317,519

(22) PCT Filed: Jun. 8, 2015
(86) PCT No.: PCT/EP2015/062652

§ 371 Date: Dec. 9, 2016

(30) Foreign Application Priority Data
Jun. 12, 2014 (EP) 14172169.6

(51) Int. Cl. C07D 405/06; A61K 31/53; C07D 409/06; C07D 413/14; C07D 409/14; C07D 413/06; A61K 45/06; C07D 405/14

(52) U.S. Cl. CPC C07D 405/06; A61K 45/06; A61K 31/53; C07D 405/14; C07D 413/14; C07D 409/14; C07D 413/06; C07D 409/06

(57) ABSTRACT

The present application relates to novel heterobicyclically substituted 4-oxobutanoic acid derivatives, to processes for their preparation, to their use alone or in combinations for the treatment and/or prevention of diseases and to their use for producing medicaments for the treatment and/or prevention of diseases, especially for the treatment and/or prevention of respiratory, pulmonary and cardiovascular disorders.

Heterobicyclically Substituted 4-Oxobutane Acid Derivatives And Use Thereof derivatives, to processes for their preparation, to their use alone or in combinations for the treatment and/or prevention of diseases and to their use for producing medicaments for the treatment and/or prevention of diseases, especially for the treatment and/or prevention of respiratory, pulmonary and cardiovascular disorders.

Human macrophage elastase (HME, EC 3.4.24.65) forms part of the family of the matrix metallopeptidases (MMPs) and is also called human matrix metallopeptidase 12 (hMMP-12). The protein is formed, activated and released to an increased degree, inter alia, by macrophages after contact with “stimulating” substances or particles. Such substances and particles may be present, for example, as extraneous substances in suspended particles as occur in cigarette smoke or industrial dusts, inter alia. In the broader sense, also counted among these stimulating particles are endogenous and exogenous cell constituents and cell fragments, as can occur in inflammation processes, sometimes in high concentration. The highly active enzyme is capable of degrading a multitude of binding tissue proteins, for example primarily the protein elastin (hence the name), and further proteins and proteoglycans such as collagen, fibronectin, laminin, chondroitin sulphate, heparan sulphate and others. This proteolytic activity of the enzyme makes macrophages capable of penetrating the basal membrane. Elastin, for example, occurs in high concentrations in all tissue types that exhibit high elasticity, for example in the lung and in arteries. In a large number of pathological processes, such as tissue damage, HME plays an important role in tissue degradation and remodelling. Furthermore, HME is an important modulator in inflammation processes. It is a key molecule in the recruitment of inflammation cells in that it, for example, releases the central inflammation mediator tumour necrosis factor alpha (TNF-α) and intervenes in the signal pathway mediated by transforming growth factor-beta (TGF-β) [Hydrolysis of a Broad Spectrum of Extracellular Matrix Proteins by Human Macrophage Elastase, Gronski et al., J. Biol. Chem. 272, 12189-12194 (1997)]. MMP-12 also plays a role in host defence, particularly in the regulation of antiviral immunity, presumably as a result of an intervention into the interferon-alpha (IFN-α)-mediated signal pathway [A new transcriptional role for matrix metalloproteinase-12 in antiviral immunity, Marchant et al., Nature Med. 20, 493-502 (2014)].

It is therefore assumed that HME plays an important role in many disorders, injuries and pathological lesions whose aetiology and/or progression is associated with an infectious or non-infectious event and/or proliferative and hypertrophic tissue and vessel remodelling. These may be in particular diseases and/or damage to the lungs, the kidneys or the cardiovascular system, or they may be cancer diseases or other inflammatory diseases [Macrophage metalloelastase (MMP-12) as a target for inflammatory respiratory diseases, Lagente et al., Expert Opin. Ther. Targets 13, 287-295 (2009); Macrophage Metalloelastase as a major Factor for Glomerular Injury in Anti-Glomerular Basement Membrane Nephritis, Kaneko et al., J. Immunol. 170, 3377-3385 (2003); A Selective Matrix Metalloelastase-12 Inhibitor Retards Atherosclerotic Plaque Development in Apolipoprotein E Knock-out Mice, Johnson et al., Arterioscler. Thromb. Vasc. Biol. 31, 528-535 (2011); Impaired Coronary Collateral Growth in the Metabolic Syndrome Is in Part Mediated by Matrix Metalloelastase 12-dependent Production of Endostatin and Angiostatin, Dodd et al., Arterioscler. Thromb. Vasc. Biol. 33, 1339-1349 (2013); Matrix metalloproteinase pharmacogenomics in non-small-cell lung carcinoma, Chetty et al., Pharmacogenomics 12, 535-546 (2011)].

Diseases and damage to the lung that should be mentioned in this context are especially chronic obstructive pulmonary disease (COPD), pulmonary emphysema, interstitial lung diseases (ILD), for example idiopathic pulmonary fibrosis (IPF) and pulmonary sarcoidosis, acute lung injury (ALI), acute respiratory distress syndrome (ARDS), cystic fibrosis (CF; also called mucoviscidosis), asthma, and also infectious, particularly virally induced, respiratory disorders. Other fibrotic disorders that should be mentioned here by way of example include hepatic fibrosis and systemic sclerosis. Diseases and damage to the cardiovascular system in which HME is involved are, for example, tissue and vascular lesions in the event of arteriosclerosis, here in particular carotid arteriosclerosis, infective endocarditis, here in particular viral myocarditis, cardiomyopathy, heart failure, cardiogenic shock, acute coronary syndrome (ACS), aneurysms, reperfusion injuries following an acute myocardial infarct (AMI), ischaemic injuries to the kidneys or the retina, and also the chronic courses thereof, for example chronic kidney disease (CKD) and Alport's syndrome. Mention should also be made here of metabolic syndrome and obesity. Diseases connected to sepsis are, for example, systemic inflammatory response syndrome (SIRS), severe sepsis, septic shock and multiple organ failure (MOF)/multiorgan dysfunction (MODS) and also disseminated intravascular coagulation (DIC). Examples of tissue degradation and remodelling during neoplastic processes are the invasion of cancer cells into healthy tissue (formation of metastases) and neovascularization (neoangiogenesis). Other inflammatory diseases in which HME plays a role are rheumatoid diseases, for example rheumatoid arthritis, and also chronic intestinal inflammation (inflammatory bowel disease (IBD); Crohn's disease CD; ulcerative colitis UC).

In general, it is assumed that elastase-mediated pathological processes are based on a shifted equilibrium between free elastase (HME) and the endogenous tissue inhibitor of metalloproteinase (TIMP). In various pathological processes, particularly inflammation processes, the concentration of free elastase (HME) is elevated, such that there is a local shift in the balance between protease and anti-protease in favour of the protease. A similar (im)balance exists between the elastase of neutrophil cells (human neutrophil elastase, HNE, a member of the serine protease family) and endogenous anti-protease AAT (alpha-1 anti-trypsin, a member of the serine protease inhibitors, SERPINs). The two equilibria are coupled to one another since HME cleaves and inactivates the inhibitor of the HNE and, conversely, HNE cleaves and inactivates the HME inhibitor, which can result in an additional shift in the respective protease/anti-protease imbalances. Moreover, in the environment of local inflammation, strongly oxidizing conditions exist (an “oxidative burst”), which further strengthens the protease/anti-protease imbalance [Pathogenic triad in COPD: oxidative stress, protease-antiprotease imbalance, and inflammation, Fischer et al., Int. J. COPD 6, 413-421 (2011)].

Currently, more than 20 MMPs are known, which are historically roughly divided into different classes with regard to their most prominent substrates, e.g. gelatinases (MMP-2, MMP-9), collagenases (MMP-1, MMP-8, MMP-13), stromelysins (MMP-3, MMP-10, MMP-11) and matrilysins (MMP-7, MMP-26). HME (MMP-12) is hitherto the only representative of metalloelastases. Moreover, further MMPs are added to the group of so-called MT-MMPs (membrane-type MMPs) since these have a characteristic domain which anchors the protein in the membrane (MMP-14, MMP-15, MMP-16, MMP-17, MMP-24, MMP-25). A common feature of all the MMPs is a preserved zinc-binding region in the active centre of the enzyme which is important for the catalytic activity and which can also be found in other metalloproteins (e.g. a disintegrin and metalloproteinase, ADAM). The complexed zinc is masked by a sulphhydryl group in the N-terminal pro-peptide domain of the protein, which leads to an enzymatically inactive pro-form of the enzyme. Only as a result of a cleaving off of this pro-peptide domain is the zinc in the active centre of the enzyme freed from this coordination and the enzyme is thereby activated (so-called activation by cysteine switch) [Matrix metalloproteinase inhibitors as therapy for inflammatory and vascular diseases, Hu et al., Nature Rev. Drug Discov. 6, 480-498 (2007)].

Most of the known synthetic MMP inhibitors have a zinc-complexing functional group, very frequently, for example, a hydroxamate, carboxylate or thiol [Recent Developments in the Design of Specific Matrix Metalloproteinase Inhibitors aided by Structural and Computational Studies, B. G. Rao, Curr. Pharm. Des. 11, 295-322 (2005)]. The scaffold of these inhibitors often still resembles peptides, in which case they are called “peptidomimetics” (generally with poor oral bioavailability), or it has no similarity to peptides, in which case they are more generally called small molecules (SMOLs). The physicochemical and pharmacokinetic properties of these inhibitors have, in quite general terms, a major influence on which target molecules (targets) and which undesired molecules (anti-targets, off-targets) are “encountered” in which tissue, in which period of time and to what extent.

It is a great challenge here to determine the specific role of a particular MMP in the course of a disease. This is made particularly difficult by the fact that there is a multitude of MMPs and further similar molecules (e.g. ADAMs), each associated with a multitude of possible physiological substrates and hence, under some circumstances, also with accompanying inhibiting or activating effects in various signal transduction pathways. Numerous in vitro and preclinical in vivo experiments have contributed to a better understanding of the MMPs in various disease models (e.g. transgenic animals, knockout animals and genetic data from human studies). A target can ultimately only be validated with respect to possible medicament therapy in clinical test series in humans or patients. In this context, the first generation of MMP inhibitors has been clinically examined in cancer studies. At this time, only a few representatives of the MMP protein family were known. None of the inhibitors examined were clinically convincing, since the side effects that occurred at effective dosages were intolerable. As emerged as further MMPs became known, the representatives of the first inhibitor generation were non-selective inhibitors, i.e. a large number of different MMPs was inhibited to the same extent (pan-MMP inhibitors, pan-MMPIs). Presumably, the desired effect on one or more MMP targets was concealed by an undesired effect on one or more MMP anti-targets or by means of an undesired effect at another target site (off-target) [Validating matrix metallo proteinases as drug targets and anti-targets for cancer therapy, Overall & Kleifeld, Nature Rev. Cancer 6, 227-239 (2006)].

Newer MMP inhibitors, which are characterized by increased selectivity, have now likewise been clinically tested, including compounds referred to explicitly as MMP-12 inhibitors, although hitherto likewise without compelling clinical success. On closer inspection, the inhibitors previously described as selective have not been found to be quite so selective here either.

For instance, for the clinical test compound “MMP408” as MMP-12 inhibitor, a certain to distinct selectivity in vitro with respect to MMP-13, MMP-3, MMP-14, MMP-9, Agg-1, MMP-1, Agg-2, MMP-7 and TACE is described [A Selective Matrix Metalloprotease 12 Inhibitor for Potential Treatment of Chronic Obstructive Pulmonary Disease (COPD): Discovery of (S)-2-(8-(Methoxycarbonylamino)dibenzo[b,d]furan-3-sulphonamido)-3-methylbutanoic acid (MIMP408), Li et al., J. Med. Chem. 52, 1799-1802 (2009)]. In vitro efficacy data for MMP-2 and MMP-8 suggest less advantageous selectivity with respect to these two MMP representatives [Matrix metalloproteinase-12 is a therapeutic target for asthma in children and young adults, Mukhopadhyay et al., J. Allergy Clin. Immunol. 126, 70-76 (2010)].

Similar observations are made with the clinical test substance AZD1236 for the treatment of COPD, which is described as a dual MMP-9/12 inhibitor [Effects of an oral MMP-9 and-12 inhibitor, AZD1236, on biomarkers in moderate/severe COPD: A randomised controlled trial, Dahl et al., Pulm. Pharmacol. Therap. 25, 169-177 (2012)]. The development of this compound was stopped in 2012; here too, noticeable inhibition of MMP-2 and MMP-13 is cited [http://www.wipo.int/research/en/details.jsp?id=2301].

In the assessment of MMP selectivity, moreover, a cautious assessment of the meaningfulness of animal models is appropriate. The test compound MMP408 for example shows an essentially reduced affinity to the orthologous murine MMP-12 target: IC₅₀ 2 nM (human MMP-12), IC₅₀ 160 nM (murine MMP-12), IC₅₀ 320 nm (MMP-12 in rat) [see above Li et al., 2009; Mukhopadhyay et al., 2010]. No figures relating to potency with respect to other murine MMPs have been published.

The situation seems to be similar for the test substance AZD1236 [see the information given relating to cross-reactivity in various animal species at http://www.wipo.int/research/en/details.jsp?id=2301].

As well as the selectivity profile across species boundaries, the potency on the MMP-12 target itself is very important. Given a comparatively similar pharmacokinetic profile, a compound of high potency will lead to a lower therapeutic dose than a less potent compound and, in general, a lower dose should be associated with a reduced probability of side effects. This is true particularly with regard to what is called the “free fraction” (fraction unbound, fᵤ) of a compound which can interact with the desired target and/or undesired anti- and off-targets (the “free fraction” is defined as the available amount of a compound which is not bound to constituents of blood plasma; these are primarily blood protein constituents, for example albumin). As well as MMP selectivity, specificity is also of major significance.

Novel active ingredients that inhibit macrophage elastase should accordingly have high selectivity and specificity in order to be able to selectively inhibit HME. For this purpose, good metabolic stability of the substances is also necessary (low clearance). Moreover, these compounds should be stable under oxidative conditions in order not to lose inhibitory potency in the course of the disease.

Chronic obstructive pulmonary disease (COPD) is a slowly progressing pulmonary disease characterized by an obstruction of respiratory flow which is caused by pulmonary emphysema and/or chronic bronchitis. The first symptoms of the disease generally manifest themselves during the fourth or fifth decade of life. In the subsequent years of life, shortness of breath frequently becomes worse, and there are instances of coughing combined with copious and purulent sputum, and stenotic respiration extending as far as breathlessness (dyspnoea). COPD is primarily a smokers' disease: smoking is the cause of 90% of all cases of COPD and of 80-90% of all COPD-related deaths. COPD is a big medical problem and constitutes the sixth most frequent cause of death worldwide. Of people over the age of 45, about 4-6% are affected.

Although the obstruction of the respiratory flow may only be partial and temporal, COPD cannot be cured. Accordingly, the aim of treatment is to improve the quality of life, to alleviate the symptoms, to prevent acute worsening and to slow the progressive impairment of lung function.

Existing pharmacotherapies, which have barely changed over the last two or three decades, are the use of bronchodilators to open blocked respiratory passages, and in certain situations corticosteroids to control the inflammation of the lung [Chronic Obstructive Pulmonary Disease, P. J. Barnes, N. Engl. J. Med. 343, 269-280 (2000)]. The chronic inflammation of the lung, caused by cigarette smoke or other irritants, is the driving force of the development of the disease. The underlying mechanism includes immune cells that excrete various chemokines in the course of the inflammation reaction of the lung. As a result, neutrophil cells and, later on, alveolar macrophages are attracted to the connective tissue of the lung and lumen. Neutrophil cells secrete a protease cocktail containing mainly HNE and proteinase 3. Activated macrophages release HME. This results in a local shift in the protease/antiprotease balance in favour of the proteases, which leads, inter alia, to uncontrolled elastase activity and, as a result of this, to an overshoot in degradation of the alveolar elastin. This tissue degradation causes a collapse of the bronchi. This is associated with reduced elasticity of the lung, which leads to impairment of respiratory flow and impaired respiration. Moreover, frequent and prolonged inflammation of the lung can lead to remodelling of the bronchi and consequently to formation of lesions. Such lesions contribute to the occurrence of chronic coughing, which is an indication of chronic bronchitis.

It is known from studies with human sputum samples that the amount of HME protein is associated with the smoking or COPD status: The detectable amounts of HME are at their lowest in the case of non-smokers, somewhat elevated in the case of former smokers and smokers, and distinctly elevated in the case of COPD patients [Elevated MMP-12 protein levels in induced sputum from patients with COPD, Demedts et al., Thorax 61, 196-201 (2006)]. Similar data were obtained with human sputum samples and bronchial alveolar washing fluid (BALF). It was possible here to detect and quantify HME on activated macrophages: amount of HME in COPD patient/smoker>COPD patient/former smoker>former smoker>non-smoker [Patterns of airway inflammation and AMMP-12 expression in smokers and ex-smokers with COPD, Babusyte et al., Respir. Res. 8, 81-90 (2007)].

An inflammatory lung disease similar to COPD in a certain way is interstitial lung disease (ILD), in particular here the manifestation as idiopathic pulmonary fibrosis (IPF) and sarcoidosis [Commonalities between the pro-fibrotic mechanisms in COPD and IPF, L. A. Murray, Pulm. Pharmacol. Therap. 25, 276-280 (2012); The pathogenesis of COPD and IPF: distinct horns of the same devil?, Chilosi et al., Respir. Res. 13:3 (2012)]. Here too, the homeostasis of the extracellular matrix is disturbed. Data from genome-wide association studies suggest a particular role of HME in the disease process of such fibrotic diseases [Gene Expression Profiling Identifies MMP-12 and ADAMDEC1 as Potential Pathogenic Mediators of Pulmonary Sarcoidosis, Crouser et al., Am. J. Respir. Crit. Care Med. 179, 929-938 (2009); Association of a Functional Polymorphism in the Matrix Metalloproteinase-12 Promoter Region with Systemic Sclerosis in an Italian Population, Manetti et al., J. Rheumatol. 37, 1852-1857 (2010); Increased serum levels and tissue expression of matrix metalloproteinase-12 in patients with systemic sclerosis: correlation with severity of skin and pulmonary fibrosis and vascular damage, Manetti et al., Ann. Rheum. Dis. 71, 1064-1070 (2012)].

Furthermore, there is further preclinical evidence of a crucial role of HME in ischaemic-inflammatory disease processes [Macrophage Metalloelastase (MMP-12) Deficiency Mitigates Retinal Inflammation and Pathological Angiogenesis in Ischemic Retinopathy, Li et al., PLoS ONE 7 (12), e52699 (2012)]. A significantly higher MMP-12 expression is also known in the case of ischaemic renal injuries, as is the involvement of MMP-12 in other inflammatory renal diseases [JNK signalling in human and experimental renal ischaemia/reperfusion injury, Kanellis et al., Nephrol. Dial. Transplant. 25, 2898-2908 (2010); Macrophage Metalloelastase as a Major Factor for Glomerular Injury in Anti-Glomerular Basement Membrane Nephritis, Kaneko et al., J. Immun. 170, 3377-3385 (2003); Role for Macrophage Metalloelastase in Glomerular Basement Membrane Damage Associated with Alport Syndrome, Rao et al., Am. J. Pathol. 169, 32-46 (2006); Differential regulation of metzincins in experimental chronic renal allograft rejection: Potential markers and novel therapeutic targets, Berthier et al., Kidney Int. 69, 358-368 (2006); Macrophage infiltration and renal damage are independent of Matrix Metalloproteinase 12 (MMP-12) in the obstructed kidney, Abraham et al., Nephrology 17, 322-329 (2012)].

The problem addressed by the present invention was thus that of identifying and providing novel substances which act as potent, selective and specific inhibitors of human macrophage elastase (HME/MMP-12) and as such are suitable for the treatment and/or prevention, particularly of disorders of the respiratory pathways, the lung and the cardiovascular system.

Patent applications WO 96/15096-A1, WO 97/43237-A1, WO 97/43238-A1, WO97/43239-A1, WO 97/43240-A1, WO 97/43245-A1 and WO 97/43247-A1 disclose 4-aryl- and 4-biaryl-substituted 4-oxobutanoic acid derivatives with inhibitory activity towards MMP-2, MMP-3, MMP-9 and, to a lesser extent, MMP-1; on account of this activity profile, the compounds were considered to be suitable particularly for the treatment of osteoarthritis, rheumatoid arthritis and tumour diseases. WO 98/09940-A1 and WO 99/18079-A1 disclose further biarylbutanoic acid derivatives as inhibitors of MMP-2, MMP-3 and/or MMP-13 which are suitable for treating a wide variety of diseases. WO 00/40539-A1 claims the use of 4-biaryl-4-oxobutanoic acids for the treatment of pulmonary and respiratory disorders, based on a different extent of inhibition of MMP-2, MMP-3, MMP-8, MMP-9, MMP-12 and MMP-13 by these compounds. Furthermore, WO 2012/014114-A1 describes 3-hydroxypropionic acid derivatives and WO 2012/038942-A1 describes oxy- or sulphonylacetic acid derivatives as dual MMP 9/12 inhibitors.

Furthermore, WO 96/38434-A1, WO 98/06711-A1, WO 00/06560-A1 and U.S. Pat. No. 6,037,361 disclose tricyclic heteroaromatic compounds as inhibitors in particular of MMP-2 and MMP-3, and WO 2005/026120-A1 claims, inter alia, tricyclically substituted 3-hydroxypentanoic acid derivatives as MMP-12 inhibitors.

Against the background of the problem described above, however, it was found that these MMP inhibitors from the prior art often have disadvantages such as, more particularly, inadequate inhibitory potency towards MMP-12, inadequate selectivity for MMP-12 compared to other MMPs and/or limited metabolic stability.

It has now been found that, surprisingly, heterobicyclically substituted 4-oxobutanoic acid derivatives have a significantly improved profile in terms of their potency and selectivity with respect to human macrophage elastase (HME/hMMP-12) compared to the compounds known from the prior art. This more targeted mode of action suggests a lower dosability of the compounds according to the invention and a reduced risk of the occurrence of undesired side-effects during therapy.

The compounds according to the invention additionally feature significant inhibitory activity with respect to the orthologous rodent MMP-12 peptidases such as murine MMP-12 (also referred to as murine macrophage elastase) and rat MMP-12. This enables preclinical evaluation of the substances in various established animal models for the above-described diseases.

The present invention provides compounds of the general formula (I)

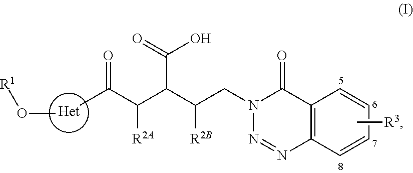

in which
Het represents bicyclic heteroaryl of the formula

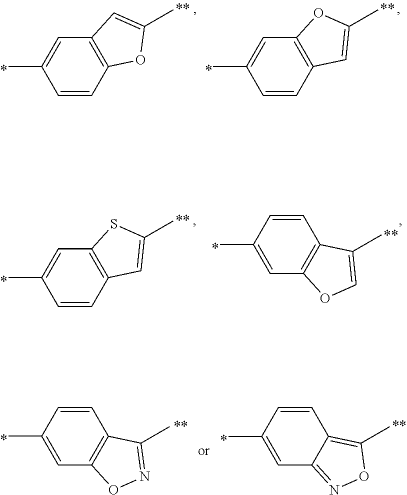

in which * labels the linkage to the R¹O group and ** labels the linkage to the carbonyl group,
R¹ represents (C₁-C₈)-alkyl which can be substituted with cyano or up to six times with fluorine,
   or
   represent a group of the formula

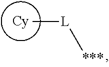

in which *** labels the linkage to the O atom,
L represents —CH₂—, —CH₂—CH₂—, —C(═O)—CH₂—*** or —CH₂—CH₂—CH₂—
and
Cy represents (C₃-C₆)-cycloalkyl, oxetanyl, tetrahydrofuranyl, tetrahydropyranyl, phenyl, thienyl, pyridyl or 2,1,3-benzoxadiazolyl,
   where (C₃-C₆)-cycloalkyl, oxetanyl, tetrahydrofuranyl and tetrahydropyranyl may be mono- or disubstituted, identically or differently, with a radical selected from the group fluorine and methyl
   and
   where phenyl, thienyl and pyridyl can be mono- or disubstituted, identically or differently, with a radical selected from the group fluorine, chlorine, cyano, methyl, difluoromethyl, trifluoromethyl, methoxy, ethoxy, trifluoromethoxy, (trifluoromethyl)sulphanyl, methylsulphonyl, aminocarbonyl, methoxycarbonylamino and 2-oxo-1,3-oxazolidin-3-yl,
R^{2A} and R^{2B} both represent hydrogen or are linked with one another and together form a —CH₂CH₂ bridge
and
R³ represents hydrogen or a substituent selected from the group fluorine, methyl, fluoromethyl, difluoromethyl and trifluoromethyl, where such a substituent can be bonded in the designated position 5, 6, 7 or 8,
   and the salts, solvates and solvates of the salts thereof.

Compounds according to the invention are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are mentioned below as working examples and the salts, solvates and solvates of the salts thereof if the compounds that are encompassed by formula (I) and are mentioned below are not already salts, solvates and solvates of the salts.

Preferred salts in the context of the present invention are physiologically acceptable salts of the compounds according to the invention. Also encompassed are salts which are not themselves suitable for pharmaceutical applications but can be used, for example, for the isolation, purification or storage of the compounds according to the invention.

Physiologically acceptable salts of the compounds according to the invention include in particular the salts derived from conventional bases, by way of example and with preference alkali metal salts (e.g. sodium and potassium salts), alkaline earth metal salts (e.g. calcium and magnesium salts), zinc salts and ammonium salts derived from ammonia or organic amines having 1 to 16 carbon atoms, by way of example and with preference ethylamine, diethylamine, triethylamine, N,N-diisopropylethylamine, monoethanolamine, diethanolamine, triethanolamine, tromethamine, dimethylaminoethanol, diethylaminoethanol, choline, procaine, dicyclohexylamine, dibenzylamine, N-methylmorpholine, N-methylpiperidine, arginine, lysine and 1,2-ethylenediamine.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else, if appropriate, as conformational isomers (enantiomers and/or diastereomers, including those in the case of atropisomers). The present invention therefore encompasses the enantiomers and diastereomers, and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

In the context of the present invention, the term “enantiomerically pure” is understood to the effect that the compound in question with respect to the absolute configuration of the chiral centre is present in an enantiomeric excess of more than 95%, preferably more than 98%. The enantiomeric excess, ee, is calculated here by evaluating an HPLC analysis chromatogram on a chiral phase using the formula below:

$$ee = \vert \frac{enantiomer 1 ( area per cent ) - enantiomer 2 ( area per cent )}{enantiomer 1 ( area per cent ) + enantiomer 2 ( area per cent )} \vert \times 100 \% .$$

If the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, 15N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to the comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore possibly also constitute a preferred embodiment of the present invention.

Isotopic variants of the compounds according to the invention can be prepared by commonly used processes known to those skilled in the art, for example by the methods described further down and the procedures described in the working examples, by using corresponding isotopic modifications of the respective reagents and/or starting compounds.

The present invention additionally also encompasses prodrugs of the compounds according to the invention. The term “prodrugs” refers here to compounds which may themselves be biologically active or inactive, but are converted while present in the body, for example by a metabolic or hydrolytic route, to compounds according to the invention.

The present invention comprises as prodrugs in particular hydrolysable ester derivatives of the carboxylic acids of the formula (I) according to the invention. These are understood to mean esters which can be hydrolysed to the free carboxylic acids, as the main biologically active compounds, in physiological media under the conditions of the biological tests described hereinbelow and in particular in vivo by an enzymatic or chemical route. (C₁-C₄)-Alkyl esters, in which the alkyl group can be straight-chain or branched, are preferred as such esters. Particular preference is given to methyl, ethyl or tert-butyl esters.

In the context of the present invention, unless specified otherwise, the substituents are defined as follows:

(C₁-C₈)-Alkyl and (C₄-C₇)-alkyl in the context of the invention are a straight-chain or branched alkyl radical having, respectively, 1 to 8 and 4 to 7 carbon atoms. The following may be mentioned by way of example: methyl, ethyl, n-propyl, isopropyl, n-butyl, isobutyl, sec-butyl, tert-butyl, n-pentyl, 2-pentyl, 3-pentyl, neopentyl, 3-methylbutyl, n-hexyl, 2-hexyl, 3-hexyl, 4-methylpentyl, n-heptyl, 5-methylhexyl, n-octyl and 6-methylheptyl. Preference is given to a straight-chain or branched alkyl radical having 4 to 7 carbon atoms such as n-butyl, isobutyl, sec-butyl, tert-butyl, n-pentyl, 2-pentyl, 3-pentyl, neopentyl, 3-methylbutyl, n-hexyl, 2-hexyl, 3-hexyl, 4-methylpentyl, n-heptyl and 5-methylhexyl.

(C₃-C₆)-Cycloalkyl in the context of the invention is a monocyclic saturated cycloalkyl group having 3 to 6 ring carbon atoms. By way of example and with preference, mention may be made of the following: cyclopropyl, cyclobutyl, cyclopentyl and cyclohexyl.

In the context of the present invention, all radicals which occur more than once are defined independently of one another. When radicals in the compounds according to the invention are substituted, the radicals may be mono- or polysubstituted, unless specified otherwise. Substitution by one substituent or by two identical or different substituents is preferred. Particular preference is given to substitution by one substituent.

Preference is given in the context of the present invention to compounds of the formula (I) in which
Het represents bicyclic heteroaryl of the formula

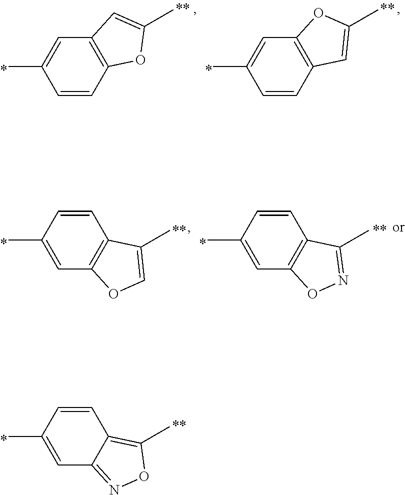

in which * labels the linkage to the R¹O group and ** labels the linkage to the carbonyl group,
R¹ represents (C₄-C₇)-alkyl or a group of the formula

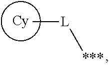

in which *** labels the linkage to the O atom,
L represents —CH₂— oder —CH₂—CH₂—
and
Cy represents (C₃-C₆)-cycloalkyl, tetrahydropyranyl or phenyl,
   where phenyl can be mono- or disubstituted, identically or differently, with a radical selected from the group fluorine, chlorine, methyl, trifluoromethyl, methoxy and aminocarbonyl,
R^{2A} and R^{2B} both represent hydrogen or are linked with one another and together form a —CH₂CH₂ bridge
and
R³ represents hydrogen or a substituent selected from the group methyl and trifluoromethyl, where such a substituent is bonded in the designated position 6 or 7,
and the salts, solvates and solvates of the salts thereof.

A particular embodiment of the present invention relates to compounds of the formula (I) in which
Het represents bicyclic heteroaryl of the formula

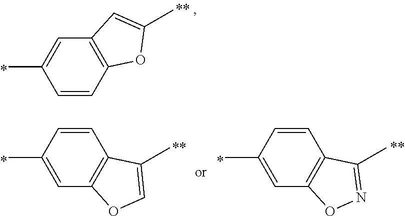

in which * labels the linkage to the R¹O group and ** labels the linkage to the carbonyl group,
   and the salts, solvates and solvates of the salts thereof.

A further particular embodiment of the present invention relates to compounds of the formula (I) in which
R¹ represents (C₄-C₇)-alkyl,
and the salts, solvates and solvates of the salts thereof.

A further particular embodiment of the present invention relates to compounds of the formula (I) in which
R¹ is a group of the formula

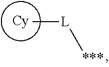

in which *** labels the linkage to the O atom,
L represents —CH₂— oder —CH₂—CH₂—
and
Cy represents (C₃-C₆)-cycloalkyl, tetrahydropyran-4-yl or phenyl,
   where phenyl can be mono- or disubstituted, identically or differently, with a radical selected from the group fluorine, chlorine, methyl, trifluoromethyl, methoxy and aminocarbonyl,
      and the salts, solvates and solvates of the salts thereof.

A further particular embodiment of the present invention relates to compounds of the formula (I) in which

R^{2A} and R^{2B} both represent hydrogen
and the salts, solvates and solvates of the salts thereof.

A further particular embodiment of the present invention relates to compounds of the formula (I) in which

R³ represents hydrogen or trifluoromethyl which is bonded in the designated position 6,
and the salts, solvates and solvates of the salts thereof.

In the context of the present invention, particular preference is given to compounds of the formula (I) in which
Het represents bicyclic heteroaryl of the formula

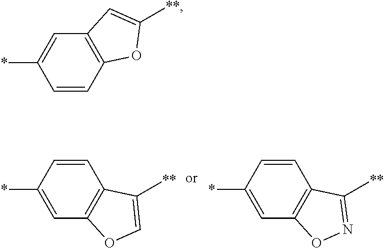

in which * labels the linkage to the R¹O group and ** labels the linkage to the carbonyl group,
R¹ represents (C₄-C₇)-alkyl or a group of the formula

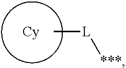

in which *** labels the linkage to the O atom,
L represents —CH₂— oder —CH₂—CH₂—
and
Cy represents (C₃-C₆)-cycloalkyl, tetrahydropyran-4-yl or phenyl,
   where phenyl can be mono- or disubstituted, identically or differently, with a radical selected from the group fluorine, chlorine, methyl, trifluoromethyl, methoxy and aminocarbonyl,
R^{2A} and R^{2B} both represent hydrogen
and
R³ represents hydrogen or trifluoromethyl which is bonded in the designated position 6,
   and the salts, solvates and solvates of the salts thereof.

Of particular significance in the context of the present invention are compounds of the formula (I-A*)

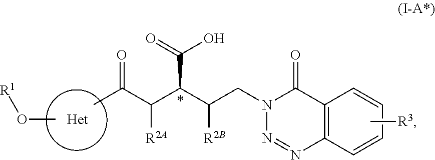

in which Het, R¹ and R³ have the meanings given above, R^{2A} and R^{2B} in each case represent hydrogen and the chiral carbon atom labelled with * in enantiomerically pure form has the depicted S configuration,
and the salts, solvates and solvates of the salts thereof.

The individual radical definitions specified in the respective combinations or preferred combinations of radicals are, independently of the respective combinations of the radicals specified, also replaced as desired by radical definitions of other combinations.

Very particular preference is given to combinations of two or more of the abovementioned preferred ranges.

The invention further provides a process for preparing compounds of the formula (I), in which R^{2A} and R^{2B} in each case represent hydrogen, which is characterized in that di-tert-butyl (2-hydroxyethyl)malonate of the formula (II)

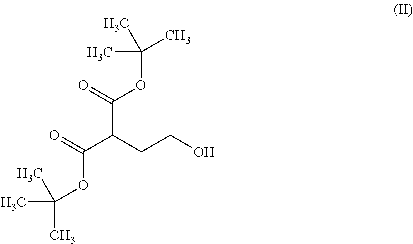 [SMILES: CC(C)(C)OC(=O)C(CCO)C(=O)OC(C)(C)C]

is reacted in the presence of a phosphine and an azodicarboxylate with a benzotriazin-4(3H)-one derivative of the formula (III)

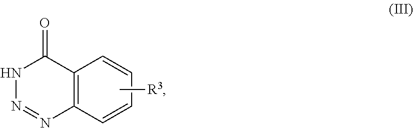

in which R³ has the meaning given above
to give a compound of the formula (IV)

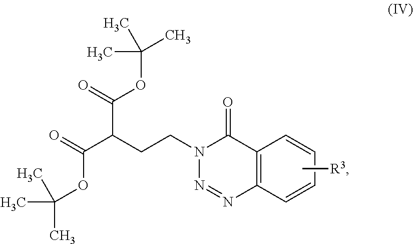

in which R³ has the meaning given above
and the compound of the formula (IV) is then either
[A] alkylated in the presence of a base with a compound of the formula (V)

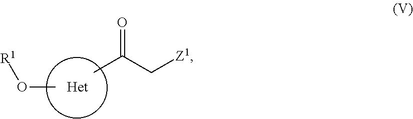

in which Het and R¹ have the meanings given above,
and
Z¹ represents a leaving group such as, for example, chlorine, bromine or iodine,
to give a compound of the formula (VI)

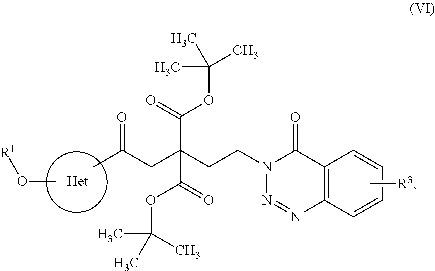

in which Het, R¹ and R³ have the meanings given above,
or
[B] alkylated firstly with a compound of the formula (VII)

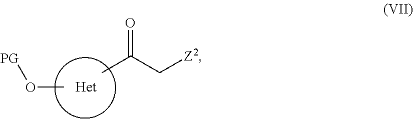

in which Het has the meaning given above,
PG represents a suitable temporary protective group such as, for example, benzyl
and
Z² represents a leaving group such as, for example, chlorine, bromine or iodine,
in the presence of a base to give a compound of the formula (VIII)

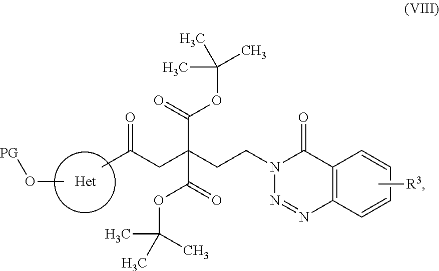

in which Het, PG and R³ have the meanings given above,
then the protective group PG is cleaved off and the resulting compound of the formula (IX)

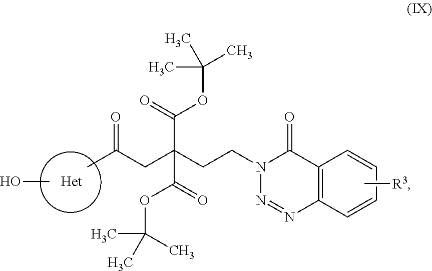

in which Het and R³ have the meanings given above,
is then in the presence of a base alkylated with a compound of the formula (X)

R¹—Z³ (X),
in which R¹ has the meaning given above
and
Z³ represents a leaving group such as, for example, chlorine, bromine, iodine, mesylate, triflate or tosylate,
to give the compound of the formula (VI)

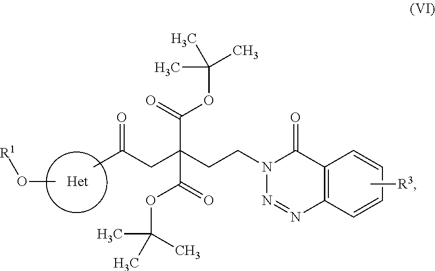

in which Het, R¹ and R³ have the meanings given above,
   and the compound of the formula (VI) thus obtained by method [A] or [B] is finally converted by treatment with an acid and subsequent heating to the carboxylic acid of the formula (I-A)

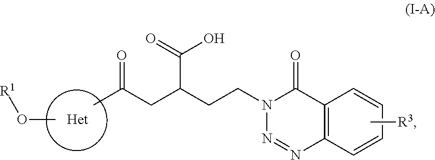

in which Het, R¹ and R³ have the meanings given above,
and the compounds of the formula (I-A) are optionally separated into their enantiomers and/or diastereomers and/or optionally reacted with the corresponding (i) solvents and/or (ii) bases to give solvates, salts and/or solvates of the salts thereof.

The reaction (II)+(III)→(IV) is carried out under the customary conditions of a “Mitsunobu reaction” in the presence of a phosphine and an azodicarboxylate [see e.g. D. L. Hughes, Org. Reactions 42, 335 (1992); D. L. Hughes, Org. Prep. Proced. Int. 28 (2), 127 (1996)]. Examples of suitable phosphine components are triphenylphosphine, tri-n-butylphosphine, 1,2-bis(diphenylphosphino)ethane (DPPE), diphenyl(2-pyridyl)phosphine, (4-dimethylaminophenyl)diphenylphosphine or tris(4-dimethylaminophenyl)phosphine. The azodicarboxylate used may, for example, be diethyl azodicarboxylate (DEAD), diisopropyl azodicarboxylate (DIAD), di-tert-butyl azodicarboxylate, N,N,N′N′-tetramethylazodicarboxamide (TMAD), 1,1′-(azodicarbonyl)dipiperidine (ADDP) or 4,7-dimethyl-3,5,7-hexahydro-1,2,4,7-tetrazocine-3,8-dione (DHTD). Here, preference is given to using triphenylphosphine in conjunction with diisopropylazodicarboxylate (DIAD).

Inert solvents for the reaction (II)+(III)→(IV) are, for example, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, or polar aprotic solvents such as acetonitrile, butyronitrile, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP). It is also possible to use mixtures of such solvents. Preference is given to using tetrahydrofuran.

The reaction (II)+(III)→(IV) generally takes place in a temperature range from −20° C. to +60° C., preferably at 0° C. to +40° C. Optionally, the use of a microwave apparatus may be advantageous for this reaction.

Especially suitable bases for the alkylation reactions (IV)+(V)→(VI) and (IV)+(VII)→(VIII) are alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amide such as lithium diisopropylamide or lithium bis(trimethylsilyl)amide, sodium bis(trimethylsilyl)amide or potassium bis(trimethylsilyl)amide, or standard organometallic bases such as phenyllithium or n-, sec- or tert-butyllithium. Preference is given to using sodium hydride or potassium tert-butoxide.

Suitable inert solvents for the process steps (IV)+(V)→(VI) and (IV)+(VII)→(VIII) are, for example, ethers such as diethyl ether, diisopropyl ether, methyl tert-butyl ether, tetrahydrofuran, 1,4-dioxane, 1,2-dimethoxyethane or bis(2-methoxyethyl) ether, hydrocarbons such as benzene, toluene, xylene, pentane, hexane or cyclohexane, or dipolar aprotic solvents such as acetonitrile, butyronitrile, N,N-dimethylformamide (DMF), N,N-dimethylacetamide (DMA), N,N′-dimethylpropyleneurea (DMPU), N-methylpyrrolidinone (NMP) or dimethyl sulphoxide (DMSO). It is also possible to use mixtures of such solvents. Preference is given to using N,N-dimethylformamide (DMF).

The reactions (IV)+(V)→(VI) and (IV)+(VII)→(VIII) are generally carried out in a temperature range from −70° C. to +100° C., preferably at −20° C. to +50° C.

The elimination of benzyl as temporary protective group PG in the process step (VIII)→(IX) is effected in a customary manner by hydrogenation with gaseous hydrogen or, in the case of a transfer hydrogenation, with the aid of a hydrogen donor such as ammonium formate, cyclohexene or cyclohexadiene, in each case in the presence of a suitable hydrogenation catalyst such as palladium on activated carbon in particular. The reaction is preferably carried out in an alcoholic solvent such as methanol or ethanol, in acetic acid, ethyl acetate, tetrahydrofuran or 1,4-dioxane or in a mixture of such solvents, optionally with the addition of water, in a temperature range from +20° C. to +80° C.

A temporary protective group PG in (VII) that can be used alternatively is also a silyl group, such as for example trimethylsilyl, triethylsilyl, triisopropylsilyl, tert-butyldimethylsilyl or tert-butyldiphenylsilyl. The elimination thereof in process step (VIII)→(IX) takes place in the customary manner with the help of a fluoride such as for example tetrabutylammonium fluoride (TBAF). A further alternative for PG is benzoyl; this protective group can be removed again by a known method by treatment with an alkali metal hydroxide, such as lithium, sodium or potassium hydroxide, or an alkali metal alkoxide, such as sodium or potassium methoxide or sodium or potassium ethoxide [see also T. W. Greene and P. G. M. Wuts, Protective Groups in Organic Synthesis, Wiley, New York, 1999].

Suitable bases for the alkylation reaction (IX)+(X)→(VI) are in particular alkali metal carbonates such as lithium carbonate, sodium carbonate, potassium carbonate or caesium carbonate, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, amide bases such as lithium diisopropylamide or lithium bis(trimethylsilyl)amide, sodium bis(trimethylsilyl)amide or potassium bis(trimethylsilyl)amide, or standard organometallic bases such as phenyllithium or n-, sec- or tert-butyllithium. Preference is given to using potassium carbonate or potassium tert-butoxide.

Inert solvents for this reaction are, for example, ethers such as diethyl ether, diisopropyl ether, methyl tert-butyl ether, tetrahydrofuran, 1,4-dioxane, 1,2-dimethoxyethane or bis(2-methoxyethyl) ether, alcohols such as methanol, ethanol, n-propanol, isopropanol, n-butanol, tert-butanol or n-pentanol, hydrocarbons such as benzene, toluene, xylene, pentane, hexane or cyclohexane, or polar-aprotic solvents such as acetone, methyl ethyl ketone, ethyl acetate, acetonitrile, butyronitrile, dimethyl sulphoxide (DMSO), N,N-dimethylformamide (DMF), N,N-dimethylacetamide (DMA), N,N′-dimethylpropyleneurea (DMPU) or N-methylpyrrolidinone (NMP). It is also possible to use mixtures of such solvents. Preference is given to using acetonitrile, N,N-dimethylformamide (DMF) or n-pentanol.

The reaction (IX)+(X)→(VI) is generally conducted, according to the reactivity of the components involved, within a temperature range from 0° C. to +120° C.

The ester cleavage and decarboxylation to the monocarboxylic acid in process step (VI)→(I-A) is achieved by treating the diester with a strong acid such as trifluoroacetic acid or hydrogen chloride and subsequent heating of the dicarboxylic acid that is formed as an intermediate. The reaction can be carried out in a “one-pot process”, without isolation of the intermediate, or in two stages. For the ester cleavage, in the case of the reaction with trifluoroacetic acid, preference is given to using dichloromethane, and in the case of the reaction with hydrogen chloride 1,4-dioxane, in each case under anhydrous conditions, as solvent. The ester cleavage is generally conducted within a temperature range from 0° C. to +30° C. The subsequent decarboxylation usually takes place in a temperature range from +80° C. to +150° C. in a correspondingly high-boiling inert solvent; preference being given to using 1,4-dioxane for this purpose. In some cases, the use of a microwave apparatus in this reaction may be advantageous.

The process steps described above can be carried out at atmospheric, elevated or reduced pressure (for example in the range from 0.5 to 5 bar); in general, the reactions are each carried out at atmospheric pressure.

The separation of stereoisomers (enantiomers and/or diastereomers) of the compounds of the formula (I-A) according to the invention can be achieved by customary methods familiar to those skilled in the art. Preference is given to employing chromatographic methods on achiral or chiral separation phases for this purpose. Alternatively, separation can also be effected via diastereomeric salts of the carboxylic acids of the formula (I-A) with chiral amine bases.

The benzotriazin-4(3H)-one derivatives of the formula (III) are accessible in a simple manner by treating 2-aminobenzamides of the formula (XI)

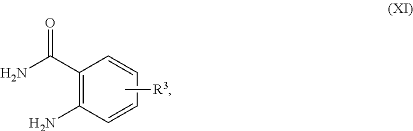

in which R³ has the meaning given above
with sodium nitrite in aqueous hydrochloric acid [see, for example, D. Fernandez-Forner et al., Tetrahedron 47 (42), 8917-8930 (1991)].

The compounds of the formulae (V) and (VII) can be obtained starting from compounds of the formula (XII) or (XIII)

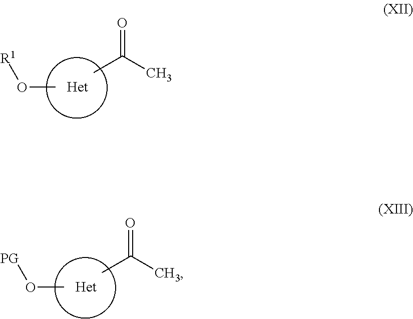

in which Het, PG and R¹ have the meanings given above,
by known methods for the α-halogenation of methyl ketones. As intermediates (V) and (VII), preference is given to using the α-bromo compounds (Z¹, Z²=Br), which are readily accessible from (XII) or (XIII) for example by treatment with phenyltrimethylammonium tribromide. The compounds of the formulae (XII) and (XIII) for their part can be prepared depending on the nature and the substitution pattern of the bicyclic heteroaryl group Het in question by various methods described in the literature [cf. e.g. as regards benzofuran derivatives: M. F. Wempe et al., J. Med. Chem. 54 (8), 2701-2713 (2011); J. Zhang et al., Org. Lett. 14 (17), 4528-4530 (2012); M. Inoue et al., Angew. Chem. Int. Ed. 39 (4), 761-764 (2000); M. Kuramoto et al., Org. Biomol. Chem. 6 (15), 2772-2781 (2008); W. Haefliger and D. Hauser, Synthesis, 3, 236-238 (1980); as regards benzothiophene derivatives: V. Fedi et al., J. Med Chem. 50 (20), 4793-4807 (2007); WO 2009/012430-A1, pp. 211-212; EP 2 351 743-A1, p. 127; as regards benzisoxazole derivatives: D. S. Kemp and K. G. Paul, J. Am. Chem. Soc. 97 (25), 7305-7312 (1975); C. V. Ramana et al., Eur. J. Org. Chem., 31, 5955-5966 (2010)]. Examples in this regard are also given in the reaction schemes 2a, 3a, 4a, 5a, 6a and 7a below.

Compounds of the formula (I) in which R^{2A} and R^{2B} are linked with one another and together form a —CH₂CH₂ bridge, i.e. compounds of the formula (I-B)

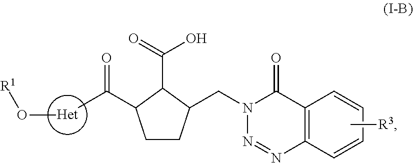

in which Het, R¹ and R³ have the meanings given above,
can be obtained following synthesis methods known in the literature, for example by coupling a lithium heteroaryl compound of the formula (XIV)

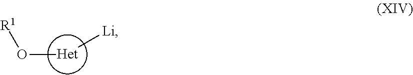

in which Het and R¹ have the meanings given above,
with a 2-acylcyclopentanecarboxylic acid derivative of the formula (XV)

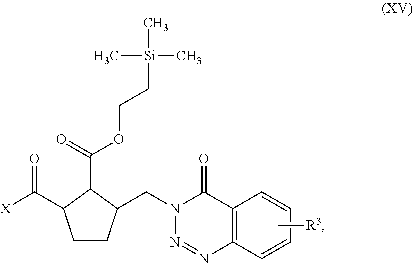

in which R³ has the meaning given above
and
X represents a suitable leaving group such as, for example, chlorine, 1H-imidazol-1-yl or methylsulphonyloxy (mesyloxy),
   to the product of the formula (XVI)

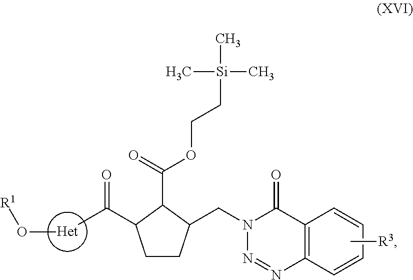

in which Het, R¹ and R³ have the meanings given above,
and subsequent elimination of the 2-(trimethylsilyl)ethyl ester group [as regards the coupling reaction cf. e.g. J. Rosen et al., Org. Lett. 9 (4), 667-669 (2007)]. Alternatively, it is also possible to use an aldehyde of the formula (XVII)

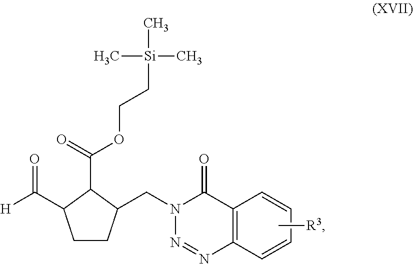

in which R³ has the meaning given above
as coupling partner, where the product of the formula (XVIII) produced here

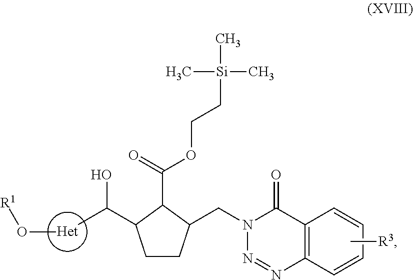

in which Het, R¹ and R³ have the meanings given above,
is then converted by subsequent oxidation to the above-described keto compound of the formula (XVI) [as regards the coupling reaction cf. e.g. C. K. Lau et al., J. Med. Chem. 35 (7), 1299-1318 (1992); P. Wipf et al., J. Am. Chem. Soc. 122 (39), 9391-9395 (2000)].

The elimination of the 2-(trimethylsilyl)ethyl ester moiety in the process step (XVI)→(I-B) is effected by standard methods either with the aid of a strong acid such as trifluoroacetic acid in particular in an inert solvent such as dichloromethane, or with the aid of a fluoride such as tetra-n-butylammonium fluoride in particular in an ethereal solvent such as tetrahydrofuran. The ester cleavage is generally conducted in a temperature range from −20° C. to +25° C.

The oxidation (XVIII)→(XVI) is preferably carried out with the help of 1,1,1-triacetoxy-1,1-dihydro-1,2-benziodoxol-3(1H)-one (“Dess-Martin periodinane”) in an inert solvent such as dichloromethane at a temperature of from 0° C. to +25° C. Alternative oxidizing agents that can be used for this reaction are for example also pyridinium chlorochromate (PCC) or the combination of dimethyl sulphoxide with oxalyl chloride or trifluoroacetanhydride in the presence of an amine base such as triethylamine or N,N-diisopropylethylamine (“Swern oxidation”).

The organolithium compounds of the formula (XIV) can be obtained by customary processes, for example by deprotonation of a compound of the formula (XIX)

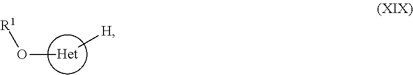

in which Het and R¹ have the meanings given above,
with a strong base, such as lithium diisopropylamide, lithium bis(trimethylsilyl)amide, phenyllithium or n-, sec- or tert-butyllithium, or by lithium-halogen exchange starting from a compound of the formula (XX)

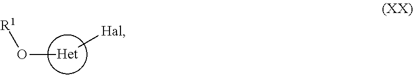

in which Het and R¹ have the meanings given above,
and
Hal represents halogen, such as chlorine, bromine or iodine.

The compounds of the formulae (XV) and (XVII) can be prepared in various ways in accordance with published synthesis processes [see e.g. the general preparative methods described in WO 96/15096-A1, pages 26-44, in particular the methods A, G, H and K]. Compounds of the formulae (XV) and (XVII) in particular that have a relative trans arrangement of the groups bonded to the central cyclopentane ring, i.e. compounds of the formulae (XV-A) and (XVII-A)

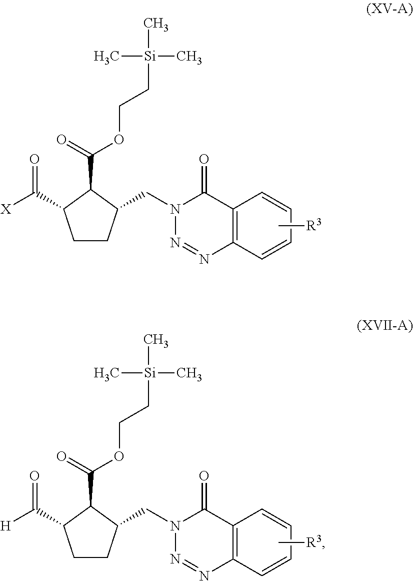

in which R³ and X have the meanings given above,
can be prepared for example by converting a) exo-2-(trimethylsilyl)ethyl 2-oxobicyclo[2.2.1]heptane-7-carboxylate of the formula (XXI)

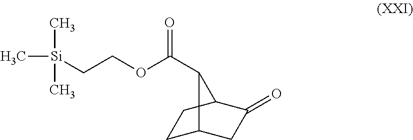 [SMILES: C[Si](C)(C)CCOC(=O)C1C2CCC1C(=O)C2]

to the enol-triflate of the formula (XXII)

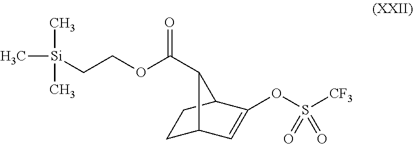 [SMILES: C[Si](C)(C)CCOC(=O)C1C2C=C(OS(=O)(=O)C(F)(F)F)C1CC2]

reacting this b) in a “one-pot process” firstly with N-methylmorpholine N-oxide in the presence of catalytic osmium tetroxide and subsequently with sodium periodate to give the 3-formylcyclopentane-1,2-dicarboxylic acid derivative of the formula (XXIII)

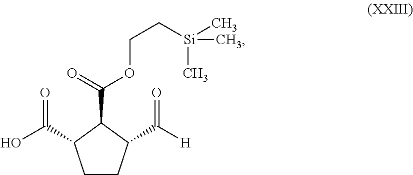 [SMILES: C[Si](C)(C)CCOC(=O)[C@H]1[C@H](C=O)CC[C@@H]1C(=O)O]

then c) converting with benzyl chloroformate in the presence of a base and 4-N,N-dimethylaminopyridine as catalyst to the corresponding benzyl ester of the formula (XXIV) CH₃

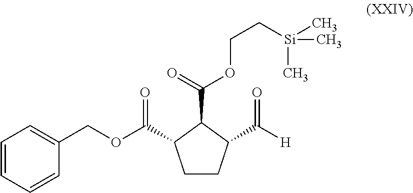 [SMILES: C[Si](C)(C)CCOC(=O)[C@H]1[C@H](C=O)CC[C@@H]1C(=O)OCc1ccccc1]

then d) reducing with sodium borohydride to the hydroxymethyl compound of the formula (XXV)

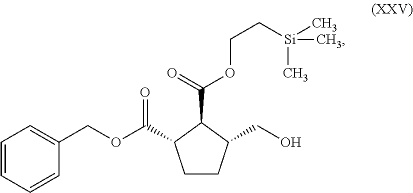 [SMILES: C[Si](C)(C)CCOC(=O)[C@H]1[C@H](CO)CC[C@@H]1C(=O)OCc1ccccc1]

then e) reacting in the presence of an alkyl- or arylphosphine and an azodicarboxylate with a benzotriazin-4(3H)-one derivative of the formula (III)

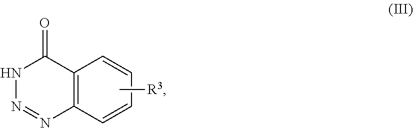

in which R³ has the meaning given above
to give a compound of the formula (XXVI)

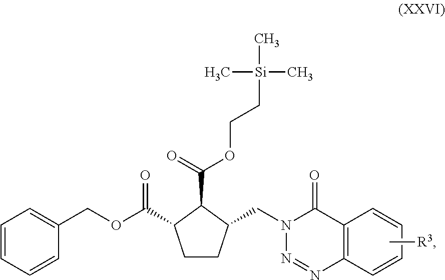

in which R³ has the meaning given above,
f) cleaving off the benzyl ester by hydrogenolysis in the presence of a palladium catalyst and finally
g1) converting the resulting carboxylic acid of the formula (XXVII)

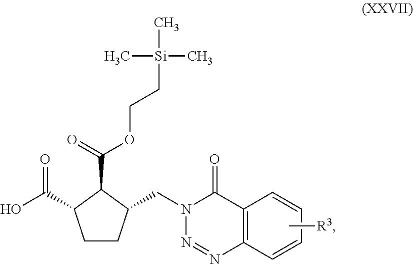

in which R³ has the meaning given above
by customary methods to the desired derivative of the formula (XV-A) or g2) reducing with borane or a borane complex to the hydroxymethyl compound of the formula (XXVIII)

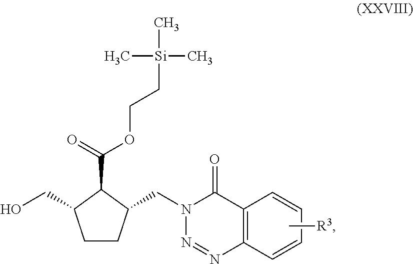

in which R³ has the meaning given above
and h) re-oxidizing this with 1,1,1-triacetoxy-1,1-dihydro-1,2-benziodoxol-3(1H)-one (“Dess-Martin periodinane”) to the formyl compound of the formula (XVII-A) [for further details on this reaction sequence, reference may be made to the corresponding procedures in the experimental section of the present application].

In the above-described synthesis sequence (XXI)→(XXII)→(XXIII)→(XXIV)→(XXV)→(XXVI)→(XXVII)→(XV-A) or (XXVII)→(XXVIII)→(XVII-A), for further simplified representation of the relative configuration of the chiral centres, only the structural formula of each enantiomer has been given, even though the compounds in question have been used or obtained in racemic form; the actual end product of a preparation process conducted in racemic form in such a way is the racemic mixture of the compounds (XV-A) and (XV-B) or (XVII-A) and (XVII-B).

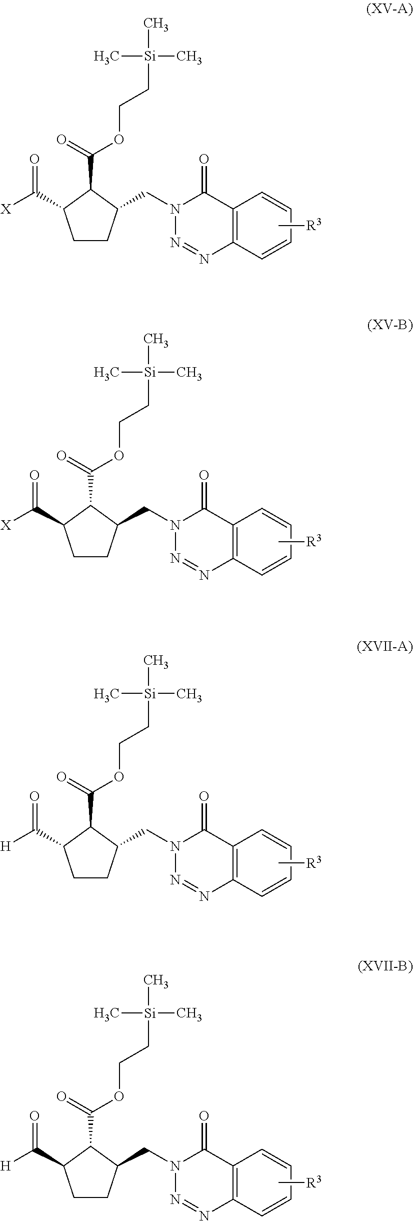

Accordingly, in such a procedure, the end product obtained is a racemic mixture (based on the configuration of the central cyclopentane ring) of the carboxylic acids (I-B1) and (I-B2) according to the invention

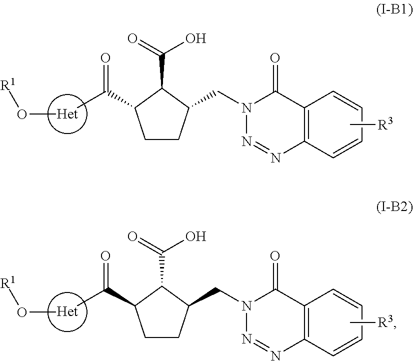

in which Het, R¹ and R³ have the meanings given above. The separation of such mixtures of stereoisomers (enantiomers and/or diastereomers) of the compounds of the formula (I-B) according to the invention can be achieved by customary methods familiar to those skilled in the art.

Preference is given to employing chromatographic methods on achiral or chiral separation phases for this purpose. In the case of the carboxylic acids (I-B1)/(I-B2) for example, separation is alternatively also possible via diastereomeric salts using chiral amine bases.

The compounds of the formulae (II), (X), (XI), (XIX), (XX) and (XXI) are either commercially available or described as such in the literature, or they can be prepared starting from other commercially available compounds by literature methods familiar to those skilled in the art.

Numerous detailed procedures and further literature references can also be found in the experimental section, in the section on the preparation of the starting compounds and intermediates.

The preparation of the compounds according to the invention can be illustrated by way of example by the following reaction schemes:

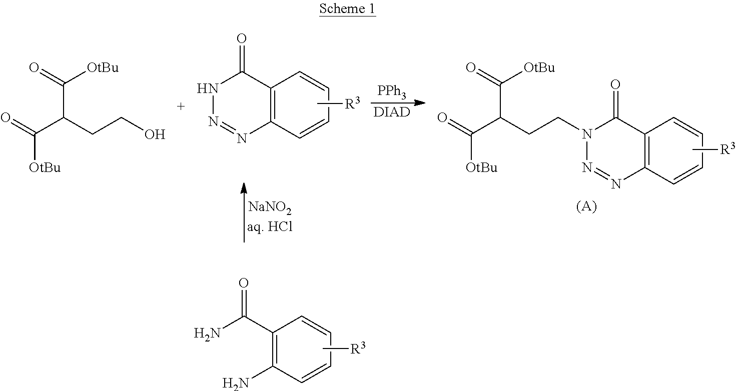

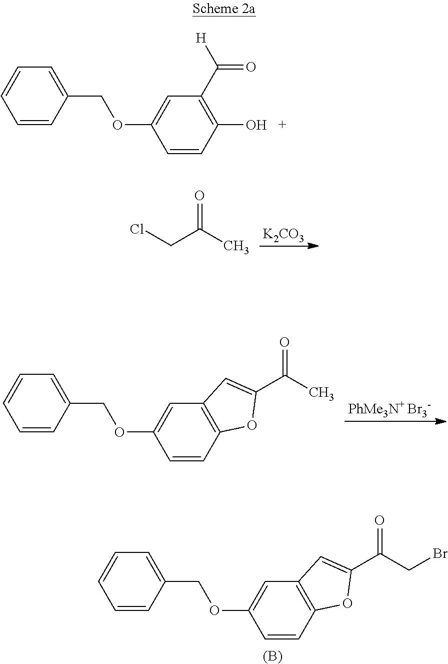

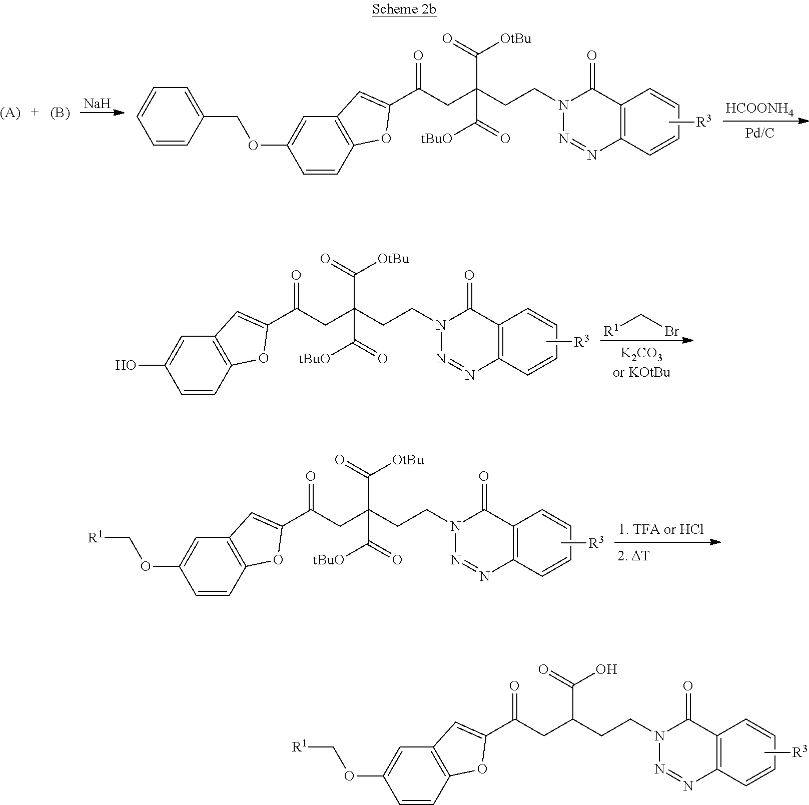

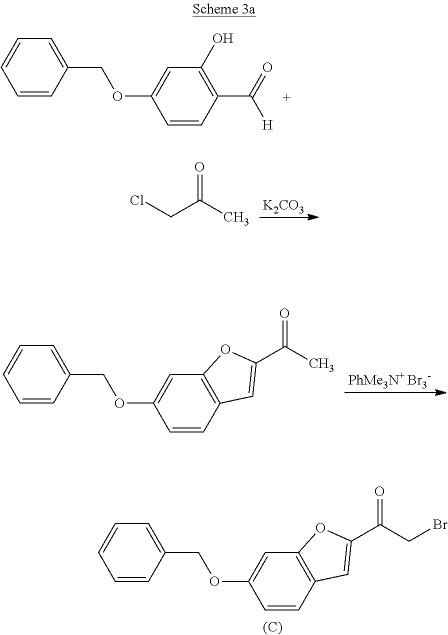

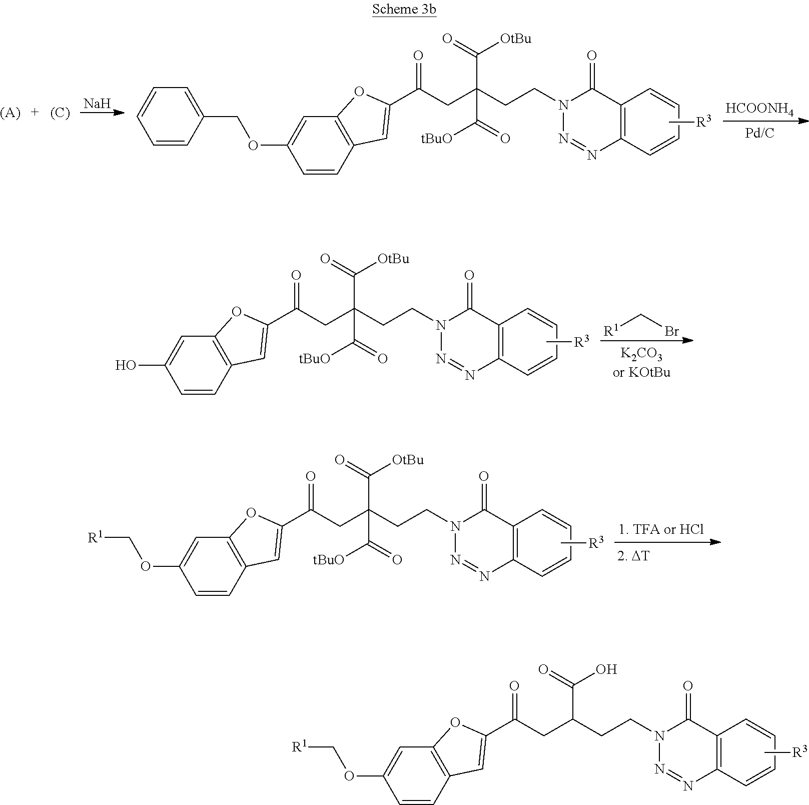

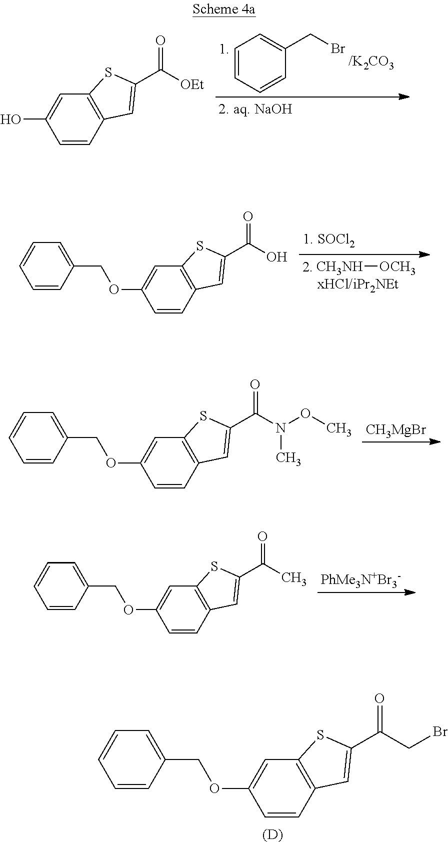

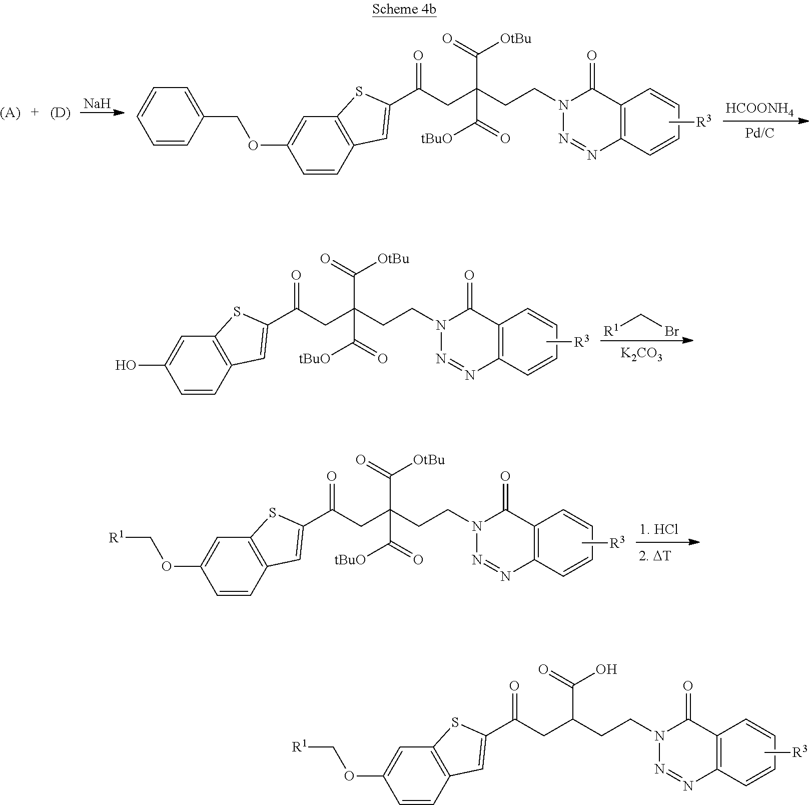

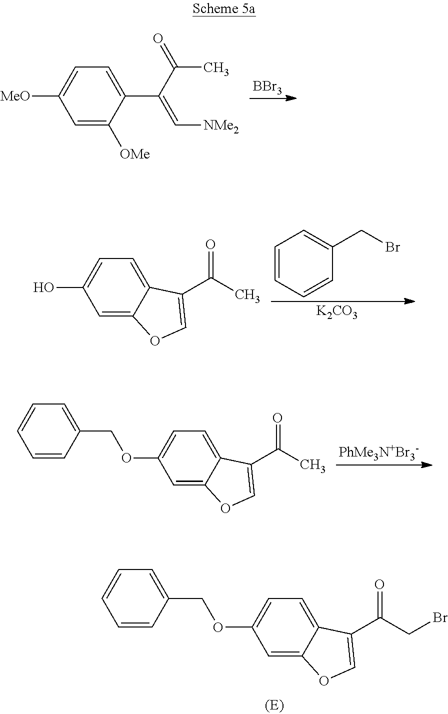

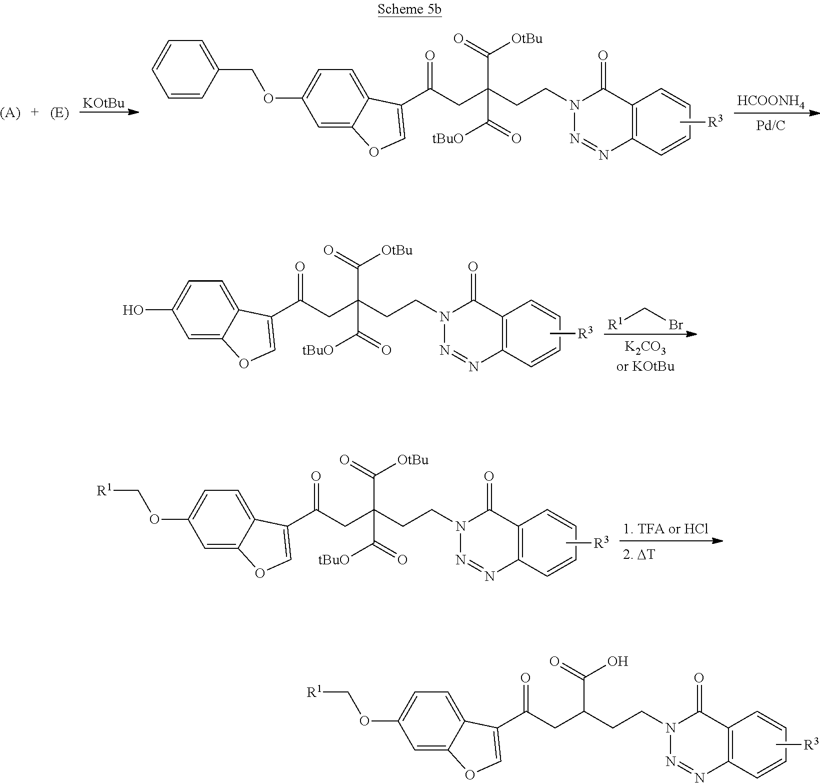

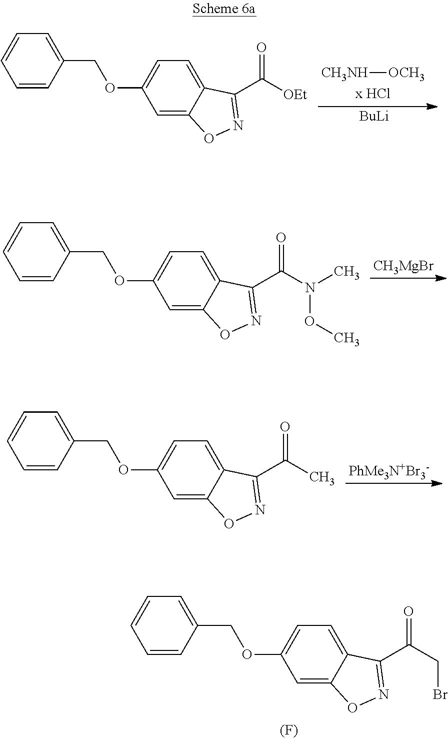

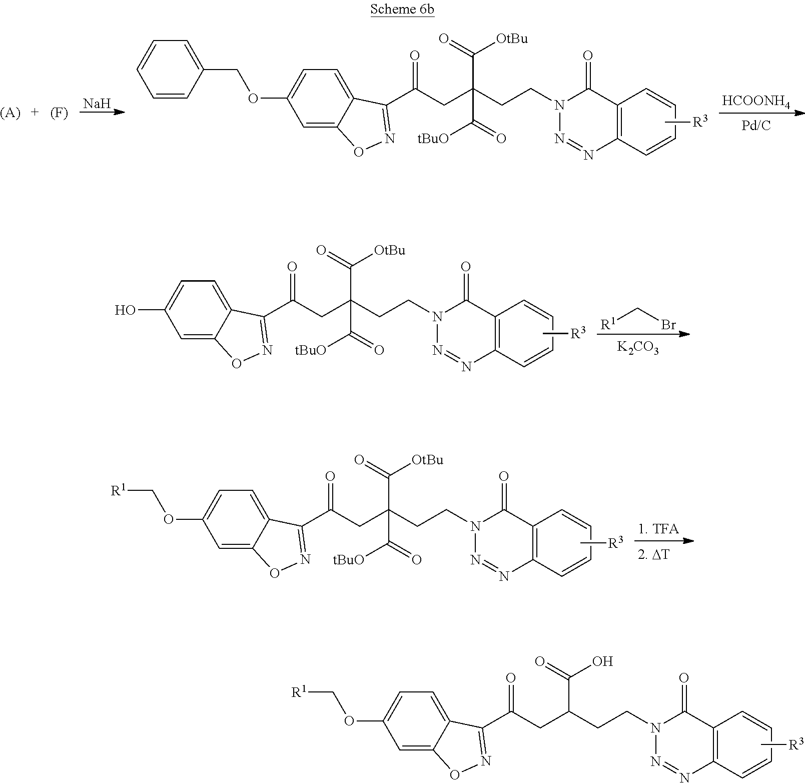

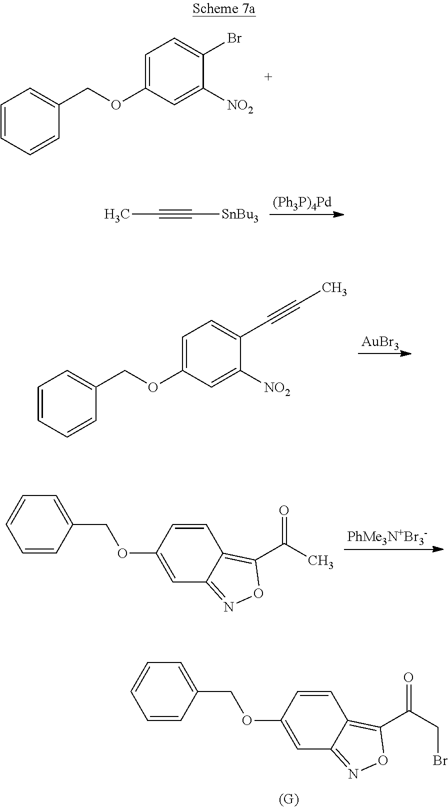

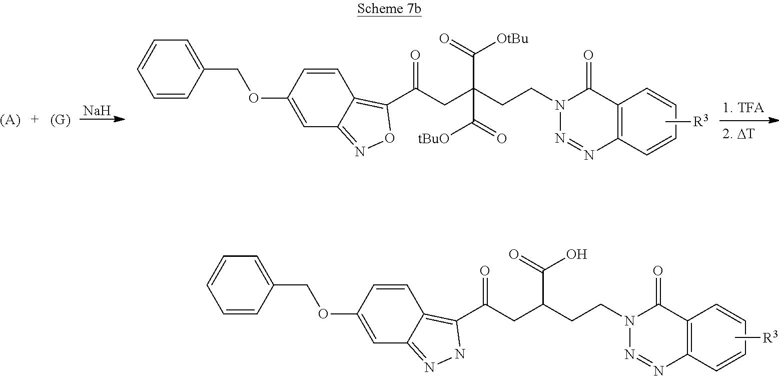

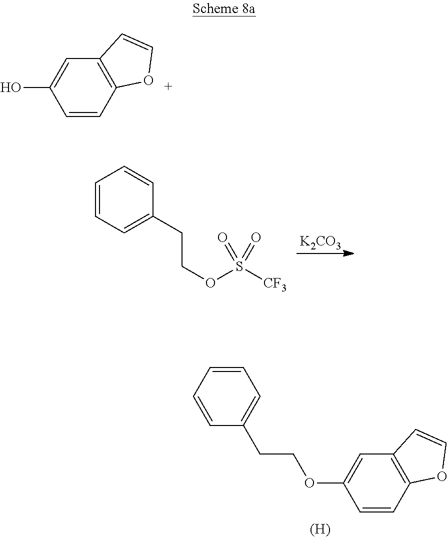

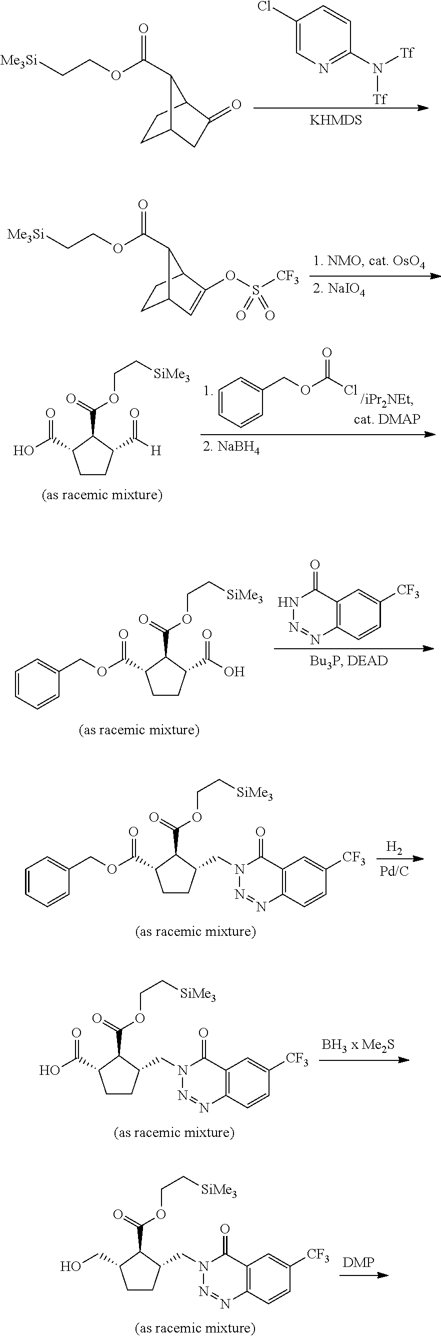

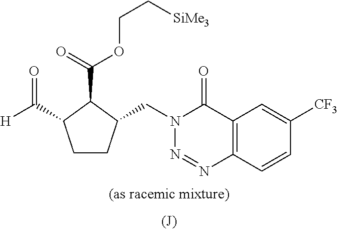 [SMILES: C[Si](C)(C)CCOC(=O)[C@H]1[C@H](Cn2nnc3ccc(C(F)(F)F)cc3c2=O)CC[C@@H]1C=O]

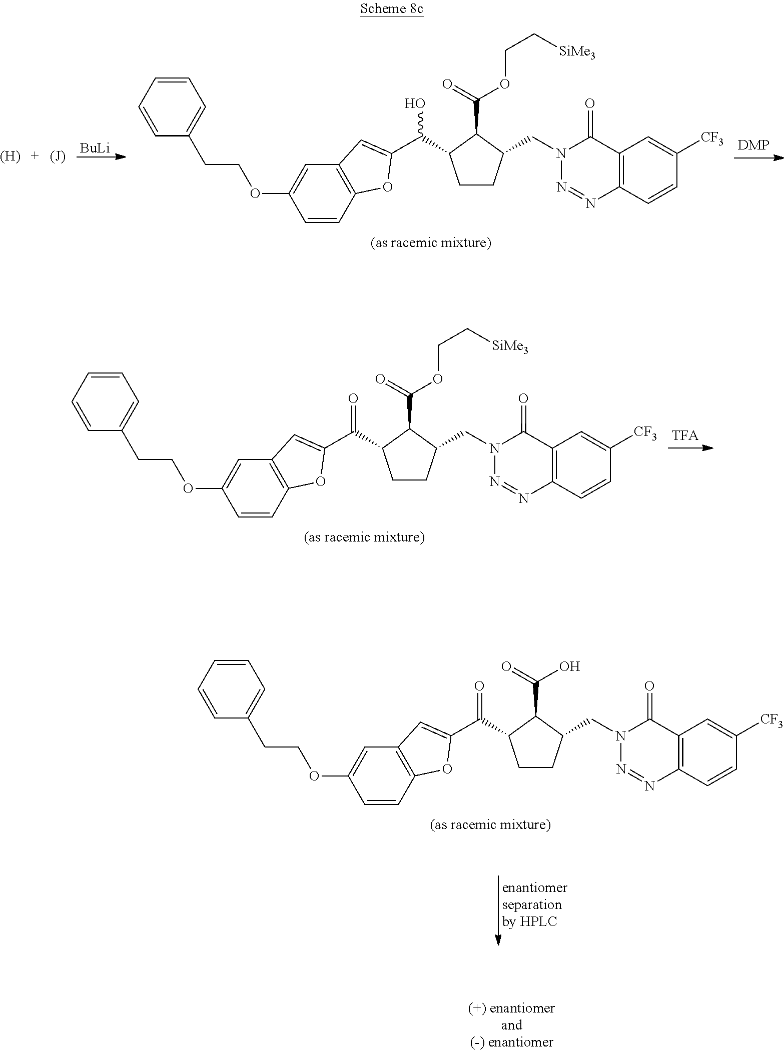

The compounds according to the invention have valuable pharmacological properties and can be used for the prevention and treatment of diseases in humans and animals.

The compounds according to the invention are potent, non-reactive and selective inhibitors of human macrophage elastase (HME/hMMP-12) having a significantly improved profile with respect to potency and selectivity compared to the compounds known from the prior art. This more targeted mode of action suggests a lower dosability of the compounds according to the invention and a reduced risk of the occurrence of undesired side effects during therapy.

The compounds according to the invention are therefore suitable to a particular degree for the treatment and/or prevention of diseases and pathological processes, in particular those in which macrophage elastase (HME/hMMP-12) is involved in the course of an infectious or noninfectious inflammatory event and/or tissue or vascular remodelling.

In the context of the present invention, these especially include disorders of the respiratory pathway and the lung, such as chronic obstructive pulmonary disease (COPD), asthma and the group of interstitial lung diseases (ILDs), and disorders of the cardiovascular system such as arteriosclerosis and aneurysms.

The forms of chronic obstructive lung disease (COPD) especially include pulmonary emphysema, for example the pulmonary emphysema induced by cigarette smoke, chronic bronchitis (CB), pulmonary hypertension in COPD (PH-COPD), bronchiectasis (BE) and combinations thereof, especially in acute exacerbating stages of the disease (AE-COPD).

The forms of asthma include asthmatic disorders of different severity with intermittent or persistent character, such as refractory asthma, bronchial asthma, allergic asthma, intrinsic asthma, extrinsic asthma and medicament- or dust-induced asthma.

The group of interstitial lung diseases (ILDs) includes idiopathic pulmonary fibrosis (IPF), pulmonary sarcoidosis and acute interstitial pneumonia, non-specific interstitial pneumonia, lymphoid interstitial pneumonia, respiratory bronchiolitis with interstitial pulmonary disorder, cryptogenic organizing pneumonia, desquamative interstitial pneumonia and non-classifiable idiopathic interstitial pneumonia, and also granulomatous interstitial pulmonary disorders, interstitial pulmonary disorders of known cause and other interstitial pulmonary disorders of unknown cause.

The compounds according to the invention can also be used for the treatment and/or prevention of further disorders of the respiratory pathways and of the lung, for example of pulmonary arterial hypertension (PAH) and other forms of pulmonary hypertension (PH), of bronchiolitis obliterans syndrome (BOS), of acute respiratory distress syndrome (ARDS), of acute lung damage (ALI), alpha-1 antitrypsin deficiency (AATD) and cystic fibrosis (CF), of various forms of bronchitis (chronic bronchitis, infectious bronchitis, eosinophilic bronchitis), of bronchiectasis, pneumonia, farmer's lung and related diseases, cough- and cold-type diseases having infectious and non-infectious causes (chronic inflammatory cough, iatrogenic cough), mucous membrane inflammation (including medicamentous rhinitis, vasomotor rhinitis and seasonally dependent allergic rhinitis, for example hay fever), and polyps.

In the context of the present invention, the group of diseases of the cardiovascular system especially includes arteriosclerosis and its sequelae, for example stroke in the case of arteriosclerosis of the neck arteries (carotid arteriosclerosis), myocardial infarction in the case of arteriosclerosis of the coronary artery, peripheral arterial occlusive disease (pAOD) as a consequence of arteriosclerosis of arteries of the legs, and also aneurysms, especially aneurysms of the aorta, for example as a consequence of arteriosclerosis, high blood pressure, injuries and inflammations, infections (for example in the case of rheumatic fever, syphilis, Lyme borreliosis), inherited connective tissue weaknesses (for example in the case of Marfan syndrome and Ehlers-Danlos syndrome) or as a consequence of a volume load on the aorta in the case of inherited heart defects with right-left shunt or shunt-dependent perfusion of the lungs, and also aneurysms at coronary arteries in the course of suffering from Kawasaki syndrome and in areas of the brain in patients with a congenital malformation of the aortic valve.

In addition, the compounds according to the invention can be used for the treatment and/or prevention of further cardiovascular disorders, for example high blood pressure (hypertension), heart failure, coronary heart disease, stable and unstable angina pectoris, renal hypertension, peripheral and cardiac vascular disorders, arrhythmias, atrial and ventricular arrhythmias and impaired conduction, for example atrioventricular blocks of degrees I-III, supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, acute coronary syndrome (ACS), autoimmune cardiac disorders (pericarditis, endocarditis, valvolitis, aortitis, cardiomyopathies), boxer cardiomyopathy, shock such as cardiogenic shock, septic shock and anaphylactic shock, and also for the treatment and/or prevention of thromboembolic disorders and ischaemias such as myocardial ischaemia, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, microalbuminuria, myocardial insufficiency, endothelial dysfunction, micro- and macrovascular damage (vasculitis), and also to prevent restenoses, for example after thrombolysis therapies, percutaneous transluminal angioplasties (PTA), percutaneous transluminal coronary angioplasties (PTCA), heart transplants and bypass operations.

In the context of the present invention, the term “heart failure” encompasses both acute and chronic forms of heart failure, and also specific or related disease types thereof, such as acute decompensated heart failure, right heart failure, left heart failure, global failure, ischaemic cardiomyopathy, dilatative cardiomyopathy, hypertrophic cardiomyopathy, idiopathic cardiomyopathy, congenital heart defects, heart valve defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders and diastolic and systolic heart failure.

The compounds according to the invention are also suitable for the treatment and/or prevention of renal disorders, in particular renal insufficiency and kidney failure. In the context of the present invention, the terms “renal insufficiency” and “kidney failure” encompass both acute and chronic manifestations thereof and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and Alport's syndrome, immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or need for dialysis. The present invention also encompasses the use of the compounds according to the invention for the treatment and/or prevention of sequelae of renal insufficiency, for example hypertension, pulmonary oedema, heart failure, uraemia, anaemia, electrolyte disturbances (for example hyperkalaemia, hyponatraemia) and disturbances in bone and carbohydrate metabolism.

In addition, the compounds according to the invention are suitable for the treatment and/or prevention of disorders of the urogenital system, for example benign prostate syndrome (BPS), benign prostate hyperplasia (BPH), benign prostate enlargement (BPE), bladder outlet obstruction (BOO), lower urinary tract syndromes (LUTS), neurogenic overactive bladder (OAB), incontinence, for example mixed urinary incontinence, urge urinary incontinence, stress urinary incontinence or overflow urinary incontinence (MUI, UUI, SUI, OUI), pelvic pain, and also erectile dysfunction and female sexual dysfunction.

In addition, the compounds according to the invention have antiinflammatory action and can therefore be used as antiinflammatory agents for the treatment and/or prevention of sepsis (SIRS), multiple organ failure (MODS, MOF), inflammatory disorders of the kidney, chronic intestinal inflammations (IBD, Crohn's disease, ulcerative colitis), pancreatitis, peritonitis, cystitis, urethritis, prostatitis, epidimytitis, oophoritis, salpingitis, vulvovaginitis, rheumatoid disorders, inflammatory disorders of the central nervous system, multiple sclerosis, inflammatory skin disorders and inflammatory eye disorders.

Furthermore, the compounds according to the invention are suitable for the treatment and/or prevention of fibrotic disorders of the internal organs, for example the lung, the heart, the kidney, the bone marrow and in particular the liver, and also dermatological fibroses and fibrotic eye disorders. In the context of the present invention, the term “fibrotic disorders” includes in particular disorders such as hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, peritoneal fibrosis and similar fibrotic disorders, scleroderma, morphoea, keloids, hypertrophic scarring, naevi, diabetic retinopathy, proliferative vitroretinopathy and disorders of the connective tissue (for example sarcoidosis). The compounds according to the invention can likewise be used for promoting wound healing, for controlling postoperative scarring, for example following glaucoma operations and cosmetically for ageing or keratinized skin.

The compounds according to the invention can also be used for the treatment and/or prevention of anaemias such as haemolytic anaemias, in particular haemoglobinopathies such as sickle cell anaemia and thalassaemias, megaloblastic anaemias, iron deficiency anaemias, anaemias owing to acute blood loss, displacement anaemias and aplastic anaemias.

Moreover, the compounds according to the invention are suitable for the treatment of cancers, for example skin cancer, brain tumours, breast cancer, bone marrow tumours, leukaemias, liposarcomas, carcinomas of the gastrointestinal tract, of the liver, the pancreas, the lung, the kidney, the ureter, the prostate and the genital tract and also of malignant tumours of the lymphoproliferative system, for example Hodgkin's and non-Hodgkin's lymphoma.

In addition, the compounds according to the invention can be used for the treatment and/or prevention of impaired lipid metabolism and dyslipidaemias (hypolipoproteinaemia, hypertriglyceridaemias, hyperlipidaemia, combined hyperlipidaemias, hypercholesterolaemia, abetalipoproteinaemia, sitosterolaemia), xanthomatosis, Tangier disease, adiposity, obesity, metabolic disorders (metabolic syndrome, hyperglycaemia, insulin-dependent diabetes, non-insulin-dependent diabetes, gestational diabetes, hyperinsulinaemia, insulin resistance, glucose intolerance and diabetic sequelae, such as retinopathy, nephropathy and neuropathy), of disorders of the gastrointestinal tract and the abdomen (glossitis, gingivitis, periodontitis, oesophagitis, eosinophilic gastroenteritis, mastocytosis, Crohn's disease, colitis, proctitis, anus pruritis, diarrhoea, coeliac disease, hepatitis, hepatic fibrosis, cirrhosis of the liver, pancreatitis and cholecystitis), of disorders of the central nervous system and neurodegenerative disorders (stroke, Alzheimer's disease, Parkinson's disease, dementia, epilepsy, depressions, multiple sclerosis), immune disorders, thyroid disorders (hyperthyreosis), skin disorders (psoriasis, acne, eczema, neurodermitis, various forms of dermatitis, for example dermatitis abacribus, actinic dermatitis, allergic dermatitis, ammonia dermatitis, facticial dermatitis, autogenic dermatitis, atopic dermatitis, dermatitis calorica, dermatitis combustionis, dermatitis congelationis, dermatitis cosmetica, dermatitis escharotica, exfoliative dermatitis, dermatitis gangraenose, stasis dermatitis, dermatitis herpetiformis, lichenoid dermatitis, dermatitis linearis, dermatitis maligna, medicinal eruption dermatitis, dermatitis palmaris and plantaris, parasitic dermatitis, photoallergic contact dermatitis, phototoxic dermatitis, dermatitis pustularis, seborrhoeic dermatitis, sunburn, toxic dermatitis, Meleney's ulcer, dermatitis veneata, infectious dermatitis, pyogenic dermatitis and rosacea-like dermatitis, and also keratitis, bullosis, vasculitis, cellulitis, panniculitis, lupus erythematosus, erythema, lymphomas, skin cancer, Sweet syndrome, Weber-Christian syndrome, scar formation, wart formation, chilblains), of inflammatory eye diseases (saccoidosis, blepharitis, conjunctivitis, iritis, uveitis, chorioiditis, ophthalmitis), viral diseases (caused by influenza, adeno and corona viruses, for example HPV, HCMV, HIV, SARS), of disorders of the skeletal bone and the joints and also the skeletal muscle (multifarious forms of arthritis, for example arthritis alcaptonurica, arthritis ankylosans, arthritis dysenterica, arthritis exsudativa, arthritis fungosa, arthritis gonorrhoica, arthritis mutilans, arthritis psoriatica, arthritis purulenta, arthritis rheumatica, arthritis serosa, arthritis syphilitica, arthritis tuberculosa, arthritis urica, arthritis villonodularis pigmentosa, atypical arthritis, haemophilic arthritis, juvenile chronic arthritis, rheumatoid arthritis and metastatic arthritis, furthermore Still syndrome, Felty syndrome, Sjörgen syndrome, Clutton syndrome, Poncet syndrome, Pott syndrome and Reiter syndrome, multifarious forms of arthropathies, for example arthropathia deformans, arthropathia neuropathica, arthropathia ovaripriva, arthropathia psoriatica and arthropathia tabica, systemic scleroses, multifarious forms of inflammatory myopathies, for example myopathie epidemica, myopathie fibrosa, myopathie myoglobinurica, myopathie ossificans, myopathie ossificans neurotica, myopathie ossificans progressiva multiplex, myopathie purulenta, myopathie rheumatica, myopathie trichinosa, myopathie tropica and myopathie typhosa, and also the Giinther syndrome and the Miinchmeyer syndrome), of inflammatory changes of the arteries (multifarious forms of arteritis, for example endarteritis, mesarteritis, periarteritis, panarteritis, arteritis rheumatica, arteritis deformans, arteritis temporalis, arteritis cranialis, arteritis gigantocellularis and arteritis granulomatosa, and also Horton syndrome, Churg-Strauss syndrome and Takayasu arteritis), of Muckle-Well syndrome, of Kikuchi disease, of polychondritis, dermatosclerosis and also other disorders having an inflammatory or immunological component, for example cataract, cachexia, osteoporosis, gout, incontinence, lepra, Sezary syndrome and paraneoplastic syndrome, for rejection reactions after organ transplants and for wound healing and angiogenesis in particular in the case of chronic wounds.

On account of their profile of properties, the compounds according to the invention are especially suitable for the treatment and/or prevention of diseases of the respiratory tract and of the lung, primarily chronic obstructive pulmonary disorder (COPD), here in particular lung emphysema, chronic bronchitis (CB), pulmonary hypertension in COPD (PH-COPD) and bronchiectasis (BE), and also of combinations of these types of illnesses, particularly in acutely exacerbating stages of COPD disease (AE COPD), furthermore of asthma and of interstitial lung diseases, here in particular idiopathic pulmonary fibrosis (IPF) and pulmonary sarcoidosis, of diseases of the cardiovascular system, in particular of arteriosclerosis, specifically of carotid arteriosclerosis, and also viral myocarditis, cardiomyopathy and aneurysms, including their sequelae such as stroke, myocardial infarction and peripheral arterial occlusive disease (pAVK), and also of chronic kidney diseases and Alport's syndrome.

The aforementioned well-characterized diseases in humans can also occur with comparable aetiology in other mammals and can likewise be treated therein with the compounds of the present invention.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The present invention further provides for the use of the compounds according to the invention for the treatment and/or prevention of disorders, especially of the aforementioned disorders.

The present invention further provides for the use of the compounds according to the invention for production of a medicament for the treatment and/or prevention of disorders, especially of the aforementioned disorders.

The present invention further provides a medicament comprising at least one of the compounds according to the invention for the treatment and/or prevention of disorders, especially of the aforementioned disorders.

The present invention further provides for the use of the compounds according to the invention in a method for the treatment and/or prevention of disorders, especially of the aforementioned disorders.

The present invention further provides a process for the treatment and/or prevention of disorders, especially of the aforementioned disorders, using an effective amount of at least one of the compounds according to the invention.

The compounds according to the invention can be used alone or, if required, in combination with one or more other pharmacologically active substances, provided that this combination does not lead to undesirable and unacceptable side effects. The present invention therefore further provides medicaments comprising at least one of the compounds according to the invention and one or more further active ingredients, especially for the treatment and/or prevention of the aforementioned disorders. Preferred examples of combination active ingredients suitable for this purpose include:
anti-obstructive/bronchodilatory agents as used, for example, for the the treatment of chronic obstructive pulmonary disease (COPD) or bronchial asthma, by way of example and with preference from the group of the inhalatively or systemically administered agonists of the beta-adrenergic receptor (beta-mimetics), the inhalatively administered anti-muscarinergic substances and the PDE 4 inhibitors;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 4 inhibitors such as roflumilast and PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat and the compounds described in WO 00/06568, WO 00/06569, WO 02/42301, WO 03/095451, WO 2011/147809, WO 2012/004258, WO 2012/028647 and WO 2012/059549;
compounds which inhibit human neutrophil elastase (HNE), such as in particular sivelestat, DX-890 (Reltran) and the compounds described in WO 2004/020410, WO 2004/020412, WO 2004/024700, WO 2004/024701, WO 2005/080372, WO 2005/082863, WO 2005/082864, WO 2009/080199, WO 2009/135599, WO 2010/078953 and WO 2010/115548;
prostacyclin analogues and IP receptor agonists, by way of example and with preference iloprost, beraprost, treprostinil, epoprostenol or NS-304;
edothelin receptor antagonists, by way of example and with preference bosentan, darusentan, ambrisentan or sitaxsentan;
antiinflammatory, immunomodulating, immunosuppressive and/or cytotoxic agents, by way of example and with preference from the group of the systemically or inhalatively administered corticosteroids and also acetylcysteine, montelukast, azathioprine, cyclophosphamide, hydroxycarbamide, azithromycin, IFN-γ, pirfenidone or etanercept;
antifibrotic agents, by way of example and with preference lysophosphatidic acid receptor 1 (LPA-1) antagonists, lysyl oxidase (LOX) inhibitors, lysyl oxidase-like-2 inhibitors, vasoactive intestinal peptide (VIP), VIP analogues, α_{vβ6}-integrin antagonists, cholchicine, IFN-3, D-penicillamine, inhibitors of the WNT signal path or CCR2 antagonists;
active ingredients which alter lipid metabolism, by way of example and with preference from the group of thyroid receptor agonists, cholesterol synthesis inhibitors, preferred examples being HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists;
hypotensive active ingredients, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
compounds which block the binding of serotonin to its receptor, by way of example and with preference antagonists of the 5-HT_{2B} receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which inhibit soluble epoxide hydrolase (sEH), for example N,N′-dicyclohexylurea, 12-(3-adamantan-1-ylureido)dodecanoic acid or 1-adamantan-1-yl-3-{5-[2-(2-ethoxyethoxy)ethoxy]pentyl}urea;
compounds which influence the energy metabolism of the heart, by way of example and with preference etomoxir, dichloroacetate, ranolazine or trimetazidine;
antithrombotic agents, by way of example and with preference from the group of platelet aggregation inhibitors, the anticoagulants and the profibrinolytic substances;
chemotherapeutics as used, for example, for the treatment of neoplasms in the lung or other organs; and/or
antibiotics, especially from the group of the fluoroquinolonecarboxylic acids, by way of example and with preference ciprofloxacin or moxifloxacin.

In a preferred embodiment of the invention, the compounds according to the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the compounds according to the invention are administered in combination with an antimuscarinergic substance, by way of example and with preference ipratropium bromide, tiotropium bromide or oxitropium bromide.

In a preferred embodiment of the invention, the compounds according to the invention are administered in combination with a corticosteroid, by way of example and with preference prednisone, prednisolone, methylprednisolone, triamcinolone, dexamethasone, beclomethasone, betamethasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the compounds according to the invention are administered in combination with a platelet aggregation inhibitor, by way of example and with preference aspirin, clopidogrel, ticlopidine or dipyridamole.

In a preferred embodiment of the invention, the compounds according to the invention are administered in combination with a thrombin inhibitor, by way of example and with preference ximelagatran, melagatran, dabigatran, bivalirudin or clexane.

In a preferred embodiment of the invention, the compounds according to the invention are administered in combination with a GPIIb/IIIa antagonist, by way of example and with preference tirofiban or abciximab.

In a preferred embodiment of the invention, the compounds according to the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embursatan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP-600 or SPP-800.

In a preferred embodiment of the invention, the compounds according to the invention are administered in combination with a mineralocorticoid receptor antagonist, by way of example and with preference spironolactone, eplerenone or finerenone.

In a preferred embodiment of the invention, the compounds according to the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment of the invention, the compounds according to the invention are administered in combination with a CETP inhibitor, by way of example and with preference torcetrapib (CP-529 414), JJT-705 or CETP vaccine (Avant).

In a preferred embodiment of the invention, the compounds according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS-188494 or TAK-475.

In a preferred embodiment of the invention, the compounds according to the invention are administered in combination with an ACAT inhibitor, by way of example and with preference avasimibe, melinamide, pactimibe, eflucimibe or SMP-797.

In a preferred embodiment of the invention, the compounds according to the invention are administered in combination with an MTP inhibitor, by way of example and with preference implitapide, BMS-201038, R-103757 or JTT-130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 68-5042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorber, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds according to the invention are administered in combination with a lipoprotein(a) antagonist, by way of example and with preference gemcabene calcium (CI-1027) or nicotinic acid.

Particular preference is given to combinations of the compounds according to the invention with one or more further active ingredients selected from the group consisting of corticosteroids, beta-adrenergic receptor agonists, anti-muscarinergic substances, PDE 4 inhibitors, PDE 5 inhibitors, sGC activators, sGC stimulators, HNE inhibitors, prostacyclin analogues, endothelin antagonists, statins, antifibrotic agents, anti-inflammatory agents, immunomodulating agents, immunosuppressive agents and cytotoxic agents.

The present invention further provides medicaments which comprise at least one compound according to the invention, typically together with one or more inert, non-toxic, pharmaceutically suitable excipients, and for the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Suitable administration forms for oral administration are those which work according to the prior art and release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can bypass an absorption step (e.g. take place intravenously, intraarterially, intracardially, intraspinally or intralumbally) or include an absorption (e.g. take place inhalatively, intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers, metered aerosols), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral, intrapulmonary (inhalative) and intravenous administration.

The compounds according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight. In the case of intrapulmonary administration, the amount is generally about 0.1 to 50 mg per inhalation.

It may nevertheless be necessary in some cases to deviate from the stated amounts, specifically as a function of body weight, route of administration, individual response to the active ingredient, nature of the preparation and time or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

The working examples which follow illustrate the invention. The invention is not restricted to the examples.

### A. EXAMPLES

Abbreviations and Acronyms:
abs. absolute
Ac acetyl
aq. aqueous, aqueous solution
br. broad (in NMR signal)
Ex. Example
Bu butyl
c concentration
approx. circa, about
cat. catalytic
CI chemical ionization (in MS)
d doublet (in NMR)
d day(s)
TLC thin-layer chromatography
DCI direct chemical ionization (in MS)
dd doublet of doublets (in NMR)
DEAD diethyl azodicarboxylate
DIAD diisopropyl azodicarboxylate
DMAP 4-N,N-dimethylaminopyridine
DMF N,N-dimethylformamide
DMP Dess-Martin periodinane (1,1,1-triacetoxy-1,1-dihydro-1,2-benziodoxol-3(1H)-one)
DMSO dimethyl sulphoxide
dt doublet of triplets (in NMR)
d. Th. der Theorie (bei chemischer Ausbeute)
ee enantiomeric excess
EI electron impact ionization (in MS)
ent enantiomerically pure, enantiomer
eq. equivalent(s)
ESI electrospray ionization (in MS)
Et ethyl
GC gas chromatography
GC/MS gas chromatography-coupled mass spectrometry
h hour(s)
HPLC high-pressure, high-performance liquid chromatography
iPr isopropyl
KHMDS potassium hexamethyldisilazide
cone concentrated (in the case of a solution)
LC liquid chromatography
LC/MS liquid chromatography-coupled mass spectrometry
lit. literature (reference)
m multiplet (in NMR)
Me methyl
min minute(s)
MPLC medium-pressure liquid chromatography (over silica gel; also called “flash chromatography”)
Ms methanesulphonyl (mesyl)
MS mass spectrometry
NMO N-methylmorpholine N-oxide
NMR nuclear magnetic resonance spectrometry
Pd/C palladium on activated carbon
Ph phenyl
Pr propyl
q (or quart) quartet (in NMR)
qd quartet of doublets (in NMR)
quant. quantitative (in chemical yield)
quint quintet (in NMR)
rac racemic, racemate
R_{f} retention index (in TLC)
RP reverse phase (in HPLC)
RT room temperature
Rₜ retention time (in HPLC, LC/MS)
s singlet (in NMR)
sept septet (in NMR)
SFC supercritical liquid chromatography
t triplet (in NMR)
tBu tert-butyl
td triplet of doublets (in NMR)
Tf trifluoromethylsulphonyl (triflyl)
TFA trifluoroacetic acid
THF tetrahydrofuran
Ts para-tolylsulphonyl (tosyl)
UV ultraviolet spectrometry
v/v volume to volume ratio (of a solution)
tog. together

HPLC, LC/MS and GC/MS Methods:

Method 1 (LC/MS):

Instrument: Waters ACQUITY SQD UPLC system; column: Waters Acquity UPLC HSS T3 1.8μ, 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 208-400 nm.

Method 2 (LC/MS):

Instrument: Micromass Quattro Premier with Waters UPLC Acquity; column: Thermo Hypersil GOLD 1.9μ, 50×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 3 (LC/MS):

MS instrument: Waters Micromass QM; Instrument HPLC: Agilent 1100 series; column: Agilent ZORBAX Extend-C18 3.5μ, 3.0×50 mm; mobile phase A: 1 l of water+0.01 mol of ammonium carbonate, mobile phase B: 1 l of acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 4 (LC/MS):

MS instrument: Waters SQD; HPLC instrument: Waters UPLC; column: Zorbax SB-Aq (Agilent), 50 mm×2.1 mm, 1.8 μm; mobile phase A: water+0.025% formic acid, mobile phase B: acetonitrile+0.025% formic acid; gradient: 0.0 min 98% A→0.9 min 25% A→1.0 min 5% A→1.4 min 5% A→1.41 min 98% A→1.5 min 98% A; oven: 40° C.; flow rate: 0.60 ml/min; UV detection: DAD, 210 nm.

Method 5 (GC-MS):

Instrument: Thermo Scientific DSQII, Thermo Scientific Trace GC Ultra; column: Restek RTX-35MS, 15 m×200 μm×0.33 μm; constant flow rate with helium: 1.20 ml/min; oven: 60° C.; inlet: 220° C.; gradient: 60° C., 30° C./min→300° C. (maintain for 3.33 min).

Method 6 (Preparative HPLC):

Column: Reprosil C18, 10 μm, 250×30 mm; mobile phase: acetonitrile/water with 0.1% TFA; gradient: 0-5.00 min 10:90, sample injection at 3.00 min; 5.00-23.00 min to 95:5; 23.00-30.00 min 95:5; 30.00-30.50 min to 10:90; 30.50-31.20 min 10:90.

Method 7 (Preparative HPLC):

Column: Reprosil C18, 10 μm, 250×40 mm; mobile phase: acetonitrile/water with 0.1% TFA; gradient: 0-6.00 min 10:90, sample injection at 3.00 min; 6.00-23.00 min to 95:5; 23.00-38.00 min 95:5; 38.00-39.00 min to 10:90; 39.00-40.20 min 10:90.

Method 8 (Preparative HPLC):

Column: Reprosil C18, 10 μm, 250×30 mm; mobile phase: acetonitrile/water with 0.1% TFA; gradient: 0-5.00 min 10:90, sample injection at 3.00 min; 5.00-20.00 min to 95:5; 20.00-30.00 min 95:5; 30.00-30.50 min to 10:90; 30.50-31.20 min 10:90.

Method 9 (Preparative HPLC):

Column: Reprosil C18, 10 μm, 250×40 mm; mobile phase: acetonitrile/water with 0.1% TFA; gradient: 0-6.00 min 10:90, sample injection at 3.00 min; 6.00-27.00 min to 95:5; 27.00-54.00 min 95:5; 54.00-55.00 min to 10:90; 55.00-56.20 min 10:90.

Method 10 (Preparative HPLC):

MS instrument: Waters; Instrument HPLC: Waters; column: Phenomenex Luna 5 g C18(2) 100A, AXIA Tech., 50 mm×21.2 mm; mobile phase A: water+0.05% formic acid, mobile phase B: acetonitrile+0.05% formic acid; gradient: 0.0 min 95% A→0.15 min 95% A→8.0 min 5% A→9.0 min 5% A; flow rate 40 ml/min; UV detection: DAD, 210-400 nm.

Further Details:

The percentages in the example and test descriptions which follow are, unless indicated otherwise, percentages by weight; parts are parts by weight. Solvent ratios, dilution ratios and concentration data for liquid/liquid solutions are based in each case on volume.

Purity figures are generally based on corresponding peak integrations in the LC/MS chromatogram, but may additionally also have been determined with the aid of the ¹H NMR spectrum. If no purity is indicated, the purity is generally 100% according to automated peak integration in the LC/MS chromatogram, or the purity has not been determined explicitly.

Stated yields in % of theory are generally corrected for purity if a purity of <100% is indicated. In solvent-containing or contaminated batches, the formal yield may be “>100%”; in these cases the yield is not corrected for solvent or purity.

The descriptions of the coupling patterns of ¹H NMR signals that follow have in some cases been taken directly from the suggestions of the ACD SpecManager (ACD/Labs Release 12.00, Product version 12.5) and have not necessarily been strictly scrutinized. In some cases, the suggestions of the SpecManager were adjusted manually. Manually adjusted or assigned descriptions are generally based on the optical appearance of the signals in question and do not necessarily correspond to a strict, physically correct interpretation. In general, the stated chemical shift refers to the centre of the signal in question. In the case of broad multiplets, an interval is given. Signals obscured by solvent or water were either tentatively assigned or have not been listed.

Melting points and melting-point ranges, if stated, are uncorrected.

All reactants or reagents whose preparation is not described explicitly hereinafter were purchased commercially from generally accessible sources. For all other reactants or reagents whose preparation likewise is not described hereinafter and which were not commercially obtainable or were obtained from sources which are not generally accessible, a reference is given to the published literature in which their preparation is described.

In the intermediates and working examples described hereinbelow, a name listed in the IUPAC name of the example in question “1RS,2SR,5RS” in conjunction with the statement “racemate”, is a racemic mixture of the 1R,2S,5R-enantiomer (→ in each case 1st letter after the positional number in “1RS,2SR,5RS”) with the corresponding 1S,2R,5S-enantiomer (→ in each case 2nd letter after the positional number). The “1RS,2SR,5RS” identifier in conjunction with the statements “enantiomer 1” and “enantiomer 2” means that these are the two enantiomers in separate, isolated form, without having undertaken an assignment of the absolute configuration (1R,2S,5R or 1S,2R,5S) to these enantiomers. Similar identifiers such as “1RS,2RS,3SR” or “1RS,2RS,5SR” that arise from the altered priority and/or sequence of named constituents owing to the IUPAC nomenclature rules should be interpreted in an analogous manner according to these instructions.

For the simplified representation of the relative stereochemical configuration of chiral centres, the structural formulae of racemic example compounds hereinbelow show only the structural formula of one of the enantiomers involved; as is evident from the term “racemate” in the associated IUPAC name, the second enantiomer with the respective opposite absolute configuration is always included in these cases.

Starting Compounds and Intermediates:

### Example 1A

### 6-(Trifluoromethyl)-1,2,3-benzotriazin-4(3H)-one

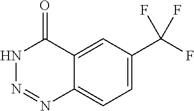 [SMILES: O=c1[nH]nnc2ccc(C(F)(F)F)cc12]

To a suspension of 24.4 g (119.51 mmol) of 2-amino-5-(trifluoromethyl)benzamide in 174 ml of a 2:1 mixture of water and conc. hydrochloric acid at 0° C. was gradually added a solution of 9.08 g (131.47 mmol) of sodium nitrite in 74 ml of water, in the course of which the internal temperature was kept below 5° C. After stirring at bath temperature 0° C. for 30 minutes, while continuing to cool with an ice bath, 74 ml (0.74 mol) of 10 M sodium hydroxide solution were added, in the course of which the internal temperature rose to about 20° C. A solution formed at first, from which a suspension then arose, which was diluted with 100 ml of water for better stirrability. After stirring at RT for 1.5 h, the mixture was cautiously acidified with conc. hydrochloric acid (pH=2). The precipitate formed was filtered off and washed three times with water. After drying under air and then in vacuo, 24.74 g (96% of theory) of the title compound were obtained.

¹H-NMR (400 MHz, DMSO-d₆): δ [ppm]=15.31 (br. s, 1H), 8.46 (s, 1H), 8.40 (d, 2H).

LC/MS (Method 1, ESIpos): Rₜ=0.78 min, m/z=216 [M+H]⁺.

### Example 2A

### Di-tert-butyl [2-(4-oxo-1,2,3-benzotriazin-3(4H)-yl)ethyl]malonate

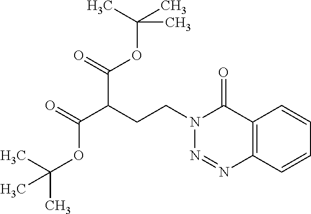 [SMILES: CC(C)(C)OC(=O)C(CCn1nnc2ccccc2c1=O)C(=O)OC(C)(C)C]

A solution of 100 g (0.321 mol, purity 84%) of di-tert-butyl (2-hydroxyethyl)malonate (U.S. Pat. No. 6,900,194, intermediate 5E) in 1 litre of THF under argon was admixed with 100.96 g (0.385 mol) of triphenylphosphine and 56.63 g (0.385 mol) of 1,2,3-benzotriazin-4(3H)-one and then stirred for 20 min at RT. Then, 75.8 ml (0.385 mol) of diisopropyl azodicarboxylate (DIAD) were slowly added, in the course of which the internal temperature rose to approx. 30° C. with gentle cooling. The mixture was then stirred overnight at RT and then stirred into 5 litres of a 10% strength aqueous sodium chloride solution. After adding 2 litres of ethyl acetate, the organic phase was separated off, washed with 750 ml of 10% strength sodium hydrogencarbonate solution, dried over sodium sulphate and concentrated. The residue was taken up in a small amount of dichloromethane, admixed with some petroleum ether and purified by means of column chromatography (5 kg of silica gel, mobile phase petroleum ether/ethyl acetate 8:2). 109.4 g (88% of theory) of the title compound were obtained.

¹H-NMR (400 MHz, DMSO-d₆): δ [ppm]=8.26 (d, 1H), 8.19 (d, 1H), 8.09 (t, 1H), 7.93 (t, 1H), 4.43 (t, 2H), 3.40 (t, 1H), 2.25 (q, 2H), 1.37 (s, 18H).

LC/MS (Method 1, ESIpos): Rₜ=1.28 min, m/z=390 [M+H]⁺.

### Example 3A

### Di-tert-butyl {2-[4-oxo-6-(trifluoromethyl)-1,2,3-benzotriazin-3(4H)-yl]ethyl}malonate

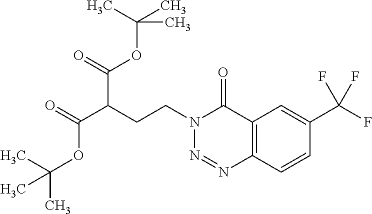 [SMILES: CC(C)(C)OC(=O)C(CCn1nnc2ccc(C(F)(F)F)cc2c1=O)C(=O)OC(C)(C)C]

A solution of 18.78 g (71.58 mmol) of triphenylphosphine in 210 ml of THF was admixed under argon at RT with 14.8 ml (71.58 mmol, purity 95%) of diisopropyl azodicarboxylate (DIAD) and briefly after-stirred. The internal temperature increased as a result to 40° C. and a precipitate was formed. Then, 14.0 g (65.08 mmol) of the compound from Example 4A were added, and the mixture was after-stirred for a further few minutes. Then, 18.64 g (71.58 mmol) of di-tert-butyl (2-hydroxyethyl)malonate (U.S. Pat. No. 6,900,194, intermediate 5E) were added, during which the internal temperature rose to 60° C. The solution was further stirred for 1 h. Then, the solvent was removed, and the residue was taken up in 200 ml of tert-butyl methyl ether. The solid formed was filtered off, after-washed with 30 ml of tert-butyl methyl ether and discarded. The filtrate was concentrated and the residue was purified by column chromatography (1 kg of silica gel, mobile phase cyclohexane/ethyl acetate 9:1). 26.44 g (89% of theory) of the title compound were obtained.

¹H-NMR (400 MHz, CDCl₃): δ [ppm]=8.65 (s, 1H), 8.28 (d, 1H), 8.15 (dd, 1H), 4.60 (t, 2H), 3.29 (t, 1H), 2.43 (q, 2H), 1.45 (s, 18H).

LC/MS (Method 1, ESIpos): Rₜ=1.37 min, m/z=457 [M+H]⁺.

### Example 4A

### 1-[5-(Benzyloxy)-1-benzofur-2-yl]ethanone

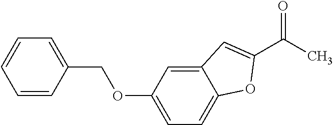 [SMILES: CC(=O)c1cc2cc(OCc3ccccc3)ccc2o1]

To a solution of 240 g (1.05 mol) of 5-benzyloxy-2-hydroxybenzaldehyde in 3.6 litres of acetone were added at RT 291 g (2.10 mol) of potassium carbonate and then 110 ml (1.31 mol, purity 96%) of chloroacetone were added dropwise. After stirring for 1 h under reflux and cooling to RT, the reaction mixture was filtered and the filtrate was concentrated. The residue was stirred with 700 ml of 90% strength aqueous methanol, then filtered off and after-washed with methanol. This gave 98 g (35% of theory) of a first batch of the title compound. The mother liquor was concentrated and produced approx. 200 g of a crude product, which was purified together with 38 g of a crude product obtained in a similar manner from a previous experiment by means of column chromatography (silica gel, mobile phase petroleum ether/ethyl acetate 9:1-7:3). In this way, 112 g (39% of theory) of a second batch of the title compound were obtained.

LC/MS (Method 1, ESIpos): Rₜ=1.13 min, m/z=267 [M+H]⁺.

¹H-NMR (400 MHz, DMSO-d₆): δ [ppm]=7.80 (d, 1H), 7.64 (d, 1H), 7.51-7.29 (m, 6H), 7.22 (dd, 1H), 5.16 (s, 2H), 2.54 (s, 3H, partially hidden).

### Example 5A

### 1-[5-(Benzyloxy)-1-benzofur-2-yl]-2-bromoethanone

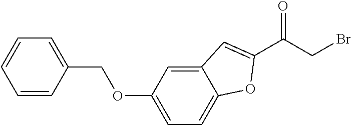 [SMILES: O=C(CBr)c1cc2cc(OCc3ccccc3)ccc2o1]

To a solution of 280 g (1.05 mol) of the compound from Example 4A in 3.9 litres of THF under argon were added at RT a total of 415 g (1.10 mol) of phenyltrimethylammonium tribromide in ten portions over the course of 30 minutes. After stirring for 1 h at RT, the mixture was admixed with 20 litres of 10% strength aqueous sodium chloride solution. 11 litres of ethyl acetate were added, and approx. 18% strength (semi-concentrated) hydrochloric acid was used to adjust the pH to 1. After phase separation, the aqueous phase was extracted once with 11 ml of ethyl acetate. The combined organic phases were dried over sodium sulphate and concentrated. The residue was stirred in methanol, filtered off and after-washed with methanol. 260 g (58% of theory, purity 81%) of the title compound were obtained.

LC/MS (Method 1, ESIpos): Rₜ=1.22 min, m/z=344/346 [M+H]⁺.

### Example 6A

### Di-tert-butyl {2-[5-(benzyloxy)-1-benzofur-2-yl]-2-oxoethyl}[2-(4-oxo-1,2,3-benzotriazin-3(4H)-yl)ethyl]malonate

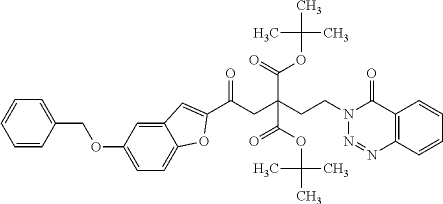 [SMILES: CC(C)(C)OC(=O)C(CCn1nnc2ccccc2c1=O)(CC(=O)c1cc2cc(OCc3ccccc3)ccc2o1)C(=O)OC(C)(C)C]

To a suspension of 5.39 g (134.79 mmol) of sodium hydride (60% in paraffin oil) in 297 ml of DMF under argon were added at RT a solution of 42.34 g (108.71 mmol) of the compound from Example 2A in 297 ml of DMF. After stirring for 30 minutes at RT, a solution of 38.00 g (86.96 mmol, purity 79%) of the compound from Example 5A in 297 ml of DMF were added quickly and the mixture was stirred for a further 2 h at RT. Then, the mixture was admixed with once 10% strength sodium chloride solution and ethyl acetate. After phase separation, the aqueous phase was extracted with ethyl acetate, and the combined organic phases were washed once with 10% strength sodium chloride solution, dried over sodium sulphate and concentrated. The residue was taken up in dichloromethane/petroleum ether and purified by column chromatography (silica gel, mobile phase petroleum ether/ethyl acetate 8:2). 16.6 g (29% of theory) of the title compound were obtained.

¹H-NMR (400 MHz, DMSO-d₆): δ [ppm]=8.20 (dd, 1H), 8.11 (d, 1H), 8.04-7.96 (m, 1H), 7.90-7.81 (m, 2H), 7.63 (d, 1H), 7.52-7.45 (m, 2H), 7.44-7.30 (m, 4H), 7.23 (dd, 1H), 5.17 (s, 2H), 4.51-4.41 (m, 2H), 3.65 (s, 2H), 2.54-2.47 (2H, hidden), 1.40 (s, 18H).

LC/MS (Method 1, ESIpos): Rₜ=1.51 min, m/z=654 [M+H]⁺.

### Example 7A

### Di-tert-butyl-[2-(5-hydroxy-1-benzofur-2-yl)-2-oxoethyl][2-(4-oxo-1,2,3-benzotriazin-3(4H)-yl)ethyl]malonate

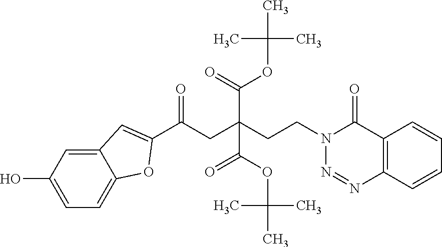 [SMILES: CC(C)(C)OC(=O)C(CCn1nnc2ccccc2c1=O)(CC(=O)c1cc2cc(O)ccc2o1)C(=O)OC(C)(C)C]

21.0 g (32.12 mmol) of the compound from Example 6A were dissolved in a mixture of 630 ml of ethyl acetate and 630 ml of ethanol, admixed with 1.71 g (1.61 mmol) of palladium catalyst (10% on activated carbon) and 10.13 g (160.62 mmol) of ammonium formate at RT and then stirred for 1 h at 70° C. After cooling to RT, the mixture was filtered through kieselguhr and the filter residue was washed with ethanol. The filtrate was concentrated and the residue was pre-purified by column chromatography (silica gel, mobile phase cyclohexane/ethyl acetate 95:5). The product obtained in this way was stirred in 190 ml of diethyl ether for 30 minutes, then filtered off and dried in vacuo. 15.0 g (82% of theory) of the title compound were obtained.

¹H-NMR (400 MHz, DMSO-d₆): δ [ppm]=9.52 (s, 1H), 8.21 (dd, 1H), 8.14 (d, 1H), 8.03 (td, 1H), 7.92-7.84 (m, 1H), 7.81 (s, 1H), 7.51 (d, 1H), 7.07 (d, 1H), 7.00 (dd, 1H), 4.49-4.42 (m, 2H), 3.63 (s, 2H), 2.52-2.47 (2H, hidden), 1.40 (s, 18H).

LC/MS (Method 1, ESIpos): Rₜ=1.25 min, m/z=564 [M+H]⁺.

### Example 8A

### Di-tert-butyl (2-{5-[2-(2-chlorophenyl)ethoxy]-1-benzofur-2-yl}-2-oxoethyl)[2-(4-oxo-1,2,3-benzotriazin-3(4H)-yl)ethyl]malonate

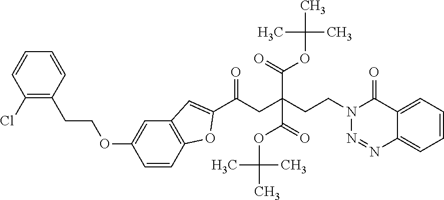 [SMILES: CC(C)(C)OC(=O)C(CCn1nnc2ccccc2c1=O)(CC(=O)c1cc2cc(OCCc3ccccc3Cl)ccc2o1)C(=O)OC(C)(C)C]

To a mixture of 100 mg (0.18 mmol) of the compound from Example 7A in 2.5 ml of acetonitrile were added 47 mg (0.21 mmol) of 1-(2-bromoethyl)-2-chlorobenzene and 49 mg (0.36 mmol) of potassium carbonate and the mixture was stirred under reflux for 1.5 h. Then, a further 47 mg (0.21 mmol) of 1-(2-bromoethyl)-2-chlorobenzene and 49 mg (0.36 mmol) of potassium carbonate were added and the mixture was stirred under reflux for a further 4.5 h. Then, the mixture was left to stand firstly overnight at RT and then, after another addition of 47 mg (0.21 mmol) of 1-(2-bromoethyl)-2-chlorobenzene, stirred for a further 5 h at 50° C. After cooling to RT, the mixture was admixed with 1 ml of water and purified directly by preparative HPLC (Method 7). The combined product-containing fractions were neutralized with saturated aqueous sodium hydrogencarbonate solution, concentrated to a residual volume of aqueous phase and extracted twice with dichloromethane. The combined organic phases were dried over magnesium sulphate and concentrated, and the residue was dried in vacuo. 21 mg (17% of theory, purity 100%) of the title compound were obtained.

LC/MS (Method 1, ESIpos): Rₜ=1.59 min, m/z=702 [M+H]⁺.

### Example 9A

### Di-tert-butyl (2-{5-[2-(4-chlorophenyl)ethoxy]-1-benzofur-2-yl}-2-oxoethyl)[2-(4-oxo-1,2,3-benzotriazin-3(4H)-yl)ethyl]malonate

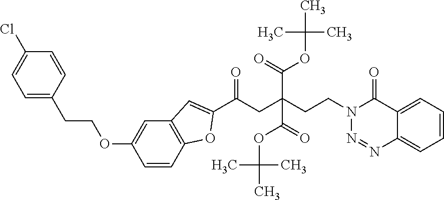 [SMILES: CC(C)(C)OC(=O)C(CCn1nnc2ccccc2c1=O)(CC(=O)c1cc2cc(OCCc3ccc(Cl)cc3)ccc2o1)C(=O)OC(C)(C)C]

To a mixture of 100 mg (0.18 mmol) of the compound from Example 7A in 2.5 ml of acetonitrile were added 47 mg (0.21 mmol) of 1-(2-bromoethyl)-4-chlorobenzene and 49 mg (0.36 mmol) of potassium carbonate and the mixture was stirred under reflux for 1.5 h. Then, a further 47 mg (0.21 mmol) of 1-(2-bromoethyl)-4-chlorobenzene and 49 mg (0.36 mmol) of potassium carbonate were added and the mixture was stirred under reflux for a further 4.5 h. Then, the mixture was left to stand firstly overnight at RT and then, after another addition of 47 mg (0.21 mmol) of 1-(2-bromoethyl)-4-chlorobenzene, stirred for a further 5 h at 50° C. After cooling to RT, the mixture was admixed with 1 ml of water and purified directly by preparative HPLC (Method 7). The combined product-containing fractions were neutralized with saturated aqueous sodium hydrogencarbonate solution, concentrated to a residual volume of aqueous phase and extracted twice with dichloromethane. The combined organic phases were dried over magnesium sulphate and concentrated, and the residue was dried in vacuo. 13 mg (10% of theory, purity 100%) of the title compound were obtained.

LC/MS (Method 1, ESIpos): Rₜ=1.57 min, m/z=702 [M+H]⁺.

### Example 10A

### Di-tert-butyl (2-{5-[2-(4-methoxyphenyl)ethoxy]-1-benzofur-2-yl}-2-oxoethyl)[2-(4-oxo-1,2,3-benzotriazin-3(4H)-yl)ethyl]malonate

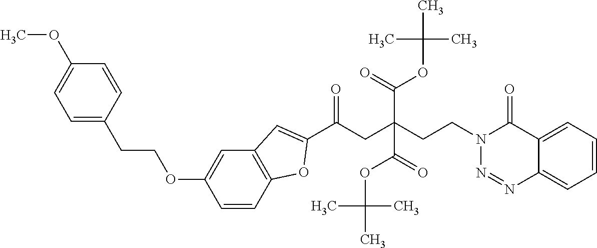 [SMILES: COc1ccc(CCOc2ccc3oc(C(=O)CC(CCn4nnc5ccccc5c4=O)(C(=O)OC(C)(C)C)C(=O)OC(C)(C)C)cc3c2)cc1]

To a mixture of 100 mg (0.18 mmol) of the compound from Example 7A in 2.5 ml of acetonitrile were added 46 mg (0.21 mmol) of 1-(2-bromoethyl)-4-methoxybenzene and 49 mg (0.36 mmol) of potassium carbonate and the mixture was stirred under reflux for 1.5 h. Then, a further 46 mg (0.21 mmol) of 1-(2-bromoethyl)-4-methoxybenzene and 49 mg (0.36 mmol) of potassium carbonate were added and the mixture was stirred under reflux for a further 4.5 h. Then, the mixture was left to stand firstly overnight at RT and then, after another addition of 46 mg (0.21 mmol) of 1-(2-bromoethyl)-4-methoxybenzene, stirred for a further 5 h at 50° C. After cooling to RT, the mixture was admixed with 1 ml of water and purified directly by preparative HPLC (Method 7). The combined product-containing fractions were neutralized with saturated aqueous sodium hydrogencarbonate solution, concentrated to a residual volume of aqueous phase and extracted twice with dichloromethane. The combined organic phases were dried over magnesium sulphate and concentrated, and the residue was dried in vacuo. 106 mg (85% of theory, purity 100%) of the title compound were obtained.

LC/MS (Method 1, ESIpos): Rₜ=1.52 min, m/z=698 [M+H]⁺.

### Example 11A

### Di-tert-butyl (2-{5-[2-(2-methylphenyl)ethoxy]-1-benzofur-2-yl}-2-oxoethyl)[2-(4-oxo-1,2,3-benzotriazin-3(4H)-yl)ethyl]malonate

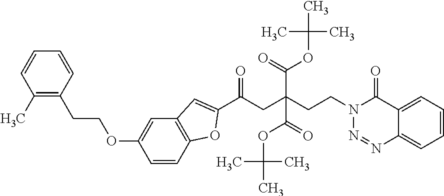 [SMILES: Cc1ccccc1CCOc1ccc2oc(C(=O)CC(CCn3nnc4ccccc4c3=O)(C(=O)OC(C)(C)C)C(=O)OC(C)(C)C)cc2c1]

To a mixture of 100 mg (0.18 mmol) of the compound from Example 7A and 98 mg (0.71 mmol) of potassium carbonate in 2.5 ml of pentanol were added at 120° C. 48 mg (0.24 mmol, purity 97%) of 1-(2-bromoethyl)-2-methylbenzene and the mixture was stirred for 1 h at 120° C. Then, a further 48 mg (0.24 mmol, purity 97%) of 1-(2-bromoethyl)-2-methylbenzene were added and the mixture was stirred for a further hour at 120° C. Thereafter, another 48 mg (0.24 mmol; purity 97%) of 1-(2-bromoethyl)-2-methylbenzene were added, and the mixture was stirred at 120° C. for a further 3 h. After cooling to RT, the mixture was concentrated, admixed with water and extracted twice with dichloromethane. The combined organic phases were dried over magnesium sulphate, filtered and concentrated. The residue was purified by means of preparative HPLC (Method 8). The combined product-containing fractions were neutralized with saturated aqueous sodium hydrogencarbonate solution, concentrated to a residual volume of aqueous phase and extracted twice with dichloromethane. The combined organic phases were dried over magnesium sulphate and concentrated, and the residue was dried in vacuo. 80 mg (64% of theory, purity 97%) of the title compound were obtained.

¹H-NMR (400 MHz, CDCl₃): δ [ppm]=8.29 (dd, 1H), 8.08 (d, 1H), 7.88 (td, 1H), 7.78-7.70 (m, 1H), 7.51-7.40 (m, 2H), 7.30-7.23 (m, 1H), 7.21-7.13 (m, 3H), 7.12-7.03 (m, 2H), 4.60-4.52 (m, 2H), 4.18 (t, 2H), 3.73 (s, 2H), 3.15 (t, 2H), 2.69-2.63 (m, 2H), 2.62 (s, 3H), 1.48 (s, 18H).

LC/MS (Method 1, ESIpos): Rₜ=1.59 min, m/z=682 [M+H]⁺.

### Example 12A

### Di-tert-butyl [2-(4-oxo-1,2,3-benzotriazin-3(4H)-yl)ethyl]{2-oxo-2-[5-(tetrahydro-2H-pyran-4-ylmethoxy)-1-benzofur-2-yl]ethyl}malonate

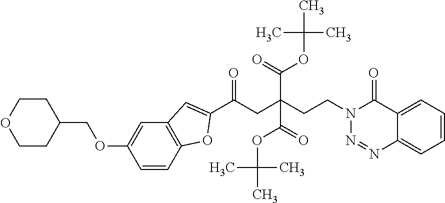 [SMILES: CC(C)(C)OC(=O)C(CCn1nnc2ccccc2c1=O)(CC(=O)c1cc2cc(OCC3CCOCC3)ccc2o1)C(=O)OC(C)(C)C]

To a mixture of 200 mg (0.36 mmol) of the compound from Example 7A in 5 ml of acetonitrile were added, at a bath temperature of 95° C., 147 mg (1.06 mmol) of potassium carbonate and 127 mg (0.71 mmol) of 4-(bromomethyl)tetrahydro-2H-pyran, and the mixture was stirred under reflux for 48 h. After cooling to RT, water was added, and the aqueous phase was extracted three times with 3 ml of ethyl acetate in each case. The combined organic phases were dried over magnesium sulphate, filtered and concentrated. The residue was taken up in 4 ml of acetonitrile and purified by means of preparative HPLC (Method 8). The combined product-containing fractions were neutralized with saturated aqueous sodium hydrogencarbonate solution and extracted with ethyl acetate. Concentration of the organic phase and drying of the residue in vacuo gave 106 mg (45% of theory, purity 100%) of the title compound.

LC/MS (Method 1, ESIpos): Rₜ=1.45 min, m/z=662 [M+H]⁺.

### Example 13A

### Di-tert-butyl [2-(4-oxo-1,2,3-benzotriazin-3(4H)-yl)ethyl](2-oxo-2-{5-[2-(tetrahydro-2H-pyran-4-yl)ethoxy]-1-benzofur-2-yl}ethyl)malonate

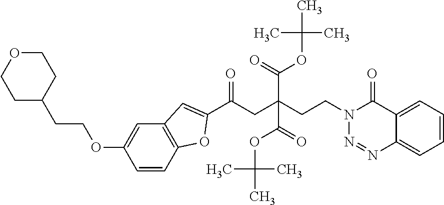 [SMILES: CC(C)(C)OC(=O)C(CCn1nnc2ccccc2c1=O)(CC(=O)c1cc2cc(OCCC3CCOCC3)ccc2o1)C(=O)OC(C)(C)C]

To a mixture of 200 mg (0.36 mmol) of the compound from Example 7A in 0.75 ml of DMF under argon were added, at RT, 45 mg (0.40 mmol) of potassium tert-butoxide and, after stirring for 5 min at RT, 86 mg (0.44 mmol) of 4-(2-bromoethyl)tetrahydro-2H-pyran, dissolved in 0.25 ml of DMF. The mixture was stirred at a bath temperature of 70° C. for 1.5 h. After cooling to RT, in each case 50 ml of water and ethyl acetate were added, and after phase separation, the aqueous phase was extracted once with 50 ml of ethyl acetate. The combined organic phases were dried over sodium sulphate, filtered and concentrated. The residue was taken up in dichloromethane and purified by column chromatography (silica gel, mobile phase: cyclohexane/ethyl acetate 7:3). 111 mg (43% of theory, purity 94%) of the title compound were obtained.

LC/MS (Method 1, ESIpos): Rₜ=1.59 min, m/z=676 [M+H]⁺.

### Example 14A

### Di-tert-butyl {2-[5-(2-cyclopentylethoxy)-1-benzofur-2-yl]-2-oxoethyl}[2-(4-oxo-1,2,3-benzotriazin-3(4H)-yl)ethyl]malonate

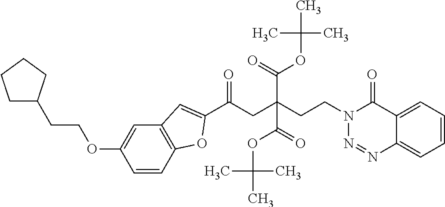 [SMILES: CC(C)(C)OC(=O)C(CCn1nnc2ccccc2c1=O)(CC(=O)c1cc2cc(OCCC3CCCC3)ccc2o1)C(=O)OC(C)(C)C]

To a mixture of 200 mg (0.36 mmol) of the compound from Example 7A in 0.75 ml of DMF under argon were added at RT 45 mg (0.40 mmol) of potassium tert-butoxide and, after stirring for 5 min at RT, 99 mg (0.44 mmol) of (2-iodoethyl)cyclopentane, dissolved in 0.25 ml of DMF. The mixture was stirred at 70° C. for 5 h. After cooling to RT, a further 52 mg (0.23 mmol) of (2-iodoethyl)cyclopentane were added, and the mixture was stirred for a further 3 h at a bath temperature of 70° C. After cooling to RT, the mixture was admixed with 5 ml of water. The solid present was filtered off, washed twice with 2 ml of water in each case and dried in vacuo to give a first crude product batch of the title compound. The filtrate was admixed with water and ethyl acetate, and, after phase separation, the aqueous phase was extracted once with ethyl acetate. The combined organic phases were dried over sodium sulphate, filtered and concentrated and thus produced a second crude product batch of the title compounds. Both batches were combined and purified by column chromatography (40 g silica gel, mobile phase cyclohexane/ethyl acetate 85:15). 159 mg (64% of theory, purity 94%) of the title compound were obtained.

LC/MS (Method 1, ESIpos): Rₜ=1.67 min, m/z=660 [M+H]⁺.

### Example 15A

### Di-tert-butyl {2-[5-(cyclohexylmethoxy)-1-benzofur-2-yl]-2-oxoethyl}[2-(4-oxo-1,2,3-benzotriazin-3(4H)-yl)ethyl]malonate

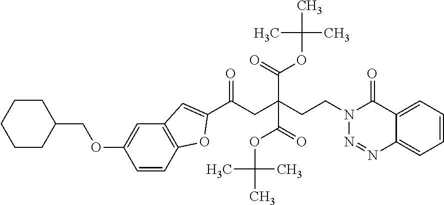 [SMILES: CC(C)(C)OC(=O)C(CCn1nnc2ccccc2c1=O)(CC(=O)c1cc2cc(OCC3CCCCC3)ccc2o1)C(=O)OC(C)(C)C]

To a mixture of 800 mg (1.42 mmol) of the compound from Example 7A in 3 ml of DMF under argon were added at RT 179 mg (1.60 mmol) of potassium tert-butoxide and, after stirring for 5 min at RT, 314 mg (1.77 mmol) of (bromomethyl)cyclohexane. The mixture was stirred at a bath temperature of 70° C. for 4 h. Then, a further 314 mg (1.77 mmol) of (bromomethyl)cyclohexane were added and the mixture was stirred for a further hour at 70° C. After cooling to RT, the mixture was admixed with in each case 100 ml of water and ethyl acetate. After phase separation, the aqueous phase was extracted once with 50 ml of ethyl acetate, and the combined organic phases were dried over sodium sulphate, filtered and concentrated. The residue was dissolved in 6 ml of acetonitrile at elevated temperature. After cooling to RT, the solid present was filtered off, washed twice with 2 ml of acetonitrile in each case and dried. In this way, 193 mg (19% of theory, purity 92%) of a first batch of the title compound were obtained. The filtrate was concentrated and the residue was purified by preparative HPLC (Method 9, two injections). The combined product-containing fractions were neutralized with saturated aqueous sodium hydrogencarbonate solution and concentrated to a residual volume of aqueous phase. After extracting twice with ethyl acetate, the combined organic phases were dried over sodium sulphate, filtered and concentrated, and the residue was dried in vacuo. This gave 314 mg (34% of theory, purity 100%) of a second batch of the title compound.

LC/MS (Method 1, ESIpos): Rₜ=1.68 min, m/z=660 [M+H]⁺.

### Example 16A

### Di-tert-butyl [2-(4-oxo-1,2,3-benzotriazin-3(4H)-yl)ethyl]{2-oxo-2-[5-(4,4,4-trifluorobutoxy)-1-benzofur-2-yl]ethyl}malonate

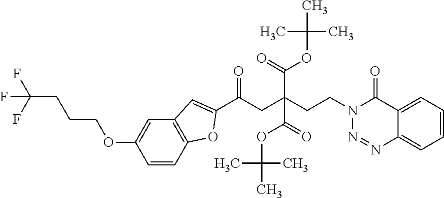 [SMILES: CC(C)(C)OC(=O)C(CCn1nnc2ccccc2c1=O)(CC(=O)c1cc2cc(OCCCC(F)(F)F)ccc2o1)C(=O)OC(C)(C)C]

To a mixture of 200 mg (0.36 mmol) of the compound from Example 7A and 147 mg (1.07 mmol) of potassium carbonate in 8 ml of acetonitrile under argon were added at RT 203 mg (1.07 mmol) of 4-bromo-1,1,1-trifluorobutane and the mixture was stirred for 1 h at RT. Then, water was added and the mixture was extracted twice with dichloromethane. The combined organic phases were dried over magnesium sulphate, filtered and concentrated, and the residue was dried in vacuo. 208 mg (84% of theory, purity 97%) of the title compound were obtained.

LC/MS (Method 1, ESIpos): Rₜ=1.49 min, m/z=674 [M+H]⁺.

### Example 17A

### Di-tert-butyl (2-{5-[(4-fluorotetrahydro-2H-pyran-4-yl)methoxy]-1-benzofur-2-yl}-2-oxoethyl)[2-(4-oxo-1,2,3-benzotriazin-3(4H)-yl)ethyl]malonate

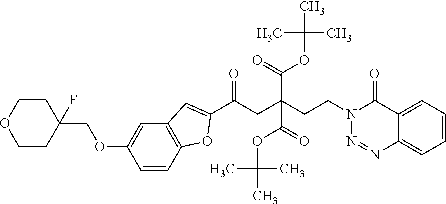 [SMILES: CC(C)(C)OC(=O)C(CCn1nnc2ccccc2c1=O)(CC(=O)c1cc2cc(OCC3(F)CCOCC3)ccc2o1)C(=O)OC(C)(C)C]

To a mixture of 200 mg (0.36 mmol) of the compound from Example 7A and 147 mg (1.07 mmol) of potassium carbonate in 8 ml of acetonitrile under argon were added at RT 283 mg (1.07 mmol) of (4-fluorotetrahydro-2H-pyran-4-yl)methyltrifluoromethanesulphonate [Lit.: EP 2 090 570-A1, Reference example 23, Step 1]. The mixture was stirred under reflux for 20 h. After cooling to RT, water was added and the mixture was extracted twice with dichloromethane. The combined organic phases were dried over magnesium sulphate, filtered and concentrated. 264 mg (purity 90%) of a crude product batch of the title compound were obtained. This was combined with 97 mg (purity 83%) of a further crude product batch of the title compound obtained from an experiment performed in a similar manner and purified together by column chromatography (silica gel, mobile phase cyclohexane/ethyl acetate 7:3). In this way, 182 mg of the title compound were obtained (purity 100%, 51% of theory, based on 300 mg (0.53 mmol) of the total amount used of the compound from Example 7A).

LC/MS (Method 1, ESIpos): Rₜ=1.41 min, m/z=680 [M+H]⁺.

### Example 18A

### Di-tert-butyl {2-[5-(3-cyanopropoxy)-1-benzofur-2-yl]-2-oxoethyl}[2-(4-oxo-1,2,3-benzotriazin-3(4H)-yl)ethyl]malonate

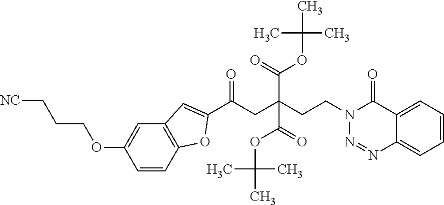 [SMILES: CC(C)(C)OC(=O)C(CCn1nnc2ccccc2c1=O)(CC(=O)c1cc2cc(OCCCC#N)ccc2o1)C(=O)OC(C)(C)C]

To a mixture of 200 mg (0.36 mmol) of the compound from Example 7A and 147 mg (1.07 mmol) of potassium carbonate in 8 ml of acetonitrile under argon were added at RT 158 mg (1.07 mmol) of 4-bromobutanonitrile. The mixture was stirred firstly at RT for 1 h and then under reflux for 3 h. After cooling to RT, water was added and the mixture was extracted twice with dichloromethane. The combined organic phases were dried over magnesium sulphate, filtered and concentrated, and the residue was dried in vacuo. 221 mg (94% of theory, purity 95%) of the title compound were obtained.

LC/MS (Method 1, ESIpos): Rₜ=1.37 min, m/z=631 [M+H]⁺.

### Example 19A

### Di-tert-butyl-{2-[5-(cyclopentylmethoxy)-1-benzofur-2-yl]-2-oxoethyl}[2-(4-oxo-1,2,3-benzotriazin-3(4H)-yl)ethyl]malonate

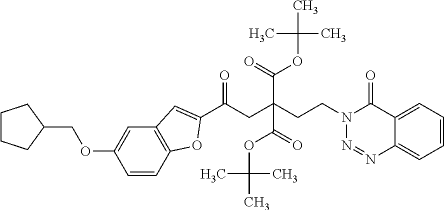 [SMILES: CC(C)(C)OC(=O)C(CCn1nnc2ccccc2c1=O)(CC(=O)c1cc2cc(OCC3CCCC3)ccc2o1)C(=O)OC(C)(C)C]

To a mixture of 750 mg (1.33 mmol) of the compound from Example 7A and 552 mg (3.99 mmol) of potassium carbonate in 10 ml of acetonitrile under argon were added at RT 759 mg (4.66 mmol) of (bromomethyl)cyclopentane and the mixture was stirred under reflux for 16 h. Then, a further 380 mg (2.33 mmol) of (bromomethyl)cyclopentane were added and the mixture was again stirred under reflux for 2 h. After cooling to RT, water was added and the mixture was extracted twice with ethyl acetate. The combined organic phases were dried over magnesium sulphate, filtered and concentrated. The residue was purified by means of column chromatography (80 g of silica gel, mobile phase cyclohexane/ethyl acetate 7:3). 580 mg (68% of theory, purity 94%) of the title compound were obtained.

LC/MS (Method 1, ESIpos): Rₜ=1.60 min, m/z=646 [M+H]⁺.

### Example 20A

### Di-tert-butyl [2-(4-oxo-1,2,3-benzotriazin-3(4H)-yl)ethyl]{2-oxo-2-[5-(2-phenylethoxy)-1-benzofur-2-yl]ethyl}malonate

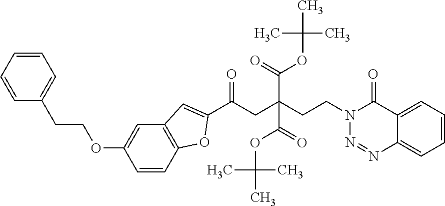 [SMILES: CC(C)(C)OC(=O)C(CCn1nnc2ccccc2c1=O)(CC(=O)c1cc2cc(OCCc3ccccc3)ccc2o1)C(=O)OC(C)(C)C]

To a mixture of 5.0 g (8.25 mmol, purity 93%) of the compound from Example 7A and 4.56 g (33.00 mmol) of potassium carbonate in 100 ml of pentanol were added at 120° C. 5 ml of a solution of 6.11 g (33.00 mmol) of (2-bromoethyl)benzene in 15 ml of pentanol. The mixture was stirred under reflux for 1 h. Then, a further 5 ml of said (2-bromoethyl)benzene solution were added and the mixture was stirred under reflux for a further hour. Finally, the remaining 5 ml of the (2-bromoethyl)benzene solution were added and the mixture was again stirred under reflux for 5 h. After cooling to RT and leaving to stand overnight, the mixture was concentrated, and the residue was admixed with water and extracted twice with dichloromethane. The combined organic phases were dried over magnesium sulphate, filtered and concentrated. The residue was purified by means of column chromatography (300 g of silica gel, mobile phase cyclohexane/ethyl acetate 8:2). 3.53 g (62% of theory, purity 97%) of the title compound were obtained.

LC/MS (Method 1, ESIpos): Rₜ=1.54 min, m/z=668 [M+H]⁺.

### Example 21A

### Di-tert-butyl (2-{5-[(4-fluorobenzyl)oxy]-1-benzofur-2-yl}-2-oxoethyl)[2-(4-oxo-1,2,3-benzotriazin-3(4H)-yl)ethyl]malonate

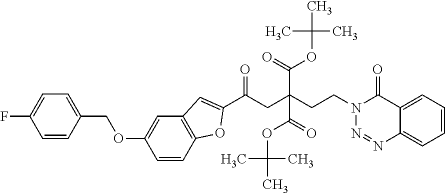 [SMILES: CC(C)(C)OC(=O)C(CCn1nnc2ccccc2c1=O)(CC(=O)c1cc2cc(OCc3ccc(F)cc3)ccc2o1)C(=O)OC(C)(C)C]

To a mixture of 400 mg (0.71 mmol) of the compound from Example 7A and 196 mg (1.41 mmol) of potassium carbonate in 4 ml of acetonitrile were added 161 mg (0.85 mmol) of 1-(bromomethyl)-4-fluorobenzene and the mixture was stirred under reflux for 1.5 h. After cooling to RT, water was added and the mixture was extracted twice with dichloromethane. The combined organic phases were dried over magnesium sulphate, filtered and concentrated, and the residue was dried in vacuo. 350 mg (65% of theory, purity 89%) of the title compound were obtained.

LC/MS (Method 1, ESIpos): Rₜ=1.50 min, m/z=672 [M+H]⁺.

### Example 22A

### Di-tert-butyl (2-{5-[(3,4-dichlorobenzyl)oxy]-1-benzofur-2-yl}-2-oxoethyl)[2-(4-oxo-1,2,3-benzotriazin-3(4H)-yl)ethyl]malonate

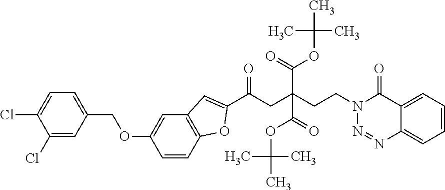 [SMILES: CC(C)(C)OC(=O)C(CCn1nnc2ccccc2c1=O)(CC(=O)c1cc2cc(OCc3ccc(Cl)c(Cl)c3)ccc2o1)C(=O)OC(C)(C)C]

To a solution of 600 mg (1.07 mmol) of the compound from Example 7A in 12 ml of acetonitrile were added at RT 294 mg (2.13 mmol) of potassium carbonate and 312 mg (1.28 mmol, purity 98%) of 1,2-dichloro-4-(chloromethyl)benzene and the mixture was stirred under reflux for 10 h. Then, the mixture was admixed with water. The solid present was filtered off, washed with a small amount of water and dried in vacuo. Stirring the crude product in methanol, renewed filtration and drying in vacuo gave 735 mg (90% of theory, purity 94%) of the title compound.

¹H-NMR (400 MHz, DMSO-d₆): δ [ppm]=8.20 (dd, 1H), 8.11 (d, 1H), 8.03-7.98 (m, 1H), 7.89-7.84 (m, 2H), 7.77-7.75 (m, 1H), 7.68 (d, 1H), 7.65 (d, 1H), 7.49 (dd, 1H), 7.36 (d, 1H), 7.25 (dd, 1H), 5.19 (s, 2H), 4.50-4.41 (m, 2H), 3.65 (s, 2H), 1.40 (s, 18H) [hidden 2H].

LC/MS (Method 1, ESIpos): Rₜ=1.62 min, m/z=722 [M+H]⁺.

### Example 23A

### Di-tert-butyl {2-[5-(heptyloxy)-1-benzofur-2-yl]-2-oxoethyl}[2-(4-oxo-1,2,3-benzotriazin-3(4H)-yl)ethyl]malonate

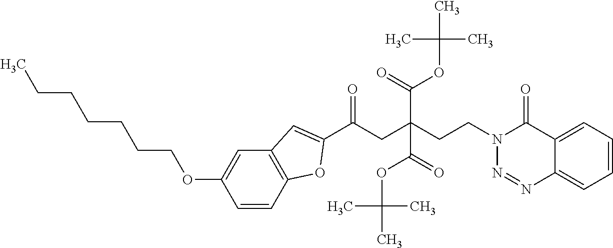 [SMILES: CCCCCCCOc1ccc2oc(C(=O)CC(CCn3nnc4ccccc4c3=O)(C(=O)OC(C)(C)C)C(=O)OC(C)(C)C)cc2c1]

To a solution of 120 mg (0.21 mmol) of the compound from Example 7A in 2.5 ml of acetonitrile were added 59 mg (0.43 mmol) of potassium carbonate and 47 mg (0.26 mmol, purity 98%) of 1-bromoheptane and the mixture was stirred under reflux for 16 h. Then, the mixture was admixed with water. The solid present was filtered off, after-washed with a small amount of water and dried in vacuo. 123 mg (83% of theory, purity 96%) of the title compound were obtained.

LC/MS (Method 1, ESIpos): Rₜ=1.67 min, m/z=662 [M+H]⁺.

### Example 24A

### Di-tert-butyl (2-{5-[(3-carbamoylbenzyl)oxy]-1-benzofur-2-yl}-2-oxoethyl)[2-(4-oxo-1,2,3-benzotriazin-3(4H)-yl)ethyl]malonate

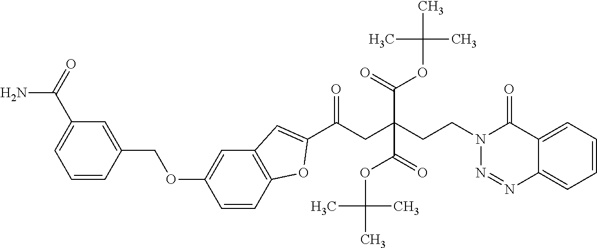 [SMILES: CC(C)(C)OC(=O)C(CCn1nnc2ccccc2c1=O)(CC(=O)c1cc2cc(OCc3cccc(C(N)=O)c3)ccc2o1)C(=O)OC(C)(C)C]

To a mixture of 250 mg (0.44 mmol) of the compound from Example 7A in 5 ml of acetonitrile were added 123 mg (0.89 mmol) of potassium carbonate and 93 mg (0.53 mmol, purity 97%) of 3-(chloromethyl)benzamide and the mixture was stirred under reflux for 16 h. Then, water was added and, after phase separation, the aqueous phase was extracted several times with ethyl acetate. The combined organic phases were dried over sodium sulphate, filtered and concentrated. The residue was purified by preparative HPLC (mobile phase: acetonitrile/water gradient with 0.05% formic acid in the water). 240 mg (77% of theory, purity 99%) of the title compound were obtained.

LC/MS (Method 1, ESIpos): Rₜ=1.29 min, m/z=697 [M+H]⁺.

### Example 25A

### Di-tert-butyl (2-{5-[(4-carbamoylbenzyl)oxy]-1-benzofur-2-yl}-2-oxoethyl)[2-(4-oxo-1,2,3-benzotriazin-3(4H)-yl)ethyl]malonate

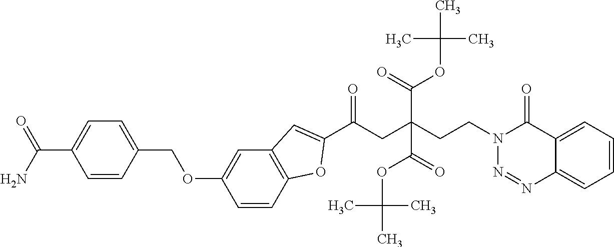 [SMILES: CC(C)(C)OC(=O)C(CCn1nnc2ccccc2c1=O)(CC(=O)c1cc2cc(OCc3ccc(C(N)=O)cc3)ccc2o1)C(=O)OC(C)(C)C]

To a mixture of 262 mg (0.47 mmol) of the compound from Example 7A in 5 ml of acetonitrile were added at RT 128 mg (0.93 mmol) of potassium carbonate and 98 mg (0.56 mmol) of 4-(chloromethyl)benzamide and the mixture was stirred under reflux for 16 h. Then, the mixture was admixed with water. The solid present was filtered off, after-washed with water and dried in vacuo. 204 mg (63% of theory, purity 87%) of the title compound were obtained.

LC/MS (Method 1, ESIpos): Rₜ=1.28 min, m/z=697 [M+H]⁺.

### Example 26A

### (2-{5-[2-(2-Chlorophenyl)ethoxy]-1-benzofur-2-yl}-2-oxoethyl)[2-(4-oxo-1,2,3-benzotriazin-3(4H)-yl)ethyl]malonic acid

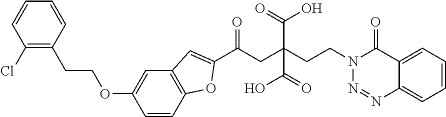 [SMILES: O=C(CC(CCn1nnc2ccccc2c1=O)(C(=O)O)C(=O)O)c1cc2cc(OCCc3ccccc3Cl)ccc2o1]

To a solution of 21 mg (0.03 mmol) of the compound from Example 8A in 1 ml of dichloromethane were added at RT 0.5 ml (6.5 mmol) of trifluoroacetic acid and the reaction mixture was stirred for 1.5 h at RT. Then, the mixture was concentrated and the residue was dried in vacuo. The title compound was obtained in a purity of approx. 90% according to LC/MS and used directly in the subsequent stage.

LC/MS (Method 1, ESIpos): Rₜ=1.13 min, m/z=590 [M+H]⁺.

### Example 27A

### (2-{5-[2-(4-Chlorophenyl)ethoxy]-1-benzofur-2-yl}-2-oxoethyl)[2-(4-oxo-1,2,3-benzotriazin-3(4H)-yl)ethyl]malonic acid

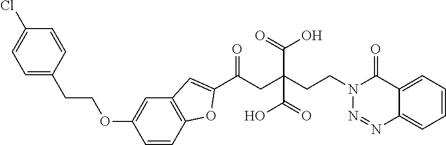 [SMILES: O=C(CC(CCn1nnc2ccccc2c1=O)(C(=O)O)C(=O)O)c1cc2cc(OCCc3ccc(Cl)cc3)ccc2o1]

To a solution of 13 mg (0.018 mmol) of the compound from Example 9A in 1 ml of dichloromethane were added at RT 0.5 ml (6.5 mmol) of trifluoroacetic acid and the reaction mixture was stirred for 1.5 h at RT. Then, the mixture was concentrated and the residue was dried in vacuo. The title compound was obtained in a purity of approx. 90% according to LC/MS and used directly in the subsequent stage.

LC/MS (Method 1, ESIpos): Rₜ=1.12 min, m/z=590 [M+H]⁺.

### Example 28A

### (2-{5-[2-(4-Methoxyphenyl)ethoxy]-1-benzofur-2-yl}-2-oxoethyl)[2-(4-oxo-1,2,3-benzotriazin-3(4H)-yl)ethyl]malonic acid

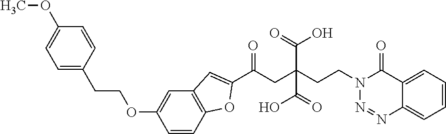 [SMILES: COc1ccc(CCOc2ccc3oc(C(=O)CC(CCn4nnc5ccccc5c4=O)(C(=O)O)C(=O)O)cc3c2)cc1]

To a solution of 105 mg (0.15 mmol) of the compound from Example 10A in 6 ml of dichloromethane were added at RT 3 ml (39.0 mmol) of trifluoroacetic acid and the reaction mixture was stirred for 1.5 h at RT. Then, the mixture was concentrated and the residue was dried in vacuo. The title compound was obtained in a purity of 88% according to LC/MS and used directly in the subsequent stage.

LC/MS (Method 1, ESIpos): Rₜ=1.04 min, m/z=586 [M+H]⁺.

### Example 29A

### (2-{5-[2-(2-Methylphenyl)ethoxy]-1-benzofur-2-yl}-2-oxoethyl)[2-(4-oxo-1,2,3-benzotriazin-3(4H)-yl)ethyl]malonic acid

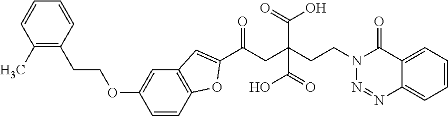 [SMILES: Cc1ccccc1CCOc1ccc2oc(C(=O)CC(CCn3nnc4ccccc4c3=O)(C(=O)O)C(=O)O)cc2c1]

To a solution of 72 mg (0.11 mmol) of the compound from Example 11A in 4 ml of dichloromethane were added at RT 2 ml (26.0 mmol) of trifluoroacetic acid and the reaction mixture was stirred for 1 h at RT. Then, the mixture was concentrated and the residue was dried in vacuo. The title compound was obtained in a purity of 89% according to LC/MS and used directly in the subsequent stage.

LC/MS (Method 1, ESIpos): Rₜ=1.12 min, m/z=570 [M+H]⁺.

### Example 30A

### [2-(4-Oxo-1,2,3-benzotriazin-3(4H)-yl)ethyl]{2-oxo-2-[5-(tetrahydro-2H-pyran-4-ylmethoxy)-1-benzofur-2-yl]ethyl}malonic acid

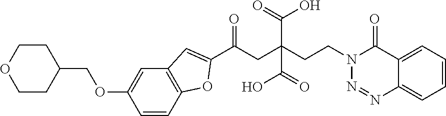 [SMILES: O=C(CC(CCn1nnc2ccccc2c1=O)(C(=O)O)C(=O)O)c1cc2cc(OCC3CCOCC3)ccc2o1]

To a solution of 99 mg (0.15 mmol) of the compound from Example 12A in 4 ml of dichloromethane were added at RT 2 ml (26.0 mmol) of trifluoroacetic acid and the reaction mixture was stirred for 30 h at RT. Then, the mixture was concentrated and the residue was dried in vacuo. The title compound was obtained in a purity of 96% according to LC/MS and used directly in the subsequent stage.

LC/MS (Method 1, ESIpos): Rₜ=0.87 min, m/z=550 [M+H]⁺.

### Example 31A

### [2-(4-Oxo-1,2,3-benzotriazin-3(4H)-yl)ethyl](2-oxo-2-{5-[2-(tetrahydro-2H-pyran-4-yl)ethoxy]-1-benzofur-2-yl}ethyl)malonic acid

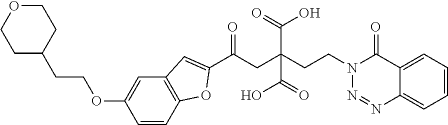 [SMILES: O=C(CC(CCn1nnc2ccccc2c1=O)(C(=O)O)C(=O)O)c1cc2cc(OCCC3CCOCC3)ccc2o1]

To a solution of 98 mg (0.15 mmol) of the compound from Example 13A in 3 ml of dichloromethane were added at RT 1.5 ml (20.0 mmol) of trifluoroacetic acid and the reaction mixture was stirred for 1 h at RT. Then, the mixture was concentrated and the residue was dried in vacuo. The title compound was obtained in a purity of 88% according to LC/MS and used directly in the subsequent stage.

LC/MS (Method 1, ESIpos): Rₜ=0.89 min, m/z=564 [M+H]⁺.

### Example 32A

### {2-[5-(2-Cyclopentylethoxy)-1-benzofur-2-yl]-2-oxoethyl}[2-(4-oxo-1,2,3-benzotriazin-3(4H)-yl)ethyl]malonic acid

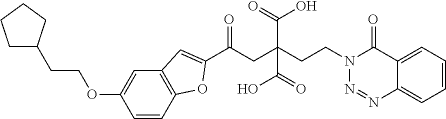 [SMILES: O=C(CC(CCn1nnc2ccccc2c1=O)(C(=O)O)C(=O)O)c1cc2cc(OCCC3CCCC3)ccc2o1]

To a solution of 147 mg (0.22 mmol) of the compound from Example 14A in 4.5 ml of dichloromethane were added at RT 2.3 ml (30.0 mmol) of trifluoroacetic acid and the reaction mixture was stirred for 1 h at RT. Then, the mixture was concentrated and the residue was dried in vacuo. The title compound was obtained in a purity of 65% according to LC/MS and used directly in the subsequent stage.

LC/MS (Method 2, ESIpos): Rₜ=2.48 min, m/z=548 [M+H]⁺.

### Example 33A

### {2-[5-(Cyclohexylmethoxy)-1-benzofur-2-yl]-2-oxoethyl}[2-(4-oxo-1,2,3-benzotriazin-3(4H)-yl)ethyl]malonic acid

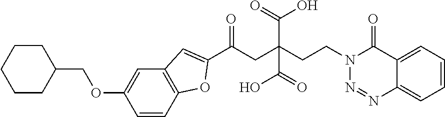 [SMILES: O=C(CC(CCn1nnc2ccccc2c1=O)(C(=O)O)C(=O)O)c1cc2cc(OCC3CCCCC3)ccc2o1]

To a solution of 496 mg (0.75 mmol) of the compound from Example 15A in 4 ml of dichloromethane were added at RT 2.0 ml (26.0 mmol) of trifluoroacetic acid and the reaction mixture was stirred for 3 h at RT. Then, the mixture was concentrated and the residue was dried in vacuo. The title compound was obtained in a purity of 77% according to LC/MS and used directly in the subsequent stage.

LC/MS (Method 1, ESIpos): Rₜ=1.12 min, m/z=548 [M+H]⁺.

### Example 34A

### [2-(4-Oxo-1,2,3-benzotriazin-3(4H)-yl)ethyl]{2-oxo-2-[5-(4,4,4-trifluorobutoxy)-1-benzofur-2-yl]ethyl}malonic acid

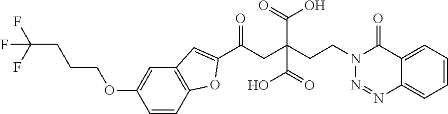 [SMILES: O=C(CC(CCn1nnc2ccccc2c1=O)(C(=O)O)C(=O)O)c1cc2cc(OCCCC(F)(F)F)ccc2o1]

To a solution of 208 mg (0.31 mmol) of the compound from Example 16A in 4 ml of dichloromethane were added at RT 2.0 ml (26.0 mmol) of trifluoroacetic acid and the reaction mixture was stirred for 1 h at RT. Then, the mixture was concentrated and the residue was dried in vacuo. The title compound was obtained in a purity of 95% according to LC/MS and used directly in the subsequent stage.

LC/MS (Method 1, ESIpos): Rₜ=1.04 min, m/z=562 [M+H]⁺.

### Example 35A

### (2-{5-[(4-Fluorotetrahydro-2H-pyran-4-yl)methoxy]-1-benzofur-2-yl}-2-oxoethyl)[2-(4-oxo-1,2,3-benzotriazin-3(4H)-yl)ethyl]malonic acid

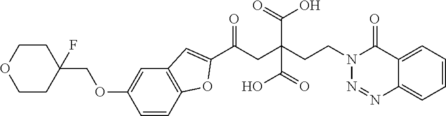 [SMILES: O=C(CC(CCn1nnc2ccccc2c1=O)(C(=O)O)C(=O)O)c1cc2cc(OCC3(F)CCOCC3)ccc2o1]

To a solution of 326 mg (0.48 mmol) of the compound from Example 17A in 3 ml of dichloromethane were added at RT 1.5 ml (20.0 mmol) of trifluoroacetic acid and the reaction mixture was stirred for 1 h at RT. Then, the mixture was concentrated and the residue was dried in vacuo. The title compound was obtained in a purity of 85% according to LC/MS and used directly in the subsequent stage.

LC/MS (Method 1, ESIpos): Rₜ=0.88 min, m/z=568 [M+H]⁺.

### Example 36A

### {2-[5-(3-Cyanopropoxy)-1-benzofur-2-yl]-2-oxoethyl}[2-(4-oxo-1,2,3-benzotriazin-3(4H)-yl)ethyl]malonic acid

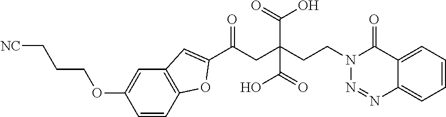 [SMILES: N#CCCCOc1ccc2oc(C(=O)CC(CCn3nnc4ccccc4c3=O)(C(=O)O)C(=O)O)cc2c1]

To a solution of 195 mg (0.31 mmol) of the compound from Example 18A in 4 ml of dichloromethane were added at RT 2 ml (26.0 mmol) of trifluoroacetic acid and the reaction mixture was stirred for 1 h at RT. Then, the mixture was concentrated and the residue was dried in vacuo. The title compound was obtained in a purity of 88% according to LC/MS and used directly in the subsequent stage.

LC/MS (Method 1, ESIpos): Rₜ=0.80 min, m/z=519 [M+H]⁺.

### Example 37A

### {2-[5-(Cyclopentylmethoxy)-1-benzofur-2-yl]-2-oxoethyl}[2-(4-oxo-1,2,3-benzotriazin-3(4H)-yl)ethyl]malonic acid

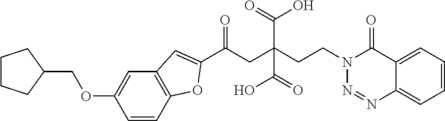 [SMILES: O=C(CC(CCn1nnc2ccccc2c1=O)(C(=O)O)C(=O)O)c1cc2cc(OCC3CCCC3)ccc2o1]

To a solution of 580 mg (0.90 mmol) of the compound from Example 19A in 12 ml of dichloromethane were added at RT 6 ml (78.0 mmol) of trifluoroacetic acid and the reaction mixture was stirred for 1 h at RT. Then, the mixture was concentrated and the residue was dried in vacuo. The title compound was obtained in a purity of 89% according to LC/MS and used directly in the subsequent stage.

LC/MS (Method 1, ESIpos): Rₜ=1.09 min, m/z=534 [M+H]⁺.

### Example 38A

### [2-(4-Oxo-1,2,3-benzotriazin-3(4H)-yl)ethyl]{2-oxo-2-[5-(2-phenylethoxy)-1-benzofur-2-yl]ethyl}malonic acid

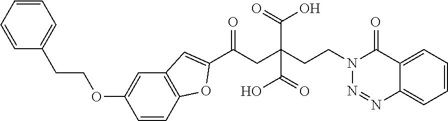 [SMILES: O=C(CC(CCn1nnc2ccccc2c1=O)(C(=O)O)C(=O)O)c1cc2cc(OCCc3ccccc3)ccc2o1]

To a solution of 5.53 g (5.29 mmol) of the compound from Example 20A in 50 ml of dichloromethane were added at RT 25 ml (0.32 mol) of trifluoroacetic acid and the reaction mixture was stirred for 1 h at RT. Then, the mixture was concentrated and the residue was dried in vacuo. The title compound was obtained in a purity of 90% according to LC/MS and used directly in the subsequent stage.

LC/MS (Method 1, ESIpos): Rₜ=1.03 min, m/z=556 [M+H]⁺.

### Example 39A

### (2-{5-[(4-Fluorobenzyl)oxy]-1-benzofur-2-yl}-2-oxoethyl)[2-(4-oxo-1,2,3-benzotriazin-3(4H)-yl)ethyl]malonic acid

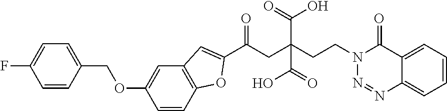 [SMILES: O=C(CC(CCn1nnc2ccccc2c1=O)(C(=O)O)C(=O)O)c1cc2cc(OCc3ccc(F)cc3)ccc2o1]

To a solution of 585 mg (0.83 mmol, purity 95%) of the compound from Example 21A in 10 ml of dichloromethane were added at RT 5 ml (0.65 mol) of trifluoroacetic acid and the reaction mixture was stirred for 1 h at RT. Then, the mixture was concentrated and the residue was dried in vacuo. The title compound was obtained in a purity of 93% according to LC/MS and used directly in the subsequent stage.

LC/MS (Method 1, ESIpos): Rₜ=1.02 min, m/z=560 [M+H]⁺.

### Example 40A

### 1-[6-(Benzyloxy)-1-benzofur-2-yl]ethanone

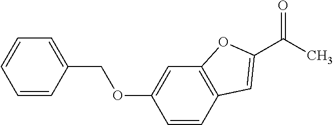 [SMILES: CC(=O)c1cc2ccc(OCc3ccccc3)cc2o1]

To a solution of 97.0 g (0.43 mol) of 4-benzyloxy-2-hydroxybenzaldehyde and 117.5 g (0.85 mol) of potassium carbonate in 200 ml of acetone were added dropwise at RT 42.6 ml (0.53 mol) of chloroacetone. The reaction mixture was stirred under reflux for 1 h. After cooling to RT, solid constituents were filtered off and the filtrate was concentrated. The residue was taken up in dichloromethane and purified by column chromatography (4.5 kg of silica gel, mobile phase: petroleum ether/ethyl acetate 9:1, 130 litres). 87.0 g (77% of theory, purity 100%) of the title compound were obtained.

¹H-NMR (400 MHz, DMSO-d₆): δ [ppm]=7.85 (s, 1H), 7.72 (d, 1H), 7.51-7.31 (m, 6H), 7.07 (dd, 1H), 5.20 (s, 2H), ca. 2.54 (s, 3H, hidden).

LC/MS (Method 1, ESIpos): Rₜ=1.16 min, m/z=267 [M+H]⁺.

### Example 41A

### 1-[6-(Benzyloxy)-1-benzofur-2-yl]-2-bromoethanone

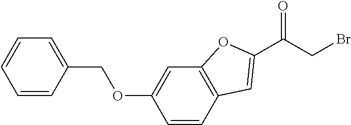 [SMILES: O=C(CBr)c1cc2ccc(OCc3ccccc3)cc2o1]

To a solution of 87.0 g (0.33 mol) of the compound from Example 40A in 1.2 litres of THF under argon were added at RT a total of 129.0 g (0.34 mmol) of phenyltrimethylammonium tribromide in ten equal portions over the course of 15 minutes. The mixture was then stirred at RT for 1 h. Then, the mixture was admixed with 6 litres of 10% strength aqueous sodium chloride solution, 3 litres of ethyl acetate were added and approx. 18% strength (semi-concentrated) hydrochloric acid was used to adjust the pH to 1. After phase separation, the aqueous phase was extracted with ethyl acetate. The combined organic phases were dried over sodium sulphate, filtered and concentrated. The residue was taken up in dichloromethane, absorbed onto silica gel and purified by column chromatography (silica gel, mobile phase: petroleum ether/ethyl acetate 95:5→90:10). 87.0 g (77% of theory, purity 100%) of the title compound were obtained.

LC/MS (Method 1, ESIpos): Rₜ=1.23 min, m/z=345/347 [M+H]⁺.

### Example 42A

### Di-tert-butyl {2-[6-(benzyloxy)-1-benzofur-2-yl]-2-oxoethyl}[2-(4-oxo-1,2,3-benzotriazin-3(4H)-yl)ethyl]malonate

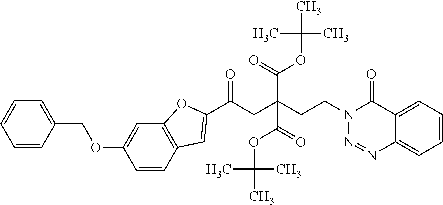 [SMILES: CC(C)(C)OC(=O)C(CCn1nnc2ccccc2c1=O)(CC(=O)c1cc2ccc(OCc3ccccc3)cc2o1)C(=O)OC(C)(C)C]

To a suspension of 0.59 g (14.82 mmol) of sodium hydride (60% in paraffin oil) in 35 ml of DMF under argon were added at RT a solution of 4.65 g (11.95 mmol) of the compound from Example 2A in 35 ml of DMF. After stirring for 30 min at RT, a solution of 3.30 g (9.56 mmol) of the compound from Example 41A in 35 ml of DMF were added quickly and the reaction mixture was stirred for a further 2 h at RT. Then, 500 ml of 10% strength aqueous sodium chloride solution and 250 ml of ethyl acetate were added. After phase separation, the aqueous phase was extracted with 250 ml of ethyl acetate. The combined organic phases were dried over sodium sulphate, filtered and concentrated. The residue was purified by means of column chromatography (250 g of silica gel, mobile phase petroleum ether/ethyl acetate 8:2). 2.8 g (68% of theory, purity 91%) of the title compound were obtained.

LC/MS (Method 1, ESIpos): Rₜ=1.53 min, m/z=654 [M+H]⁺.

### Example 43A

### Di-tert-butyl [2-(6-hydroxy-1-benzofur-2-yl)-2-oxoethyl][2-(4-oxo-1,2,3-benzotriazin-3(4H)-yl)ethyl]malonate

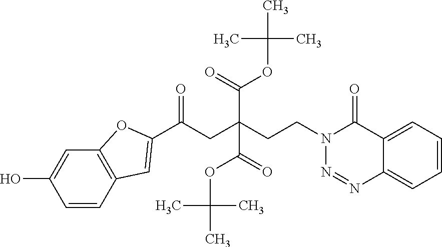 [SMILES: CC(C)(C)OC(=O)C(CCn1nnc2ccccc2c1=O)(CC(=O)c1cc2ccc(O)cc2o1)C(=O)OC(C)(C)C]

A solution of 4.19 g (6.41 mmol) of the compound from Example 42A in 260 ml of a 1:1 mixture of ethyl acetate and ethanol was admixed at RT with 0.34 g (0.32 mmol) of palladium catalyst (10% on activated carbon) and 2.02 g (32.05 mmol) of ammonium formate and stirred for 1 h at 70° C. After cooling to RT, the mixture was filtered through kieselguhr and the filter residue was after-washed with ethanol. The filtrate was concentrated and the residue was dried in vacuo. 3.40 g (94% of theory) of the title compound were obtained.

¹H-NMR (400 MHz, DMSO-d₆): δ [ppm]=10.17 (br. s, 1H), 8.21 (d, 1H), 8.13 (d, 1H), 8.06-7.98 (m, 1H), 7.92-7.82 (m, 2H), 7.60 (d, 1H), 6.96 (s, 1H), 6.87 (dd, 1H), 4.49-4.41 (m, 2H), 3.58 (s, 2H), 1.40 (s, 18H) [hidden 2H].

LC/MS (Method 1, ESIpos): Rₜ=1.26 min, m/z=564 [M+H]⁺.

### Example 44A

### Di-tert-butyl [2-(4-oxo-1,2,3-benzotriazin-3(4H)-yl)ethyl](2-oxo-2-{6-[2-(tetrahydro-2H-pyran-4-yl)ethoxy]-1-benzofur-2-yl}ethyl)malonate

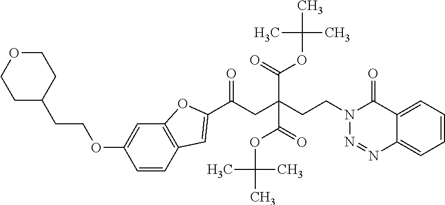 [SMILES: CC(C)(C)OC(=O)C(CCn1nnc2ccccc2c1=O)(CC(=O)c1cc2ccc(OCCC3CCOCC3)cc2o1)C(=O)OC(C)(C)C]

To a mixture of 200 mg (0.36 mmol) of the compound from Example 43A in 0.75 ml of DMF under argon were added, at RT, 45 mg (0.40 mmol) of potassium tert-butoxide and, after stirring for 5 min at RT, 86 mg (0.44 mmol) of 4-(2-bromoethyl)tetrahydro-2H-pyran, dissolved in 0.25 ml of DMF. The mixture was stirred at a bath temperature of 70° C. for 1.5 h. After cooling to RT, in each case 50 ml of water and ethyl acetate were added, and after phase separation, the aqueous phase was extracted once with 50 ml of ethyl acetate. The combined organic phases were dried over sodium sulphate, filtered and concentrated. The residue was taken up in dichloromethane and purified by column chromatography (silica gel, mobile phase: cyclohexane/ethyl acetate 7:3). 82 mg (32% of theory, purity 94%) of the title compound were obtained.

¹H-NMR (400 MHz, CDCl₃): δ [ppm]=8.30 (dd, 1H), 8.08 (d, 1H), 7.89 (td, 1H), 7.77-7.72 (m, 1H), 7.57-7.51 (m, 2H), 7.01 (d, 1H), 6.93 (dd, 1H), 4.60-4.53 (m, 2H), 4.07 (t, 2H), 3.98 (dd, 2H), 3.71 (s, 2H), 3.42 (td, 2H), 2.69-2.63 (m, 2H), 1.84-1.76 (m, 3H), 1.73-1.65 (m, 2H), 1.54-1.30 (m, 2H, hidden), 1.49 (s, 18H).

LC/MS (Method 1, ESIpos): Rₜ=1.48 min, m/z=676 [M+H]⁺.

### Example 45A

### Di-tert-butyl [2-(4-oxo-1,2,3-benzotriazin-3(4H)-yl)ethyl]{2-oxo-2-[6-(pentyloxy)-1-benzofur-2-yl]ethyl}malonate

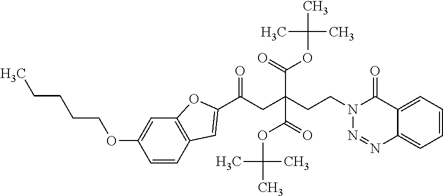 [SMILES: CCCCCOc1ccc2cc(C(=O)CC(CCn3nnc4ccccc4c3=O)(C(=O)OC(C)(C)C)C(=O)OC(C)(C)C)oc2c1]

To a solution of 220 mg (0.39 mmol) of the compound from Example 43A in 4 ml of acetonitrile were added at RT 108 mg (0.78 mmol) of potassium carbonate and 72 mg (0.47 mmol, purity 98%) of 1-bromopentane. The mixture was stirred under reflux overnight. Then, the mixture was admixed with water. The solid present was filtered off and dried in vacuo. 222 mg (90% of theory, purity 100%) of the title compound were obtained.

¹H-NMR (400 MHz, DMSO-d₆): δ [ppm]=8.21 (dd, 1H), 8.14-8.11 (m, 1H), 8.02 (td, 1H), 7.90-7.85 (m, 2H), 7.68 (d, 1H), 7.28 (d, 1H), 6.98 (dd, 1H), 4.49-4.43 (m, 2H), 4.06 (t, 2H), 3.61 (s, 2H), 1.80-1.71 (m, 2H), 1.49-1.30 (m, 6H, partially hidden), 1.40 (s, 18H), 0.91 (t, 3H).

LC/MS (Method 1, ESIpos): Rₜ=1.58 min, m/z=634 [M+H]⁺.

### Example 46A

### Di-tert-butyl [2-(4-oxo-1,2,3-benzotriazin-3(4H)-yl)ethyl][2-oxo-2-(6-propoxy-1-benzofur-2-yl)ethyl]malonate

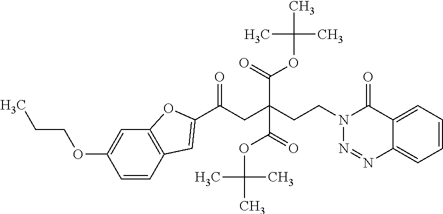 [SMILES: CCCOc1ccc2cc(C(=O)CC(CCn3nnc4ccccc4c3=O)(C(=O)OC(C)(C)C)C(=O)OC(C)(C)C)oc2c1]

To a solution of 110 mg (0.20 mmol) of the compound from Example 43A in 2 ml of acetonitrile were added at RT 53 mg (0.39 mmol) of potassium carbonate and 29 mg (0.23 mmol, purity 98%) of 1-bromopropane. The mixture was stirred under reflux overnight. Then, the mixture was admixed with a further 10 mg (0.08 mmol) of 1-bromopropane and stirred under reflux for a further 4 h. The mixture was then admixed with water. The solid present was filtered off and dried in vacuo. 105 mg (88% of theory, purity 100%) of the title compound were obtained.

¹H-NMR (400 MHz, DMSO-d₆): δ [ppm]=8.21 (dd, 1H), 8.15-8.11 (m, 1H), 8.05-8.00 (m, 1H), 7.90-7.85 (m, 2H), 7.68 (d, 1H), 7.28 (d, 1H), 6.99 (dd, 1H), 4.49-4.43 (m, 2H), 4.03 (t, 2H), 3.61 (s, 2H), 1.81-1.73 (m, 2H), 1.43-1.34 (m, 2H, hidden), 1.40 (s, 18H), 1.01 (t, 3H).

LC/MS (Method 1, ESIpos): Rₜ=1.51 min, m/z=606 [M+H]⁺.

### Example 47A

### Di-tert-butyl [2-(6-butoxy-1-benzofur-2-yl)-2-oxoethyl][2-(4-oxo-1,2,3-benzotriazin-3(4H)-yl)ethyl]malonate

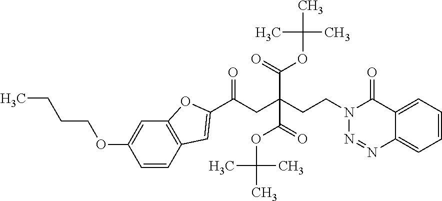 [SMILES: CCCCOc1ccc2cc(C(=O)CC(CCn3nnc4ccccc4c3=O)(C(=O)OC(C)(C)C)C(=O)OC(C)(C)C)oc2c1]

To a solution of 110 mg (0.20 mmol) of the compound from Example 43A in 2 ml of acetonitrile were added at RT 54 mg (0.39 mmol) of potassium carbonate and 33 mg (0.23 mmol, purity 98%) of 1-bromobutane. The mixture was stirred under reflux overnight. Then, the mixture was admixed with water. The solid present was filtered off and dried in vacuo. 101 mg (84% of theory, purity 100%) of the title compound were obtained.

¹H-NMR (400 MHz, DMSO-d₆): δ [ppm]=8.21 (dd, 1H), 8.14-8.11 (m, 1H), 8.05-8.00 (m, 1H), 7.90-7.85 (m, 2H), 7.68 (d, 1H), 7.28 (d, 1H), 6.98 (dd, 1H), 4.49-4.43 (m, 2H), 4.07 (t, 2H), 3.61 (s, 2H), 1.79-1.70 (m, 2H), 1.52-1.36 (m, 4H, partially hidden), 1.40 (s, 18H), 0.95 (t, 3H).

LC/MS (Method 1, ESIpos): Rₜ=1.56 min, m/z=620 [M+H]⁺.

### Example 48A

### Di-tert-butyl {2-[6-(hexyloxy)-1-benzofur-2-yl]-2-oxoethyl}[2-(4-oxo-1,2,3-benzotriazin-3(4H)-yl)ethyl]malonate

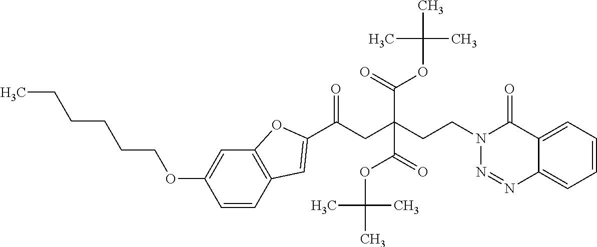 [SMILES: CCCCCCOc1ccc2cc(C(=O)CC(CCn3nnc4ccccc4c3=O)(C(=O)OC(C)(C)C)C(=O)OC(C)(C)C)oc2c1]

To a solution of 110 mg (0.20 mmol) of the compound from Example 43A in 2 ml of acetonitrile were added at RT 54 mg (0.39 mmol) of potassium carbonate and 39 mg (0.23 mmol, purity 98%) of 1-bromohexane. The mixture was stirred under reflux overnight. Then, the mixture was admixed with water. The solid present was filtered off and dried in vacuo. 117 mg (93% of theory, purity 100%) of the title compound were obtained.

¹H-NMR (400 MHz, DMSO-d₆): δ [ppm]=8.21 (dd, 1H), 8.13 (d, 1H), 8.05-7.99 (m, 1H), 7.90-7.85 (m, 2H), 7.67 (d, 1H), 7.28 (d, 1H), 6.98 (dd, 1H), 4.49-4.43 (m, 2H), 4.06 (t, 2H), 3.61 (s, 2H), 1.80-1.70 (m, 2H), 1.49-1.28 (m, 8H, partially hidden), 1.40 (s, 18H), 0.93-0.83 (m, 3H).

LC/MS (Method 1, ESIpos): Rₜ=1.64 min, m/z=648 [M+H]⁺.

### Example 49A

### Di-tert-butyl {2-[6-(heptyloxy)-1-benzofur-2-yl]-2-oxoethyl}[2-(4-oxo-1,2,3-benzotriazin-3(4H)-yl)ethyl]malonate

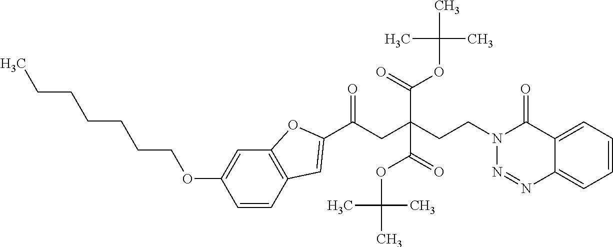 [SMILES: CCCCCCCOc1ccc2cc(C(=O)CC(CCn3nnc4ccccc4c3=O)(C(=O)OC(C)(C)C)C(=O)OC(C)(C)C)oc2c1]

To a solution of 110 mg (0.20 mmol) of the compound from Example 43A in 2 ml of acetonitrile were added at RT 54 mg (0.39 mmol) of potassium carbonate and 43 mg (0.23 mmol, purity 98%) of 1-bromoheptane. The mixture was stirred under reflux overnight. Then, the mixture was admixed with water. The solid present was filtered off and dried in vacuo. 123 mg (95% of theory, purity 100%) of the title compound were obtained.

¹H-NMR (400 MHz, DMSO-d₆): δ [ppm]=8.21 (d, 1H), 8.14-8.11 (m, 1H), 8.05-8.00 (m, 1H), 7.91-7.85 (m, 2H), 7.67 (d, 1H), 7.28 (d, 1H), 6.98 (dd, 1H), 4.49-4.42 (m, 2H), 4.06 (t, 2H), 3.61 (s, 2H), 1.80-1.70 (m, 2H), 1.50-1.22 (m, 10H, partially hidden), 1.40 (s, 18H), 0.92-0.83 (m, 3H).

LC/MS (Method 1, ESIpos): Rₜ=1.67 min, m/z=662 [M+H]⁺.

### Example 50A

### [2-(4-Oxo-1,2,3-benzotriazin-3(4H)-yl)ethyl](2-oxo-2-{6-[2-(tetrahydro-2H-pyran-4-yl)ethoxy]-1-benzofur-2-yl}ethyl)malonic acid

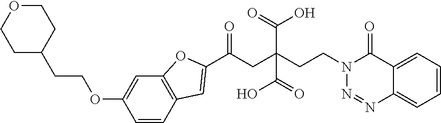 [SMILES: O=C(CC(CCn1nnc2ccccc2c1=O)(C(=O)O)C(=O)O)c1cc2ccc(OCCC3CCOCC3)cc2o1]

To a solution of 71 mg (0.11 mmol) of the compound from Example 44A in 2 ml of dichloromethane was added at RT 1 ml (14.0 mmol) of trifluoroacetic acid and the reaction mixture was stirred for 1 h at RT. Then, the mixture was concentrated and the residue was dried in vacuo. The title compound was obtained in a purity of 88% according to LC/MS and used directly in the subsequent stage.

LC/MS (Method 1, ESIpos): Rₜ=0.90 min, m/z=564 [M+H]⁺.

### Example 51A

### 1-(6-Hydroxy-1-benzofur-3-yl)ethanone

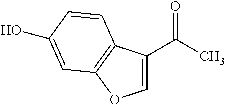

To a solution of 113.0 g (0.45 mol) of 3-(2,4-dimethoxyphenyl)-4-(dimethylamino)but-3-en-2-one in 750 ml of dichloromethane were slowly added dropwise 199.0 g (0.79 mol) of tribromoborane at 0° C. The mixture was then stirred at 0° C. for 4 h. Then, the mixture was admixed with saturated aqueous sodium hydrogencarbonate solution, and, after phase separation, the aqueous phase was extracted three times with ethyl acetate. The combined organic phases were dried over sodium sulphate, filtered and concentrated. The residue was purified by column chromatography (silica gel, mobile phase dichloromethane/methanol 95:5). 45.0 g (53% of theory, purity 95%) of the title compound were obtained.

¹H-NMR (400 MHz, DMSO-d₆): δ [ppm]=9.77 (s, 1H), 8.81 (s, 1H), 7.84 (d, 1H), 6.99 (d, 1H), 6.85 (dd, 1H), 2.48 (s, 3H).

LC/MS (Method 1, ESIpos): Rₜ=0.70 min, m/z=177 [M+H]⁺.

### Example 52A

### 1-[6-(Benzyloxy)-1-benzofur-3-yl]ethanone

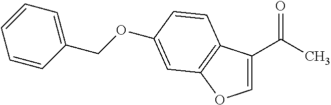 [SMILES: CC(=O)c1coc2cc(OCc3ccccc3)ccc12]

To a mixture of 159.0 g (0.90 mol) of the compound from Example 51A in 2.1 litres of acetonitrile were added at RT 249.5 g (1.81 mol) of potassium carbonate and 185.2 g (1.08 mol) of benzyl bromide and the mixture was stirred under reflux for 4 h. Then, a further 23.1 g (0.13 mol) of benzyl bromide were added and the mixture was again stirred under reflux for 4 h. After cooling to RT, the mixture was filtered and the filtrate was concentrated. The residue was admixed with saturated aqueous sodium chloride solution and extracted three times with ethyl acetate. The combined organic phases were dried over sodium sulphate, filtered and concentrated. The residue was purified by column chromatography (silica gel, mobile phase petroleum ether/ethyl acetate 8:2). 206.0 g (79% of theory, purity 79%) of the title compound were obtained.

¹H-NMR (400 MHz, DMSO-d₆): δ [ppm]=8.90 (s, 1H), 7.98-7.91 (m, 1H), 7.50-7.45 (m, 2H), 7.43-7.31 (m, 4H), 7.08 (dd, 1H), 5.17 (s, 2H), 2.50 (s, 3H, partially hidden).

LC/MS (Method 1, ESIpos): Rₜ=1.16 min, m/z=267 [M+H]⁺.

### Example 53A

### 1-[6-(Benzyloxy)-1-benzofur-3-yl]-2-bromoethanone

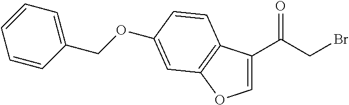 [SMILES: O=C(CBr)c1coc2cc(OCc3ccccc3)ccc12]

To a solution of 135.0 g (0.51 mol) of the compound from Example 52A in 1.9 litres of THF under argon were added at RT a total of 200.0 g (0.53 mol) of phenyltrimethylammonium tribromide in ten portions over the course of 15 minutes. After stirring for 1 h at RT, the mixture was admixed with 10% strength aqueous sodium chloride solution. Ethyl acetate was added, and approx. 18% strength (semi-concentrated) hydrochloric acid was used to adjust the pH to 1. After phase separation, the aqueous phase was extracted once with ethyl acetate. The combined organic phases were dried over sodium sulphate, filtered and concentrated. The crude product was taken up in dichloromethane, absorbed onto silica gel and purified by column chromatography (silica gel, mobile phase: petroleum ether/ethyl acetate 95:5→90:10). 30 g (17% of theory) of a pure fraction of the title compound were obtained, as were 50 g (6% of theory) of a mixed fraction in which the title compound was present in an amount of 22% according to LC/MS.

LC/MS (Method 1, ESIpos): Rₜ=1.25 min, m/z=345/347 [M+H]⁺.

In an experiment carried out analogously, 2.97 g (11.15 mmol) of the compound from Example 52A and 4.23 g (11.26 mmol) of phenyltrimethylammonium tribromide were used. In this case, the crude product was stirred with methanol, filtered off, after-washed with methanol and dried. This gave 3.17 g (74% of theory; purity 88%) of the title compound.

¹H-NMR (400 MHz, DMSO-d₆): δ [ppm]=9.01 (s, 1H), 7.93 (d, 1H), 7.53-7.30 (m, 6H), 7.12 (dd, 1H), 5.18 (s, 2H), 4.76 (s, 2H).

LC/MS (Method 1, ESIpos): Rₜ=1.24 min, m/z=345/347 [M+H]⁺.

### Example 54A

### Di-tert-butyl {2-[6-(benzyloxy)-1-benzofur-3-yl]-2-oxoethyl}[2-(4-oxo-1,2,3-benzotriazin-3(4H)-yl)ethyl]malonate

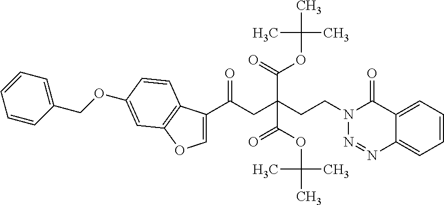 [SMILES: CC(C)(C)OC(=O)C(CCn1nnc2ccccc2c1=O)(CC(=O)c1coc2cc(OCc3ccccc3)ccc12)C(=O)OC(C)(C)C]

To a mixture of 28.21 g (72.42 mmol) of the compound from Example 2A in 440 ml of DMF under argon were added at RT 8.45 g (75.32 mmol) of potassium tert-butoxide. After stirring for 30 min at RT, a solution of 20.0 g (57.94 mmol) of the compound from Example 53A in 220 ml of DMF were added quickly and the reaction mixture was stirred for a further hour at RT. Then, the mixture was admixed with 4 litres of 10% strength aqueous sodium chloride solution and 1.2 litres of ethyl acetate. After phase separation, the aqueous phase was extracted once with 1.2 litres of ethyl acetate. The combined organic phases were dried over sodium sulphate, filtered and concentrated. The residue was taken up in dichloromethane/petroleum ether and purified by column chromatography (2 kg of silica gel, mobile phase 40 litres of petroleum ether/ethyl acetate 8:2). 25.0 g (56% of theory, purity 85%) of the title compound were obtained.

¹H-NMR (400 MHz, DMSO-d₆): δ [ppm]=9.01 (s, 1H), 8.21-8.16 (m, 1H), 8.13-8.09 (m, 1H), 8.03-7.98 (m, 1H), 7.90-7.83 (m, 2H), 7.51-7.31 (m, 6H), 7.06 (dd, 1H), 5.17 (s, 2H), 4.51-4.43 (m, 2H), 3.60 (s, 2H), 1.41 (s, 18H) [hidden 2H].

LC/MS (Method 1, ESIpos): Rₜ=1.52 min, m/z=654 [M+H]⁺.

### Example 55A

### Di-tert-butyl [2-(6-hydroxy-1-benzofur-3-yl)-2-oxoethyl][2-(4-oxo-1,2,3-benzotriazin-3(4H)-yl)ethyl]malonate

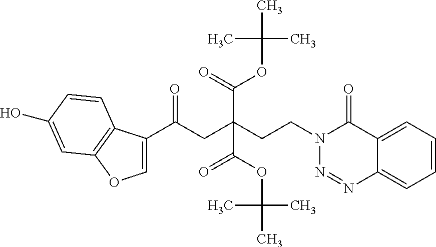 [SMILES: CC(C)(C)OC(=O)C(CCn1nnc2ccccc2c1=O)(CC(=O)c1coc2cc(O)ccc12)C(=O)OC(C)(C)C]

A solution of 31.30 g (47.88 mmol) of the compound from Example 54A in 1.9 litres of a 1:1 mixture of ethyl acetate and ethanol was admixed at RT with 2.55 g (2.39 mmol) of palladium catalyst (10% on activated carbon) and 15.09 g (0.24 mol) of ammonium formate and stirred for 1 h at 70° C. After cooling to RT, the mixture was filtered through kieselguhr and the filter residue was after-washed with ethanol. The filtrate was concentrated and the residue was purified by column chromatography (silica gel, mobile phase dichloromethane/methanol 95:5). The product-containing fractions were combined and concentrated. The residue was stirred in 250 ml of diethyl ether, filtered off and dried in vacuo. 17.50 g (59% of theory, purity approx. 90% according to ¹H-NMR) of the title compound were obtained.

¹H-NMR (400 MHz, DMSO-d₆): δ [ppm]=9.79 (s, 1H), 8.93 (s, 1H), 8.20 (d, 1H), 8.16-8.11 (m, 1H), 8.07-8.01 (m, 1H), 7.91-7.85 (m, 1H), 7.77 (d, 1H), 6.98 (d, 1H), 6.84 (dd, 1H), 4.50-4.42 (m, 2H), 3.58 (s, 2H), 2.55-2.46 (2H, hidden), 1.41 (s, 18H).

LC/MS (Method 1, ESIpos): Rₜ=1.27 min, m/z=564 [M+H]⁺.

### Example 56A

### Di-tert-butyl [2-(4-oxo-1,2,3-benzotriazin-3(4H)-yl)ethyl](2-oxo-2-{6-[2-(tetrahydro-2H-pyran-4-yl)ethoxy]-1-benzofur-3-yl}ethyl)malonate

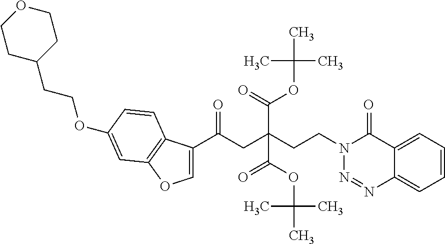 [SMILES: CC(C)(C)OC(=O)C(CCn1nnc2ccccc2c1=O)(CC(=O)c1coc2cc(OCCC3CCOCC3)ccc12)C(=O)OC(C)(C)C]

To a mixture of 200 mg (0.36 mmol) of the compound from Example 55A in 0.75 ml of DMF under argon were added, at RT, 45 mg (0.40 mmol) of potassium tert-butoxide and, after stirring for 5 min at RT, 86 mg (0.44 mmol) of 4-(2-bromoethyl)tetrahydro-2H-pyran, dissolved in 0.25 ml of DMF. The mixture was stirred at a bath temperature of 70° C. for 1.5 h. After cooling to RT, in each case 50 ml of water and ethyl acetate were added, and after phase separation, the aqueous phase was extracted once with 50 ml of ethyl acetate. The combined organic phases were dried over sodium sulphate, filtered and concentrated. The residue was taken up in dichloromethane and purified by column chromatography (40 g of silica gel, mobile phase cyclohexane/ethyl acetate 7:3). 158 mg (63% of theory, purity 95%) of the title compound were obtained.

¹H-NMR (400 MHz, CDCl₃): δ [ppm]=8.31-8.27 (m, 2H), 8.08 (d, 1H), 8.02 (d, 1H), 7.91-7.86 (m, 1H), 7.77-7.71 (m, 1H), 6.99 (d, 1H), 6.94 (dd, 1H), 4.59-4.53 (m, 2H), 4.05 (t, 2H), 3.98 (dd, 2H), 3.64 (s, 2H), 3.42 (td, 2H), 2.73-2.67 (m, 2H), 1.88-1.74 (m, 3H), 1.72-1.64 (m, 2H), 1.47 (s, 18H), 1.41-1.33 (m, 2H).

LC/MS (Method 1, ESIpos): Rₜ=1.49 min, m/z=676 [M+H]⁺.

### Example 57A

### Di-tert-butyl {2-[6-(2-cyclopentylethoxy)-1-benzofur-3-yl]-2-oxoethyl}[2-(4-oxo-1,2,3-benzotriazin-3(4H)-yl)ethyl]malonate

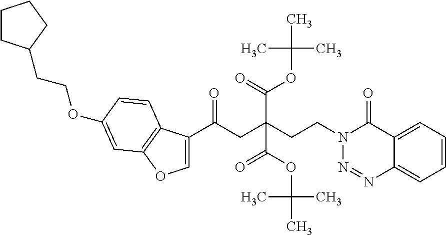 [SMILES: CC(C)(C)OC(=O)C(CCn1nnc2ccccc2c1=O)(CC(=O)c1coc2cc(OCCC3CCCC3)ccc12)C(=O)OC(C)(C)C]

To a mixture of 200 mg (0.36 mmol) of the compound from Example 55A in 0.75 ml of DMF under argon were added at RT 45 mg (0.40 mmol) of potassium tert-butoxide and, after stirring for 5 min at RT, 99 mg (0.44 mmol) of (2-iodoethyl)cyclopentane, dissolved in 0.25 ml of DMF. The mixture was stirred at a bath temperature of 70° C. for 1 h. After cooling to RT, in each case 50 ml of water and ethyl acetate were added, and after phase separation, the aqueous phase was extracted once with 50 ml of ethyl acetate. The combined organic phases were washed once with saturated sodium chloride solution, dried over sodium sulphate, filtered and concentrated. The residue was taken up in dichloromethane and purified by column chromatography (40 g of silica gel, mobile phase: cyclohexane/ethyl acetate 85:15). 178 mg (73% of theory, purity 95%) of the title compound were obtained.

¹H-NMR (400 MHz, CDCl₃): δ [ppm]=8.30-8.25 (m, 2H), 8.07 (d, 1H), 8.00 (d, 1H), 7.91-7.84 (m, 1H), 7.76-7.70 (m, 1H), 6.99 (d, 1H), 6.94 (dd, 1H), 4.60-4.53 (m, 2H), 4.01 (t, 2H), 3.64 (s, 2H), 2.74-2.67 (m, 2H), 2.06-1.92 (m, 1H), 1.90-1.79 (m, 4H), 1.70-1.50 (m, 4H), 1.47 (s, 18H), 1.24-1.11 (m, 2H).

LC/MS (Method 1, ESIpos): Rₜ=1.69 min, m/z=660 [M+H]⁺.

### Example 58A

### Di-tert-butyl {2-[6-(2-cyclohexylethoxy)-1-benzofur-3-yl]-2-oxoethyl}[2-(4-oxo-1,2,3-benzotriazin-3(4H)-yl)ethyl]malonate

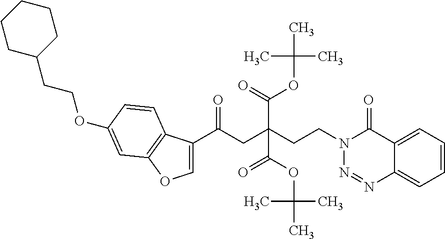 [SMILES: CC(C)(C)OC(=O)C(CCn1nnc2ccccc2c1=O)(CC(=O)c1coc2cc(OCCC3CCCCC3)ccc12)C(=O)OC(C)(C)C]

To a mixture of 300 mg (0.53 mmol) of the compound from Example 55A in 3 ml of acetonitrile were added at RT 221 mg (1.60 mmol) of potassium carbonate, followed by 306 mg (1.60 mmol) of 2-(bromoethyl)cyclohexane and the mixture was stirred under reflux for 4 h. After cooling to RT, the mixture was admixed with water and extracted twice with ethyl acetate. The combined organic phases were washed once with saturated sodium chloride solution, dried over magnesium sulphate, filtered and concentrated. The residue was purified by means of column chromatography (40 g of silica gel, mobile phase: cyclohexane/ethyl acetate 85:15). 140 mg (37% of theory, purity 96%) of the title compound were obtained.

LC/MS (Method 1, ESIpos): Rₜ=1.71 min, m/z=674 [M+H]⁺.

### Example 59A

### Di-tert-butyl (2-{6-[(3-chlorobenzyl)oxy]-1-benzofur-3-yl}-2-oxoethyl)[2-(4-oxo-1,2,3-benzotriazin-3(4H)-yl)ethyl]malonate

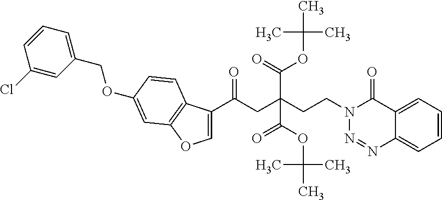 [SMILES: CC(C)(C)OC(=O)C(CCn1nnc2ccccc2c1=O)(CC(=O)c1coc2cc(OCc3cccc(Cl)c3)ccc12)C(=O)OC(C)(C)C]

To a mixture of 200 mg (0.36 mmol) of the compound from Example 55A in 2 ml of acetonitrile were added at RT 147 mg (1.07 mmol) of potassium carbonate, followed by 146 mg (0.71 mmol) of 1-(bromomethyl)-3-chlorobenzene and the mixture was stirred under reflux for 2 h. Then, a further 58 mg (0.28 mmol) of 1-(bromomethyl)-3-chlorobenzene were added and the mixture was stirred under reflux for a further hour. After cooling to RT, the mixture was admixed with water and extracted twice with ethyl acetate. The combined organic phases were washed once with saturated sodium chloride solution, dried over magnesium sulphate, filtered and concentrated. The residue was purified by means of column chromatography (40 g of silica gel, mobile phase: cyclohexane/ethyl acetate 7:3). 137 mg (53% of theory, purity 94%) of the title compound were obtained.

LC/MS (Method 1, ESIpos): Rₜ=1.58 min, m/z=688 [M+H]⁺.

### Example 60A

### Di-tert-butyl {2-[6-(cyclopentylmethoxy)-1-benzofur-3-yl]-2-oxoethyl}[2-(4-oxo-1,2,3-benzotriazin-3(4H)-yl)ethyl]malonate

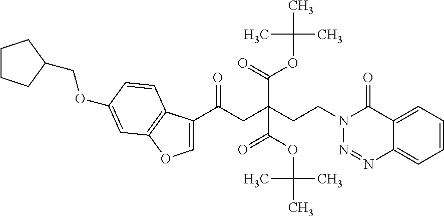 [SMILES: CC(C)(C)OC(=O)C(CCn1nnc2ccccc2c1=O)(CC(=O)c1coc2cc(OCC3CCCC3)ccc12)C(=O)OC(C)(C)C]

To a mixture of 600 mg (1.07 mmol) of the compound from Example 55A in 6 ml of acetonitrile were added at RT 441 mg (3.19 mmol) of potassium carbonate, followed by 521 mg (3.19 mmol) of (bromomethyl)cyclopentane and the mixture was stirred under reflux for 20 h. After cooling to RT, the mixture was admixed with water and extracted twice with ethyl acetate. The combined organic phases were dried over magnesium sulphate, filtered and concentrated. The residue was purified by means of column chromatography (80 g of silica gel, mobile phase: cyclohexane/ethyl acetate 7:3). 467 mg (64% of theory, purity 94%) of the title compound were obtained.

LC/MS (Method 1, ESIpos): Rₜ=1.60 min, m/z=646 [M+H]⁺.

### Example 61A

### Di-tert-butyl {2-[6-(cyclohexylmethoxy)-1-benzofur-3-yl]-2-oxoethyl}[2-(4-oxo-1,2,3-benzotriazin-3(4H)-yl)ethyl]malonate

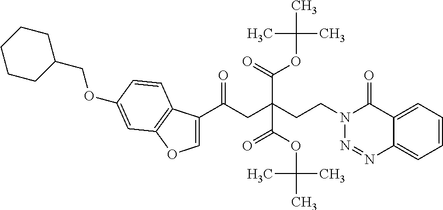 [SMILES: CC(C)(C)OC(=O)C(CCn1nnc2ccccc2c1=O)(CC(=O)c1coc2cc(OCC3CCCCC3)ccc12)C(=O)OC(C)(C)C]

To a mixture of 600 mg (1.07 mmol) of the compound from Example 55A in 3 ml of DMF under argon were added at RT 134 mg (1.20 mmol) of potassium tert-butoxide and, after stirring for 5 min at RT, 236 mg (1.33 mmol) of (bromomethyl)cyclohexane. The mixture was stirred at a bath temperature of 70° C. for 2 h. Then, a further 141 mg (0.80 mmol) of (bromomethyl)cyclohexane were added and the mixture was stirred at a bath temperature of 70° C. for 2 h. Then, a further 72 mg (0.64 mmol) of potassium tert-butoxide were added, and the mixture was again stirred at a bath temperature of 70° C. for 2 h. After cooling to RT, in each case 50 ml of water and ethyl acetate were added, and after phase separation, the aqueous phase was extracted once with 50 ml of ethyl acetate. The combined organic phases were washed once with saturated sodium chloride solution, dried over sodium sulphate, filtered and concentrated. The residue was taken up in dichloromethane and purified by column chromatography (80 g of silica gel, mobile phase: cyclohexane/ethyl acetate 85:15). 383 mg (51% of theory, purity 94%) of the title compound were obtained.

LC/MS (Method 1, ESIpos): Rₜ=1.67 min, m/z=660 [M+H]⁺.

### Example 62A

### Di-tert-butyl [2-(4-oxo-1,2,3-benzotriazin-3(4H)-yl)ethyl][2-oxo-2-(6-propoxy-1-benzofur-3-yl)ethyl]malonate

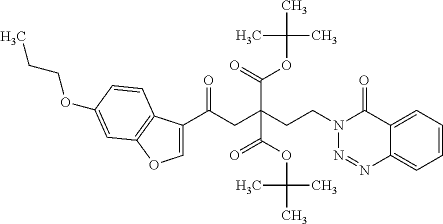 [SMILES: CCCOc1ccc2c(C(=O)CC(CCn3nnc4ccccc4c3=O)(C(=O)OC(C)(C)C)C(=O)OC(C)(C)C)coc2c1]

To a mixture of 160 mg (0.27 mmol purity 95%) of the compound from Example 55A in 3 ml of acetonitrile were added at RT 75 mg (0.54 mmol) of potassium carbonate and 40 mg (0.32 mmol) of 1-bromopropane and the mixture was stirred under reflux for 3 h. Then, a further 40 mg (0.32 mmol) of 1-bromopropane were added, and the mixture was stirred under reflux overnight. Then, a further 40 mg (0.32 mmol) of 1-bromopropane were added and the mixture was stirred under reflux for a further 3 h. Then, a further 40 mg (0.32 mmol) of 1-bromopropane were added and the mixture was stirred under reflux for a further 5 h. After cooling to RT, the mixture was admixed with water, and the aqueous phase was extracted three times with ethyl acetate. The combined organic phases were dried over sodium sulphate, filtered and concentrated. The residue was purified by preparative HPLC (mobile phase: acetonitrile/water gradient with 0.05% formic acid in the water). 115 mg (70% of theory, purity 99%) of the title compound were obtained.

LC/MS (Method 1, ESIpos): Rₜ=1.59 min, m/z=606 [M+H]⁺.

### Example 63A

### Di-tert-butyl [2-(4-oxo-1,2,3-benzotriazin-3(4H)-yl)ethyl]{2-oxo-2-[6-(pentyloxy)-1-benzofur-3-yl]ethyl}malonate

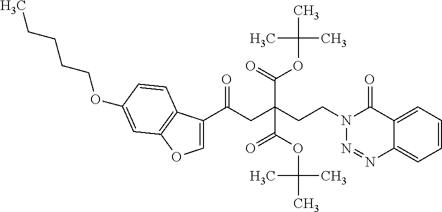 [SMILES: CCCCCOc1ccc2c(C(=O)CC(CCn3nnc4ccccc4c3=O)(C(=O)OC(C)(C)C)C(=O)OC(C)(C)C)coc2c1]

To a mixture of 160 mg (0.27 mmol, purity 95%) of the compound from Example 55A in 3 ml of acetonitrile were added at RT 75 mg (0.54 mmol) of potassium carbonate and 49 mg (0.32 mmol) of 1-bromopentane. The mixture was stirred under reflux overnight. Then, a further 49 mg (0.32 mmol) of 1-bromopentane were added and the mixture was stirred under reflux for a further 3 h. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 125 mg (73% of theory, purity 100%) of the title compound were obtained.

LC/MS (Method 2, ESIpos): Rₜ=3.26 min, m/z=634 [M+H]⁺.

### Example 64A

### Di-tert-butyl (2-{6-[(3,4-dichlorobenzyl)oxy]-1-benzofur-3-yl}-2-oxoethyl)[2-(4-oxo-1,2,3-benzotriazin-3(4H)-yl)ethyl]malonate

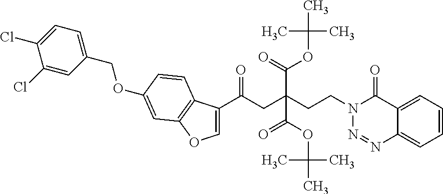 [SMILES: CC(C)(C)OC(=O)C(CCn1nnc2ccccc2c1=O)(CC(=O)c1coc2cc(OCc3ccc(Cl)c(Cl)c3)ccc12)C(=O)OC(C)(C)C]

To a mixture of 160 mg (0.27 mmol, purity 95%) of the compound from Example 55A in 3 ml of acetonitrile were added at RT 75 mg (0.54 mmol) of potassium carbonate and 80 mg (0.32 mmol, purity 97%) of 3,4-dichlorobenzyl bromide. The mixture was stirred under reflux overnight. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 109 mg (55% of theory, purity 98%) of the title compound were obtained.

LC/MS (Method 1, ESIpos): Rₜ=1.63 min, m/z=722 [M+H]⁺.

### Example 65A

### Di-tert-butyl [2-(4-oxo-1,2,3-benzotriazin-3(4H)-yl)ethyl]{2-oxo-2-[6-(tetrahydro-2H-pyran-4-ylmethoxy)-1-benzofur-3-yl]ethyl}malonate

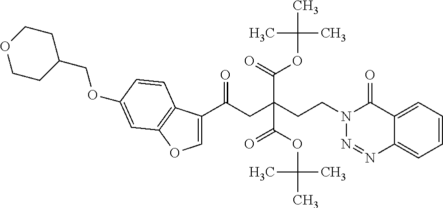 [SMILES: CC(C)(C)OC(=O)C(CCn1nnc2ccccc2c1=O)(CC(=O)c1coc2cc(OCC3CCOCC3)ccc12)C(=O)OC(C)(C)C]

To a mixture of 200 mg (0.36 mmol) of the compound from Example 55A in 4 ml of acetonitrile were added at RT 98 mg (0.71 mmol) of potassium carbonate and 79 mg (0.43 mmol, purity 97%) of 4-(bromomethyl)tetrahydro-2H-pyran. The mixture was stirred under reflux overnight. Then, a further 79 mg (0.43 mmol, purity 97%) of 4-(bromomethyl)tetrahydro-2H-pyran were added, and the mixture was stirred under reflux once again for 24 h. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 89 mg (34% of theory, purity 98%) of the title compound were obtained.

LC/MS (Method 1, ESIpos): Rₜ=1.46 min, m/z=662 [M+H]⁺.

### Example 66A

### Di-tert-butyl {2-[6-(hexyloxy)-1-benzofur-3-yl]-2-oxoethyl}[2-(4-oxo-1,2,3-benzotriazin-3(4H)-yl)ethyl]malonate

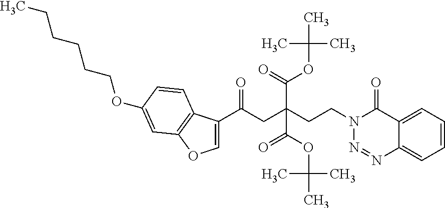 [SMILES: CCCCCCOc1ccc2c(C(=O)CC(CCn3nnc4ccccc4c3=O)(C(=O)OC(C)(C)C)C(=O)OC(C)(C)C)coc2c1]

To a mixture of 160 mg (0.27 mmol, purity 95%) of the compound from Example 55A in 3 ml of acetonitrile were added at RT 75 mg (0.54 mmol) of potassium carbonate and 56 mg (0.32 mmol, purity 98%) of 1-bromohexane. The mixture was stirred under reflux overnight. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 127 mg (73% of theory, purity 100%) of the title compound were obtained.

LC/MS (Method 2, ESIpos): Rₜ=3.34 min, m/z=648 [M+H]⁺.

### Example 67A

### Di-tert-butyl {2-[6-(heptyloxy)-1-benzofur-3-yl]-2-oxoethyl}[2-(4-oxo-1,2,3-benzotriazin-3(4H)-yl)ethyl]malonate

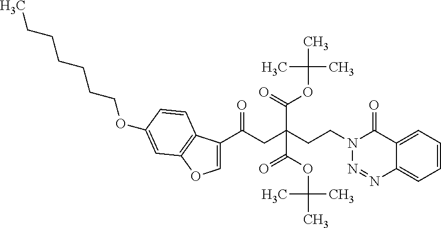 [SMILES: CCCCCCCOc1ccc2c(C(=O)CC(CCn3nnc4ccccc4c3=O)(C(=O)OC(C)(C)C)C(=O)OC(C)(C)C)coc2c1]

To a mixture of 177 mg (0.30 mmol, purity 95%) of the compound from Example 55A in 3.3 ml of acetonitrile were added at RT 83 mg (0.60 mmol) of potassium carbonate and 64 mg (0.36 mmol) of 1-bromoheptane. The mixture was stirred under reflux overnight. After cooling to RT, the mixture was admixed with water, and the aqueous phase was extracted three times with ethyl acetate. The combined organic phases were dried over sodium sulphate, filtered and concentrated.

The residue was taken up in THF and purified by preparative HPLC (mobile phase: acetonitrile/water gradient with 0.05% formic acid in the water). 183 mg (90% of theory, purity 97%) of the title compound were obtained.

LC/MS (Method 2, ESIpos): Rₜ=3.42 min, m/z=662 [M+H]⁺.

### Example 68A

### Di-tert-butyl [2-(6-isobutoxy-1-benzofur-3-yl)-2-oxoethyl][2-(4-oxo-1,2,3-benzotriazin-3(4H)-yl)ethyl]malonate

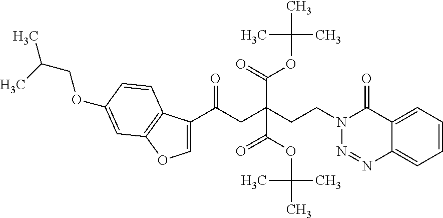 [SMILES: CC(C)COc1ccc2c(C(=O)CC(CCn3nnc4ccccc4c3=O)(C(=O)OC(C)(C)C)C(=O)OC(C)(C)C)coc2c1]

To a mixture of 160 mg (0.27 mmol, purity 95%) of the compound from Example 55A in 3 ml of acetonitrile were added at RT 75 mg (0.54 mmol) of potassium carbonate and 44 mg (0.32 mmol) of 1-bromo-2-methylpropane and the mixture was stirred under reflux for 1 h. Then, a further 44 mg (0.32 mmol) of 1-bromo-2-methylpropane were added, and the mixture was stirred under reflux overnight. Then, a further 63 mg (0.46 mmol) of 1-bromo-2-methylpropane were added, and the mixture was stirred under reflux for a further 4 h. Then, once again 63 mg (0.46 mmol) of 1-bromo-2-methylpropane were added, and the mixture was again stirred under reflux overnight. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 85 mg (50% of theory, purity 99%) of the title compound were obtained.

LC/MS (Method 1, ESIpos): Rₜ=1.56 min, m/z=620 [M+H]⁺.

### Example 69A

### Di-tert-butyl [2-(6-butoxy-1-benzofur-3-yl)-2-oxoethyl][2-(4-oxo-1,2,3-benzotriazin-3(4H)-yl)ethyl]malonate

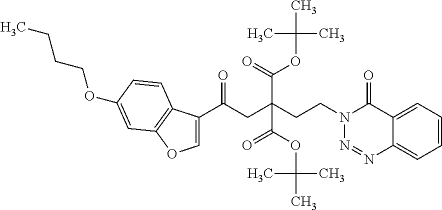 [SMILES: CCCCOc1ccc2c(C(=O)CC(CCn3nnc4ccccc4c3=O)(C(=O)OC(C)(C)C)C(=O)OC(C)(C)C)coc2c1]

To a mixture of 160 mg (0.27 mmol, purity 95%) of the compound from Example 55A in 3 ml of acetonitrile were added at RT 75 mg (0.54 mmol) of potassium carbonate and 44 mg (0.32 mmol) of 1-bromobutane. The mixture was stirred under reflux for 3 h. Then, a further 44 mg (0.32 mmol) of 1-bromobutane were added, and the mixture was stirred under reflux overnight. Then, once again 44 mg (0.32 mmol) of 1-bromobutane were added and the mixture was stirred under reflux for a further 5 h. After cooling to RT, the mixture was admixed with water, and the aqueous phase was extracted three times with ethyl acetate. The combined organic phases were dried over sodium sulphate, filtered and concentrated. The residue was taken up in THF and purified by preparative HPLC (mobile phase: acetonitrile/water gradient with 0.05% formic acid in the water). 41 mg (24% of theory, purity 99%) of the title compound were obtained.

LC/MS (Method 1, ESIpos): Rₜ=1.59 min, m/z=620 [M+H]⁺.

### Example 70A

### Di-tert-butyl [2-(4-oxo-1,2,3-benzotriazin-3(4H)-yl)ethyl]{2-oxo-2-[6-(2-phenylethoxy)-1-benzofur-3-yl]ethyl}malonate

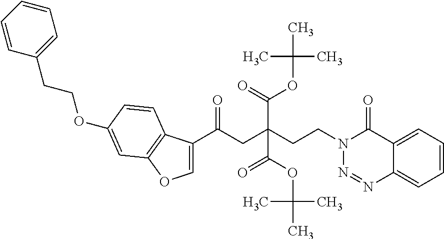 [SMILES: CC(C)(C)OC(=O)C(CCn1nnc2ccccc2c1=O)(CC(=O)c1coc2cc(OCCc3ccccc3)ccc12)C(=O)OC(C)(C)C]

To a mixture of 200 mg (0.30 mmol, purity 86%) of the compound from Example 55A in 3.4 ml of acetonitrile were added at RT 84 mg (0.61 mmol) of potassium carbonate and 69 mg (0.37 mmol, purity 98%) of (2-bromoethyl)benzene. The mixture was stirred under reflux overnight. Then, once again 69 mg (0.37 mmol; purity 98%) of (2-bromoethyl)benzene were added, and the mixture was stirred under reflux for a further four days. After cooling to RT, the mixture was admixed with water and then completely concentrated. The residue was taken up in dichloromethane and filtered over silica gel. The filtrate was concentrated again, and the residue was taken up in THF and purified by preparative HPLC (mobile phase: acetonitrile/water gradient with 0.05% formic acid in the water). 56 mg (22% of theory, purity 97%) of the title compound were obtained.

LC/MS (Method 1, ESIpos): Rₜ=1.57 min, m/z=668 [M+H]⁺.

### Example 71A

### [2-(4-Oxo-1,2,3-benzotriazin-3(4H)-yl)ethyl](2-oxo-2-{6-[2-(tetrahydro-2H-pyran-4-yl)ethoxy]-1-benzofur-3-yl}ethyl)malonic acid

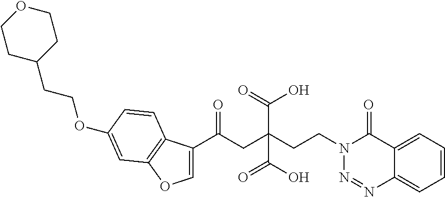 [SMILES: O=C(CC(CCn1nnc2ccccc2c1=O)(C(=O)O)C(=O)O)c1coc2cc(OCCC3CCOCC3)ccc12]

To a solution of 147 mg (0.22 mmol) of the compound from Example 56A in 4.5 ml of dichloromethane were added at RT 2.2 ml (29.1 mmol) of trifluoroacetic acid and the reaction mixture was stirred for 1 h at RT. Then, the mixture was concentrated and the residue was dried in vacuo. The title compound was obtained in a purity of 84% according to LC/MS and used directly in the subsequent stage.

LC/MS (Method 1, ESIpos): Rₜ=0.89 min, m/z=564 [M+H]⁺.

### Example 72A

### {2-[6-(2-Cyclopentylethoxy)-1-benzofur-3-yl]-2-oxoethyl}[2-(4-oxo-1,2,3-benzotriazin-3(4H)-yl)ethyl]malonic acid

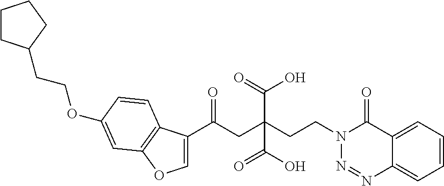 [SMILES: O=C(CC(CCn1nnc2ccccc2c1=O)(C(=O)O)C(=O)O)c1coc2cc(OCCC3CCCC3)ccc12]

To a solution of 167 mg (0.25 mmol) of the compound from Example 57A in 5.2 ml of dichloromethane were added at RT 2.6 ml (33.9 mmol) of trifluoroacetic acid and the reaction mixture was stirred for 1 h at RT. Then, the mixture was concentrated and the residue was dried in vacuo. The title compound was obtained in a purity of 95% according to LC/MS and used directly in the subsequent stage.

LC/MS (Method 2, ESIpos): Rₜ=2.48 min, m/z=548 [M+H]⁺.

### Example 73A

### {2-[6-(2-Cyclohexylethoxy)-1-benzofur-3-yl]-2-oxoethyl}[2-(4-oxo-1,2,3-benzotriazin-3(4H)-yl)ethyl]malonic acid

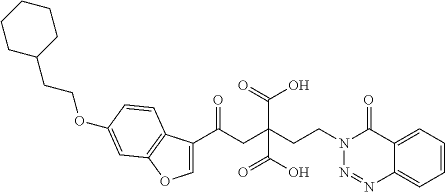 [SMILES: O=C(CC(CCn1nnc2ccccc2c1=O)(C(=O)O)C(=O)O)c1coc2cc(OCCC3CCCCC3)ccc12]

To a solution of 140 mg (0.21 mmol) of the compound from Example 58A in 3.8 ml of dichloromethane were added at RT 1.9 ml (24.8 mmol) of trifluoroacetic acid and the reaction mixture was stirred for 1 h at RT. Then, the mixture was concentrated and the residue was dried in vacuo. The title compound was obtained in a purity of 97% according to LC/MS and used directly in the subsequent stage.

LC/MS (Method 1, ESIpos): Rₜ=1.30 min, m/z=562 [M+H]⁺.

### Example 74A

### (2-{6-[(3-Chlorobenzyl)oxy]-1-benzofur-3-yl}-2-oxoethyl)[2-(4-oxo-1,2,3-benzotriazin-3(4H)-yl)ethyl]malonic acid

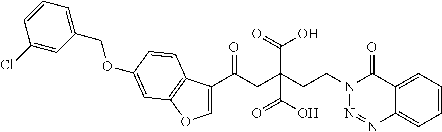 [SMILES: O=C(CC(CCn1nnc2ccccc2c1=O)(C(=O)O)C(=O)O)c1coc2cc(OCc3cccc(Cl)c3)ccc12]

To a solution of 137 mg (0.20 mmol) of the compound from Example 59A in 3.6 ml of dichloromethane were added at RT 1.8 ml (23.5 mmol) of trifluoroacetic acid and the reaction mixture was stirred for 1 h at RT. Then, the mixture was concentrated and the residue was dried in vacuo. The title compound was obtained in a purity of 92% according to LC/MS and used directly in the subsequent stage.

LC/MS (Method 1, ESIpos): Rₜ=1.08 min, m/z=576 [M+H]⁺.

### Example 75A

### {2-[6-(Cyclopentylmethoxy)-1-benzofur-3-yl]-2-oxoethyl}[2-(4-oxo-1,2,3-benzotriazin-3(4H)-yl)ethyl]malonic acid

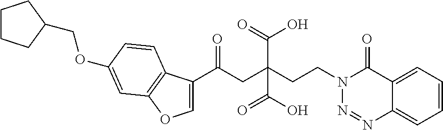 [SMILES: O=C(CC(CCn1nnc2ccccc2c1=O)(C(=O)O)C(=O)O)c1coc2cc(OCC3CCCC3)ccc12]

To a solution of 467 mg (0.65 mmol, purity 90%) of the compound from Example 60A in 7.9 ml of dichloromethane were added at RT 3.9 ml (50.9 mmol) of trifluoroacetic acid and the reaction mixture was stirred for 1 h at RT. Then, the mixture was concentrated and the residue was dried in vacuo. The title compound was obtained in a purity of 92% according to LC/MS and used directly in the subsequent stage.

LC/MS (Method 1, ESIpos): Rₜ=1.10 min, m/z=534 [M+H]⁺.

### Example 76A

### {2-[6-(Cyclohexylmethoxy)-1-benzofur-3-yl]-2-oxoethyl}[2-(4-oxo-1,2,3-benzotriazin-3(4H)-yl)ethyl]malonic acid

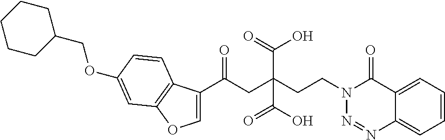 [SMILES: O=C(CC(CCn1nnc2ccccc2c1=O)(C(=O)O)C(=O)O)c1coc2cc(OCC3CCCCC3)ccc12]

To a solution of 380 mg (0.58 mmol) of the compound from Example 61A in 2.6 ml of dichloromethane were added at RT 1.3 ml (17.0 mmol) of trifluoroacetic acid and the reaction mixture was stirred for 1 h at RT. Then, the mixture was concentrated and the residue was dried in vacuo. The title compound was obtained in a purity of 87% according to LC/MS and used directly in the subsequent stage.

LC/MS (Method 1, ESIpos): Rₜ=1.12 min, m/z=548 [M+H]⁺.

### Example 77A

### 6-(Benzyloxy)-1-benzothiophene-2-carboxylic acid

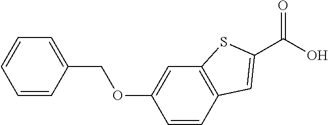 [SMILES: O=C(O)c1cc2ccc(OCc3ccccc3)cc2s1]

To a solution of 22.17 g (99.75 mmol) of ethyl 6-hydroxy-1-benzothiophene-2-carboxylate [EP 2 351 743-A1, Reference Example 39] in 220 ml of ethanol were added 15.15 g (119.70 mmol) of benzyl chloride and 20.69 g (149.62 mmol) of potassium carbonate. The mixture was stirred at 80° C. for 7.5 h. Then, 500 ml of a 1 M aqueous potassium hydroxide solution were slowly added, and the mixture was further stirred overnight at 80° C. After cooling to RT, with ice cooling, about 400 ml of 2 M hydrochloric acid was used to acidify the pH to 3. After dilution with water, the solid present was filtered off, washed with water and dried firstly in vacuo and then in a drying cabinet at 80° C. 26.23 g (80% of theory, purity 86%) of the title compound were obtained.

¹H-NMR (400 MHz, DMSO-d₆): δ [ppm]=13.29 (br. s, 1H), 8.02 (s, 1H), 7.90 (d, 1H), 7.71 (d, 1H), 7.51-7.45 (m, 2H), 7.44-7.31 (m, 3H), 7.15 (dd, 1H), 5.20 (s, 2H).

LC/MS (Method 1, ESIpos): Rₜ=1.07 min, m/z=285 [M+H]⁺.

### Example 78A

### 6-(Benzyloxy)-N-methoxy-N-methyl-1-benzothiophene-2-carboxamide

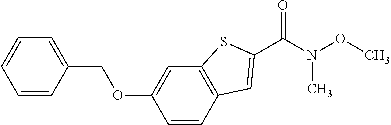 [SMILES: CON(C)C(=O)c1cc2ccc(OCc3ccccc3)cc2s1]

To a mixture of 27.21 g (95.69 mmol) of the compound from Example 77A in 300 ml of dichloromethane were added 20.9 ml (287.08 mmol) of thionyl chloride. The mixture was stirred first at 40° C. for 6 h and then at RT for 2 d. Then, the mixture was concentrated and the residue was dried in vacuo. Then, the residue was taken up in 250 ml of dichloromethane and slowly admixed with 12.14 g (124.40 mmol) of N,O-dimethylhydroxylamine hydrochloride and 66.7 ml (382.77 mmol) of N,N-diisopropylethylamine. After stirring for 30 min at RT, the mixture was diluted with dichloromethane and washed in succession with saturated aqueous ammonium chloride solution and saturated aqueous sodium chloride solution. The organic phase was dried over magnesium sulphate, filtered and concentrated. The residue was purified by column chromatography (silica gel, mobile phase cyclohexane/ethyl acetate 5:1→2:1). 31.34 g (95% of theory, purity 95%) of the title compound were obtained.

¹H-NMR (400 MHz, DMSO-d₆): δ [ppm]=8.11 (s, 1H), 7.91 (d, 1H), 7.67 (d, 1H), 7.51-7.46 (m, 2H), 7.45-7.31 (m, 3H), 7.13 (dd, 1H), 5.20 (s, 2H), 3.81 (s, 3H), 3.32 (s, 3H).

LC/MS (Method 1, ESIpos): Rₜ=1.18 min, m/z=328 [M+H]⁺.

### Example 79A

### 1-[6-(Benzyloxy)-1-benzothiophen-2-yl]ethanone

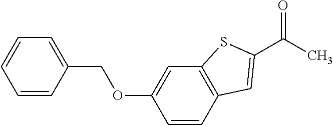 [SMILES: CC(=O)c1cc2ccc(OCc3ccccc3)cc2s1]

To a mixture of 33.10 g (101.09 mmol) of the compound from Example 78A in 250 ml of THF under argon were added at 0° C. 47.4 ml (151.65 mmol) of methylmagnesium bromide solution (3.2 M in 2-methyltetrahydrofuran). After removing the cooling bath, the mixture was stirred at RT for 1.5 h. Then, the mixture was slowly admixed with saturated aqueous ammonium chloride solution and extracted three times with ethyl acetate. The combined organic phases were dried over magnesium sulphate, filtered and concentrated. The residue was dried in vacuo. 25.05 g (79% of theory, purity 90%) of the title compound were obtained.

¹H-NMR (400 MHz, DMSO-d₆): δ [ppm]=8.26 (s, 1H), 7.92 (d, 1H), 7.71 (d, 1H), 7.51-7.46 (m, 2H), 7.45-7.31 (m, 3H), 7.16 (dd, 1H), 5.21 (s, 2H), 2.61 (s, 3H).

LC/MS (Method 1, ESIpos): Rₜ=1.21 min, m/z=283 [M+H]⁺.

### Example 80A

### 1-[6-(Benzyloxy)-1-benzothiophen-2-yl]-2-bromoethanone

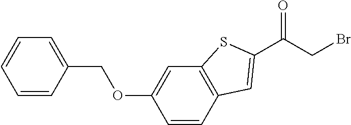 [SMILES: O=C(CBr)c1cc2ccc(OCc3ccccc3)cc2s1]

To a mixture of 10.0 g (33.29 mmol, purity 94%) of the compound from Example 79A in 24 ml of THF were added 12.52 g (33.29 mmol) of phenyltrimethylammonium tribromide. The mixture was stirred at RT for 5 h. Then, the mixture was diluted with ethyl acetate and washed with water. The aqueous phase was back-extracted twice with ethyl acetate. The combined organic phases were washed with saturated sodium chloride solution, dried over magnesium sulphate, filtered and concentrated. After stirring in methanol, the solid present was filtered off and dried in vacuo. 11.29 g (64% of theory, purity 68%) of the title compound were obtained.

LC/MS (Method 1, ESIpos): Rₜ=1.28 min, m/z=361/363 [M+H]⁺.

### Example 81A

### Di-tert-butyl {2-[6-(benzyloxy)-1-benzothiophen-2-yl]-2-oxoethyl}[2-(4-oxo-1,2,3-benzotriazin-3(4H)-yl)ethyl]malonate

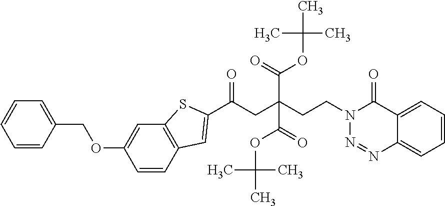 [SMILES: CC(C)(C)OC(=O)C(CCn1nnc2ccccc2c1=O)(CC(=O)c1cc2ccc(OCc3ccccc3)cc2s1)C(=O)OC(C)(C)C]

To a mixture of 10.76 g (27.63 mmol) of the compound from Example 2A in 60 ml of DMF under argon were added 1.11 g (27.63 mmol) of sodium hydride (60% in paraffin oil). After brief heating to 50° C., the mixture was cooled to −10° C. and admixed with 11.29 g (21.25 mmol, purity 68%) of the compound from Example 80A, dissolved in 40 ml of DMF. After removal of the cooling bath and stirring for 1 h at RT, the mixture was admixed with water and extracted with ethyl acetate. The organic phase was washed twice with water and with saturated sodium chloride solution, dried over magnesium sulphate, filtered and concentrated. The residue was purified by column chromatography (silica gel, mobile phase cyclohexane/ethyl acetate 5:1). 11.32 g (54% of theory, purity 88%) of the title compound were obtained.

LC/MS (Method 1, ESIpos): Rₜ=1.54 min, m/z=670 [M+H]⁺.

### Example 82A

### Di-tert-butyl {2-[6-(benzyloxy)-1-benzothiophen-2-yl]-2-oxoethyl}{2-[4-oxo-6-(trifluoromethyl)-1,2,3-benzotriazin-3(4H)-yl]ethyl}malonate

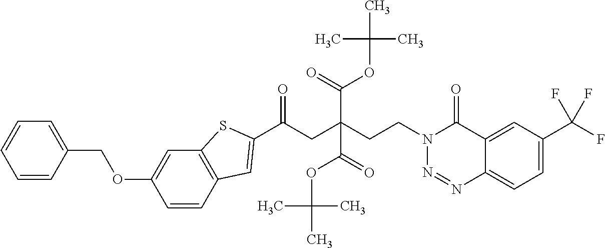 [SMILES: CC(C)(C)OC(=O)C(CCn1nnc2ccc(C(F)(F)F)cc2c1=O)(CC(=O)c1cc2ccc(OCc3ccccc3)cc2s1)C(=O)OC(C)(C)C]

To a mixture of 2.07 g (4.22 mmol, purity 93%) of the compound from Example 3A in 30 ml of DMF under argon were added 202 mg (5.06 mmol) of sodium hydride (60% in paraffin oil). After brief heating to 50° C., the mixture was cooled to 0° C. and admixed with a solution, precooled to 0° C., of 1.88 g (4.22 mmol, purity 81%) of the compound from Example 80A in 30 ml of DMF. After removal of the cooling bath and stirring for 16 h at RT, the mixture was admixed with water and extracted with ethyl acetate. The organic phase was washed twice with water and with saturated sodium chloride solution, dried over magnesium sulphate, filtered and concentrated. The residue was stirred in methanol. The solid formed was filtered off and dried in vacuo. 2.26 g (66% of theory, purity 90%) of the title compound were obtained.

LC/MS (Method 1, ESIpos): Rₜ=1.58 min, m/z=738 [M+H]⁺.

### Example 83A

### Di-tert-butyl [2-(6-hydroxy-1-benzothiophen-2-yl)-2-oxoethyl][2-(4-oxo-1,2,3-benzotriazin-3(4H)-yl)ethyl]malonate

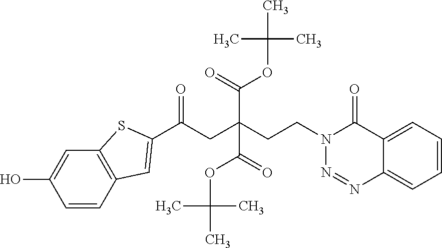 [SMILES: CC(C)(C)OC(=O)C(CCn1nnc2ccccc2c1=O)(CC(=O)c1cc2ccc(O)cc2s1)C(=O)OC(C)(C)C]

To a mixture of 10.88 g (16.24 mmol) of the compound from Example 81A in 400 ml of a 1:1 mixture of acetic acid and ethyl acetate were added 5.12 g (81.22 mmol) of ammonium formate, followed by 5.0 g (4.69 mmol) of palladium catalyst (10% on activated carbon) under argon. After stirring for 16 h at 80° C., once again 5.12 g (81.22 mmol) of ammonium formate were added, followed by a further 4.5 g (4.22 mmol) of palladium catalyst (10% on activated carbon). After stirring for a further 7 h at 80° C. and cooling to RT, the mixture was filtered over Celite, the filter residue was after-washed with a THF/ethyl acetate mixture (1:1) and the filtrate was concentrated. The residue was taken up in saturated aqueous sodium hydrogencarbonate solution and extracted three times with ethyl acetate. The combined organic phases were washed with saturated sodium chloride solution and dried over magnesium sulphate, filtered and concentrated. The residue was taken up in a mixture of 10 ml of methanol and 10 ml of dioxane and purified by means of preparative SFC [column with 2-ethylpyridine-modified silica gel, 5 μm, 150 mm×30 mm; flow rate: 100 ml/min; detection: 210 nm; injection volume 0.50 ml, temperature: 40° C.; gradient 80% CO₂/20% methanol→70% CO₂/30% methanol; total run time 5.5 min]. 5.72 g (61% of theory, purity 100%) of the title compound were obtained.

¹H-NMR (400 MHz, DMSO-d₆): δ [ppm]=10.19 (s, 1H), 8.28 (s, 1H), 8.20 (d, 1H), 8.14-8.10 (m, 1H), 8.05-7.98 (m, 1H), 7.90-7.82 (m, 2H), 7.27 (d, 1H), 6.97 (dd, 1H), 4.50-4.43 (m, 2H), 3.70 (s, 2H), 1.40 (s, 18H) [hidden 2H].

LC/MS (Method 1, ESIpos): Rₜ=1.29 min, m/z=580 [M+H]⁺.

### Example 84A

### Di-tert-butyl [2-(6-hydroxy-1-benzothiophen-2-yl)-2-oxoethyl]{2-[4-oxo-6-(trifluoromethyl)-1,2,3-benzotriazin-3(4H)-yl]ethyl}malonate

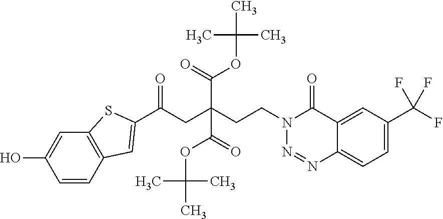 [SMILES: CC(C)(C)OC(=O)C(CCn1nnc2ccc(C(F)(F)F)cc2c1=O)(CC(=O)c1cc2ccc(O)cc2s1)C(=O)OC(C)(C)C]

To a mixture of 1.46 g (1.98 mmol) of the compound from Example 82A in 160 ml of a 1:1 mixture of acetic acid and ethyl acetate were added 1.25 g (19.79 mmol) of ammonium formate, followed by 1.25 g (1.17 mmol) of palladium catalyst (10% on activated carbon) under argon. After stirring for 3 d at 80° C. and cooling to RT, the mixture was filtered over Celite, the filter residue was after-washed with a THF/ethyl acetate mixture (1:1) and the filtrate was concentrated. The residue was taken up in saturated aqueous sodium hydrogencarbonate solution and extracted three times with ethyl acetate. The combined organic phases were washed with saturated sodium chloride solution, dried over magnesium sulphate, filtered and concentrated. The residue was dried in vacuo. 1.18 g (87% of theory, purity 94%) of the title compound were obtained.

LC/MS (Method 1, ESIpos): Rₜ=1.36 min, m/z=648 [M+H]⁺.

### Example 85A

### Di-tert-butyl {2-[6-(cyclopropylmethoxy)-1-benzothiophen-2-yl]-2-oxoethyl}[2-(4-oxo-1,2,3-benzotriazin-3(4H)-yl)ethyl]malonate

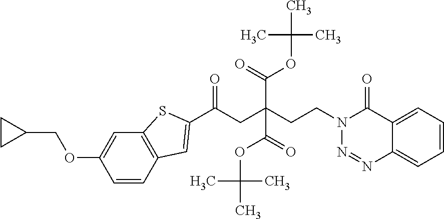 [SMILES: CC(C)(C)OC(=O)C(CCn1nnc2ccccc2c1=O)(CC(=O)c1cc2ccc(OCC3CC3)cc2s1)C(=O)OC(C)(C)C]

A mixture of 500 mg (0.86 mmol) of the compound from Example 83A, 359 mg (2.59 mmol) of 1-(bromomethyl)cyclopropane and 358 mg (2.59 mmol) of potassium carbonate in 15 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 634 mg (91% of theory, purity 98%) of the title compound were obtained.

LC/MS (Method 1, ESIpos): Rₜ=1.50 min, m/z=634 [M+H]⁺.

### Example 86A

### Di-tert-butyl [2-(4-oxo-1,2,3-benzotriazin-3(4H)-yl)ethyl]{2-oxo-2-[6-(2-phenylethoxy)-1-benzothiophen-2-yl]ethyl}malonate

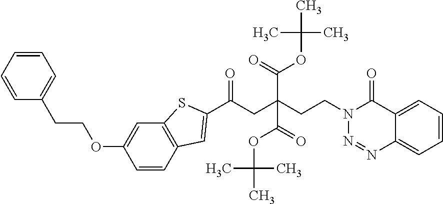 [SMILES: CC(C)(C)OC(=O)C(CCn1nnc2ccccc2c1=O)(CC(=O)c1cc2ccc(OCCc3ccccc3)cc2s1)C(=O)OC(C)(C)C]

A mixture of 100 mg (0.17 mmol) of the compound from Example 83A, 96 mg (0.52 mmol) of (2-bromomethyl)benzene and 72 mg (0.52 mmol) of potassium carbonate in 3 ml of acetonitrile was stirred for 16 h at 80° C. After cooling to RT, the mixture was admixed with water. The resulting solid was filtered off, washed with diethyl ether and dried in vacuo. 71 mg (60% of theory, purity 100%) of the title compound were obtained.

LC/MS (Method 1, ESIpos): Rₜ=1.58 min, m/z=684 [M+H]⁺.

### Example 87A

### Di-tert-butyl {2-[6-(2-cyclopropyl-2-oxoethoxy)-1-benzothiophen-2-yl]-2-oxoethyl}[2-(4-oxo-1,2,3-benzotriazin-3(4H)-yl)ethyl]malonate

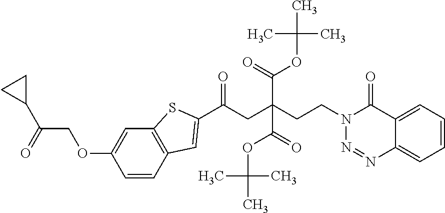 [SMILES: CC(C)(C)OC(=O)C(CCn1nnc2ccccc2c1=O)(CC(=O)c1cc2ccc(OCC(=O)C3CC3)cc2s1)C(=O)OC(C)(C)C]

A mixture of 100 mg (0.17 mmol) of the compound from Example 83A, 84 mg (0.52 mmol) of 2-bromo-1-cyclopropylethanone and 72 mg (0.52 mmol) of potassium carbonate in 3 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 125 mg (100% of theory, purity 92%) of the title compound were obtained.

LC/MS (Method 1, ESIpos): Rₜ=1.41 min, m/z=662 [M+H]⁺.

### Example 88A

### Di-tert-butyl [2-(4-oxo-1,2,3-benzotriazin-3(4H)-yl)ethyl][2-oxo-2-(6-propoxy-1-benzothiophen-2-yl)ethyl]malonate

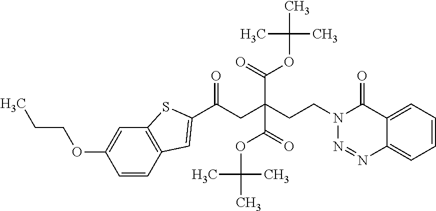 [SMILES: CCCOc1ccc2cc(C(=O)CC(CCn3nnc4ccccc4c3=O)(C(=O)OC(C)(C)C)C(=O)OC(C)(C)C)sc2c1]

A mixture of 100 mg (0.17 mmol) of the compound from Example 83A, 64 mg (0.52 mmol) of 1-bromopropane and 72 mg (0.52 mmol) of potassium carbonate in 3 ml of acetonitrile was stirred for 16 h at 80° C. After cooling to RT, the mixture was admixed with water. The resulting solid was filtered off, washed with diethyl ether and dried in vacuo. 43 mg (40% of theory, purity 100%) of the title compound were obtained.

LC/MS (Method 1, ESIpos): Rₜ=1.54 min, m/z=622 [M+H]⁺.

### Example 89A

### Di-tert-butyl [2-(4-oxo-1,2,3-benzotriazin-3(4H)-yl)ethyl]{2-oxo-2-[6-(tetrahydrofuran-2-ylmethoxy)-1-benzothiophen-2-yl]ethyl}malonate

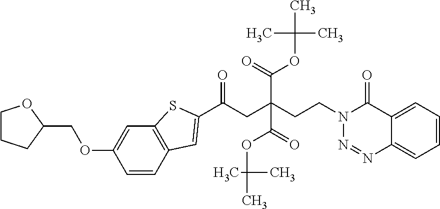 [SMILES: CC(C)(C)OC(=O)C(CCn1nnc2ccccc2c1=O)(CC(=O)c1cc2ccc(OCC3CCCO3)cc2s1)C(=O)OC(C)(C)C]

A mixture of 100 mg (0.17 mmol) of the compound from Example 83A, 85 mg (0.52 mmol) of 2-(bromomethyl)tetrahydrofuran and 72 mg (0.52 mmol) of potassium carbonate in 3 ml of acetonitrile was stirred for 16 h at 80° C. Subsequently, the reaction mixture was heated first in a microwave oven for 10 min at 150° C. and then for 10 min at 180° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 111 mg (76% of theory, purity 79%) of the title compound were obtained.

LC/MS (Method 1, ESIpos): Rₜ=1.46 min, m/z=664 [M+H]⁺.

### Example 90A

### Di-tert-butyl [2-(4-oxo-1,2,3-benzotriazin-3(4H)-yl)ethyl]{2-oxo-2-[6-(pentyloxy)-1-benzothiophen-2-yl]ethyl}malonate

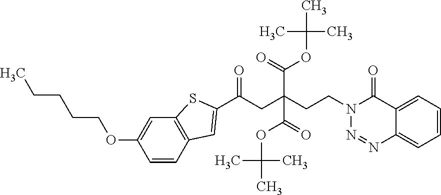 [SMILES: CCCCCOc1ccc2cc(C(=O)CC(CCn3nnc4ccccc4c3=O)(C(=O)OC(C)(C)C)C(=O)OC(C)(C)C)sc2c1]

A mixture of 100 mg (0.17 mmol) of the compound from Example 83A, 78 mg (0.52 mmol) of 1-bromopentane and 72 mg (0.52 mmol) of potassium carbonate in 3 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 56 mg (47% of theory, purity 95%) of the title compound were obtained.

### Example 91A

### Di-tert-butyl {2-[6-(cyclopropylmethoxy)-1-benzothiophen-2-yl]-2-oxoethyl}{2-[4-oxo-6-(trifluoromethyl)-1,2,3-benzotriazin-3(4H)-yl]ethyl}malonate

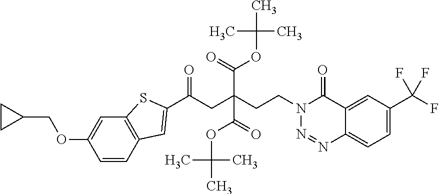 [SMILES: CC(C)(C)OC(=O)C(CCn1nnc2ccc(C(F)(F)F)cc2c1=O)(CC(=O)c1cc2ccc(OCC3CC3)cc2s1)C(=O)OC(C)(C)C]

A mixture of 500 mg (0.77 mmol) of the compound from Example 84A, 313 mg (2.32 mmol) of 1-(bromomethyl)cyclopropane and 320 mg (2.32 mmol) of potassium carbonate in 14 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 471 mg (70% of theory, purity 8195%) of the title compound were obtained. in vacuo. 471 mg (70% of theory, purity 81%) of the title compound were obtained.

LC/MS (Method 1, ESIpos): Rₜ=1.59 min, m/z=702 [M+H]⁺.

### Example 92A

### Di-tert-butyl [2-(6-methoxy-1-benzothiophen-2-yl)-2-oxoethyl]{2-[4-oxo-6-(trifluoromethyl)-1,2,3-benzotriazin-3(4H)-yl]ethyl}malonate

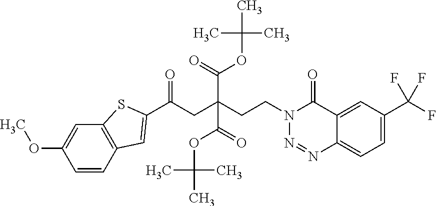 [SMILES: COc1ccc2cc(C(=O)CC(CCn3nnc4ccc(C(F)(F)F)cc4c3=O)(C(=O)OC(C)(C)C)C(=O)OC(C)(C)C)sc2c1]

A mixture of 100 mg (0.15 mmol) of the compound from Example 84A, 66 mg (0.46 mmol) of iodomethane and 64 mg (0.46 mmol) of potassium carbonate in 2 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 93 mg (86% of theory, purity 95%) of the title compound were obtained.

LC/MS (Method 1, ESIpos): Rₜ=1.53 min, m/z=662 [M+H]⁺.

### Example 93A

### Di-tert-butyl [2-oxo-2-(6-propoxy-1-benzothiophen-2-yl)ethyl]{2-[4-oxo-6-(trifluoromethyl)-1,2,3-benzotriazin-3(4H)-yl]ethyl}malonate

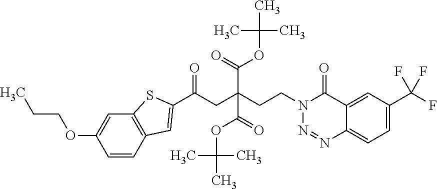 [SMILES: CCCOc1ccc2cc(C(=O)CC(CCn3nnc4ccc(C(F)(F)F)cc4c3=O)(C(=O)OC(C)(C)C)C(=O)OC(C)(C)C)sc2c1]

A mixture of 100 mg (0.15 mmol) of the compound from Example 84A, 79 mg (0.46 mmol) of 1-iodopropane and 64 mg (0.46 mmol) of potassium carbonate in 2 ml of acetonitrile was stirred for 16 h at 80° C. After cooling to RT, the mixture was admixed with water and extracted with ethyl acetate. The organic phase was washed once with saturated sodium chloride solution, dried over magnesium sulphate, filtered and concentrated. The residue was dried in vacuo. 104 mg (75% of theory, purity 76%) of the title compound were obtained.

LC/MS (Method 1, ESIpos): Rₜ=1.60 min, m/z=690 [M+H]⁺.

### Example 94A

### 6-(Benzyloxy)-N-methoxy-N-methyl-1, 2-benzoxazole-3-carboxamide

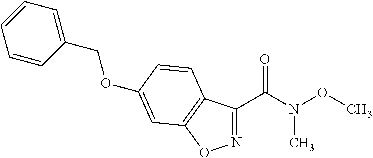 [SMILES: CON(C)C(=O)c1noc2cc(OCc3ccccc3)ccc12]

To a mixture of 40.03 g (0.41 mol) of (methoxyamino)methane hydrochloride in 1 litre of THF under argon were slowly added, at −78° C., 513 ml (0.82 mol) of a 1.6 M solution of butyllithium in THF. After stirring for 5 min at a bath temperature of −78° C., the cooling bath was removed and the mixture was stirred for a further 15 min. Then, cooling was effected again to a bath temperature of −78° C., and a solution of 24.40 g (82.07 mmol) of ethyl 6-(benzyloxy)-1,2-benzoxazole-3-carboxylate [WO 2008/009487-A1, Example 1, Stage 1.1] in 500 ml of THF was slowly added. After stirring for 1 h at −78° C., the cooling bath was removed and the mixture was stirred for a further 30 min, during which the internal temperature increased to approx. −20° C. Then, the mixture was admixed with 790 ml of saturated aqueous citric acid solution, diluted with water and rapidly extracted twice with ethyl acetate. The combined organic phases were washed once with saturated sodium chloride solution, dried over magnesium sulphate, filtered and concentrated. Drying in vacuo gave 26.6 g (87% of theory, purity 84%) of the title compound.

LC/MS (Method 1, ESIpos): Rₜ=1.11 min, m/z=313 [M+H]⁺.

In an experiment carried out analogously, 7.38 g (75.69 mmol) of (methoxyamino)methane hydrochloride and 4.50 g (15.14 mmol) of ethyl 6-(benzyloxy)-1,2-benzoxazole-3-carboxylate produced 4.67 g (97% of theory, purity 98%) of the title compound.

¹H-NMR (400 MHz, DMSO-d₆): δ [ppm]=7.78 (d, 1H), 7.54-7.33 (m, 6H), 7.14 (dd, 1H), 5.25 (s, 2H), 3.70 (s, 3H), 3.39 (s, 3H).

LC/MS (Method 1, ESIpos): Rₜ=1.11 min, m/z=313 [M+H]⁺.

### Example 95A

### 1-[6-(Benzyloxy)-1,2-benzoxazol-3-yl]ethanone

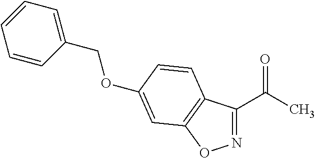 [SMILES: CC(=O)c1noc2cc(OCc3ccccc3)ccc12]

To a mixture of 39.65 g (109.18 mmol, purity 86%) of the compound from Example 94A in 365 ml of THF were slowly added at 0° C. 109 ml (0.33 mol) of a 3 M solution of methylmagnesium bromide in diethyl ether. After stirring for 20 min at a bath temperature of 0° C., with ice cooling, approx. 150 ml of saturated aqueous ammonium chloride solution were slowly added. Then, the mixture was diluted with water and extracted twice with ethyl acetate. The combined organic phases were dried over magnesium sulphate, filtered and concentrated. Drying in vacuo gave 35 g of a crude product, which was combined with the crude product (37 g) from an experiment of identical batch size carried out analogously. This material was then purified by means of flash chromatography (1.2 kg of silica gel, mobile phase cyclohexane/ethyl acetate 9:1). 16.2 g (27% of theory, purity 98%) of a first batch of the title compound and 36.3 g (57% of theory, purity 92%) of a second and 7.1 g (9% of theory, purity 73%) of a third batch of the title compound were obtained [yields based on 79.3 g (218.36 mmol, purity 86%) of the compound used from Example 94A].

LC/MS (Method 1, ESIpos): Rₜ=1.23 min, m/z=268 [M+H]⁺.

In an experiment carried out analogously, 11.65 g (37.30 mmol) of the compound from Example 94A and 37.3 ml (0.11 mmol) of a 3 M solution of methylmagnesium bromide in diethyl ether produced 10.40 g (quant., purity 96%) of the title compound.

¹H-NMR (400 MHz, DMSO-d₆): δ [ppm]=7.97 (d, 1H), 7.57 (d, 1H), 7.53-7.32 (m, 5H), 7.19 (dd, 1H), 5.25 (s, 2H), 2.70 (s, 3H).

LC/MS (Method 1, ESIpos): Rₜ=1.22 min, m/z=268 [M+H]⁺.

### Example 96A

### 1-[6-(Benzyloxy)-1,2-benzoxazol-3-yl]-2-bromoethanone

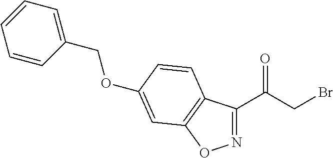 [SMILES: O=C(CBr)c1noc2cc(OCc3ccccc3)ccc12]

To a mixture of 36.3 g (125.21 mmol, purity 92%) of the compound from Example 95A in 950 ml of THF were added at RT 50.96 g (131.48 mmol, purity 97%) of phenyltrimethylammonium tribromide. The mixture was stirred at RT for 3 h. Then, the mixture was diluted with water and extracted twice with ethyl acetate. The combined organic phases were washed once with saturated sodium chloride solution, dried over magnesium sulphate, filtered and concentrated. The residue was stirred in 270 ml of ethanol under reflux for approx. 15 min. The resulting solid was filtered off and washed once with a small amount of ethanol and washed once with a small amount of pentane. Drying in the air gave 35.28 g (77% of theory, purity 94%) of the title compound.

¹H-NMR (400 MHz, CDCl₃): δ [ppm]=8.07-8.01 (m, 1H), 7.48-7.33 (m, 5H), 7.17-7.12 (m, 2H), 5.17 (s, 2H), 4.70 (s, 2H).

LC/MS (Method 2, ESIpos): Rₜ=2.88 min, m/z=346 [M+H]⁺.

### Example 97A

### Di-tert-butyl {2-[6-(benzyloxy)-1,2-benzoxazol-3-yl]-2-oxoethyl}[2-(4-oxo-1,2,3-benzotriazin-3(4H)-yl)ethyl]malonate

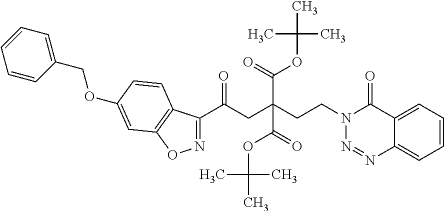 [SMILES: CC(C)(C)OC(=O)C(CCn1nnc2ccccc2c1=O)(CC(=O)c1noc2cc(OCc3ccccc3)ccc12)C(=O)OC(C)(C)C]

To a suspension of 642 mg (16.05 mmol) of sodium hydride (60% in paraffin oil) in 50 ml of DMF under argon were added at RT a solution of 5.0 g (12.84 mmol) of the compound from Example 2A in 50 ml of DMF. After stirring for approx. 30 minutes at RT, the mixture was cooled to −20° C., and a solution of 5.31 g (14.12 mmol, purity 92%) of the compound from Example 96A in 50 ml of DMF was quickly added. After stirring for 5 minutes at −20° C., the cooling bath was removed, and the mixture was admixed with 250 ml of water and 250 ml of 10% strength aqueous citric acid and extracted twice with tert-butyl methyl ether. The combined organic phases were dried over magnesium sulphate and concentrated. The crude product was purified together with the crude product from an experiment carried out analogously, in which 1.0 g (2.57 mmol) of the compound from Example 2A and 1.06 g (2.82 mmol, purity 92%) of the compound from Example 96A had been used, by means of column chromatography (700 g of silica gel, mobile phase toluene/ethyl acetate 9:1). 5.38 mg of the title compound were obtained (49% of theory, based on in total 6.06 g (15.41 mmol) of the compound from Example 2A).

LC/MS (Method 1, ESIpos): Rₜ=1.54 min, m/z=655 [M+H]⁺.

In an experiment carried out analogously, in which 1.0 g (2.57 mmol) of the compound from Example 2A had been used, purification of the crude product by preparative HPLC (method 6) gave 170 mg (10% of theory) of the title compound.

¹H-NMR (400 MHz, DMSO-d₆): δ [ppm]=8.19 (d, 1H), 8.12 (d, 1H), 8.05-7.98 (m, 1H), 7.93-7.84 (m, 2H), 7.56 (d, 1H), 7.53-7.48 (m, 2H), 7.46-7.33 (m, 3H), 7.19 (dd, 1H), 5.26 (s, 2H), 4.53-4.45 (m, 2H), 3.84 (s, 2H), 1.39 (s, 18H) [hidden 2H].

LC/MS (Method 1, ESIpos): Rₜ=1.55 min, m/z=655 [M+H]⁺.

### Example 98A

### Di-tert-butyl {2-[6-(benzyloxy)-1,2-benzoxazol-3-yl]-2-oxoethyl}{2-[4-oxo-6-(trifluoromethyl)-1,2,3-benzotriazin-3(4H)-yl]ethyl}malonate

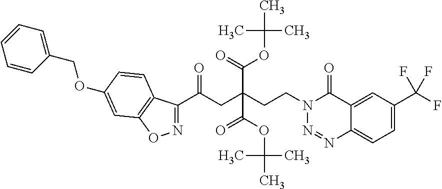 [SMILES: CC(C)(C)OC(=O)C(CCn1nnc2ccc(C(F)(F)F)cc2c1=O)(CC(=O)c1noc2cc(OCc3ccccc3)ccc12)C(=O)OC(C)(C)C]

To a suspension of 22 mg (0.55 mmol) of sodium hydride (60% in paraffin oil) in 2 ml of DMF under argon were added at RT a solution of 200 mg (0.44 mmol) of the compound from Example 3A in 2 ml of DMF. After stirring for approx. 30 minutes at 50° C., the mixture was cooled to −20° C., and a solution of 183 mg (0.48 mmol, purity 91%) of the compound from Example 96A in 2 ml of DMF was quickly added. After stirring for 5 minutes at −20° C., the cooling bath was removed, and, after stirring for a further 15 min, the mixture was admixed with 10 ml of water and 10 ml of 10% strength aqueous citric acid and extracted twice with tert-butyl methyl ether. The combined organic phases were dried over magnesium sulphate, filtered and concentrated. The residue was purified by means of preparative HPLC (Method 8). The product-containing fractions were combined, neutralized with saturated aqueous sodium hydrogencarbonate solution and concentrated down to a residual volume of aqueous phase. Then, the mixture was extracted twice with dichloromethane, and the combined organic phases were dried over magnesium sulphate, filtered and concentrated. Drying in vacuo gave 83 mg (24% of theory, purity 93%) of the title compound.

¹H-NMR (400 MHz, CDCl₃): δ [ppm]=8.58 (s, 1H), 8.21 (d, 1H), 8.08 (dd, 1H), 8.00 (d, 1H), 7.49-7.33 (m, 5H), 7.13-7.05 (m, 2H), 5.16 (s, 2H), 4.69-4.60 (m, 2H), 3.91 (s, 2H), 2.67-2.62 (m, 2H), 1.50 (s, 18H).

LC/MS (Method 1, ESIpos): Rₜ=1.61 min, m/z=723 [M+H]⁺.

### Example 99A

### Di-tert-butyl [2-(6-hydroxy-1,2-benzoxazol-3-yl)-2-oxoethyl][2-(4-oxo-1,2,3-benzotriazin-3(4H)-yl)ethyl]malonate

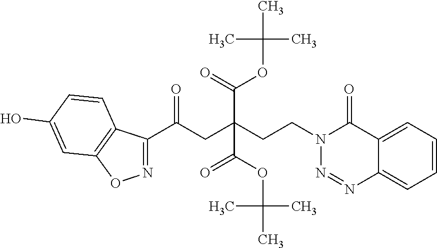 [SMILES: CC(C)(C)OC(=O)C(CCn1nnc2ccccc2c1=O)(CC(=O)c1noc2cc(O)ccc12)C(=O)OC(C)(C)C]

A solution of 5.35 g (7.52 mmol, purity 92%) of the compound from Example 97A in 330 ml of a 1:1 mixture of ethyl acetate and ethanol was admixed at RT with 0.40 g (0.38 mmol) of palladium catalyst (10% on activated carbon) and 2.37 g (37.59 mmol) of ammonium formate and stirred for 1 h at 70° C. After cooling to RT, solid constituents were filtered off over kieselguhr and the filter residue was after-washed with ethyl acetate. The filtrate was concentrated, the residue was then taken up again in dichloromethane, the solution was washed once with saturated sodium chloride solution and then dried over magnesium sulphate, filtered and concentrated. The residue was purified by means of column chromatography (200 g of silica gel, mobile phase cyclohexane/ethyl acetate 7:3). 1.89 g (45% of theory, purity 100%) of the title compound were obtained.

¹H-NMR (400 MHz, CDCl₃): δ [ppm]=8.31 (dd, 1H), 8.14 (d, 1H), 7.97-7.92 (m, 1H), 7.84-7.76 (m, 2H), 7.48 (br. s, 1H), 6.93 (d, 1H), 6.84 (dd, 1H), 4.68-4.59 (m, 2H), 3.96 (s, 2H), 2.75-2.65 (m, 2H), 1.51 (s, 18H).

LC/MS (Method 1, ESIpos): Rₜ=1.31 min, m/z=565 [M+H]⁺.

### Example 100A

### Di-tert-butyl [2-(6-hydroxy-1,2-benzoxazol-3-yl)-2-oxoethyl]{2-[4-oxo-6-(trifluoromethyl)-1,2,3-benzotriazin-3(4H)-yl]ethyl}malonate

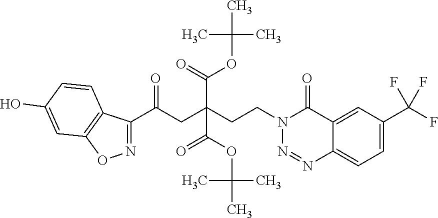 [SMILES: CC(C)(C)OC(=O)C(CCn1nnc2ccc(C(F)(F)F)cc2c1=O)(CC(=O)c1noc2cc(O)ccc12)C(=O)OC(C)(C)C]

A solution of 282 mg (0.37 mmol, purity 94%) of the compound from Example 98A in 16 ml of a 1:1 mixture of ethyl acetate and ethanol was admixed at RT with 20 mg (0.018 mmol) of palladium catalyst (10% on activated carbon) and 116 mg (1.83 mmol) of ammonium formate and stirred for 2 h at 70° C. After cooling to RT, solid constituents were filtered off over kieselguhr and the filter residue was after-washed with ethyl acetate. The filtrate was concentrated and the residue was purified by column chromatography (25 g of silica gel, mobile phase cyclohexane/ethyl acetate 8:2). 113 mg (49% of theory, purity 100%) of the title compound were obtained.

¹H-NMR (400 MHz, DMSO-d₆): δ [ppm]=10.66 (br. s, 1H), 8.42 (s, 1H), 8.35 (d, 2H), 7.82 (d, 1H), 7.07 (d, 1H), 6.98 (dd, 1H), 4.55-4.47 (m, 2H), 3.81 (s, 2H), 2.59-2.54 (m, 2H), 1.40 (s, 18H).

LC/MS (Method 1, ESIpos): Rₜ=1.39 min, m/z=633 [M+H]⁺.

### Example 101A

### Di-tert-butyl [2-(4-oxo-1,2,3-benzotriazin-3(4H)-yl)ethyl]{2-oxo-2-[6-(tetrahydro-2H-pyran-4-ylmethoxy)-1,2-benzoxazol-3-yl]ethyl}malonate

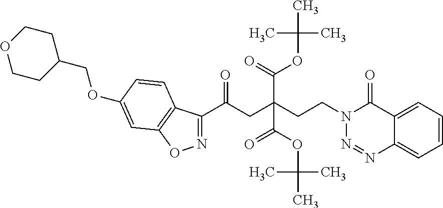 [SMILES: CC(C)(C)OC(=O)C(CCn1nnc2ccccc2c1=O)(CC(=O)c1noc2cc(OCC3CCOCC3)ccc12)C(=O)OC(C)(C)C]

To a mixture of 1.30 g (2.30 mmol) of the compound from Example 99A in 35 ml of acetonitrile were added at RT 955 mg (6.91 mmol) of potassium carbonate and 825 mg (4.61 mmol) of 4-(bromomethyl)tetrahydro-2H-pyran. The mixture was stirred under reflux overnight. After cooling to RT, water was added, and the aqueous phase was extracted twice with ethyl acetate. The combined organic phases were washed once with saturated sodium chloride solution, dried over magnesium sulphate, filtered and concentrated. The residue was purified by means of column chromatography (120 g of silica gel, mobile phase cyclohexane/ethyl acetate 7:3). 550 mg (28% of theory, purity 78%) of the title compound were obtained.

LC/MS (Method 1, ESIpos): Rₜ=1.46 min, m/z=663 [M+H]⁺.

### Example 102A

### Di-tert-butyl (2-{6-[(4-fluorobenzyl)oxy]-1,2-benzoxazol-3-yl}-2-oxoethyl)[2-(4-oxo-1,2,3-benzotriazin-3(4H)-yl)ethyl]malonate

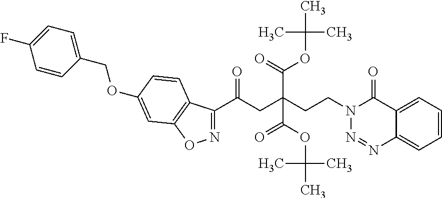 [SMILES: CC(C)(C)OC(=O)C(CCn1nnc2ccccc2c1=O)(CC(=O)c1noc2cc(OCc3ccc(F)cc3)ccc12)C(=O)OC(C)(C)C]

To a mixture of 150 mg (0.27 mmol) of the compound from Example 99A in 1.5 ml of acetonitrile were added at RT 73 mg (0.53 mmol) of potassium carbonate and 60 mg (0.32 mmol) of 1-(bromomethyl)-4-fluorobenzene. The mixture was stirred under reflux for 30 min. After cooling to RT, water was added, and the aqueous phase was extracted twice with dichloromethane. The combined organic phases were dried over magnesium sulphate, filtered and concentrated. The residue was dried in vacuo. 163 mg (75% of theory, purity 82%) of the title compound were obtained.

LC/MS (Method 1, ESIpos): Rₜ=1.55 min, m/z=673 [M+H]⁺.

### Example 103A

### Di-tert-butyl (2-{6-[(3-carbamoylbenzyl)oxy]-1,2-benzoxazol-3-yl}-2-oxoethyl)[2-(4-oxo-1,2,3-benzotriazin-3(4H)-yl)ethyl]malonate

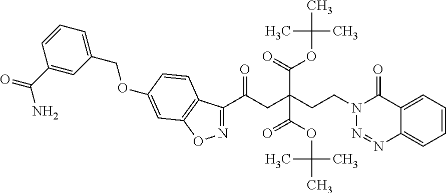 [SMILES: CC(C)(C)OC(=O)C(CCn1nnc2ccccc2c1=O)(CC(=O)c1noc2cc(OCc3cccc(C(N)=O)c3)ccc12)C(=O)OC(C)(C)C]

To a mixture of 150 mg (0.27 mmol) of the compound from Example 99A in 1.5 ml of acetonitrile were added at RT 73 mg (0.53 mmol) of potassium carbonate and 54 mg (0.32 mmol) of 3-(chloromethyl)benzamide. The mixture was stirred under reflux for 1.5 h. After cooling to RT, water was added, the mixture was neutralized with 10% strength citric acid and the aqueous phase was extracted twice with dichloromethane. The combined organic phases were dried over magnesium sulphate, filtered and concentrated. The residue was dried in vacuo. 196 mg (74% of theory, purity 70%) of the title compound were obtained.

LC/MS (Method 1, ESIpos): Rₜ=1.32 min, m/z=698 [M+H]⁺.

### Example 104A

### Di-tert-butyl [2-(4-oxo-1,2,3-benzotriazin-3(4H)-yl)ethyl]{2-oxo-2-[6-(pentyloxy)-1,2-benzoxazol-3-yl]ethyl}malonate

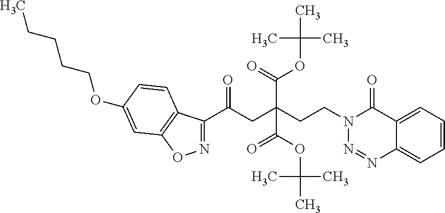 [SMILES: CCCCCOc1ccc2c(C(=O)CC(CCn3nnc4ccccc4c3=O)(C(=O)OC(C)(C)C)C(=O)OC(C)(C)C)noc2c1]

To a mixture of 150 mg (0.27 mmol) of the compound from Example 99A in 1.5 ml of acetonitrile were added at RT 110 mg (0.80 mmol) of potassium carbonate and 120 mg (0.80 mmol) of 1-bromopentane. The mixture was stirred under reflux for 2 h. After addition of ice water, the aqueous phase was extracted twice with ethyl acetate. The combined organic phases were washed with saturated sodium chloride solution, dried over magnesium sulphate, filtered and concentrated. The residue was purified by means of column chromatography (25 g of silica gel, mobile phase cyclohexane/ethyl acetate 85:15). 109 mg (57% of theory, purity 88%) of the title compound were obtained.

LC/MS (Method 1, ESIpos): Rₜ=1.64 min, m/z=635 [M+H]⁺.

### Example 105A

### Di-tert-butyl{2-[6-(2-cyclohexylethoxy)-1,2-benzoxazol-3-yl]-2-oxoethyl}[2-(4-oxo-1,2,3-benzotriazin-3(4H)-yl)ethyl]malonate

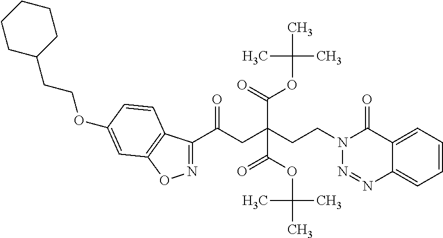 [SMILES: CC(C)(C)OC(=O)C(CCn1nnc2ccccc2c1=O)(CC(=O)c1noc2cc(OCCC3CCCCC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 152 mg (0.80 mmol) of (2-bromoethyl)cyclohexane. The mixture was stirred under reflux for 2 h. After addition of ice water, the aqueous phase was extracted twice with ethyl acetate. The combined organic phases were washed with saturated sodium chloride solution, dried over magnesium sulphate, filtered and concentrated. The residue was purified by means of column chromatography (25 g of silica gel, mobile phase cyclohexane/ethyl acetate 85:15). 134 mg (72% of theory, purity 97%) of the title compound were obtained.

LC/MS (Method 1, ESIpos): Rₜ=1.72 min, m/z=675 [M+H]⁺.

### Example 106A

### Di-tert-butyl [2-(4-oxo-1,2,3-benzotriazin-3(4H)-yl)ethyl]{2-oxo-2-[6-(2-phenylethoxy)-1,2-benzoxazol-3-yl]ethyl}malonate

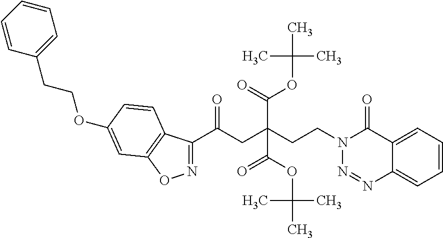 [SMILES: CC(C)(C)OC(=O)C(CCn1nnc2ccccc2c1=O)(CC(=O)c1noc2cc(OCCc3ccccc3)ccc12)C(=O)OC(C)(C)C]

To a mixture of 150 mg (0.27 mmol) of the compound from Example 99A in 1.5 ml of acetonitrile were added at RT 73 mg (0.53 mmol) of potassium carbonate and 89 mg (0.32 mmol, purity 92%) of 2-phenylethyltrifluoromethanesulphonate [Lit.: Synthesis 1985, 8, 759-760]. The mixture was stirred under reflux for 1.5 h. After cooling to RT, water was added and the aqueous phase was extracted twice with dichloromethane. The combined organic phases were dried over magnesium sulphate, filtered and concentrated. The residue was purified by thick-layer chromatography (PLC silica gel 60 F 254, 1 mm; 20×20 cm with concentration zone; mobile phase cyclohexane/ethyl acetate 7:3). The product-containing zone was extracted with dichloromethane/methanol (95:5), and the solvent was removed again and the residue was dried in vacuo. 141 mg (78% of theory, purity 99%) of the title compound were obtained.

LC/MS (Method 1, ESIpos): Rₜ=1.59 min, m/z=669 [M+H]⁺.

### Example 107A

### Di-tert-butyl (2-{6-[(3-chlorobenzyl)oxy]-1,2-benzoxazol-3-yl}-2-oxoethyl)[2-(4-oxo-1,2,3-benzotriazin-3(4H)-yl)ethyl]malonate

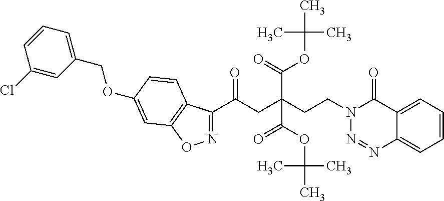 [SMILES: CC(C)(C)OC(=O)C(CCn1nnc2ccccc2c1=O)(CC(=O)c1noc2cc(OCc3cccc(Cl)c3)ccc12)C(=O)OC(C)(C)C]

To a mixture of 150 mg (0.27 mmol) of the compound from Example 99A in 1.5 ml of acetonitrile were added at RT 73 mg (0.53 mmol) of potassium carbonate and 65 mg (0.32 mmol) of 1-(bromomethyl)-3-chlorobenzene. The mixture was stirred under reflux for 1 h. After admixing with ice water, the aqueous phase was extracted twice with ethyl acetate. The combined organic phases were dried over magnesium sulphate, filtered and concentrated. The residue was purified by means of preparative HPLC (Method 8). Die combined product-containing fractions were neutralized with saturated aqueous sodium hydrogencarbonate solution and concentrated to a residual volume of aqueous phase. The mixture was extracted twice with dichloromethane, and the combined organic phases were dried over magnesium sulphate, filtered and concentrated. The residue was dried in vacuo. 168 mg (92% of theory, purity 100%) of the title compound were obtained.

LC/MS (Method 1, ESIpos): Rₜ=1.58 min, m/z=689 [M+H]⁺.

### Example 108A

### Di-tert-butyl (2-{6-[(4-chlorobenzyl)oxy]-1,2-benzoxazol-3-yl}-2-oxoethyl)[2-(4-oxo-1,2,3-benzotriazin-3(4H)-yl)ethyl]malonate

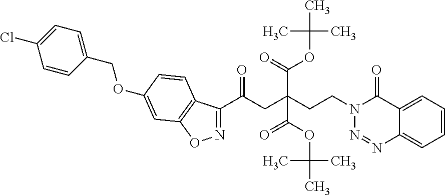 [SMILES: CC(C)(C)OC(=O)C(CCn1nnc2ccccc2c1=O)(CC(=O)c1noc2cc(OCc3ccc(Cl)cc3)ccc12)C(=O)OC(C)(C)C]

To a mixture of 150 mg (0.27 mmol) of the compound from Example 99A in 1.5 ml of acetonitrile were added at RT 73 mg (0.53 mmol) of potassium carbonate and 65 mg (0.32 mmol) of 1-(bromomethyl)-4-chlorobenzene. The mixture was stirred under reflux for 1 h. After admixing with ice water, the aqueous phase was extracted twice with ethyl acetate. The combined organic phases were dried over magnesium sulphate, filtered and concentrated. The residue was purified by means of preparative HPLC (Method 8). Die combined product-containing fractions were neutralized with saturated aqueous sodium hydrogencarbonate solution and concentrated to a residual volume of aqueous phase. The mixture was extracted twice with dichloromethane, and the combined organic phases were dried over magnesium sulphate, filtered and concentrated. The residue was dried in vacuo. 241 mg (99% of theory, purity 75%) of the title compound were obtained.

LC/MS (Method 1, ESIpos): Rₜ=1.58 min, m/z=689 [M+H]⁺.

### Example 109A

### Di-tert-butyl [2-(4-oxo-1,2,3-benzotriazin-3(4H)-yl)ethyl]{2-oxo-2-[6-(pyridin-3-ylmethoxy)-1,2-benzoxazol-3-yl]ethyl}malonate

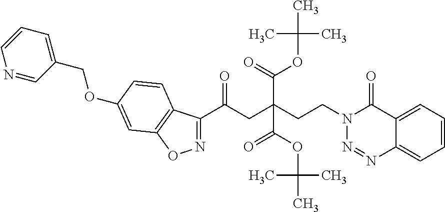 [SMILES: CC(C)(C)OC(=O)C(CCn1nnc2ccccc2c1=O)(CC(=O)c1noc2cc(OCc3cccnc3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 81 mg (0.32 mmol) of 3-(bromomethyl)pyridine hydrobromide. The mixture was stirred under reflux for 2 h. Then, a further 110 mg (0.80 mmol) of potassium carbonate and 81 mg (0.32 mmol) of 3-(bromomethyl)pyridine hydrobromide were added, and the mixture was stirred under reflux once again for 2 h. After cooling to RT, water was added and the aqueous phase was extracted three times with ethyl acetate. The combined organic phases were dried over magnesium sulphate, filtered and concentrated. The residue was purified by means of preparative HPLC (Method 6). The combined product-containing fractions were neutralized with saturated aqueous sodium hydrogencarbonate solution and concentrated to a residual volume of aqueous phase. The mixture was extracted twice with dichloromethane, and the combined organic phases were dried over magnesium sulphate, filtered and concentrated. The residue was dried in vacuo. 44 mg (25% of theory, purity 100%) of the title compound were obtained.

LC/MS (Method 1, ESIpos): Rₜ=1.34 min, m/z=656 [M+H]⁺.

### Example 110A

### Di-tert-butyl [2-(4-oxo-1,2,3-benzotriazin-3(4H)-yl)ethyl](2-oxo-2-{6-[2-(tetrahydro-2H-pyran-4-yl)ethoxy]-1,2-benzoxazol-3-yl}ethyl)malonate

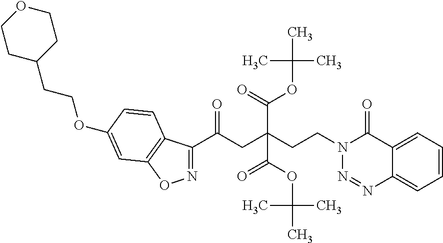 [SMILES: CC(C)(C)OC(=O)C(CCn1nnc2ccccc2c1=O)(CC(=O)c1noc2cc(OCCC3CCOCC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 154 mg (0.80 mmol) of 4-(2-bromoethyl)tetrahydro-2H-pyran. The mixture was stirred under reflux for 2 h. After admixing with ice water, the aqueous phase was extracted twice with ethyl acetate. The combined organic phases were dried over magnesium sulphate, filtered and concentrated. The residue was purified by means of column chromatography (40 g of silica gel, mobile phase cyclohexane/ethyl acetate 7:3). 132 mg (68% of theory, purity 92%) of the title compound were obtained.

LC/MS (Method 1, ESIpos): Rₜ=1.51 min, m/z=677 [M+H]⁺.

### Example 111A

### Di-tert-butyl {2-[6-(cyclopropylmethoxy)-1,2-benzoxazol-3-yl]-2-oxoethyl}[2-(4-oxo-1,2,3-benzotriazin-3(4H)-yl)ethyl]malonate

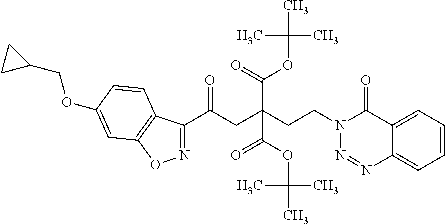 [SMILES: CC(C)(C)OC(=O)C(CCn1nnc2ccccc2c1=O)(CC(=O)c1noc2cc(OCC3CC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 108 mg (0.80 mmol) of (bromomethyl)cyclopropane. The mixture was stirred under reflux for 2 h. After admixing with ice water, the aqueous phase was extracted twice with ethyl acetate. The combined organic phases were dried over magnesium sulphate, filtered and concentrated. The residue was purified by means of column chromatography (40 g of silica gel, mobile phase: cyclohexane/ethyl acetate 8:2). 119 mg (67% of theory, purity 93%) of the title compound were obtained.

LC/MS (Method 1, ESIpos): Rₜ=1.52 min, m/z=619 [M+H]⁺.

### Example 112A

### Di-tert-butyl {2-[6-(2-cyclopentylethoxy)-1,2-benzoxazol-3-yl]-2-oxoethyl}[2-(4-oxo-1,2,3-benzotriazin-3(4H)-yl)ethyl]malonate

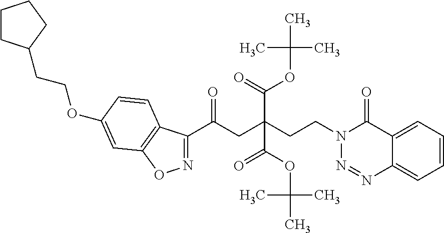 [SMILES: CC(C)(C)OC(=O)C(CCn1nnc2ccccc2c1=O)(CC(=O)c1noc2cc(OCCC3CCCC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 179 mg (0.80 mmol) of (2-iodoethyl)cyclopentane. The mixture was stirred under reflux for 2 h. After admixing with ice water, the aqueous phase was extracted twice with ethyl acetate. The combined organic phases were dried over magnesium sulphate, filtered and concentrated. The residue was purified by means of column chromatography (40 g of silica gel, mobile phase: cyclohexane/ethyl acetate 85:15). 131 mg (73% of theory, purity 99%) of the title compound were obtained.

LC/MS (Method 1, ESIpos): Rₜ=1.68 min, m/z=661 [M+H]⁺.

### Example 113A

### Di-tert-butyl {2-[6-(cyclopentylmethoxy)-1,2-benzoxazol-3-yl]-2-oxoethyl}[2-(4-oxo-1,2,3-benzotriazin-3(4H)-yl)ethyl]malonate

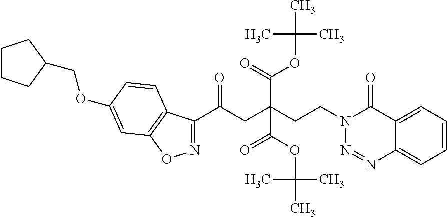 [SMILES: CC(C)(C)OC(=O)C(CCn1nnc2ccccc2c1=O)(CC(=O)c1noc2cc(OCC3CCCC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 130 mg (0.80 mmol) of (bromomethyl)cyclopentane. The mixture was stirred under reflux for 4 h. After cooling to RT, the aqueous phase was extracted twice with ethyl acetate. The combined organic phases were dried over magnesium sulphate, filtered and concentrated. The crude product was combined with the crude product of an experiment of identical batch size carried out analogously and then purified by column chromatography (40 g of silica gel, mobile phase cyclohexane/ethyl acetate 85:15). 143 mg of the title compound were obtained (31% of theory, based on in total 300 mg (0.54 mmol) of the compound from Example 99A, purity 75%).

LC/MS (Method 1, ESIpos): Rₜ=1.64 min, m/z=647 [M+H]⁺.

### Example 114A

### Di-tert-butyl {2-[6-(cyclohexylmethoxy)-1,2-benzoxazol-3-yl]-2-oxoethyl}[2-(4-oxo-1,2,3-benzotriazin-3(4H)-yl)ethyl]malonate

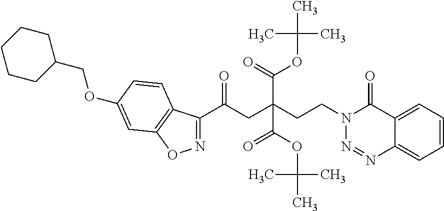 [SMILES: CC(C)(C)OC(=O)C(CCn1nnc2ccccc2c1=O)(CC(=O)c1noc2cc(OCC3CCCCC3)ccc12)C(=O)OC(C)(C)C]

To a mixture of 150 mg (0.27 mmol) of the compound from Example 99A in 1.5 ml of acetonitrile were added at RT 110 mg (0.80 mmol) of potassium carbonate and 141 mg (0.80 mmol) of (bromomethyl)cyclohexane. The mixture was stirred under reflux for 5 h. After cooling to RT, the aqueous phase was extracted twice with ethyl acetate. The combined organic phases were dried over magnesium sulphate, filtered and concentrated. The crude product was combined with the crude product of an experiment of identical batch size carried out analogously and then purified by column chromatography (40 g of silica gel, mobile phase cyclohexane/ethyl acetate 85:15). 113 mg of the title compound were obtained (25% of theory, based on in total 300 mg (0.54 mmol) of the compound from Example 99A, purity 75%).

LC/MS (Method 1, ESIpos): Rₜ=1.68 min, m/z=661 [M+H]⁺.

### Example 115A

### Di-tert-butyl {2-oxo-2-[6-(tetrahydro-2H-pyran-4-ylmethoxy)-1,2-benzoxazol-3-yl]ethyl}{2-[4-oxo-6-(trifluoromethyl)-1,2,3-benzotriazin-3(4H)-yl]ethyl}malonate

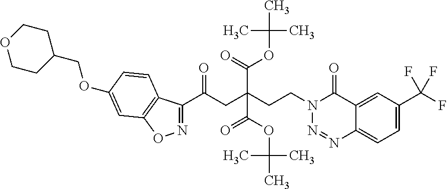 [SMILES: CC(C)(C)OC(=O)C(CCn1nnc2ccc(C(F)(F)F)cc2c1=O)(CC(=O)c1noc2cc(OCC3CCOCC3)ccc12)C(=O)OC(C)(C)C]

To a mixture of 100 mg (0.16 mmol) of the compound from Example 100A in 1.0 ml of acetonitrile were added at RT 66 mg (0.47 mmol) of potassium carbonate and 118 mg (0.47 mmol) of tetrahydro-2H-pyran-4-ylmethyltrifluoromethanesulphonate. The mixture was stirred at RT for 1 h. Then, the mixture was purified directly by thick-layer chromatography (PLC silica gel 60 F 254, 1 mm; 20×20 cm with concentration zone; mobile phase cyclohexane/ethyl acetate 3:2). The product-containing zone was extracted with dichloromethane/methanol (95:5), and the solvent was removed again and the residue was dried in vacuo. 75 mg (50% of theory, purity 76%) of the title compound were obtained.

LC/MS (Method 1, ESIpos): Rₜ=1.56 min, m/z=731 [M+H]⁺.

### Example 116A

### {2-[6-(Benzyloxy)-1,2-benzoxazol-3-yl]-2-oxoethyl}[2-(4-oxo-1,2,3-benzotriazin-3(4H)-yl)ethyl]malonic acid

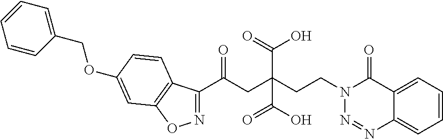 [SMILES: O=C(CC(CCn1nnc2ccccc2c1=O)(C(=O)O)C(=O)O)c1noc2cc(OCc3ccccc3)ccc12]

To a solution of 60 mg (0.092 mmol) of the compound from Example 97A in 1 ml of dichloromethane were added at RT 0.5 ml (6.5 mmol) of trifluoroacetic acid and the reaction mixture was stirred for 2.5 h at RT. Then, the mixture was concentrated and the residue was dried in vacuo. The title compound was obtained in a purity of 69% according to LC/MS and used directly in the subsequent stage.

LC/MS (Method 1, ESIpos): Rₜ=1.01 min, m/z=543 [M+H]⁺.

### Example 117A

### {2-[6-(Benzyloxy)-1,2-benzoxazol-3-yl]-2-oxoethyl}{2-[4-oxo-6-(trifluoromethyl)-1,2,3-benzotriazin-3(4H)-yl]ethyl}malonic acid

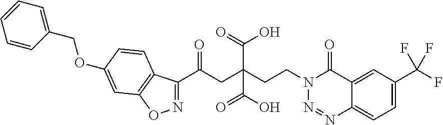 [SMILES: O=C(CC(CCn1nnc2ccc(C(F)(F)F)cc2c1=O)(C(=O)O)C(=O)O)c1noc2cc(OCc3ccccc3)ccc12]

To a solution of 80 mg (0.103 mmol, purity 93%) of the compound from Example 98A in 1.25 ml of dichloromethane were added at RT 0.625 ml (8.11 mmol) of trifluoroacetic acid and the reaction mixture was stirred for 1 h at RT. Then, the mixture was concentrated and the residue was dried in vacuo. The title compound was obtained in a purity of 85% according to LC/MS and used directly in the subsequent stage.

LC/MS (Method 1, ESIpos): Rₜ=1.15 min, m/z=611 [M+H]⁺.

### Example 118A

### [2-(4-Oxo-1,2,3-benzotriazin-3(4H)-yl)ethyl]{2-oxo-2-[6-(tetrahydro-2H-pyran-4-ylmethoxy)-1,2-benzoxazol-3-yl]ethyl}malonic acid

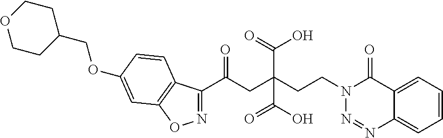 [SMILES: O=C(CC(CCn1nnc2ccccc2c1=O)(C(=O)O)C(=O)O)c1noc2cc(OCC3CCOCC3)ccc12]

To a solution of 550 mg (0.65 mmol, purity 78%) of the compound from Example 101A in 8.0 ml of dichloromethane were added at RT 4.0 ml (51.92 mmol) of trifluoroacetic acid and the reaction mixture was stirred for 1 h at RT. Then, the mixture was concentrated and the residue was dried in vacuo. The title compound was obtained in a purity of 68% according to LC/MS and used directly in the subsequent stage.

LC/MS (Method 2, ESIpos): Rₜ=1.97 min, m/z=551 [M+H]⁺.

### Example 119A

### (2-{6-[(4-Fluorobenzyl)oxy]-1,2-benzoxazol-3-yl}-2-oxoethyl)[2-(4-oxo-1,2,3-benzotriazin-3(4H)-yl)ethyl]malonic acid

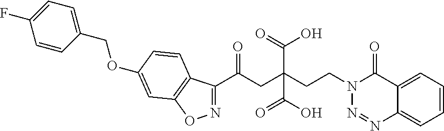 [SMILES: O=C(CC(CCn1nnc2ccccc2c1=O)(C(=O)O)C(=O)O)c1noc2cc(OCc3ccc(F)cc3)ccc12]

To a solution of 155 mg (0.21 mmol, purity 90%) of the compound from Example 102A in 2.5 ml of dichloromethane were added at RT 1.25 ml (16.23 mmol) of trifluoroacetic acid and the reaction mixture was stirred for 1 h at RT. Then, the mixture was concentrated and the residue was dried in vacuo. The title compound was obtained in a purity of 88% according to LC/MS and used directly in the subsequent stage.

LC/MS (Method 1, ESIpos): Rₜ=1.05 min, m/z=561 [M+H]⁺.

### Example 120A

### (2-{6-[(3-Carbamoylbenzyl)oxy]-1,2-benzoxazol-3-yl}-2-oxoethyl)[2-(4-oxo-1-benzotriazin-3(4H)-yl)ethyl]malonic acid

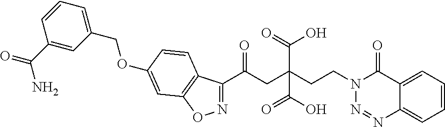 [SMILES: NC(=O)c1cccc(COc2ccc3c(C(=O)CC(CCn4nnc5ccccc5c4=O)(C(=O)O)C(=O)O)noc3c2)c1]

To a solution of 190 mg (0.19 mmol, purity 70%) of the compound from Example 103A in 3 ml of dichloromethane were added at RT 1.5 ml (19.47 mmol) of trifluoroacetic acid and the reaction mixture was stirred for 1 h at RT. Then, the mixture was concentrated and the residue was dried in vacuo. The title compound was obtained in a purity of 70% according to LC/MS and used directly in the subsequent stage.

LC/MS (Method 1, ESIpos): Rₜ=0.83 min, m/z=586 [M+H]⁺.

### Example 121A

### [2-(4-Oxo-1,2,3-benzotriazin-3(4H)-yl)ethyl]{2-oxo-2-[6-(pentyloxy)-1,2-benzoxazol-3-yl]ethyl}malonic acid

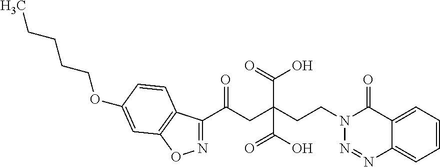 [SMILES: CCCCCOc1ccc2c(C(=O)CC(CCn3nnc4ccccc4c3=O)(C(=O)O)C(=O)O)noc2c1]

To a solution of 105 mg (0.15 mmol, purity 90%) of the compound from Example 104A in 2.5 ml of dichloromethane were added at RT 1.25 ml (16.23 mmol) of trifluoroacetic acid and the reaction mixture was stirred for 1 h at RT. Then, the mixture was concentrated and the residue was dried in vacuo. The title compound was obtained in a purity of 84% according to LC/MS and used directly in the subsequent stage.

LC/MS (Method 1, ESIpos): Rₜ=1.11 min, m/z=523 [M+H]⁺.

### Example 122A

### {2-[6-(2-Cyclohexylethoxy)-1,2-benzoxazol-3-yl]-2-oxoethyl}[2-(4-oxo-1,2,3-benzotriazin-3(4H)-yl)ethyl]malonic acid

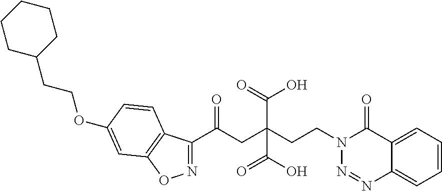 [SMILES: O=C(CC(CCn1nnc2ccccc2c1=O)(C(=O)O)C(=O)O)c1noc2cc(OCCC3CCCCC3)ccc12]

To a solution of 130 mg (0.19 mmol, purity 97%) of the compound from Example 105A in 3.5 ml of dichloromethane were added at RT 1.75 ml (22.71 mmol) of trifluoroacetic acid and the reaction mixture was stirred for 1 h at RT. Then, the mixture was concentrated and the residue was dried in vacuo. The title compound was obtained in a purity of 90% according to LC/MS and used directly in the subsequent stage.

LC/MS (Method 1, ESIpos): Rₜ=1.28 min, m/z=563 [M+H]⁺.

### Example 123A

### [2-(4-Oxo-1,2,3-benzotriazin-3(4H)-yl)ethyl]{2-oxo-2-[6-(2-phenylethoxy)-1,2-benzoxazol-3-yl]ethyl}malonic acid

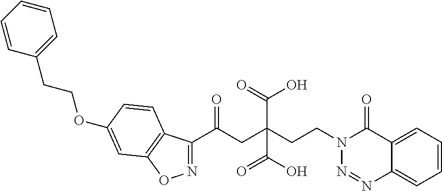 [SMILES: O=C(CC(CCn1nnc2ccccc2c1=O)(C(=O)O)C(=O)O)c1noc2cc(OCCc3ccccc3)ccc12]

To a solution of 125 mg (0.19 mmol) of the compound from Example 106A in 4 ml of dichloromethane were added at RT 2 ml (25.96 mmol) of trifluoroacetic acid and the reaction mixture was stirred for 1 h at RT. Then, the mixture was concentrated and the residue was dried in vacuo. The title compound was obtained in a purity of 96% according to LC/MS and used directly in the subsequent stage.

LC/MS (Method 1, ESIpos): Rₜ=1.06 min, m/z=557 [M+H]⁺.

### Example 124A

### (2-{6-[(3-Chlorobenzyl)oxy]-1,2-benzoxazol-3-yl}-2-oxoethyl)[2-(4-oxo-1,2,3-benzotriazin-3(4H)-yl)ethyl]malonic acid

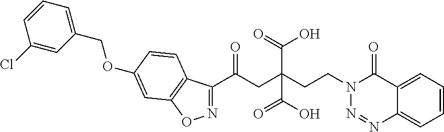 [SMILES: O=C(CC(CCn1nnc2ccccc2c1=O)(C(=O)O)C(=O)O)c1noc2cc(OCc3cccc(Cl)c3)ccc12]

To a solution of 150 mg (0.22 mmol) of the compound from Example 107A in 4.5 ml of dichloromethane were added at RT 2.25 ml (29.21 mmol) of trifluoroacetic acid and the reaction mixture was stirred for 2 h at RT. Then, the mixture was concentrated and the residue was dried in vacuo. The title compound was obtained in a purity of 98% according to LC/MS and used directly in the subsequent stage.

LC/MS (Method 1, ESIpos): Rₜ=1.08 min, m/z=577 [M+H]⁺.

### Example 125A

### (2-{6-[(4-Chlorobenzyl)oxy]-1,2-benzoxazol-3-yl}-2-oxoethyl)[2-(4-oxo-1,2,3-benzotriazin-3(4H)-yl)ethyl]malonic acid

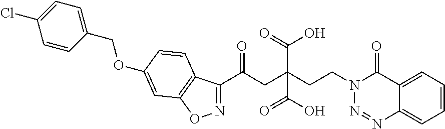 [SMILES: O=C(CC(CCn1nnc2ccccc2c1=O)(C(=O)O)C(=O)O)c1noc2cc(OCc3ccc(Cl)cc3)ccc12]

To a solution of 150 mg (0.22 mmol) of the compound from Example 108A in 4.5 ml of dichloromethane were added at RT 2.25 ml (29.21 mmol) of trifluoroacetic acid and the reaction mixture was stirred for 2 h at RT. Then, the mixture was concentrated and the residue was dried in vacuo. The title compound was obtained in a purity of 98% according to LC/MS and used directly in the subsequent stage.

LC/MS (Method 1, ESIpos): Rₜ=1.08 min, m/z=577 [M+H]⁺.

### Example 126A

### [2-(4-Oxo-1,2,3-benzotriazin-3(4H)-yl)ethyl]{2-oxo-2-[6-(pyridin-3-ylmethoxy)-1,2-benzoxazol-3-yl]ethyl}malonic acid

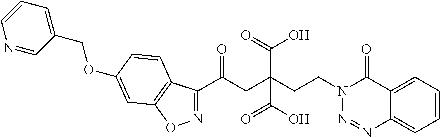 [SMILES: O=C(CC(CCn1nnc2ccccc2c1=O)(C(=O)O)C(=O)O)c1noc2cc(OCc3cccnc3)ccc12]

To a solution of 44 mg (0.067 mmol) of the compound from Example 109A in 1.5 ml of dichloromethane were added at RT 0.75 ml (9.74 mmol) of trifluoroacetic acid and the reaction mixture was stirred for 2 h at RT. Then, the mixture was concentrated and the residue was dried in vacuo. The title compound was obtained in a purity of 97% according to LC/MS and used directly in the subsequent stage.

LC/MS (Method 1, ESIpos): Rₜ=0.69 min, m/z=544 [M+H]⁺.

### Example 127A

### [2-(4-Oxo-1,2,3-benzotriazin-3(4H)-yl)ethyl](2-oxo-2-{6-[2-(tetrahydro-2H-pyran-4-yl)ethoxy]-1,2-benzoxazol-3-yl}ethyl)malonic acid

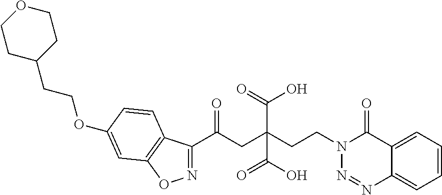 [SMILES: O=C(CC(CCn1nnc2ccccc2c1=O)(C(=O)O)C(=O)O)c1noc2cc(OCCC3CCOCC3)ccc12]

To a solution of 126 mg (0.17 mmol, purity 92%) of the compound from Example 110A in 3 ml of dichloromethane were added at RT 1.5 ml (19.47 mmol) of trifluoroacetic acid and the reaction mixture was stirred for 2 h at RT. Then, the mixture was concentrated and the residue was dried in vacuo. The title compound was obtained in a purity of 89% according to LC/MS and used directly in the subsequent stage.

LC/MS (Method 1, ESIpos): Rₜ=0.94 min, m/z=565 [M+H]⁺.

### Example 128A

### {2-[6-(Cyclopropylmethoxy)-1,2-benzoxazol-3-yl]-2-oxoethyl}[2-(4-oxo-1,2,3-benzotriazin-3(4H)-yl)ethyl]malonic acid

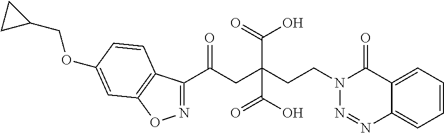 [SMILES: O=C(CC(CCn1nnc2ccccc2c1=O)(C(=O)O)C(=O)O)c1noc2cc(OCC3CC3)ccc12]

To a solution of 112 mg (0.17 mmol, purity 93%) of the compound from Example 111A in 3 ml of dichloromethane were added at RT 1.5 ml (19.47 mmol) of trifluoroacetic acid and the reaction mixture was stirred for 2 h at RT. Then, the mixture was concentrated and the residue was dried in vacuo. The title compound was obtained in a purity of 86% according to LC/MS and used directly in the subsequent stage.

LC/MS (Method 1, ESIpos): Rₜ=0.97 min, m/z=507 [M+H]⁺.

### Example 129A

### {2-[6-(2-Cyclopentylethoxy)-1,2-benzoxazol-3-yl]-2-oxoethyl}[2-(4-oxo-1,2,3-benzotriazin-3(4H)-yl)ethyl]malonic acid

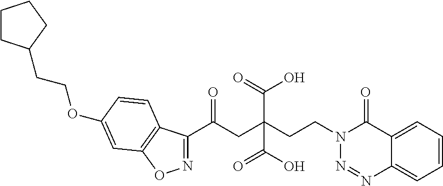 [SMILES: O=C(CC(CCn1nnc2ccccc2c1=O)(C(=O)O)C(=O)O)c1noc2cc(OCCC3CCCC3)ccc12]

To a solution of 120 mg (0.18 mmol, purity 99%) of the compound from Example 112A in 3 ml of dichloromethane were added at RT 1.5 ml (19.47 mmol) of trifluoroacetic acid and the reaction mixture was stirred for 2 h at RT. Then, the mixture was concentrated and the residue was dried in vacuo. The title compound was obtained in a purity of 95% according to LC/MS and used directly in the subsequent stage.

LC/MS (Method 1, ESIpos): Rₜ=1.20 min, m/z=549 [M+H]⁺.

### Example 130A

### {2-[6-(Cyclopentylmethoxy)-1,2-benzoxazol-3-yl]-2-oxoethyl}[2-(4-oxo-1,2,3-benzotriazin-3(4H)-yl)ethyl]malonic acid

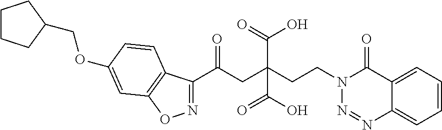 [SMILES: O=C(CC(CCn1nnc2ccccc2c1=O)(C(=O)O)C(=O)O)c1noc2cc(OCC3CCCC3)ccc12]

To a solution of 143 mg (0.17 mmol, purity 75%) of the compound from Example 113A in 3 ml of dichloromethane were added at RT 1.5 ml (19.47 mmol) of trifluoroacetic acid and the reaction mixture was stirred for 2 h at RT. Then, the mixture was concentrated and the residue was dried in vacuo. The title compound was obtained in a purity of 70% according to LC/MS and used directly in the subsequent stage.

LC/MS (Method 1, ESIpos): Rₜ=1.07 min, m/z=535 [M+H]⁺.

### Example 131A

### {2-[6-(Cyclohexylmethoxy)-1,2-benzoxazol-3-yl]-2-oxoethyl}[2-(4-oxo-1,2,3-benzotriazin-3(4H)-yl)ethyl]malonic acid

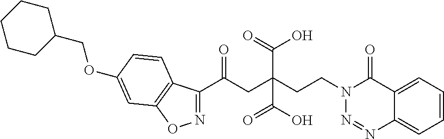 [SMILES: O=C(CC(CCn1nnc2ccccc2c1=O)(C(=O)O)C(=O)O)c1noc2cc(OCC3CCCCC3)ccc12]

To a solution of 113 mg (0.13 mmol, purity 79%) of the compound from Example 114A in 2 ml of dichloromethane were added at RT 1 ml (12.98 mmol) of trifluoroacetic acid and the reaction mixture was stirred for 2 h at RT. Then, the mixture was concentrated and the residue was dried in vacuo. The title compound was obtained in a purity of 75% according to LC/MS and used directly in the subsequent stage.

LC/MS (Method 1, ESIpos): Rₜ=1.15 min, m/z=549 [M+H]⁺.

### Example 132A

### {2-Oxo-2-[6-(tetrahydro-2H-pyran-4-ylmethoxy)-1,2-benzoxazol-3-yl]ethyl}{2-[4-oxo-6-(trifluoromethyl)-1,2,3-benzotriazin-3(4H)-yl]ethyl}malonic acid

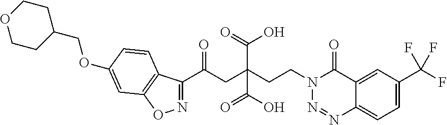 [SMILES: O=C(CC(CCn1nnc2ccc(C(F)(F)F)cc2c1=O)(C(=O)O)C(=O)O)c1noc2cc(OCC3CCOCC3)ccc12]

To a solution of 75 mg (0.10 mmol) of the compound from Example 115A in 1.5 ml of dichloromethane were added at RT 0.75 ml (9.74 mmol) of trifluoroacetic acid and the reaction mixture was stirred for 1 h at RT. Then, the mixture was concentrated and the residue was dried in vacuo. The title compound was obtained in a purity of 92% according to LC/MS and used directly in the subsequent stage.

LC/MS (Method 1, ESIpos): Rₜ=1.04 min, m/z=619 [M+H]⁺.

### Example 133A

### 4-(Benzyloxy)-2-nitro-1-(prop-1-yn-1-yl)benzene

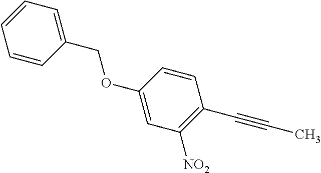 [SMILES: CC#Cc1ccc(OCc2ccccc2)cc1[N+](=O)[O-]]

To a solution of 4.68 g (15.19 mmol) of 4-(benzyloxy)-1-bromo-2-nitrobenzene [Lit.: Bioorg. Med. Chem. 2003, 11 (4), 521-528] in 30 ml of toluene under argon were added 878 mg (0.76 mmol) of tetrakis(triphenylphosphine)palladium(0) and the mixture was stirred for 10 min at RT. Then, 10.0 g (30.39 mmol) of tributyl(prop-1-yn-1-yl)stannane were added, and the mixture was stirred under reflux for 2 h. After cooling to RT, 10% strength aqueous potassium fluoride solution and ethyl acetate were added. The solid present was filtered off, whereupon phase separation ensued. The aqueous phase was extracted once with ethyl acetate, and the combined organic phases were washed once with saturated sodium chloride solution, dried over magnesium sulphate, filtered and concentrated. The crude product thus obtained was combined with the crude product from an experiment carried out analogously [amount of 4-(benzyloxy)-1-bromo-2-nitrobenzene used: 1.40 g (4.54 mmol)] and purified by column chromatography (300 g of silica gel, mobile phase cyclohexane/ethyl acetate 9:1). 3.25 g of a first batch of the title compound were obtained (purity 90%, 55% of theory, based on 6.08 g (19.93 mmol) of 4-(benzyloxy)-1-bromo-2-nitrobenzene used in total). A mixed fraction, which was after-purified by renewed column chromatography under analogous conditions, produced a further 813 mg (15% of theory, purity 99%) of the title compound.

¹H-NMR (400 MHz, DMSO-d₆): δ [ppm]=7.66 (d, 1H), 7.59 (d, 1H), 7.50-7.30 (m, 6H), 5.22 (s, 2H), 2.07 (s, 3H).

LC/MS (Method 2, ESIpos): Rₜ=2.71 min, m/z=268 [M+H]⁺.

### Example 134A

### 1-[6-(Benzyloxy)-2,1-benzoxazol-3-yl]ethanone

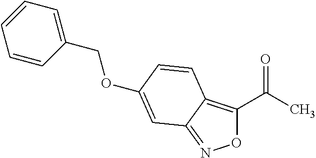 [SMILES: CC(=O)c1onc2cc(OCc3ccccc3)ccc12]

To a solution of 4.0 g (13.77 mmol, purity 92%) of the compound from Example 133A in 125 ml of dichloromethane were added 301 mg (0.69 mmol) of gold(III) bromide. The mixture was stirred at RT for 15 min. Then, the solution was washed once with water, dried over magnesium sulphate, filtered and concentrated. The residue was purified by means of column chromatography (300 g of silica gel, mobile phase: cyclohexane/ethyl acetate 85:15). 2.65 g (70% of theory, purity 97%) of the title compound were obtained.

¹H-NMR (400 MHz, DMSO-d₆): δ [ppm]=7.87 (d, 1H), 7.54-7.47 (m, 2H), 7.46-7.33 (m, 3H), 7.19 (d, 1H), 7.11 (dd, 1H), 5.23 (s, 2H), 2.72 (s, 3H).

LC/MS (Method 1, ESIpos): Rₜ=1.16 min, m/z=268 [M+H]⁺.

### Example 135A

### 1-[6-(Benzyloxy)-2,1-benzoxazol-3-yl]-2-bromoethanone

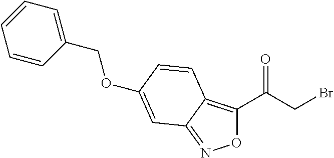 [SMILES: O=C(CBr)c1onc2cc(OCc3ccccc3)ccc12]

To a solution of 2.65 g (9.57 mmol, purity 97%) of the compound from Example 134A in 70 ml of THF were added at RT 3.98 g (10.05 mmol, purity 97%) of phenyltrimethylammonium tribromide. After stirring for 30 min at RT, the mixture was admixed with ethyl acetate and washed with water. The aqueous phase was back-extracted once with ethyl acetate, and the combined organic phases were washed with saturated sodium chloride solution, dried over magnesium sulphate, filtered and concentrated. The residue was taken up in dichloromethane and purified by column chromatography (200 g of silica gel, mobile phase cyclohexane/ethyl acetate 20:1→10:1). 1.57 g (42% of theory, purity 89%) of a first batch and 1.44 g (40% of theory, purity 91%) of a second batch of the title compound were obtained.

¹H-NMR (400 MHz, DMSO-d₆): δ [ppm]=7.89 (d, 1H), 7.53-7.48 (m, 2H), 7.46-7.34 (m, 3H), 7.23 (d, 1H), 7.17 (dd, 1H), 5.24 (s, 2H), 4.98 (s, 2H).

LC/MS (Method 1, ESIpos): Rₜ=1.24 min, m/z=346/348 [M+H]⁺.

### Example 136A

### Di-tert-butyl {2-[6-(benzyloxy)-2,1-benzoxazol-3-yl]-2-oxoethyl}[2-(4-oxo-1,2,3-benzotriazin-3(4H)-yl)ethyl]malonate

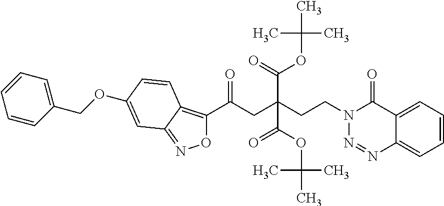 [SMILES: CC(C)(C)OC(=O)C(CCn1nnc2ccccc2c1=O)(CC(=O)c1onc2cc(OCc3ccccc3)ccc12)C(=O)OC(C)(C)C]

To a suspension of 21 mg (0.53 mmol) of sodium hydride (60% in paraffin oil) in 6 ml of DMF under argon were added at RT a solution of 165 mg (0.42 mmol) of the compound from Example 2A in 2 ml of DMF. After stirring for approx. 30 minutes at 50° C., the mixture was cooled to −20° C., and a solution of 170 mg (0.47 mmol, purity 95%) of the compound from Example 135A in 2 ml of DMF was quickly added. After removing the cooling bath, the mixture was after-stirred for 5 minutes at RT and the mixture was then admixed with ethyl acetate and water. After phase separation, the aqueous phase was extracted once with ethyl acetate, and the combined organic phases were washed with saturated sodium chloride solution, dried over magnesium sulphate, filtered and concentrated. The residue was purified by means of preparative HPLC (Method 6). 27 mg (10% of theory, purity 100%) of the title compound were obtained.

LC/MS (Method 1, ESIpos): Rₜ=1.51 min, m/z=655 [M+H]⁺.

### Example 137A

### {2-[6-(Benzyloxy)-2,1-benzoxazol-3-yl]-2-oxoethyl}[2-(4-oxo-1,2,3-benzotriazin-3(4H)-yl)ethyl]malonic acid

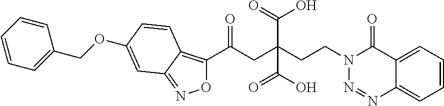 [SMILES: O=C(CC(CCn1nnc2ccccc2c1=O)(C(=O)O)C(=O)O)c1onc2cc(OCc3ccccc3)ccc12]

To a solution of 29 mg (0.044 mmol) of the compound from Example 136A in 1.0 ml of dichloromethane were added at RT 0.5 ml (6.5 mmol) of trifluoroacetic acid and the reaction mixture was stirred for 2 h at RT. Then, the mixture was concentrated and the residue was dried in vacuo. The title compound was obtained in a purity of 97% according to LC/MS and used directly in the subsequent stage.

LC/MS (Method 1, ESIpos): Rₜ=1.08 min, m/z=543 [M+H]⁺.

### Example 138A

### 2-(Trimethylsilyl)ethyl 2-{[(trifluoromethyl)sulphonyl]oxy}bicyclo[2.2.1]hept-2-ene-7-carboxylate

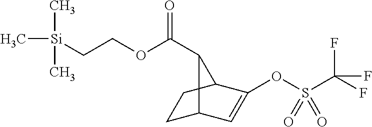 [SMILES: C[Si](C)(C)CCOC(=O)C1C2C=C(OS(=O)(=O)C(F)(F)F)C1CC2]

To a solution of 20.0 g (79.62 mmol) of 2-(trimethylsilyl)ethyl-2-oxobicyclo[2.2.1]heptane-7-carboxylate [WO 96/15096, Example 360/stage 1] in 130 ml of THF under argon were added at −78° C. slowly 173 ml (86.48 mmol) of a 0.5 M solution of potassium bis(trimethylsilyl)amide in THF and then a solution of 40.13 g (0.10 mol) of N-(5-chloropyridin-2-yl)-1,1,1-trifluoro-N-[(trifluoromethyl)sulphonyl]methanesulphonamide in 130 ml of THF. After stirring for 2 h at −78° C., the mixture was admixed with water and extracted twice with ethyl acetate. The combined organic phases were dried over magnesium sulphate, filtered and concentrated. The residue was purified by flash chromatography (silica gel, mobile phase cyclohexane/ethyl acetate 95:5-90:10→85:15). 32.4 g (98% of theory, purity 92%) of the title compound were obtained.

¹H-NMR (400 MHz, DMSO-d₆): δ [ppm]=6.04 (dd, 1H), 4.14-4.06 (m, 2H), 3.16-3.10 (m, 2H), 2.67 (s, 1H), 1.84-1.72 (m, 2H), 1.41-1.28 (m, 1H), 1.22-1.11 (m, 1H), 0.97-0.87 (m, 2H), 0.02 (s, 9H).

The product of an experiment carried out in a similar way [amount of 2-(trimethylsilyl)ethyl-2-oxobicyclo[2.2.1]heptane-7-carboxylate used: 312 mg (1.23 mmol)] produced the following LC/MS analysis:

LC/MS (Method 1, ESIneg): Rₜ=1.49 min, m/z=385 [M−H]−.

### Example 139A

### (+/−)-1-Benzyl-2-[2-(trimethylsilyl)ethyl]-(1RS,2RS,3SR)-3-(hydroxymethyl)cyclopentane-1,2-dicarboxylate (racemate)

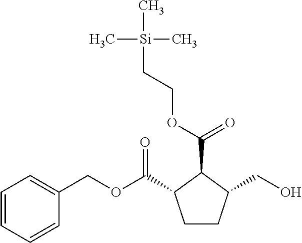 [SMILES: C[Si](C)(C)CCOC(=O)[C@H]1[C@H](CO)CC[C@@H]1C(=O)OCc1ccccc1]

To a solution of 11.30 g (26.75 mmol, purity 92%) of the compound from Example 138A in 67 ml of acetone under argon were added at 0° C. 7.05 g (60.20 mmol) of N-methylmorpholine N-oxide, dissolved in 19 ml of degassed water. Then, 51.4 ml of a 2.5% strength solution of osmium tetroxide in tert-butanol were slowly added, and the mixture was stirred firstly for 1 h at 0° C. and then for 1 h at RT. Then, the reaction mixture was admixed at RT with 17.17 g (80.26 mmol) of sodium periodate, during which a solid precipitated out. After stirring for 1 h at RT, the mixture was diluted with ethyl acetate and washed once with 10% strength aqueous citric acid solution and once with saturated sodium chloride solution. The organic phase was dried over magnesium sulphate, filtered and concentrated. 14.8 g of a residue were obtained [intermediate (+/−)-(1RS,2RS,3SR)-3-formyl-2-{[2-(trimethylsilyl)ethoxy]carbonyl}cyclopentanecarboxylic acid (racemate)].

The residue obtained above was taken up in 500 ml of dichloromethane and admixed with 327 mg (2.68 mmol) of 4-N,N-dimethylaminopyridine and 9.6 ml (55.11 mmol) of N,N-diisopropylethylamine. Then, at 0° C., 4.70 g (27.56 mmol) of benzyl chloroformate were added. After stirring at RT for 2 h, the mixture was diluted with dichloromethane and washed in each case once with 5% strength aqueous citric acid solution, saturated sodium hydrogencarbonate solution and saturated sodium chloride solution. The organic phase was dried over magnesium sulphate, filtered and concentrated. The residue was purified by means of flash chromatography (silica gel, mobile phase cyclohexane/ethyl acetate 85:15). 3.0 g of a solid were obtained [approx. 70% purity according to thin-layer chromatography; intermediate (+/−)-1-benzyl-2-[2-(trimethylsilyl)ethyl]-(1RS,2RS,3SR)-3-formylcyclopentane-1,2-dicarboxylate (racemate)].

The solid obtained above was taken up in 15 ml of ethanol and slowly admixed at RT with 56 mg (1.49 mmol) of sodium borohydride. After stirring for 30 min at RT, the mixture was admixed with 40 ml of saturated aqueous ammonium chloride solution, 40 ml of water and with ethyl acetate. After phase separation, the aqueous phase was extracted once with ethyl acetate. The combined organic phases were washed with saturated sodium chloride solution, dried over magnesium sulphate, filtered and concentrated. The residue was purified by means of column chromatography (80 g of silica gel, mobile phase: cyclohexane/ethyl acetate 85:15). In this way, a total of 1.26 g (12% of theory, purity 71%) of the title compound were obtained.

LC/MS (Method 1, ESIpos): Rₜ=1.24 min, m/z=379 [M+H]⁺.

### Example 140A

### (+/−)-1-Benzyl-2-[2-(trimethylsilyl)ethyl]-(1RS,2RS,3SR)-3-{[4-oxo-6-(trifluoromethyl)-1,2,3-benzotriazin-3(4H)-yl]methyl}cyclopentane-1,2-dicarboxylate (racemate)

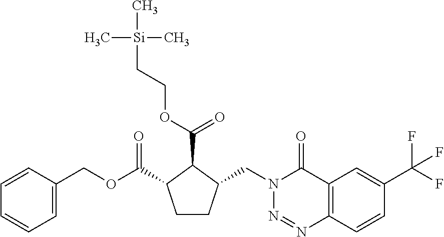 [SMILES: C[Si](C)(C)CCOC(=O)[C@H]1[C@H](Cn2nnc3ccc(C(F)(F)F)cc3c2=O)CC[C@@H]1C(=O)OCc1ccccc1]

To a mixture of 4.95 g (10.46 mmol, purity 80%) of the compound from Example 139A in 85 ml of toluene under argon were added 2.70 g (12.55 mmol) of the compound from Example 1A. Then, the mixture was cooled to 0° C., and 3.39 g (16.74 mmol) of tributylphosphane and 4.56 g (10.46 mmol) of diethyl azodicarboxylate (as 40% strength solution in toluene) were added. The mixture was then stirred at RT for 20 h. Then, the mixture was diluted with ethyl acetate and washed once with water. The aqueous phase was back-extracted once with ethyl acetate, and the combined organic phases were washed with saturated sodium chloride solution, dried over magnesium sulphate, filtered and concentrated. The residue was purified by means of column chromatography (300 g of silica gel, mobile phase: cyclohexane/ethyl acetate 85:15). 3.46 g (51% of theory, purity 88%) of the title compound were obtained.

LC/MS (Method 2, ESIpos): Rₜ=3.09 min, m/z=576 [M+H]⁺.

### Example 141A

### (+/−)-(1RS,2RS,3SR)-3-{[4-Oxo-6-(trifluoromethyl)-1,2,3-benzotriazin-3(4H)-yl]methyl}-2-{[2-(trimethylsilyl)ethoxy]carbonyl}cyclopentanecarboxylic acid (racemate)

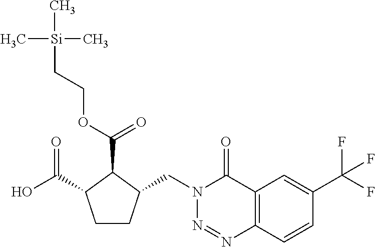 [SMILES: C[Si](C)(C)CCOC(=O)[C@H]1[C@H](Cn2nnc3ccc(C(F)(F)F)cc3c2=O)CC[C@@H]1C(=O)O]

To 602 mg (0.57 mmol) of palladium catalyst (10% on activated carbon, moistened with a small amount of ethanol) were added under argon 3.46 g (5.23 mmol, purity 87%) of the compound from Example 140A, dissolved in 70 ml of methanol. The mixture was hydrogenated at RT under standard pressure for 20 min. Then, the mixture was admixed with ethyl acetate, any solid present was filtered off over kieselguhr and the filter residue was after-washed with ethyl acetate. The filtrate was concentrated and the residue was dried in vacuo. 2.89 g of the title compound were obtained (>100%, still solvent-containing, purity 90% according to LC/MS).

LC/MS (Method 1, ESIpos): Rₜ=1.22 min, m/z=486 [M+H]⁺.

### Example 142A

### (+/−)-2-(Trimethylsilyl)ethyl-(1RS,2RS,5SR)-2-(hydroxymethyl)-5-{[4-oxo-6-(trifluoromethyl)-1,2,3-benzotriazin-3(4H)-yl]methyl}cyclopentanecarboxylate (racemate)

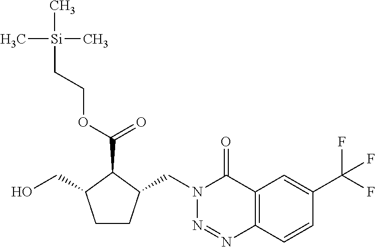 [SMILES: C[Si](C)(C)CCOC(=O)[C@@H]1[C@@H](CO)CC[C@H]1Cn1nnc2ccc(C(F)(F)F)cc2c1=O]

To 2.89 g (5.36 mmol, purity 90%) of the compound from Example 141A in 80 ml of THF under argon were slowly added at 0° C. 13.4 ml of a 2 M solution of borane dimethylsulphide in THF. After stirring for 5 min at 0° C. and for 1 h at RT, 400 ml of 0.1 N hydrochloric acid were added and the mixture was extracted twice with ethyl acetate. The combined organic phases were washed with saturated sodium chloride solution, dried over magnesium sulphate, filtered and concentrated. 2.93 g (purity 75%) of the title compound were obtained.

LC/MS (Method 1, ESIpos): Rₜ=1.28 min, m/z=472 [M+H]⁺.

### Example 143A

### (+/−)-2-(Trimethylsilyl)ethyl-(1RS,2RS,5SR)-2-formyl-5-{[4-oxo-6-(trifluoromethyl)-1,2,3-benzotriazin-3(4H)-yl]methyl}cyclopentanecarboxylate (racemate)

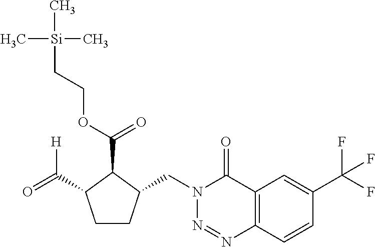 [SMILES: C[Si](C)(C)CCOC(=O)[C@H]1[C@H](Cn2nnc3ccc(C(F)(F)F)cc3c2=O)CC[C@@H]1C=O]

To 2.93 g (5.28 mmol, purity 85%) of the compound from Example 142A in 70 ml of dichloromethane were added 4.48 g (10.56 mmol) of 1,1,1-triacetoxy-1,1-dihydro-1,2-benziodoxol-3(1H)-one (Dess-Martin periodinane) and the mixture was stirred for 1 h at RT. Then, the mixture was diluted with dichloromethane and washed once with saturated aqueous sodium hydrogencarbonate solution. The organic phase was dried over magnesium sulphate, filtered and concentrated. The residue was purified by means of column chromatography (300 g of silica gel, mobile phase: cyclohexane/ethyl acetate 8:2). 1.59 g (63% of theory, purity 98%) of the title compound were obtained.

LC/MS (Method 1, ESIpos): Rₜ=1.34 min, m/z=470 [M+H]⁺.

### Example 144A

### 5-(2-Phenylethoxy)-1-benzofuran

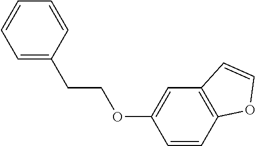 [SMILES: c1ccc(CCOc2ccc3occc3c2)cc1]

To a mixture of 1.06 g (7.90 mmol) of 1-benzofuran-5-ol and 2.18 g (15.82 mmol) of potassium carbonate in 40 ml of acetonitrile were added 2.69 g (9.48 mmol, purity 90%) of 2-phenylethyltrifluoromethanesulphonate [Lit.: Synthesis 1985, 8, 759-760] and the mixture was stirred for 2 h at RT. Then, the mixture was diluted with ethyl acetate and washed once with water. The aqueous phase was back-extracted once with ethyl acetate. The combined organic phases were washed with saturated sodium chloride solution, dried over magnesium sulphate, filtered and concentrated. The residue was purified by means of column chromatography (120 g of silica gel, mobile phase: cyclohexane/ethyl acetate 95:5). 1.39 g (72% of theory, purity 98%) of the title compound were obtained.

¹H-NMR (400 MHz, DMSO-d₆): δ [ppm]=7.93 (s, 1H), 7.47 (d, 1H), 7.38-7.15 (m, 6H), 6.92-6.83 (m, 2H), 4.20 (t, 2H), 3.05 (t, 2H).

LC/MS (Method 2, ESIpos): Rₜ=2.62 min, m/z=239 [M+H]⁺.

### Example 145A

### (+/−)-2-(Trimethylsilyl)ethyl-(1RS,2SR,5RS)-2-{[4-oxo-6-(trifluoromethyl)-1,2,3-benzotriazin-3(4H)-yl]methyl}-5-{[5-(2-phenylethoxy)-1-benzofur-2-yl]carbonyl}cyclopentanecarboxylate (racemate)

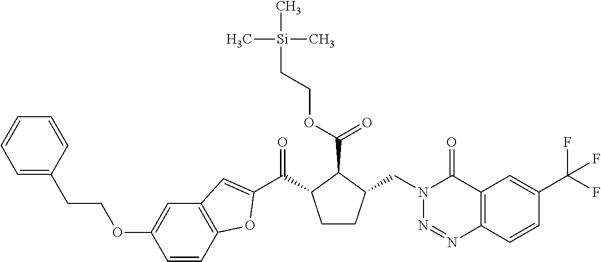 [SMILES: C[Si](C)(C)CCOC(=O)[C@H]1[C@H](Cn2nnc3ccc(C(F)(F)F)cc3c2=O)CC[C@@H]1C(=O)c1cc2cc(OCCc3ccccc3)ccc2o1]

To a mixture of 883 mg (3.63 mmol, purity 98%) of the compound from Example 144A in 30 ml of THF under argon were slowly added, at an internal temperature of −70° C., 2.3 ml (3.63 mmol) of a butyllithium solution (1.6 M in THF), and the mixture was stirred for 5 min at −70° C. and for a further 30 min at −30° C. Then, the mixture was cooled again to −70° C., and a solution of 1.59 g (3.30 mmol, purity 98%) of the compound from Example 143A in 20 ml of THF was added. After stirring for a further 20 min at −70° C., saturated aqueous ammonium chloride solution and water were added, and the mixture was extracted twice with ethyl acetate. The combined organic phases were dried over magnesium sulphate, filtered and concentrated. The residue was taken up in 30 ml of dichloromethane, admixed with 598 mg (1.41 mmol) of 1,1,1-triacetoxy-1,1-dihydro-1,2-benziodoxol-3(1H)-one (Dess-Martin periodinane) and stirred for 15 min at RT. Then, the mixture was diluted with dichloromethane and washed once with saturated aqueous sodium hydrogencarbonate solution. The aqueous phase was back-extracted once with dichloromethane. The combined organic phases were dried over magnesium sulphate, filtered and concentrated. The residue was purified by means of column chromatography (200 g of silica gel, mobile phase: cyclohexane/ethyl acetate 85:15). 695 mg (19% of theory, purity 69%) of the title compound were obtained.

LC/MS (Method 1, ESIpos): Rₜ=1.56 min, m/z=706 [M+H]⁺.

### Example 146A

### [2-(4-Oxo-1,2,3-benzotriazin-3(4H)-yl)ethyl]{2-oxo-2-[6-(tetrahydro-2H-pyran-4-ylmethoxy)-1-benzofur-3-yl]ethyl}malonic acid

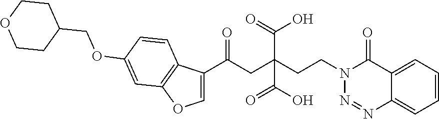 [SMILES: O=C(CC(CCn1nnc2ccccc2c1=O)(C(=O)O)C(=O)O)c1coc2cc(OCC3CCOCC3)ccc12]

To a solution of 405 mg (0.61 mmol) of the compound from Example 65A in 2.8 ml of dichloromethane were added at RT 1.4 ml (18.1 mmol) of trifluoroacetic acid and the reaction mixture was stirred for 1 h at RT. Then, the mixture was concentrated and the residue was dried in vacuo. The title compound was obtained in a purity of 86% according to LC/MS and used directly in the subsequent stage.

LC/MS (Method 1, ESIpos): Rₜ=0.86 min, m/z=550 [M+H]⁺.

### Example 147A

### [2-(6-Isobutoxy-1-benzofur-3-yl)-2-oxoethyl][2-(4-oxo-1,2,3-benzotriazin-3(4H)-yl)ethyl]malonic acid

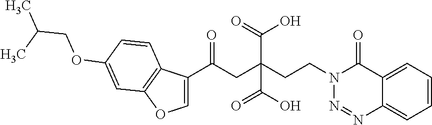 [SMILES: CC(C)COc1ccc2c(C(=O)CC(CCn3nnc4ccccc4c3=O)(C(=O)O)C(=O)O)coc2c1]

To a solution of 326 mg (0.53 mmol) of the compound from Example 68A in 2.4 ml of dichloromethane were added at RT 1.2 ml (15.6 mmol) of trifluoroacetic acid and the reaction mixture was stirred for 2 h at RT. Then, the mixture was concentrated and the residue was dried in vacuo. The title compound was obtained in a purity of 89% according to LC/MS and used directly in the subsequent stage.

LC/MS (Method 1, ESIpos): Rₜ=1.03 min, m/z=508 [M+H]⁺.

### WORKING EXAMPLES

### Example 1

### (+/−)-4-{5-[2-(2-Chlorophenyl)ethoxy]-1-benzofur-2-yl}-4-oxo-2-[2-(4-oxo-1,2,3-benzotriazin-3(4H)-yl)ethyl]butanoic acid

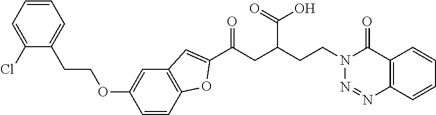 [SMILES: O=C(CC(CCn1nnc2ccccc2c1=O)C(=O)O)c1cc2cc(OCCc3ccccc3Cl)ccc2o1]

A solution of 17 mg (0.03 mmol) of the compound from Example 26A in 2 ml of dioxane was stirred under reflux for 3 h. After cooling to RT and concentration, the residue was purified by thick-layer chromatography (PLC silica gel 60 F 254, 1 mm; 20×20 cm with concentration zone; mobile phase dichloromethane/methanol 95:5). 14 mg (78% of theory, purity 90%) of the title compound were obtained.

¹H-NMR (400 MHz, DMSO-d₆): δ [ppm]=12.45 (br. s, 1H), 8.41-6.98 (m, 12H), 4.63-4.42 (m, 2H), 4.33-4.12 (m, 2H), 3.84-2.74 (m, 5H), 2.35-1.92 (m, 2H).

LC/MS (Method 1, ESIpos): Rₜ=1.25 min, m/z=546 [M+H]⁺.

### Example 2

### (+/−)-4-{5-[2-(4-Chlorophenyl)ethoxy]-1-benzofur-2-yl}-4-oxo-2-[2-(4-oxo-1,2,3-benzotriazin-3(4H)-yl)ethyl]butanoic acid

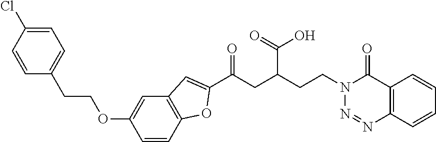 [SMILES: O=C(CC(CCn1nnc2ccccc2c1=O)C(=O)O)c1cc2cc(OCCc3ccc(Cl)cc3)ccc2o1]

A solution of 11 mg (0.02 mmol) of the compound from Example 27A in 2 ml of dioxane was stirred under reflux for 3 h. After cooling to RT and concentration, the residue was purified by thick-layer chromatography (PLC silica gel 60 F 254, 1 mm; 20×20 cm with concentration zone; mobile phase dichloromethane/methanol 95:5). 10 mg (77% of theory, purity 80%) of the title compound were obtained.

¹H-NMR (400 MHz, DMSO-d₆): δ [ppm]=12.43 (br. s, 1H), 8.23 (dd, 2H), 8.13-8.04 (m, 1H), 7.96-7.90 (m, 1H), 7.82 (s, 1H), 7.61 (d, 1H), 7.40-7.36 (m, 4H), 7.31 (d, 1H), 7.12 (dd, 1H), 4.58-4.43 (m, 2H), 4.24 (t, 2H), 3.44 (dd, 1H), 3.26-3.19 (m, 1H), 3.07 (t, 2H), 3.02-2.92 (m, 1H), 2.29-2.17 (m, 1H), 2.13-2.03 (m, 1H).

LC/MS (Method 1, ESIpos): Rₜ=1.24 min, m/z=546 [M+H]⁺.

### Example 3

### (+/−)-4-{5-[2-(4-Methoxyphenyl)ethoxy]-1-benzofur-2-yl}-4-oxo-2-[2-(4-oxo-1,2,3-benzotriazin-3(4H)-yl)ethyl]butanoic acid

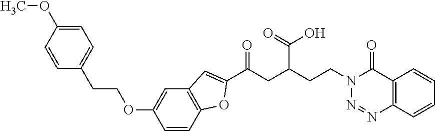 [SMILES: COc1ccc(CCOc2ccc3oc(C(=O)CC(CCn4nnc5ccccc5c4=O)C(=O)O)cc3c2)cc1]

A solution of 88 mg (0.15 mmol) of the compound from Example 28A in 4 ml of dioxane was stirred under reflux for 3 h. After cooling to RT and concentration, the residue was purified by thick-layer chromatography (PLC silica gel 60 F 254, 1 mm; 20×20 cm with concentration zone; mobile phase dichloromethane/methanol 95:5). 58 mg (72% of theory, purity 100%) of the title compound were obtained.

¹H-NMR (400 MHz, DMSO-d₆): δ [ppm]=12.46 (br. s, 1H), 8.26 (d, 1H), 8.20 (d, 1H), 8.12-8.04 (m, 1H), 7.97-7.89 (m, 1H), 7.81 (s, 1H), 7.61 (d, 1H), 7.30 (d, 1H), 7.26 (d, 2H), 7.13 (dd, 1H), 6.88 (d, 2H), 4.59-4.42 (m, 2H), 4.19 (t, 2H), 3.73 (s, 3H), 3.44 (dd, 1H), 3.23 (dd, 1H), 3.04-2.93 (m, 3H), 2.30-2.17 (m, 1H), 2.14-2.01 (m, 1H).

LC/MS (Method 1, ESIpos): Rₜ=1.17 min, m/z=542 [M+H]⁺.

### Example 4

### (+/−)-4-{5-[2-(2-Methylphenyl)ethoxy]-1-benzofur-2-yl}-4-oxo-2-[2-(4-oxo-1,2,3-benzotriazin-3(4H)-yl)ethyl]butanoic acid

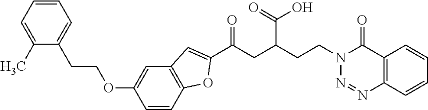 [SMILES: Cc1ccccc1CCOc1ccc2oc(C(=O)CC(CCn3nnc4ccccc4c3=O)C(=O)O)cc2c1]

A solution of 60 mg (0.11 mmol) of the compound from Example 29A in 5 ml of dioxane was stirred under reflux for 6 h. After cooling to RT and concentration, the residue was purified by means of preparative HPLC (Method 6). 38 mg (68% of theory, purity 100%) of the title compound were obtained.

¹H-NMR (400 MHz, DMSO-d₆): δ [ppm]=12.44 (br. s, 1H), 8.26 (dd, 1H), 8.20 (d, 1H), 8.12-8.05 (m, 1H), 7.96-7.89 (m, 1H), 7.81 (s, 1H), 7.61 (d, 1H), 7.33 (d, 1H), 7.30-7.25 (m, 1H), 7.21-7.10 (m, 4H), 4.59-4.41 (m, 2H), 4.22 (t, 2H), 3.44 (dd, 1H), 3.23 (dd, 1H), 3.07 (t, 2H), 3.02-2.92 (m, 1H), 2.34 (s, 3H), 2.30-2.16 (m, 1H), 2.13-2.02 (m, 1H).

LC/MS (Method 1, ESIpos): Rₜ=1.23 min, m/z=526 [M+H]⁺.

### Example 5

### (+/−)-4-Oxo-2-[2-(4-oxo-1,2,3-benzotriazin-3(4H)-yl)ethyl]-4-[5-(tetrahydro-2H-pyran-4-ylmethoxy)-1-benzofur-2-yl]butanoic acid

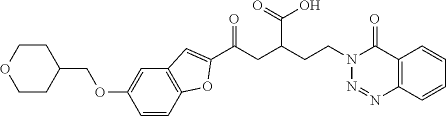 [SMILES: O=C(CC(CCn1nnc2ccccc2c1=O)C(=O)O)c1cc2cc(OCC3CCOCC3)ccc2o1]

A solution of 82 mg (0.15 mmol) of the compound from Example 30A in 3 ml of dioxane was stirred under reflux for 2 h. After cooling to RT and concentration, the residue was purified by thick-layer chromatography (PLC silica gel 60 F 254, 1 mm; 20×20 cm with concentration zone; mobile phase dichloromethane/methanol 9:1). 51 mg (65% of theory, purity 97%) of the title compound were obtained.

¹H-NMR (400 MHz, DMSO-d₆): δ [ppm]=12.46 (br. s, 1H), 8.26 (dd, 1H), 8.21 (d, 1H), 8.13-8.04 (m, 1H), 7.97-7.89 (m, 1H), 7.82 (s, 1H), 7.62 (d, 1H), 7.30 (d, 1H), 7.15 (dd, 1H), 4.58-4.42 (m, 2H), 3.94-3.83 (m, 4H), 3.49-3.39 (m, 1H), 3.38-3.32 (m, 2H, partially hidden), 3.27-3.18 (m, 1H), 3.03-2.92 (m, 1H), 2.29-2.17 (m, 1H), 2.13-1.96 (m, 2H), 1.75-1.65 (m, 2H), 1.43-1.28 (m, 2H).

LC/MS (Method 1, ESIpos): Rₜ=0.87 min, m/z=506 [M+H]⁺.

### Example 6

### (+/−)-4-Oxo-2-[2-(4-oxo-1,2,3-benzotriazin-3(4H)-yl)ethyl]-4-{5-[2-(tetrahydro-2H-pyran-4-yl)ethoxy]-1-benzofur-2-yl}butanoic acid

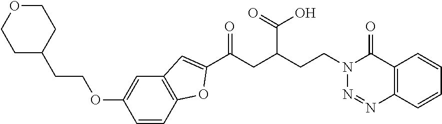 [SMILES: O=C(CC(CCn1nnc2ccccc2c1=O)C(=O)O)c1cc2cc(OCCC3CCOCC3)ccc2o1]

A solution of 103 mg (0.15 mmol, purity 80%) of the compound from Example 31A in 1.5 ml of dioxane was stirred under reflux for 3 h. After cooling to RT and concentration, the residue was purified by thick-layer chromatography (PLC silica gel 60 F 254, 1 mm; 20×20 cm with concentration zone; mobile phase dichloromethane/methanol 9:1). 53 mg (70% of theory, purity 100%) of the title compound were obtained.

¹H-NMR (400 MHz, DMSO-d₆): δ [ppm]=12.45 (br. s, 1H), 8.26 (d, 1H), 8.21 (d, 1H), 8.09 (dd, 1H), 7.94 (dd, 1H), 7.82 (br. s, 1H), 7.61 (d, 1H), 7.30 (d, 1H), 7.14 (dd, 1H), 4.57-4.45 (m, 2H), 4.06 (dd, 2H), 3.84 (dd, 2H), 3.44 (dd, 1H), 3.30-3.20 (m, 3H), 3.01-2.95 (m, 1H), 2.28-2.20 (m, 1H), 2.12-2.04 (m, 1H), 1.74-1.61 (m, 5H), 1.29-1.19 (m, 2H).

LC/MS (Method 1, ESIpos): Rₜ=1.04 min, m/z=520 [M+H]⁺.

### Example 7

### (+/−)-4-[5-(2-Cyclopentylethoxy)-1-benzofur-2-yl]-4-oxo-2-[2-(4-oxo-1,2,3-benzotriazin-3(4H)-yl)ethyl]butanoic acid

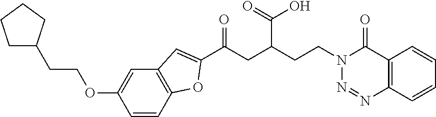 [SMILES: O=C(CC(CCn1nnc2ccccc2c1=O)C(=O)O)c1cc2cc(OCCC3CCCC3)ccc2o1]

A solution of 132 mg (0.22 mmol, purity 90%) of the compound from Example 32A in 2.5 ml of dioxane was stirred under reflux for 1 h. After cooling to RT and concentration, the residue was purified by thick-layer chromatography (PLC silica gel 60 F 254, 1 mm; 20×20 cm with concentration zone; mobile phase dichloromethane/methanol 9:1). 94 mg (86% of theory, purity 100%) of the title compound were obtained.

¹H-NMR (400 MHz, DMSO-d₆): δ [ppm]=12.47 (br. s, 1H), 8.26 (d, 1H), 8.21 (d, 1H), 8.09 (dd, 1H), 7.94 (dd, 1H), 7.82 (s, 1H), 7.61 (d, 1H), 7.30 (d, 1H), 7.14 (dd, 1H), 4.57-4.45 (m, 2H), 4.02 (dd, 2H), 3.44 (dd, 1H), 3.22 (dd, 1H), 3.01-2.95 (m, 1H), 2.27-2.20 (m, 1H), 2.12-2.04 (m, 1H), 1.98-1.93 (m, 1H), 1.83-1.74 (m, 4H), 1.62-1.49 (m, 4H), 1.21-1.12 (m, 2H).

LC/MS (Method 1, ESIpos): Rₜ=1.29 min, m/z=504 [M+H]⁺.

### Example 8

### (+/−)-4-[5-(Cyclohexylmethoxy)-1-benzofur-2-yl]-4-oxo-2-[2-(4-oxo-1,2,3-benzotriazin-3(4H)-yl)ethyl]butanoic acid

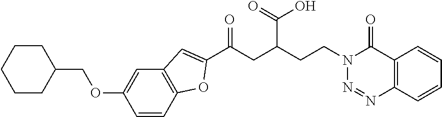 [SMILES: O=C(CC(CCn1nnc2ccccc2c1=O)C(=O)O)c1cc2cc(OCC3CCCCC3)ccc2o1]

A solution of 465 mg (0.75 mmol, purity 88%) of the compound from Example 33A in 4.5 ml of dioxane was stirred under reflux for 4 h. After cooling to RT and concentration, the residue was purified by means of preparative HPLC (Method 6, two injections). 310 mg (82% of theory, purity 100%) of the title compound were obtained.

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.82 (s, 1H), 7.61 (d, 1H), 7.27 (d, 1H), 7.14 (dd, 1H), 4.57-4.45 (m, 2H), 3.82 (d, 2H), 3.44 (dd, 1H), 3.23 (dd, 1H), 3.01-2.95 (m, 1H), 2.27-2.20 (m, 1H), 2.12-2.04 (m, 1H), 1.84 (d, 2H), 1.77-1.65 (m, 4H), 1.31-1.16 (m, 3H), 1.13-1.02 (m, 2H).

LC/MS (Method 1, ESIpos): Rₜ=1.32 min, m/z=504 [M+H]⁺.

Separation of the Enantiomers:

246 mg of the compound from Example 8 were dissolved in 3 ml of acetonitrile and 3 ml of ethanol and separated into the enantiomers by preparative HPLC on a chiral phase (see Examples 9 and 10) [column: Daicel Chiralpak AD-H, 5 μm, 250 mm×20 mm; flow rate: 20 ml/min; detection: 290 nm; injection volume: 0.5 ml; temperature: 25° C.; mobile phase 70% ethanol/30% acetonitrile+0.2% acetic acid, isocratic, run time 17 min]:

### Example 9

### (−)-4-[5-(Cyclohexylmethoxy)-1-benzofur-2-yl]-4-oxo-2-[2-(4-oxo-1,2,3-benzotriazin-3(4H)-yl)ethyl]butanoic acid (enantiomer 1)

87 mg (chemical purity 100%) of the title compound were obtained.

ee value=100%; [α]_{D}²⁰=−33.5°, 589 nm, c=0.37 g/100 ml, chloroform

¹H-NMR (400 MHz, DMSO-d₆): δ [ppm]=12.45 (br. s, 1H), 8.26 (d, 1H), 8.21 (d, 1H), 8.09 (dd, 1H), 7.94 (dd, 1H), 7.81 (s, 1H), 7.61 (d, 1H), 7.27 (d, 1H), 7.13 (dd, 1H), 4.57-4.45 (m, 2H), 3.82 (d, 2H), 3.44 (dd, 1H), 3.23 (dd, 1H), 3.01-2.95 (m, 1H), 2.28-2.20 (m, 1H), 2.12-2.04 (m, 1H), 1.84 (d, 2H), 1.77-1.65 (m, 4H), 1.31-1.16 (m, 3H), 1.13-1.02 (m, 2H).

LC/MS (Method 1, ESIpos): Rₜ=1.29 min, m/z=504 [M+H]⁺.

### Example 10

### (+)-4-[5-(Cyclohexylmethoxy)-1-benzofur-2-yl]-4-oxo-2-[2-(4-oxo-1,2,3-benzotriazin-3(4H)-yl)ethyl]butanoic acid (enantiomer 2)

90 mg (chemical purity 100%) of the title compound were obtained.

ee value=100%; [α]_{D}²⁰=+30.1°, 589 nm, c=0.38 g/100 ml, chloroform

¹H-NMR (400 MHz, DMSO-d₆): δ [ppm]=12.45 (br. s, 1H), 8.26 (d, 1H), 8.21 (d, 1H), 8.09 (dd, 1H), 7.94 (dd, 1H), 7.81 (s, 1H), 7.61 (d, 1H), 7.27 (d, 1H), 7.13 (dd, 1H), 4.57-4.45 (m, 2H), 3.82 (d, 2H), 3.44 (dd, 1H), 3.23 (dd, 1H), 3.01-2.95 (m, 1H), 2.28-2.20 (m, 1H), 2.12-2.04 (m, 1H), 1.84 (d, 2H), 1.77-1.65 (m, 4H), 1.31-1.16 (m, 3H), 1.13-1.02 (m, 2H).

LC/MS (Method 1, ESIpos): Rₜ=1.29 min, m/z=504 [M+H]⁺.

### Example 11

### (+/−)-4-Oxo-2-[2-(4-oxo-1,2,3-benzotriazin-3(4H)-yl)ethyl]-4-[5-(4,4,4-trifluorobutoxy)-1-benzofur-2-yl]butanoic acid

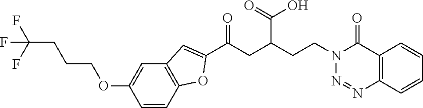 [SMILES: O=C(CC(CCn1nnc2ccccc2c1=O)C(=O)O)c1cc2cc(OCCCC(F)(F)F)ccc2o1]

A solution of 173 mg (0.31 mmol) of the compound from Example 34A in 8 ml of dioxane was stirred under reflux for 18 h. After cooling to RT and concentration, the residue was purified by means of preparative HPLC (Method 6). 91 mg (57% of theory, purity 100%) of the title compound were obtained.

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.85 (s, 1H), 7.64 (d, 1H), 7.31 (d, 1H), 7.17 (dd, 1H), 4.57-4.45 (m, 2H), 4.09 (dd, 2H), 3.44 (dd, 1H), 3.23 (dd, 1H), 3.01-2.95 (m, 1H), 2.48-2.40 (m, 2H), 2.29-2.20 (m, 1H), 2.13-2.04 (m, 1H), 2.01-1.94 (m, 2H).

LC/MS (Method 1, ESIpos): Rₜ=1.13 min, m/z=518 [M+H]⁺.

Separation of the Enantiomers:

77 mg of the compound from Example 11 were dissolved in 4 ml of ethanol and separated into the enantiomers by preparative HPLC on a chiral phase (see Examples 12 and 13) [column: Daicel Chiralpak AD-H, 5 μm, 250 mm×20 mm; flow rate: 20 ml/min; detection: 230 nm; temperature: 25° C.; mobile phase: 30% acetonitrile+0.2% acetic acid/70% ethanol+0.2% acetic acid, isocratic, run time 10 min]:

### Example 12

### (−)-4-Oxo-2-[2-(4-oxo-1,2,3-benzotriazin-3(4H)-yl)ethyl]-4-[5-(4,4,4-trifluorobutoxy)-1-benzofur-2-yl]butanoic acid (enantiomer 1)

24 mg (chemical purity 100%) of the title compound were obtained.

ee value=98%; [α]_{D}²⁰=−25.8°, 589 nm, c=0.32 g/100 ml, chloroform

¹H-NMR (400 MHz, DMSO-d₆): δ [ppm]=12.50 (br. s, 1H), 8.26 (d, 1H), 8.21 (d, 1H), 8.09 (dd, 1H), 7.94 (dd, 1H), 7.85 (s, 1H), 7.63 (d, 1H), 7.31 (d, 1H), 7.17 (dd, 1H), 4.57-4.45 (m, 2H), 4.09 (dd, 2H), 3.44 (dd, 1H), 3.23 (dd, 1H), 3.01-2.95 (m, 1H), 2.48-2.40 (m, 2H), 2.28-2.19 (m, 1H), 2.13-2.04 (m, 1H), 2.01-1.94 (m, 2H).

LC/MS (Method 1, ESIpos): Rₜ=1.12 min, m/z=518 [M+H]⁺.

### Example 13

### (+)-4-Oxo-2-[2-(4-oxo-1,2,3-benzotriazin-3(4H)-yl)ethyl]-4-[5-(4,4,4-trifluorobutoxy)-1-benzofur-2-yl]butanoic acid (enantiomer 2)

23 mg (chemical purity 96%) of the title compound were obtained.

ee value=98%; [α]_{D}²⁰=+23.9°, 589 nm, c=0.31 g/100 ml, chloroform

¹H-NMR (400 MHz, DMSO-d₆): δ [ppm]=12.50 (br. s, 1H), 8.26 (d, 1H), 8.21 (d, 1H), 8.09 (dd, 1H), 7.94 (dd, 1H), 7.85 (s, 1H), 7.63 (d, 1H), 7.31 (d, 1H), 7.17 (dd, 1H), 4.57-4.45 (m, 2H), 4.09 (dd, 2H), 3.44 (dd, 1H), 3.23 (dd, 1H), 3.01-2.95 (m, 1H), 2.48-2.40 (m, 2H), 2.28-2.19 (m, 1H), 2.13-2.04 (m, 1H), 2.01-1.94 (m, 2H).

LC/MS (Method 1, ESIpos): Rₜ=1.12 min, m/z=518 [M+H]+.

### Example 14

### (+/−)-4-{5-[(4-Fluorotetrahydro-2H-pyran-4-yl)methoxy]-1-benzofur-2-yl}-4-oxo-2-[2-(4-oxo-1,2,3-benzotriazin-3(4H)-yl)ethyl]butanoic acid

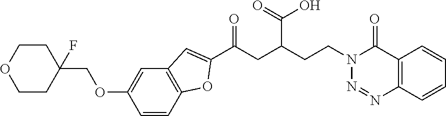 [SMILES: O=C(CC(CCn1nnc2ccccc2c1=O)C(=O)O)c1cc2cc(OCC3(F)CCOCC3)ccc2o1]

A solution of 275 mg (0.48 mmol) of the compound from Example 35A in 13 ml of dioxane was stirred under reflux for 18 h. After cooling to RT and concentration, the residue was purified by means of preparative HPLC (Method 6). 195 mg (77% of theory, purity 100%) of the title compound were obtained.

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.85 (s, 1H), 7.64 (d, 1H), 7.35 (d, 1H), 7.20 (dd, 1H), 4.57-4.45 (m, 2H), 4.18 (s, 1H), 4.13 (s, 1H), 3.81-3.76 (m, 2H), 3.65-3.58 (m, 2H), 3.44 (dd, 1H), 3.24 (dd, 1H), 3.01-2.95 (m, 1H), 2.29-2.20 (m, 1H), 2.13-2.04 (m, 1H), 1.97-1.79 (m, 4H).

LC/MS (Method 1, ESIpos): Rₜ=0.98 min, m/z=524 [M+H]⁺.

### Example 15

### (+/−)-4-[5-(3-Cyanopropoxy)-1-benzofur-2-yl]-4-oxo-2-[2-(4-oxo-1,2,3-benzotriazin-3(4H)-yl)ethyl]butanoic acid

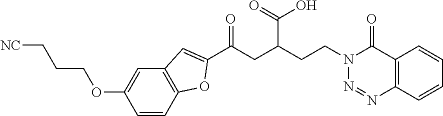 [SMILES: N#CCCCOc1ccc2oc(C(=O)CC(CCn3nnc4ccccc4c3=O)C(=O)O)cc2c1]

A solution of 164 mg (0.32 mmol) of the compound from Example 36A in 8 ml of dioxane was stirred under reflux for 18 h. After cooling to RT and concentration, the residue was purified by means of preparative HPLC (Method 6). 74 mg (48% of theory, purity 96%) of the title compound were obtained.

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.84 (s, 1H), 7.64 (d, 1H), 7.33 (d, 1H), 7.17 (dd, 1H), 4.58-4.45 (m, 2H), 4.09 (dd, 2H), 3.44 (dd, 1H), 3.24 (dd, 1H), 3.01-2.95 (m, 1H), 2.29 (t, 2H), 2.29-2.20 (m, 1H), 2.13-2.03 (m, 3H).

LC/MS (Method 1, ESIpos): Rₜ=0.95 min, m/z=475 [M+H]⁺.

### Example 16

### (+/−)-4-[5-(Cyclopentylmethoxy)-1-benzofur-2-yl]-4-oxo-2-[2-(4-oxo-1,2,3-benzotriazin-3(4H)-yl)ethyl]butanoic acid

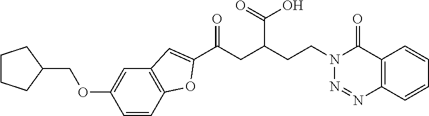 [SMILES: O=C(CC(CCn1nnc2ccccc2c1=O)C(=O)O)c1cc2cc(OCC3CCCC3)ccc2o1]

A solution of 479 mg (0.90 mmol) of the compound from Example 37A in 28 ml of dioxane was stirred under reflux for 18 h. After cooling to RT and concentration, the residue was purified by column chromatography (80 g of silica gel, mobile phase cyclohexane/ethyl acetate 9:1). 284 mg (62% of theory, purity 96%) of the title compound were obtained.

¹H-NMR (400 MHz, DMSO-d₆): δ [ppm]=12.47 (br. s, 1H), 8.26 (d, 1H), 8.21 (d, 1H), 8.09 (dd, 1H), 7.94 (dd, 1H), 7.82 (s, 1H), 7.61 (d, 1H), 7.29 (d, 1H), 7.14 (dd, 1H), 4.57-4.45 (m, 2H), 3.89 (d, 2H), 3.44 (d, 1H), 3.23 (dd, 1H), 3.01-2.95 (m, 1H), 2.37-2.20 (m, 2H), 2.12-2.04 (m, 1H), 1.83-1.75 (m, 2H), 1.64-1.51 (m, 4H), 1.40-1.32 (m, 2H).

LC/MS (Method 1, ESIpos): Rₜ=1.24 min, m/z=490 [M+H]⁺.

Separation of the Enantiomers:

236 mg of the compound from Example 16 were dissolved in 5 ml of methanol and separated into the enantiomers by preparative HPLC on a chiral phase (see Examples 17 and 18) [column: Daicel Chiralpak AD-H, 5 μm, 250 mm×20 mm; flow rate: 20 ml/min; detection: 290 nm; injection volume: 0.5 ml; temperature: 20° C.; mobile phase: 30% acetonitrile/70% ethanol+0.2% diethylamine, isocratic, run time 14 min]:

### Example 17

### (−)-4-[5-(Cyclopentylmethoxy)-1-benzofur-2-yl]-4-oxo-2-[2-(4-oxo-1,2,3-benzotriazin-3(4H)-yl)ethyl]butanoic acid (enantiomer 1)

79 mg (chemical purity 100%) of the title compound were obtained.

ee value=100%; [α]_{D}²⁰=−33.7°, 589 nm, c=0.32 g/100 ml, chloroform

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.82 (s, 1H), 7.61 (d, 1H), 7.29 (d, 1H), 7.14 (dd, 1H), 4.57-4.45 (m, 2H), 3.90 (d, 2H), 3.44 (dd, 1H), 3.23 (dd, 1H), 3.01-2.95 (m, 1H), 2.37-2.20 (m, 2H), 2.12-2.04 (m, 1H), 1.83-1.75 (m, 2H), 1.64-1.51 (m, 4H), 1.40-1.32 (m, 2H).

LC/MS (Method 1, ESIpos): Rₜ=1.23 min, m/z=490 [M+H]⁺.

### Example 18

### (+)-4-[5-(Cyclopentylmethoxy)-1-benzofur-2-yl]-4-oxo-2-[2-(4-oxo-1,2,3-benzotriazin-3(4H)-yl)ethyl]butanoic acid (enantiomer 2)

84 mg (chemical purity 100%) of the title compound were obtained.

ee value=97%; [α]_{D}²⁰=+30.9°, 589 nm, c=0.33 g/100 ml, chloroform

¹H-NMR (400 MHz, DMSO-d₆): δ [ppm]=12.46 (br. s, 1H), 8.26 (d, 1H), 8.21 (d, 1H), 8.09 (dd, 1H), 7.94 (dd, 1H), 7.82 (s, 1H), 7.61 (d, 1H), 7.29 (d, 1H), 7.14 (dd, 1H), 4.57-4.45 (m, 2H), 3.90 (d, 2H), 3.44 (dd, 1H), 3.23 (dd, 1H), 3.01-2.95 (m, 1H), 2.37-2.20 (m, 2H), 2.12-2.04 (m, 1H), 1.83-1.75 (m, 2H), 1.64-1.51 (m, 4H), 1.40-1.32 (m, 2H).

LC/MS (Method 1, ESIpos): Rₜ=1.23 min, m/z=490 [M+H]⁺.

### Example 19

### (+/−)-4-Oxo-2-[2-(4-oxo-1,2,3-benzotriazin-3(4H)-yl)ethyl]-4-[5-(2-phenylethoxy)-1-benzofur-2-yl]butanoic acid

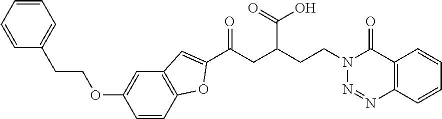 [SMILES: O=C(CC(CCn1nnc2ccccc2c1=O)C(=O)O)c1cc2cc(OCCc3ccccc3)ccc2o1]

Method A:

A solution of 3.23 g (5.29 mmol, purity 91%) of the compound from Example 38A in 50 ml of dioxane was stirred under reflux for 6 h. After cooling to RT and concentration, the residue was purified by column chromatography (silica gel, mobile phase dichloromethane/methanol 95:5). 2.20 g (81% of theory, purity 84%) of the title compound were obtained.

Method B:

88 mg (0.13 mmol) of the compound from Example 20A were stirred in 1.5 ml of a 4 N solution of hydrogen chloride in dioxane firstly for 1 h at RT and then for 3 h under reflux. After cooling to RT and concentration, the residue was admixed with dichloromethane, the solution was again evaporated to dryness and the residue was further dried in vacuo. 35 mg (49% of theory, purity 98%) of the title compound were obtained.

¹H-NMR (400 MHz, DMSO-d₆): δ [ppm]=12.43 (br. s, 1H), 8.26 (dd, 1H), 8.20 (d, 1H), 8.09 (td, 1H), 7.96-7.89 (m, 1H), 7.81 (s, 1H), 7.61 (d, 1H), 7.38-7.29 (m, 5H), 7.26-7.20 (m, 1H), 7.13 (dd, 1H), 4.59-4.41 (m, 2H), 4.24 (t, 2H), 3.48-3.39 (m, 1H), 3.26-3.18 (m, 1H), 3.07 (t, 2H), 3.02-2.93 (m, 1H), 2.29-2.17 (m, 1H), 2.14-2.01 (m, 1H).

LC/MS (Method 1, ESIpos): Rₜ=1.18 min, m/z=512 [M+H]⁺.

Separation of the Enantiomers:

2.20 g of the compound from Example 19 were dissolved in 30 ml of acetonitrile and separated into the enantiomers by preparative HPLC on a chiral phase (see Examples 20 and 21) [column: Daicel Chiralpak AD-H, 5 μm, 250 mm×20 mm; flow rate: 30 ml/min; detection: 220 nm; injection volume: 0.11 ml; temperature: 25° C.; mobile phase: 62% acetonitrile/30% methanol/8% acetonitrile/glacial acetic acid mixture (95:5), isocratic, run time 15 min]:

### Example 20

### (−)-4-Oxo-2-[2-(4-oxo-1,2,3-benzotriazin-3(4H)-yl)ethyl]-4-[5-(2-phenylethoxy)-1-benzofur-2-yl]butanoic acid (enantiomer 1)

870 mg (chemical purity 97%) of the title compound were obtained.

ee value=100%; [α]_{D}²⁰=−30.3°, 589 nm, c=0.35 g/100 ml, chloroform

¹H-NMR (400 MHz, DMSO-d₆): δ [ppm]=12.45 (br. s, 1H), 8.26 (d, 1H), 8.20 (d, 1H), 8.12-8.05 (m, 1H), 7.96-7.89 (m, 1H), 7.82 (s, 1H), 7.61 (d, 1H), 7.38-7.29 (m, 5H), 7.27-7.19 (m, 1H), 7.13 (dd, 1H), 4.59-4.42 (m, 2H), 4.24 (t, 2H), 3.44 (dd, 1H), 3.23 (dd, 1H), 3.07 (t, 2H), 3.02-2.93 (m, 1H), 2.30-2.17 (m, 1H), 2.13-2.02 (m, 1H).

LC/MS (Method 1, ESIpos): Rₜ=1.19 min, m/z=512 [M+H]⁺.

### Example 21

### (+)-4-Oxo-2-[2-(4-oxo-1,2,3-benzotriazin-3(4H)-yl)ethyl]-4-[5-(2-phenylethoxy)-1-benzofur-2-yl]butanoic acid (enantiomer 2)

Firstly, 800 mg of still contaminated product were obtained, which was post-purified by preparative HPLC (method 6). 681 mg (chemical purity 100%) of the title compound were obtained.

ee value=99.6%; [α]_{D}²⁰=+34.1°, 589 nm, c=0.31 g/100 ml, chloroform

¹H-NMR (400 MHz, DMSO-d₆): δ [ppm]=12.45 (br. s, 1H), 8.26 (dd, 1H), 8.20 (d, 1H), 8.11-8.05 (m, 1H), 7.96-7.89 (m, 1H), 7.81 (s, 1H), 7.61 (d, 1H), 7.37-7.29 (m, 5H), 7.26-7.20 (m, 1H), 7.13 (dd, 1H), 4.59-4.43 (m, 2H), 4.24 (t, 2H), 3.44 (dd, 1H), 3.23 (dd, 1H), 3.07 (t, 2H), 3.02-2.92 (m, 1H), 2.30-2.17 (m, 1H), 2.14-2.01 (m, 1H).

LC/MS (Method 1, ESIpos): Rₜ=1.17 min, m/z=512 [M+H]+.

### Example 22

### (+/−)-4-{5-[(4-Fluorobenzyl)oxy]-1-benzofur-2-yl}-4-oxo-2-[2-(4-oxo-1,2,3-benzotriazin-3(4H)-yl)ethyl]butanoic acid

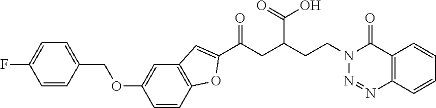 [SMILES: O=C(CC(CCn1nnc2ccccc2c1=O)C(=O)O)c1cc2cc(OCc3ccc(F)cc3)ccc2o1]

A solution of 463 mg (0.83 mmol) of the compound from Example 39A in 20 ml of dioxane was stirred under reflux for 3 h. After cooling to RT and concentration, the residue was purified by column chromatography (silica gel, mobile phase dichloromethane/methanol 95:5). 233 mg (55% of theory, purity 100%) of the title compound were obtained.

¹H-NMR (400 MHz, DMSO-d₆): δ [ppm]=12.46 (br. s, 1H), 8.26 (d, 1H), 8.21 (d, 1H), 8.09 (dd, 1H), 7.93 (dd, 1H), 7.85 (s, 1H), 7.64 (d, 1H), 7.55 (d, 1H), 7.53 (d, 1H), 7.39 (d, 1H), 7.26-7.21 (m, 3H), 5.14 (s, 2H), 4.57-4.44 (m, 2H), 3.43 (dd, 1H), 3.23 (dd, 1H), 3.02-2.94 (m, 1H), 2.28-2.19 (m, 1H), 2.13-2.03 (m, 1H).

LC/MS (Method 1, ESIpos): Rₜ=1.14 min, m/z=516 [M+H]⁺.

Separation of the Enantiomers:

223 mg of the compound from Example 22 were dissolved in 5 ml of a 1:1 mixture of methanol and ethanol and separated into the enantiomers by preparative HPLC on a chiral phase (see Examples 23 and 24) [column: Daicel Chiralpak AD-H, 5 μm, 250 mm×20 mm; flow rate: 30 ml/min; detection: 290 nm; injection volume: 0.5 ml; temperature: 21° C.; mobile phase: 30% acetonitrile/70% ethanol+0.2% diethylamine, isocratic, run time 14 min]:

### Example 23

### (+)-4-{5-[(4-Fluorobenzyl)oxy]-1-benzofur-2-yl}-4-oxo-2-[2-(4-oxo-1,2,3-benzotriazin-3(4H)-yl)ethyl]butanoic acid (enantiomer 1)

101 mg (chemical purity 100%) of the title compound were obtained.

ee value=99.6%; [α]_{D}²⁰=+28.4°, 589 nm, c=0.31 g/100 ml, chloroform

¹H-NMR (400 MHz, DMSO-d₆): δ [ppm]=12.38 (br. s, 1H), 8.26 (d, 1H), 8.20 (d, 1H), 8.09 (dd, 1H), 7.93 (dd, 1H), 7.85 (s, 1H), 7.64 (d, 1H), 7.55 (d, 1H), 7.53 (d, 1H), 7.39 (d, 1H), 7.26-7.21 (m, 3H), 5.14 (s, 2H), 4.57-4.44 (m, 2H), 3.43 (dd, 1H), 3.23 (dd, 1H), 3.02-2.94 (m, 1H), 2.28-2.19 (m, 1H), 2.13-2.03 (m, 1H).

LC/MS (Method 1, ESIpos): Rₜ=1.14 min, m/z=516 [M+H]+.

### Example 24

### (−)-4-{5-[(4-Fluorobenzyl)oxy]-1-benzofur-2-yl}-4-oxo-2-[2-(4-oxo-1,2,3-benzotriazin-3(4H)-yl)ethyl]butanoic acid (enantiomer 2)

63 mg (chemical purity 97%) of the title compound were obtained.

ee value=99.6%; [c]_{D}²⁰=−31.2°, 589 nm, c=0.33 g/100 ml, chloroform

¹H-NMR (400 MHz, DMSO-d₆): δ [ppm]=12.38 (br. s, 1H), 8.26 (d, 1H), 8.20 (d, 1H), 8.09 (dd, 1H), 7.93 (dd, 1H), 7.85 (s, 1H), 7.64 (d, 1H), 7.55 (d, 1H), 7.53 (d, 1H), 7.39 (d, 1H), 7.26-7.21 (m, 3H), 5.14 (s, 2H), 4.57-4.44 (m, 2H), 3.43 (dd, 1H), 3.23 (dd, 1H), 3.02-2.94 (m, 1H), 2.28-2.19 (m, 1H), 2.13-2.03 (m, 1H).

LC/MS (Method 1, ESIpos): Rₜ=1.14 min, m/z=516 [M+H]⁺.

### Example 25

### (+/−)-4-{5-[(3,4-Dichlorobenzyl)oxy]-1-benzofur-2-yl}-4-oxo-2-[2-(4-oxo-1,2,3-benzotriazin-3(4H)-yl)ethyl]butanoic acid

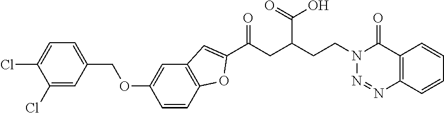 [SMILES: O=C(CC(CCn1nnc2ccccc2c1=O)C(=O)O)c1cc2cc(OCc3ccc(Cl)c(Cl)c3)ccc2o1]

A solution of 735 mg (0.96 mmol, purity 94%) of the compound from Example 22A were stirred in 10 ml of a 4 N solution of hydrogen chloride in dioxane firstly for 16 h at RT and then for 5 h under reflux. After cooling to RT and concentration, the residue was admixed with dichloromethane, the solution was again evaporated to dryness and the residue was finally dried in vacuo. 523 mg (90% of theory, purity 93%) of the title compound were obtained.

¹H-NMR (400 MHz, DMSO-d₆): δ [ppm]=12.43 (br. s, 1H), 8.26 (dd, 1H), 8.20 (d, 1H), 8.12-8.05 (m, 1H), 7.96-7.89 (m, 1H), 7.85 (d, 1H), 7.76 (d, 1H), 7.66 (t, 2H), 7.49 (dd, 1H), 7.38 (d, 1H), 7.25 (dd, 1H), 5.19 (s, 2H), 4.59-4.43 (m, 2H), 3.48-3.39 (m, 1H), 3.27-3.19 (m, 1H), 3.03-2.93 (m, 1H), 2.30-2.18 (m, 1H), 2.14-2.02 (m, 1H).

LC/MS (Method 1, ESIpos): Rₜ=1.28 min, m/z=566 [M+H]⁺.

Separation of the Enantiomers:

483 mg of the compound from Example 25 were dissolved in 70 ml of a mixture of ethanol and acetonitrile and separated into the enantiomers by preparative HPLC on a chiral phase (see Examples 26 and 27) [column: Daicel Chiralpak AD-H, 5 μm, 250 mm×20 mm; flow rate: 20 ml/min; detection: 230 nm; injection volume: 1.5 ml; temperature: 25° C.; mobile phase: 50% acetonitrile/50% ethanol+0.2% acetic acid, isocratic, run time 16 min]:

### Example 26

### (−)-4-{5-[(3,4-Dichlorobenzyl)oxy]-1-benzofur-2-yl}-4-oxo-2-[2-(4-oxo-1,2,3-benzotriazin-3(4H)-yl)ethyl]butanoic acid (enantiomer 1)

230 mg (chemical purity 96%) of the title compound were obtained.

ee value=100%; [α]_{D}²⁰=−26.9°, 589 nm, c=0.40 g/100 ml, chloroform

¹H-NMR (400 MHz, DMSO-d₆): δ [ppm]=12.42 (br. s, 1H), 8.26 (d, 1H), 8.20 (d, 1H), 8.12-8.05 (m, 1H), 7.97-7.90 (m, 1H), 7.85 (s, 1H), 7.76 (d, 1H), 7.68-7.64 (m, 2H), 7.48 (dd, 1H), 7.38 (d, 1H), 7.25 (dd, 1H), 5.19 (s, 2H), 4.59-4.43 (m, 2H), 3.43 (dd, 1H), 3.22 (dd, 1H), 3.03-2.94 (m, 1H), 2.29-2.20 (m, 1H), 2.15-2.04 (m, 1H).

LC/MS (Method 1, ESIpos): Rₜ=1.28 min, m/z=566 [M+H]⁺.

### Example 27

### (+)-4-{5-[(3,4-Dichlorobenzyl)oxy]-1-benzofur-2-yl}-4-oxo-2-[2-(4-oxo-1,2,3-benzotriazin-3(4H)-yl)ethyl]butanoic acid (enantiomer 2)

172 mg (chemical purity 98%) of the title compound were obtained.

ee value=100%; [α]_{D}²⁰=+24.6°, 589 nm, c=0.35 g/100 ml, chloroform

¹H-NMR (400 MHz, DMSO-d₆): δ [ppm]=12.42 (br. s, 1H), 8.26 (d, 1H), 8.20 (d, 1H), 8.12-8.05 (m, 1H), 7.97-7.90 (m, 1H), 7.85 (s, 1H), 7.76 (d, 1H), 7.68-7.64 (m, 2H), 7.48 (dd, 1H), 7.38 (d, 1H), 7.25 (dd, 1H), 5.19 (s, 2H), 4.59-4.43 (m, 2H), 3.43 (dd, 1H), 3.22 (dd, 1H), 3.03-2.94 (m, 1H), 2.29-2.20 (m, 1H), 2.15-2.04 (m, 1H).

LC/MS (Method 1, ESIpos): Rₜ=1.28 min, m/z=566 [M+H]⁺.

### Example 28

### (+/−)-4-[5-(Benzyloxy)-1-benzofur-2-yl]-4-oxo-2-[2-(4-oxo-1,2,3-benzotriazin-3(4H)-yl)ethyl]butanoic acid

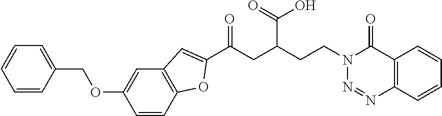 [SMILES: O=C(CC(CCn1nnc2ccccc2c1=O)C(=O)O)c1cc2cc(OCc3ccccc3)ccc2o1]

A solution of 77 mg (0.12 mmol) of the compound from Example 6A in 1.5 ml of a 4 N solution of hydrogen chloride in dioxane was stirred firstly for 16 h at RT and then for 4 h under reflux. After cooling to RT and concentration, the residue was admixed with dichloromethane, the solution was again evaporated to dryness and the residue was finally dried in vacuo. 62 mg (99% of theory, purity 95%) of the title compound were obtained.

¹H-NMR (400 MHz, DMSO-d₆): δ [ppm]=12.43 (br. s, 1H), 8.26 (d, 1H), 8.20 (d, 1H), 8.13-8.05 (m, 1H), 7.97-7.90 (m, 1H), 7.84 (s, 1H), 7.64 (d, 1H), 7.53-7.46 (m, 2H), 7.45-7.31 (m, 4H), 7.23 (dd, 1H), 5.16 (s, 2H), 4.60-4.42 (m, 2H), 3.44 (dd, 1H), 3.23 (dd, 1H), 3.02-2.93 (m, 1H), 2.29-2.20 (m, 1H), 2.13-2.04 (m, 1H).

LC/MS (Method 1, ESIpos): Rₜ=1.13 min, m/z=498 [M+H]⁺.

Separation of the Enantiomers:

43 mg of the compound from Example 28 were dissolved in 4 ml of acetonitrile and separated into the enantiomers by preparative HPLC on a chiral phase (see Examples 29 and 30) [column: Daicel Chiralpak AD-H, 5 μm, 250 mm×30 mm; flow rate: 20 ml/min; detection: 220 nm; injection volume: 0.25 ml; temperature: 20° C.; mobile phase: 30% ethanol/70% acetonitrile, isocratic, run time 13 min]:

### Example 29

### (−)-4-[5-(Benzyloxy)-1-benzofur-2-yl]-4-oxo-2-[2-(4-oxo-1,2,3-benzotriazin-3(4H)-yl)ethyl]butanoic acid (enantiomer 1)

17 mg (chemical purity 95%) of the title compound were obtained.

ee value=100%; [α]_{D}²⁰=−15.3°, 589 nm, c=0.31 g/100 ml, chloroform

¹H-NMR (400 MHz, DMSO-d₆): δ [ppm]=12.43 (br. s, 1H), 8.26 (d, 1H), 8.20 (d, 1H), 8.13-8.05 (m, 1H), 7.97-7.90 (m, 1H), 7.84 (s, 1H), 7.64 (d, 1H), 7.53-7.46 (m, 2H), 7.45-7.31 (m, 4H), 7.23 (dd, 1H), 5.16 (s, 2H), 4.60-4.42 (m, 2H), 3.44 (dd, 1H), 3.23 (dd, 1H), 3.02-2.93 (m, 1H), 2.29-2.20 (m, 1H), 2.13-2.04 (m, 1H).

LC/MS (Method 1, ESIpos): Rₜ=1.16 min, m/z=498 [M+H]⁺.

### Example 30

### (+)-4-[5-(Benzyloxy)-1-benzofur-2-yl]-4-oxo-2-[2-(4-oxo-1,2,3-benzotriazin-3(4H)-yl)ethyl]butanoic acid (enantiomer 2)

13 mg (chemical purity 100%) of the title compound were obtained.

ee value=100%; [α]_{D}²⁰=+29.6°, 589 nm, c=0.30 g/100 ml, chloroform

¹H-NMR (400 MHz, DMSO-d₆): δ [ppm]=12.43 (br. s, 1H), 8.26 (d, 1H), 8.20 (d, 1H), 8.13-8.05 (m, 1H), 7.97-7.90 (m, 1H), 7.84 (s, 1H), 7.64 (d, 1H), 7.53-7.46 (m, 2H), 7.45-7.31 (m, 4H), 7.23 (dd, 1H), 5.16 (s, 2H), 4.60-4.42 (m, 2H), 3.44 (dd, 1H), 3.23 (dd, 1H), 3.02-2.93 (m, 1H), 2.29-2.20 (m, 1H), 2.13-2.04 (m, 1H).

LC/MS (Method 1, ESIpos): Rₜ=1.16 min, m/z=498 [M+H]⁺.

### Example 31

### (+/−)-4-[5-(Heptyloxy)-1-benzofur-2-yl]-4-oxo-2-[2-(4-oxo-1,2,3-benzotriazin-3(4H)-yl)ethyl]butanoic acid

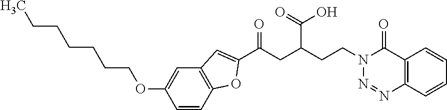 [SMILES: CCCCCCCOc1ccc2oc(C(=O)CC(CCn3nnc4ccccc4c3=O)C(=O)O)cc2c1]

A solution of 112 mg (0.17 mmol) of the compound from Example 23A in 1 ml of a 4 N solution of hydrogen chloride in dioxane was stirred firstly for 16 h at RT and then for 3 h under reflux. After cooling to RT and concentration, the residue was purified by column chromatography (silica gel, mobile phase dichloromethane/methanol 10:1). 17 mg (20% of theory, purity 98%) of the title compound were obtained.

¹H-NMR (400 MHz, DMSO-d₆): δ [ppm]=12.42 (br. s, 1H), 8.25 (d, 1H), 8.20 (d, 1H), 8.13-8.05 (m, 1H), 7.97-7.90 (m, 1H), 7.81 (s, 1H), 7.60 (d, 1H), 7.27 (d, 1H), 7.13 (dd, 1H), 4.59-4.43 (m, 2H), 4.01 (t, 2H), 3.49-3.38 (m, 1H), 3.26-3.17 (m, 1H), 3.02-2.93 (m, 1H), 2.29-2.18 (m, 1H), 2.13-2.02 (m, 1H), 1.80-1.67 (m, 2H), 1.49-1.20 (m, 8H), 0.92-0.83 (m, 3H).

LC/MS (Method 1, ESIpos): Rₜ=1.37 min, m/z=506 [M+H]⁺.

### Example 32

### (+/−)-4-{5-[(3-Carbamoylbenzyl)oxy]-1-benzofur-2-yl}-4-oxo-2-[2-(4-oxo-1,2,3-benzotriazin-3(4H)-yl)ethyl]butanoic acid

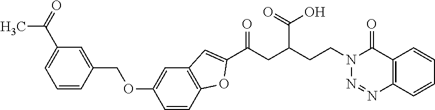 [SMILES: CC(=O)c1cccc(COc2ccc3oc(C(=O)CC(CCn4nnc5ccccc5c4=O)C(=O)O)cc3c2)c1]

A solution of 240 mg (0.34 mmol) of the compound from Example 24A in 4 ml of a 4 N solution of hydrogen chloride in dioxane was stirred firstly for 16 h at RT and then for 4 h under reflux. After cooling to RT and concentration, the residue was admixed with dichloromethane, the solution was again evaporated to dryness and the residue was finally dried in vacuo. 190 mg (99% of theory, purity 96%) of the title compound were obtained.

¹H-NMR (400 MHz, DMSO-d₆): δ [ppm]=12.47 (br. s, 1H), 8.26 (dd, 1H), 8.20 (d, 1H), 8.12-8.05 (m, 1H), 8.04-7.97 (m, 2H), 7.96-7.90 (m, 1H), 7.87-7.80 (m, 2H), 7.64 (t, 2H), 7.52-7.45 (m, 1H), 7.40 (d, 2H), 7.25 (dd, 1H), 5.21 (s, 2H), 4.61-4.41 (m, 2H), 3.44 (dd, 1H), 3.23 (dd, 1H), 3.03-2.92 (m, 1H), 2.29-2.17 (m, 1H), 2.14-2.02 (m, 1H).

LC/MS (Method 1, ESIpos): Rₜ=0.92 min, m/z=541 [M+H]⁺.

Separation of the Enantiomers:

110 mg of the compound from Example 32 were dissolved in 5 ml of a mixture of methanol, acetonitrile and ethanol and separated into the enantiomers by preparative HPLC on a chiral phase (see Examples 33 and 34) [column: Daicel Chiralpak AD-H, 5 μm, 250 mm×20 mm; flow rate: 20 ml/min; detection: 220 nm; injection volume: 0.5 ml; temperature: 20° C.; mobile phase: 30% acetonitrile/70% ethanol, isocratic, run time 14 min]:

### Example 33

### (−)-4-{5-[(3-Carbamoylbenzyl)oxy]-1-benzofur-2-yl}-4-oxo-2-[2-(4-oxo-1,2,3-benzotriazin-3(4H)-yl)ethyl]butanoic acid (enantiomer 1)

32 mg (chemical purity 99%) of the title compound were obtained.

ee value=97%; [α]_{D}²⁰=−12.4°, 589 nm, c=0.31 g/100 ml, chloroform

¹H-NMR (400 MHz, DMSO-d₆): δ [ppm]=12.46 (br. s, 1H), 8.26 (d, 1H), 8.20 (d, 1H), 8.12-8.05 (m, 1H), 8.04-7.98 (m, 2H), 7.96-7.90 (m, 1H), 7.88-7.82 (m, 2H), 7.69-7.60 (m, 2H), 7.50-7.47 (m, 1H), 7.43-7.36 (m, 2H), 7.25 (dd, 1H), 5.21 (s, 2H), 4.59-4.44 (m, 2H), 3.44 (m, 1H), 3.23 (m, 1H), 3.01-2.94 (m, 1H), 2.29-2.18 (m, 1H), 2.14-2.03 (m, 1H).

LC/MS (Method 1): Rₜ=0.93 min; MS (ESIpos): m/z=541 [M+H]⁺.

### Example 34

### (+)-4-{5-[(3-Carbamoylbenzyl)oxy]-1-benzofur-2-yl}-4-oxo-2-[2-(4-oxo-1,2,3-benzotriazin-3(4H)-yl)ethyl]butanoic acid (enantiomer 2)

33 mg (chemical purity 97%) of the title compound were obtained.

ee value=100%; [α]_{D}²⁰=+18.2°, 589 nm, c=0.35 g/100 ml, chloroform

¹H-NMR (400 MHz, DMSO-d₆): δ [ppm]=12.46 (br. s, 1H), 8.26 (d, 1H), 8.20 (d, 1H), 8.12-8.05 (m, 1H), 8.04-7.98 (m, 2H), 7.96-7.90 (m, 1H), 7.88-7.82 (m, 2H), 7.69-7.60 (m, 2H), 7.50-7.47 (m, 1H), 7.43-7.36 (m, 2H), 7.25 (dd, 1H), 5.21 (s, 2H), 4.59-4.44 (m, 2H), 3.44 (m, 1H), 3.23 (m, 1H), 3.01-2.94 (m, 1H), 2.29-2.18 (m, 1H), 2.14-2.03 (m, 1H).

LC/MS (Method 1): Rₜ=0.91 min; MS (ESIpos): m/z=541 [M+H]⁺.

### Example 35

### (+/−)-4-{5-[(4-Carbamoylbenzyl)oxy]-1-benzofur-2-yl}-4-oxo-2-[2-(4-oxo-1,2,3-benzotriazin-3(4H)-yl)ethyl]butanoic acid

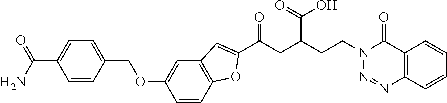 [SMILES: NC(=O)c1ccc(COc2ccc3oc(C(=O)CC(CCn4nnc5ccccc5c4=O)C(=O)O)cc3c2)cc1]

A solution of 204 mg (0.29 mmol) of the compound from Example 25A in 3 ml of a 4 N solution of hydrogen chloride in dioxane was stirred firstly for 16 h at RT and then for 7 h under reflux. After cooling to RT and concentration, the residue was admixed with dichloromethane, the solution was again concentrated and the residue was finally dried in vacuo. 50 mg (32% of theory, purity 92%) of the title compound were obtained.

¹H-NMR (400 MHz, DMSO-d₆): δ [ppm]=12.42 (br. s, 1H), 8.26 (d, 1H), 8.20 (d, 1H), 8.13-8.05 (m, 1H), 8.01-7.82 (m, 5H), 7.65 (d, 1H), 7.55 (d, 2H), 7.38 (d, 2H), 7.25 (dd, 1H), 5.23 (s, 2H), 4.58-4.42 (m, 2H), 3.48-3.39 (m, 1H), 3.27-3.18 (m, 1H), 3.04-2.90 (m, 1H), 2.30-2.18 (m, 1H), 2.14-2.02 (m, 1H).

LC/MS (Method 1): Rₜ=0.88 min; MS (ESIpos): m/z=541 [M+H]⁺.

### Example 36

### (+/−)-4-[5-(2,1,3-Benzoxadiazol-5-ylmethoxy)-1-benzofur-2-yl]-4-oxo-2-[2-(4-oxo-1,2,3-benzotriazin-3(4H)-yl)ethyl]butanoic acid

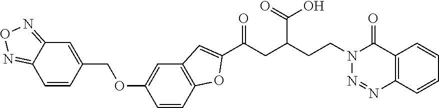 [SMILES: O=C(CC(CCn1nnc2ccccc2c1=O)C(=O)O)c1cc2cc(OCc3ccc4nonc4c3)ccc2o1]

38 mg (0.18 mmol) of 5-(bromomethyl)-2,1,3-benzoxadiazole were initially introduced onto a metal mutititre plate and admixed with 85 mg (0.15 mmol) of the compound from Example 7A, dissolved in 0.7 ml of DMF. Then, 104 mg (0.75 mmol) of potassium carbonate were added. The mixture was shaken at 70° C. for 18 h. After cooling to RT, the mixture was filtered and the filtrate was concentrated. The residue was admixed with 1.6 ml of a 4 N solution of hydrogen chloride in dioxane and shaken firstly for 18 h at RT, then for 18 h at 80° C. After cooling to RT, the mixture was concentrated. The residue was taken up in 0.7 ml of DMSO and purified by means of preparative HPLC (Method 10). 21 mg (26% of theory, purity 100%) of the title compound were obtained.

LC/MS (Method 4): Rₜ=1.17 min; MS (ESIpos): m/z=540 [M+H]⁺.

### Example 37

### (+/−)-4-Oxo-2-[2-(4-oxo-1,2,3-benzotriazin-3(4H)-yl)ethyl]-4-[5-(3-phenylpropoxy)-1-benzofur-2-yl]butanoic acid

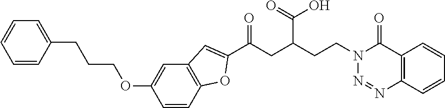 [SMILES: O=C(CC(CCn1nnc2ccccc2c1=O)C(=O)O)c1cc2cc(OCCCc3ccccc3)ccc2o1]

Analogously to the method described in Example 36, 36 mg (0.18 mmol) of (3-bromopropyl)benzene were reacted with 85 mg (0.15 mmol) of the compound from Example 7A. Purification of the crude product by preparative HPLC [Method 10] gave 42 mg (54% of theory, purity 100%) of the title compound.

LC/MS (Method 4): Rₜ=1.23 min; MS (ESIpos): m/z=526 [M+H]⁺.

### Example 38

### (+/−)-4-(5-{[4-(Methylsulphonyl)benzyl]oxy}-1-benzofur-2-yl)-4-oxo-2-[2-(4-oxo-1,2,3-benzotriazin-3(4H)-yl)ethyl]butanoic acid

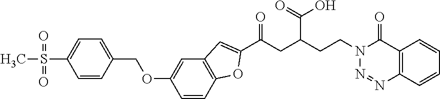 [SMILES: CS(=O)(=O)c1ccc(COc2ccc3oc(C(=O)CC(CCn4nnc5ccccc5c4=O)C(=O)O)cc3c2)cc1]

Analogously to the method described in Example 36, 45 mg (0.18 mmol) of 1-(bromomethyl)-4-(methylsulphonyl)benzene were reacted with 85 mg (0.15 mmol) of the compound from Example 7A. Purification of the crude product by preparative HPLC [Method 10] gave 20 mg (23% of theory, purity 95%) of the title compound.

LC/MS (Method 4): Rₜ=1.08 min; MS (ESIpos): m/z=576 [M+H]⁺.

### Example 39

### (+/−)-4-Oxo-2-[2-(4-oxo-1,2,3-benzotriazin-3(4H)-yl)ethyl]-4-(5-{[3-(trifluoromethyl)benzyl]oxy}-1-benzofur-2-yl)butanoic acid

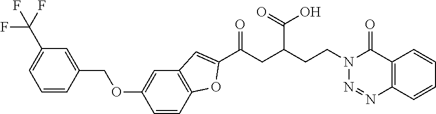 [SMILES: O=C(CC(CCn1nnc2ccccc2c1=O)C(=O)O)c1cc2cc(OCc3cccc(C(F)(F)F)c3)ccc2o1]

Analogously to the method described in Example 36, 43 mg (0.18 mmol) of 1-(bromomethyl-3-trifluoromethyl)benzene were reacted with 85 mg (0.15 mmol) of the compound from Example 7A. Purification of the crude product by preparative HPLC [Method 10] gave 30 mg (35% of theory, purity 98%) of the title compound.

LC/MS (Method 4): Rₜ=1.23 min; MS (ESIpos): m/z=566 [M+H]⁺.

### Example 40

### (+/−)-4-{5-[(4-Methylpentyl)oxy]-1-benzofur-2-yl}-4-oxo-2-[2-(4-oxo-1,2,3-benzotriazin-3(4H)-yl)ethyl]butanoic acid

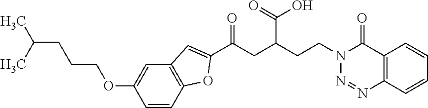 [SMILES: CC(C)CCCOc1ccc2oc(C(=O)CC(CCn3nnc4ccccc4c3=O)C(=O)O)cc2c1]

Analogously to the method described in Example 36, 30 mg (0.18 mmol) of 1-bromo-4-methylpentane were reacted with 85 mg (0.15 mmol) of the compound from Example 7A. Purification of the crude product by preparative HPLC [Method 10] gave 30 mg (40% of theory, purity 100%) of the title compound.

LC/MS (Method 4): Rₜ=1.25 min; MS (ESIpos): m/z=492 [M+H]⁺.

### Example 41

### (+/−)-4-{5-[(5-Methylhexyl)oxy]-1-benzofur-2-yl}-4-oxo-2-[2-(4-oxo-1,2,3-benzotriazin-3(4H)-yl)ethyl]butanoic acid

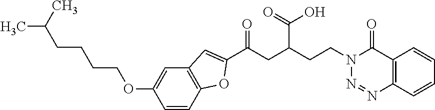 [SMILES: CC(C)CCCCOc1ccc2oc(C(=O)CC(CCn3nnc4ccccc4c3=O)C(=O)O)cc2c1]

Analogously to the method described in Example 36, 32 mg (0.18 mmol) of 1-bromo-5-methylhexane were reacted with 85 mg (0.15 mmol) of the compound from Example 7A. Purification of the crude product by preparative HPLC [Method 10] gave 31 mg (41% of theory, purity 100%) of the title compound.

LC/MS (Method 4): Rₜ=1.28 min; MS (ESIpos): m/z=506 [M+H]⁺.

### Example 42

### (+/−)-4-Oxo-2-[2-(4-oxo-1,2,3-benzotriazin-3(4H)-yl)ethyl]-4-(5-{3-[4-(trifluoromethyl)phenyl]propoxy}-1-benzofur-2-yl)butanoic acid

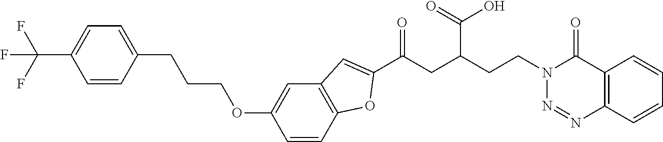 [SMILES: O=C(CC(CCn1nnc2ccccc2c1=O)C(=O)O)c1cc2cc(OCCCc3ccc(C(F)(F)F)cc3)ccc2o1]

Analogously to the method described in Example 36, 48 mg (0.18 mmol) of 1-(3-bromopropyl)-4-(trifluoromethyl)benzene were reacted with 85 mg (0.15 mmol) of the compound from Example 7A. Purification of the crude product by preparative HPLC [Method 10] gave 21 mg (24% of theory, purity 100%) of the title compound.

LC/MS (Method 4): Rₜ=1.27 min; MS (ESIpos): m/z=594 [M+H]⁺.

### Example 43

### (+/−)-4-{5-[(3-Fluorobenzyl)oxy]-1-benzofur-2-yl}-4-oxo-2-[2-(4-oxo-1,2,3-benzotriazin-3(4H)-yl)ethyl]butanoic acid

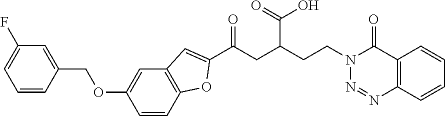 [SMILES: O=C(CC(CCn1nnc2ccccc2c1=O)C(=O)O)c1cc2cc(OCc3cccc(F)c3)ccc2o1]

Analogously to the method described in Example 36, 34 mg (0.18 mmol) of 1-(bromomethyl-3-fluorobenzene were reacted with 85 mg (0.15 mmol) of the compound from Example 7A. Purification of the crude product by preparative HPLC [Method 10] gave 27 mg (34% of theory, purity 97%) of the title compound.

LC/MS (Method 4): Rₜ=1.18 min; MS (ESIpos): m/z=516 [M+H]⁺.

### Example 44

### (+/−)-4-Oxo-2-[2-(4-oxo-1,2,3-benzotriazin-3(4H)-yl)ethyl]-4-(5-{[4-(trifluoromethyl)benzyl]oxy}-1-benzofur-2-yl)butanoic acid

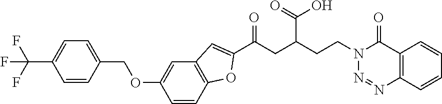 [SMILES: O=C(CC(CCn1nnc2ccccc2c1=O)C(=O)O)c1cc2cc(OCc3ccc(C(F)(F)F)cc3)ccc2o1]

Analogously to the method described in Example 36, 43 mg (0.18 mmol) of 1-(bromomethyl-4-(trifluoromethyl)benzene were reacted with 85 mg (0.15 mmol) of the compound from Example 7A. Purification of the crude product by preparative HPLC [Method 10] gave 27 mg (30% of theory, purity 95%) of the title compound.

LC/MS (Method 4): Rₜ=1.23 min; MS (ESIpos): m/z=566 [M+H]⁺.

### Example 45

### (+/−)-4-{5-[(2-Fluorobenzyl)oxy]-1-benzofur-2-yl}-4-oxo-2-[2-(4-oxo-1,2,3-benzotriazin-3(4H)-yl)ethyl]butanoic acid

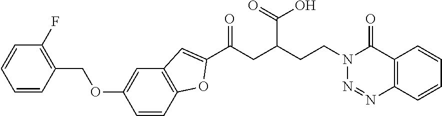 [SMILES: O=C(CC(CCn1nnc2ccccc2c1=O)C(=O)O)c1cc2cc(OCc3ccccc3F)ccc2o1]

Analogously to the method described in Example 36, 34 mg (0.18 mmol) of 1-(bromomethyl)-2-fluorobenzene were reacted with 85 mg (0.15 mmol) of the compound from Example 7A. Purification of the crude product by preparative HPLC [Method 10] gave 27 mg (30% of theory, purity 95%) of the title compound.

LC/MS (Method 4): Rₜ=1.18 min; MS (ESIpos): m/z=516 [M+H]⁺.

### Example 46

### (+/−)-4-Oxo-2-[2-(4-oxo-1,2,3-benzotriazin-3(4H)-yl)ethyl]-4-{5-[3-(2-thienyl)propoxy]-1-benzofur-2-yl}butanoic acid

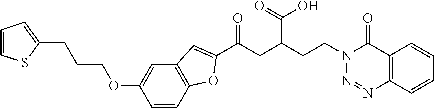 [SMILES: O=C(CC(CCn1nnc2ccccc2c1=O)C(=O)O)c1cc2cc(OCCCc3cccs3)ccc2o1]

Analogously to the method described in Example 36, 37 mg (0.18 mmol) of 2-(3-bromopropyl)thiophene were reacted with 85 mg (0.15 mmol) of the compound from Example 7A. Purification of the crude product by preparative HPLC [Method 10] gave 18 mg (22% of theory, purity 100%) of the title compound.

LC/MS (Method 4): Rₜ=1.22 min; MS (ESIpos): m/z=532 [M+H]⁺.

### Example 47

### (+/−)-4-Oxo-2-[2-(4-oxo-1,2,3-benzotriazin-3(4H)-yl)ethyl]-4-[5-(pyridin-4-ylmethoxy)-1-benzofur-2-yl]butanoic acid

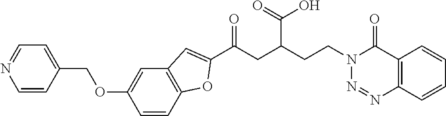 [SMILES: O=C(CC(CCn1nnc2ccccc2c1=O)C(=O)O)c1cc2cc(OCc3ccncc3)ccc2o1]

Analogously to the method described in Example 36, 31 mg (0.18 mmol) of 4-(bromomethyl)pyridine were reacted with 85 mg (0.15 mmol) of the compound from Example 7A. Purification of the crude product by preparative HPLC [Method 10] gave 1.4 mg (2% of theory, purity 83%) of the title compound.

LC/MS (Method 4): Rₜ=0.90 min; MS (ESIpos): m/z=499 [M+H]⁺.

### Example 48

### (+/−)-4-(5-{[2-Methyl-3-(trifluoromethyl)benzyl]oxy}-1-benzofur-2-yl)-4-oxo-2-[2-(4-oxo-1,2,3-benzotriazin-3(4H)-yl)ethyl]butanoic acid

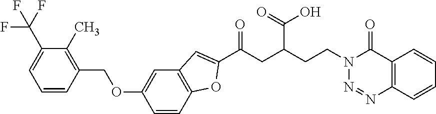 [SMILES: Cc1c(COc2ccc3oc(C(=O)CC(CCn4nnc5ccccc5c4=O)C(=O)O)cc3c2)cccc1C(F)(F)F]

Analogously to the method described in Example 36, 46 mg (0.18 mmol) of 1-(bromomethyl)-2-methyl-3-(trifluoromethyl)benzene were reacted with 85 mg (0.15 mmol) of the compound from Example 7A. Purification of the crude product by preparative HPLC [Method 10] gave 14 mg (16% of theory, purity 100%) of the title compound.

LC/MS (Method 4): Rₜ=1.25 min; MS (ESIpos): m/z=580 [M+H]⁺.

### Example 49

### (+/−)-4-{5-[(2,5-Difluorobenzyl)oxy]-1-benzofur-2-yl}-4-oxo-2-[2-(4-oxo-1,2,3-benzotriazin-3(4H)-yl)ethyl]butanoic acid

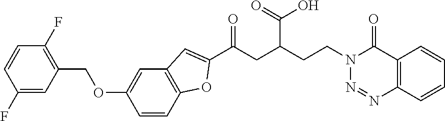 [SMILES: O=C(CC(CCn1nnc2ccccc2c1=O)C(=O)O)c1cc2cc(OCc3cc(F)ccc3F)ccc2o1]

Analogously to the method described in Example 36, 37 mg (0.18 mmol) of 2-(bromomethyl)-1.4-difluorobenzene were reacted with 85 mg (0.15 mmol) of the compound from Example 7A. Purification of the crude product by preparative HPLC [Method 10] gave 18 mg (21% of theory, purity 93%) of the title compound.

LC/MS (Method 4): Rₜ=1.19 min; MS (ESIpos): m/z=534 [M+H]⁺.

### Example 50

### (+/−)-4-{5-[(3-Chlorobenzyl)oxy]-1-benzofur-2-yl}-4-oxo-2-[2-(4-oxo-1,2,3-benzotriazin-3(4H)-yl)ethyl]butanoic acid

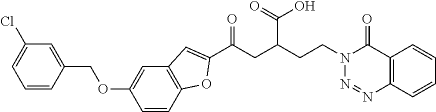 [SMILES: O=C(CC(CCn1nnc2ccccc2c1=O)C(=O)O)c1cc2cc(OCc3cccc(Cl)c3)ccc2o1]

Analogously to the method described in Example 36, 37 mg (0.18 mmol) of 1-(bromomethyl)-3-chlorobenzene were reacted with 85 mg (0.15 mmol) of the compound from Example 7A. Purification of the crude product by preparative HPLC [Method 10] gave 11 mg (14% of theory, purity 100%) of the title compound.

LC/MS (Method 4): Rₜ=1.21 min; MS (ESIpos): m/z=532 [M+H]⁺.

### Example 51

### (+/−)-4-{5-[(3-Methylbenzyl)oxy]-1-benzofur-2-yl}-4-oxo-2-[2-(4-oxo-1,2,3-benzotriazin-3(4H)-yl)ethyl]butanoic acid

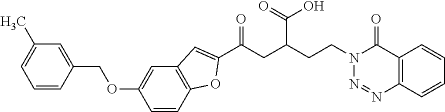 [SMILES: Cc1cccc(COc2ccc3oc(C(=O)CC(CCn4nnc5ccccc5c4=O)C(=O)O)cc3c2)c1]

Analogously to the method described in Example 36, 33 mg (0.18 mmol) of 1-(bromomethyl)-3-methylbenzene were reacted with 85 mg (0.15 mmol) of the compound from Example 7A. Purification of the crude product by preparative HPLC [Method 10] gave 8 mg (10% of theory, purity 100%) of the title compound.

LC/MS (Method 4): Rₜ=1.20 min; MS (ESIpos): m/z=512 [M+H]⁺.

### Example 52

### (+/−)-4-{5-[(2,4-Difluorobenzyl)oxy]-1-benzofur-2-yl}-4-oxo-2-[2-(4-oxo-1,2,3-benzotriazin-3(4H)-yl)ethyl]butanoic acid

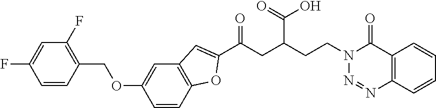 [SMILES: O=C(CC(CCn1nnc2ccccc2c1=O)C(=O)O)c1cc2cc(OCc3ccc(F)cc3F)ccc2o1]

Analogously to the method described in Example 36, 37 mg (0.18 mmol) of 1-(bromomethyl)-2.4-difluorobenzene were reacted with 85 mg (0.15 mmol) of the compound from Example 7A. Purification of the crude product by preparative HPLC [Method 10] gave 16 mg (19% of theory, purity 100%) of the title compound.

LC/MS (Method 4): Rₜ=1.19 min; MS (ESIpos): m/z=534 [M+H]⁺.

### Example 53

### (+/−)-4-[5-(4-Cyanobutoxy)-1-benzofur-2-yl]-4-oxo-2-[2-(4-oxo-1,2,3-benzotriazin-3(4H)-yl)ethyl]butanoic acid

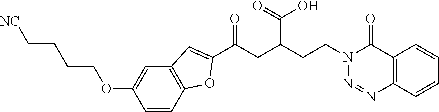 [SMILES: N#CCCCCOc1ccc2oc(C(=O)CC(CCn3nnc4ccccc4c3=O)C(=O)O)cc2c1]

Analogously to the method described in Example 36, 29 mg (0.18 mmol) of 5-(bromopentanonitrile were reacted with 85 mg (0.15 mmol) of the compound from Example 7A. Purification of the crude product by preparative HPLC [Method 10] gave 10 mg (13% of theory, purity 100%) of the title compound.

LC/MS (Method 4): Rₜ=1.08 min; MS (ESIpos): m/z=489 [M+H]⁺.

### Example 54

### (+/−)-4-Oxo-2-[2-(4-oxo-1,2,3-benzotriazin-3(4H)-yl)ethyl]-4-{6-[2-(tetrahydro-2H-pyran-4-yl)ethoxy]-1-benzofur-2-yl}butanoic acid

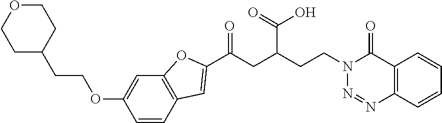 [SMILES: O=C(CC(CCn1nnc2ccccc2c1=O)C(=O)O)c1cc2ccc(OCCC3CCOCC3)cc2o1]

A solution of 79 mg (0.11 mmol, purity 75%) of the compound from Example 50A in 1 ml of dioxane was stirred under reflux for 3 h. After cooling to RT and concentration, the residue was purified by thick-layer chromatography (PLC silica gel 60 F 254, 1 mm; 20×20 cm with concentration zone; mobile phase dichloromethane/methanol 9:1). 44 mg (81% of theory, purity 100%) of the title compound were obtained.

¹H-NMR (400 MHz, DMSO-d₆): δ [ppm]=12.45 (br. s, 1H), 8.26 (dd, 1H), 8.20 (d, 1H), 8.09 (td, 1H), 7.97-7.90 (m, 1H), 7.87 (s, 1H), 7.69 (d, 1H), 7.31 (d, 1H), 6.99 (dd, 1H), 4.58-4.42 (m, 2H), 4.11 (t, 2H), 3.84 (dd, 2H), 3.45-3.35 (m, 1H), 3.31-3.26 (m, 2H), 3.23-3.15 (m, 1H), 3.02-2.91 (m, 1H), 2.29-2.17 (m, 1H), 2.12-2.01 (m, 1H), 1.71 (t, 3H), 1.64 (d, 2H), 1.31-1.15 (m, 2H).

LC/MS (Method 1, ESIpos): Rₜ=1.05 min, m/z=520 [M+H]⁺.

### Example 55

### (+/−)-4-Oxo-2-[2-(4-oxo-1,2,3-benzotriazin-3(4H)-yl)ethyl]-4-[6-(pentyloxy)-1-benzofur-2-yl]butanoic acid

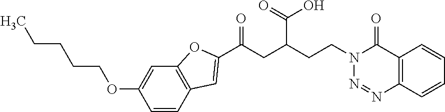 [SMILES: CCCCCOc1ccc2cc(C(=O)CC(CCn3nnc4ccccc4c3=O)C(=O)O)oc2c1]

A solution of 198 mg (0.31 mmol) of the compound from Example 45A in 3.5 ml of a 4 N solution of hydrogen chloride in dioxane was stirred firstly for 16 h at RT and then for 3 h under reflux. After cooling to RT and concentration, the mixture was admixed with dioxane, concentrated again, then admixed several times with dichloromethane and in each case again evaporated to dryness. Final drying in vacuo gave 137 mg (90% of theory, purity 98%) of the title compound.

¹H-NMR (400 MHz, DMSO-d₆): δ [ppm]=12.41 (br. s, 1H), 8.26 (dd, 1H), 8.20 (d, 1H), 8.12-8.05 (m, 1H), 7.97-7.90 (m, 1H), 7.87 (s, 1H), 7.69 (d, 1H), 7.28 (d, 1H), 6.98 (dd, 1H), 4.59-4.40 (m, 2H), 4.05 (t, 2H), 3.39 (dd, 1H), 3.20 (dd, 1H), 3.03-2.91 (m, 1H), 2.29-2.17 (m, 1H), 2.13-2.00 (m, 1H), 1.81-1.68 (m, 2H), 1.48-1.27 (m, 4H), 0.91 (t, 3H).

LC/MS (Method 1, ESIpos): Rₜ=1.23 min, m/z=478 [M+H]⁺.

Separation of the Enantiomers:

128 mg of the compound from Example 55 were dissolved in 5 ml of acetonitrile and separated into the enantiomers by preparative HPLC on a chiral phase (see Examples 56 and 57) [column: Daicel Chiralpak AD-H, 5 μm, 250 mm×20 mm; flow rate: 20 ml/min; detection: 220 nm; injection volume: 0.5 ml; temperature: 20° C.; mobile phase: 30% methanol/70% acetonitrile+0.2% acetic acid, isocratic, run time 20 min]:

### Example 56

### (−)-4-Oxo-2-[2-(4-oxo-1,2,3-benzotriazin-3(4H)-yl)ethyl]-4-[6-(pentyloxy)-1-benzofur-2-yl]butanoic acid (enantiomer 1)

54 mg (chemical purity 100%) of the title compound were obtained.

ee value=100%; [α]_{D}²⁰=−38.0°, 589 nm, c=0.39 g/100 ml, chloroform

¹H-NMR (400 MHz, DMSO-d₆): δ [ppm]=12.38 (br. s, 1H), 8.26 (dd, 1H), 8.20 (d, 1H), 8.09 (td, 1H), 7.97-7.90 (m, 1H), 7.86 (s, 1H), 7.68 (d, 1H), 7.28 (d, 1H), 6.98 (dd, 1H), 4.59-4.40 (m, 2H), 4.05 (t, 2H), 3.39 (dd, 1H), 3.19 (dd, 1H), 3.03-2.91 (m, 1H), 2.29-2.17 (m, 1H), 2.13-2.01 (m, 1H), 1.81-1.69 (m, 2H), 1.47-1.30 (m, 4H), 0.91 (t, 3H).

LC/MS (Method 1, ESIpos): Rₜ=1.24 min, m/z=478 [M+H]⁺.

### Example 57

### (+)-4-Oxo-2-[2-(4-oxo-1,2,3-benzotriazin-3(4H)-yl)ethyl]-4-[6-(pentyloxy)-1-benzofur-2-yl]butanoic acid (enantiomer 2)

60 mg (chemical purity 100%) of the title compound were obtained.

ee value=100%; [α]_{D}²⁰=+29.7°, 589 nm, c=0.32 g/100 ml, chloroform

¹H-NMR (400 MHz, DMSO-d₆): δ [ppm]=12.39 (br. s, 1H), 8.26 (d, 1H), 8.20 (d, 1H), 8.09 (td, 1H), 7.96-7.90 (m, 1H), 7.86 (s, 1H), 7.68 (d, 1H), 7.28 (d, 1H), 6.98 (dd, 1H), 4.59-4.41 (m, 2H), 4.05 (t, 2H), 3.39 (dd, 1H), 3.18 (dd, 1H), 3.03-2.91 (m, 1H), 2.30-2.15 (m, 1H), 2.13-1.99 (m, 1H), 1.81-1.69 (m, 2H), 1.46-1.31 (m, 4H), 0.91 (t, 3H).

LC/MS (Method 1, ESIpos): Rₜ=1.24 min, m/z=478 [M+H]⁺.

### Example 58

### (+/−)-4-Oxo-2-[2-(4-oxo-1,2,3-benzotriazin-3(4H)-yl)ethyl]-4-(6-propoxy-1-benzofur-2-yl)butanoic acid

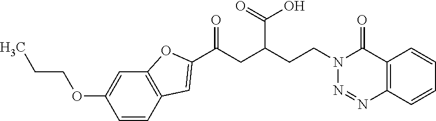 [SMILES: CCCOc1ccc2cc(C(=O)CC(CCn3nnc4ccccc4c3=O)C(=O)O)oc2c1]

A solution of 94 mg (0.16 mmol) of the compound from Example 46A in 4 ml of a 4 N solution of hydrogen chloride in dioxane was stirred firstly for 16 h at RT and then for 2.5 h under reflux. After cooling to RT and concentration, the residue was admixed with dioxane, the solution was again concentrated and the residue was finally dried in vacuo. 65 mg (93% of theory, purity 100%) of the title compound were obtained.

¹H-NMR (400 MHz, DMSO-d₆): δ [ppm]=12.41 (br. s, 1H), 8.26 (d, 1H), 8.20 (d, 1H), 8.11-8.07 (m, 1H), 7.95-7.91 (m, 1H), 7.87 (d, 1H), 7.69 (d, 1H), 7.28 (d, 1H), 6.99 (dd, 1H), 4.58-4.41 (m, 2H), 4.02 (t, 2H), 3.37 (dd, 1H), 3.19 (dd, 1H), 3.03-2.92 (m, 1H), 2.28-2.19 (m, 1H), 2.13-2.00 (m, 1H), 1.81-1.73 (m, 2H), 1.00 (t, 3H).

LC/MS (Method 1, ESIpos): Rₜ=1.10 min, m/z=450 [M+H]⁺.

Separation of the Enantiomers:

47 mg of the compound from Example 58 were dissolved in 30 ml of a mixture of methanol, acetonitrile, isopropanol and ethanol and separated into the enantiomers by preparative HPLC on a chiral phase (see Examples 59 and 60) [column: Daicel Chiralpak AD-H, 5 μm, 250 mm×30 mm; flow rate: 20 ml/min; detection: 220 nm; injection volume: 3 ml; temperature: 20° C.; mobile phase: 70% ethanol/30% acetonitrile, isocratic, run time 11 min]:

### Example 59

### 4-Oxo-2-[2-(4-oxo-1,2,3-benzotriazin-3(4H)-yl)ethyl]-4-(6-propoxy-1-benzofur-2-yl)butanoic acid (enantiomer 1)

19 mg (chemical purity 100%) of the title compound were obtained.

Rₜ=3.99 min, ee value=100% [column: Daicel Chiralpak AD-H, 250 mm×4.6 mm, 5 μm; flow rate: 1.0 ml/min; detection: 270 nm; temperature: 30° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.41 (br. s, 1H), 8.26 (d, 1H), 8.20 (d, 1H), 8.11-8.07 (m, 1H), 7.95-7.91 (m, 1H), 7.87 (d, 1H), 7.69 (d, 1H), 7.28 (d, 1H), 6.99 (dd, 1H), 4.58-4.41 (m, 2H), 4.02 (t, 2H), 3.37 (dd, 1H), 3.19 (dd, 1H), 3.03-2.92 (m, 1H), 2.28-2.19 (m, 1H), 2.13-2.00 (m, 1H), 1.81-1.73 (m, 2H), 1.00 (t, 3H).

LC/MS (Method 1, ESIpos): Rₜ=1.12 min, m/z=450 [M+H]⁺.

### Example 60

### 4-Oxo-2-[2-(4-oxo-1,2,3-benzotriazin-3(4H)-yl)ethyl]-4-(6-propoxy-1-benzofur-2-yl)butanoic acid (enantiomer 2)

9 mg (chemical purity 94%) of the title compound were obtained.

Rₜ=6.59 min, ee value=100% [column: Daicel Chiralpak AD-H, 250 mm×4.6 mm, 5 μm; flow rate: 1.0 ml/min; detection: 270 nm; temperature: 30° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.41 (br. s, 1H), 8.26 (d, 1H), 8.20 (d, 1H), 8.11-8.07 (m, 1H), 7.95-7.91 (m, 1H), 7.87 (d, 1H), 7.69 (d, 1H), 7.28 (d, 1H), 6.99 (dd, 1H), 4.58-4.41 (m, 2H), 4.02 (t, 2H), 3.37 (dd, 1H), 3.19 (dd, 1H), 3.03-2.92 (m, 1H), 2.28-2.19 (m, 1H), 2.13-2.00 (m, 1H), 1.81-1.73 (m, 2H), 1.00 (t, 3H).

LC/MS (Method 1, ESIpos): Rₜ=1.12 min, m/z=450 [M+H]⁺.

### Example 61

### (+/−)-4-(6-Butoxy-1-benzofur-2-yl)-4-oxo-2-[2-(4-oxo-1,2,3-benzotriazin-3(4H)-yl)ethyl]butanoic acid

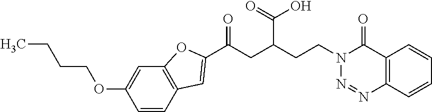 [SMILES: CCCCOc1ccc2cc(C(=O)CC(CCn3nnc4ccccc4c3=O)C(=O)O)oc2c1]

A solution of 89 mg (0.14 mmol) of the compound from Example 47A in 1.5 ml of a 4 N solution of hydrogen chloride in dioxane was stirred firstly for 16 h at RT and then for 2.5 h under reflux. After cooling to RT, concentration and repeated reuptake and concentration with dioxane, dichloromethane and acetonitrile, the residue was finally dried in vacuo. 57 mg (86% of theory, purity 100%) of the title compound were obtained.

¹H-NMR (400 MHz, DMSO-d₆): δ [ppm]=12.41 (br. s, 1H), 8.26 (d, 1H), 8.20 (d, 1H), 8.12-8.06 (m, 1H), 7.96-7.90 (m, 1H), 7.87 (s, 1H), 7.69 (d, 1H), 7.30-7.25 (m, 1H), 6.98 (dd, 1H), 4.58-4.43 (m, 2H), 4.06 (t, 2H), 3.39 (dd, 1H), 3.19 (dd, 1H), 3.03-2.93 (m, 1H), 2.29-2.18 (m, 1H), 2.14-2.02 (m, 1H), 1.79-1.69 (m, 2H), 1.52-1.40 (m, 2H), 0.95 (t, 3H).

LC/MS (Method 1, ESIpos): Rₜ=1.17 min, m/z=464 [M+H]⁺.

Separation of the Enantiomers:

40 mg of the compound from Example 61 were dissolved in 5 ml of methanol/acetonitrile and separated into the enantiomers by preparative HPLC on a chiral phase (see Examples 62 and 63) [column: Daicel Chiralpak AD-H, 5 μm, 250 mm×20 mm; flow rate: 20 ml/min; detection: 220 nm; injection volume: 1 ml; temperature: 20° C.; mobile phase: 30% methanol/70% acetonitrile, isocratic, run time 18 min]:

### Example 62

### (−)-4-(6-Butoxy-1-benzofur-2-yl)-4-oxo-2-[2-(4-oxo-1,2,3-benzotriazin-3(4H)-yl)ethyl]butanoic acid (enantiomer 1)

16 mg (chemical purity 100%) of the title compound were obtained.

ee value=100%; [α]_{D}²⁰=−37.0°, 589 nm, c=0.30 g/100 ml, chloroform

¹H-NMR (400 MHz, DMSO-d₆): δ [ppm]=12.41 (br. s, 1H), 8.26 (d, 1H), 8.20 (d, 1H), 8.12-8.06 (m, 1H), 7.96-7.90 (m, 1H), 7.87 (s, 1H), 7.69 (d, 1H), 7.30-7.25 (m, 1H), 6.98 (dd, 1H), 4.58-4.43 (m, 2H), 4.06 (t, 2H), 3.39 (dd, 1H), 3.19 (dd, 1H), 3.03-2.93 (m, 1H), 2.29-2.18 (m, 1H), 2.14-2.02 (m, 1H), 1.79-1.69 (m, 2H), 1.52-1.40 (m, 2H), 0.95 (t, 3H).

LC/MS (Method 1, ESIpos): Rₜ=1.19 min, m/z=464 [M+H]⁺.

### Example 63

### (+)-4-(6-Butoxy-1-benzofur-2-yl)-4-oxo-2-[2-(4-oxo-1,2,3-benzotriazin-3(4H)-yl)ethyl]butanoic acid (enantiomer 2)

14 mg (chemical purity 100%) of the title compound were obtained.

ee value=100%; [α]_{D}²⁰=+32.2°, 589 nm, c=0.30 g/100 ml, chloroform

¹H-NMR (400 MHz, DMSO-d₆): δ [ppm]=12.41 (br. s, 1H), 8.26 (d, 1H), 8.20 (d, 1H), 8.12-8.06 (m, 1H), 7.96-7.90 (m, 1H), 7.87 (s, 1H), 7.69 (d, 1H), 7.30-7.25 (m, 1H), 6.98 (dd, 1H), 4.58-4.43 (m, 2H), 4.06 (t, 2H), 3.39 (dd, 1H), 3.19 (dd, 1H), 3.03-2.93 (m, 1H), 2.29-2.18 (m, 1H), 2.14-2.02 (m, 1H), 1.79-1.69 (m, 2H), 1.52-1.40 (m, 2H), 0.95 (t, 3H).

LC/MS (Method 1, ESIpos): Rₜ=1.19 min, m/z=464 [M+H]⁺.

### Example 64

### (+/−)-4-[6-(Hexyloxy)-1-benzofur-2-yl]-4-oxo-2-[2-(4-oxo-1,2,3-benzotriazin-3(4H)-yl)ethyl]butanoic acid

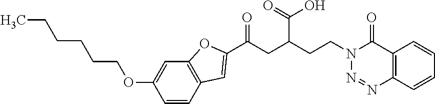 [SMILES: CCCCCCOc1ccc2cc(C(=O)CC(CCn3nnc4ccccc4c3=O)C(=O)O)oc2c1]

A solution of 109 mg (0.17 mmol) of the compound from Example 48A in 2 ml of a 4 N solution of hydrogen chloride in dioxane was stirred firstly for 16 h at RT and then for 3 h under reflux. After cooling to RT and concentration, the residue was admixed with dioxane, the solution was again concentrated and the residue was finally dried in vacuo. 72 mg (84% of theory, purity 97%) of the title compound were obtained.

¹H-NMR (400 MHz, DMSO-d₆): δ [ppm]=12.41 (br. s, 1H), 8.26 (dd, 1H), 8.20 (d, 1H), 8.09 (td, 1H), 7.97-7.90 (m, 1H), 7.86 (s, 1H), 7.68 (d, 1H), 7.28 (d, 1H), 6.98 (dd, 1H), 4.58-4.41 (m, 2H), 4.05 (t, 2H), 3.44-3.34 (m, 1H), 3.23-3.14 (m, 1H), 3.04-2.90 (m, 1H), 2.29-2.17 (m, 1H), 2.15-2.01 (m, 1H), 1.80-1.67 (m, 2H), 1.49-1.38 (m, 2H), 1.35-1.27 (m, 4H), 0.91-0.85 (m, 3H).

LC/MS (Method 1, ESIpos): Rₜ=1.29 min, m/z=492 [M+H]⁺.

Separation of the Enantiomers:

53 mg of the compound from Example 64 were dissolved in 5 ml of ethanol/acetonitrile and separated into the enantiomers by preparative HPLC on a chiral phase (see Examples 65 and 66) [column: Daicel Chiralpak AD-H, 5 μm, 250 mm×20 mm; flow rate: 20 ml/min; detection: 220 nm; injection volume: 0.5 ml; temperature: 20° C.; mobile phase: 70% ethanol/30% acetonitrile, isocratic, run time 22 min]:

### Example 65

### (−)-4-[6-(Hexyloxy)-1-benzofur-2-yl]-4-oxo-2-[2-(4-oxo-1,2,3-benzotriazin-3(4H)-yl)ethyl]butanoic acid (enantiomer 1)

21 mg (chemical purity 100%) of the title compound were obtained.

ee value=100%; [α]_{D}²⁰=−35.7°, 589 nm, c=0.33 g/100 ml, chloroform

¹H-NMR (400 MHz, DMSO-d₆): δ [ppm]=12.42 (br. s, 1H), 8.26 (dd, 1H), 8.20 (d, 1H), 8.11-8.06 (m, 1H), 7.96-7.90 (m, 1H), 7.86 (s, 1H), 7.68 (d, 1H), 7.28 (d, 1H), 6.98 (dd, 1H), 4.60-4.42 (m, 2H), 4.05 (t, 2H), 3.44-3.33 (m, 1H), 3.18 (dd, 1H), 3.02-2.91 (m, 1H), 2.30-2.17 (m, 1H), 2.14-2.00 (m, 1H), 1.81-1.67 (m, 2H), 1.49-1.38 (m, 2H), 1.36-1.27 (m, 4H), 0.92-0.82 (m, 3H).

LC/MS (Method 1, ESIpos): Rₜ=1.31 min, m/z=492 [M+H]⁺.

### Example 66

### (+)-4-[6-(Hexyloxy)-1-benzofur-2-yl]-4-oxo-2-[2-(4-oxo-1,2,3-benzotriazin-3(4H)-yl)ethyl]butanoic acid (enantiomer 2)

21 mg (chemical purity 100%) of the title compound were obtained.

Ee value=100%; [α]²⁰=+88.9°, 436 nm, c=0.06 g/100 ml, chloroform

¹H-NMR (400 MHz, DMSO-d₆): δ [ppm]=12.42 (br. s, 1H), 8.26 (d, 1H), 8.20 (d, 1H), 8.12-8.05 (m, 1H), 7.97-7.90 (m, 1H), 7.86 (s, 1H), 7.68 (d, 1H), 7.28 (s, 1H), 6.98 (dd, 1H), 4.59-4.40 (m, 2H), 4.05 (t, 2H), 3.44-3.33 (m, 1H), 3.18 (dd, 1H), 3.03-2.90 (m, 1H), 2.29-2.16 (m, 1H), 2.14-2.00 (m, 1H), 1.80-1.67 (m, 2H), 1.48-1.39 (m, 2H), 1.38-1.28 (m, 4H), 0.92-0.84 (m, 3H).

LC/MS (Method 1, ESIpos): Rₜ=1.31 min, m/z=492 [M+H]⁺.

### Example 67

### (+/−)-4-[6-(Heptyloxy)-1-benzofur-2-yl]-4-oxo-2-[2-(4-oxo-1,2,3-benzotriazin-3(4H)-yl)ethyl]butanoic acid

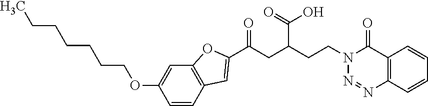 [SMILES: CCCCCCCOc1ccc2cc(C(=O)CC(CCn3nnc4ccccc4c3=O)C(=O)O)oc2c1]

A solution of 113 mg (0.17 mmol) of the compound from Example 49A in 1.5 ml of a 4 N solution of hydrogen chloride in dioxane was stirred firstly for 16 h at RT and then for 3 h under reflux. After cooling to RT, concentration and repeated reuptake and concentration with dioxane and dichloromethane, the residue was finally dried in vacuo. 47 mg (34% of theory, purity 63%) of the title compound were obtained.

¹H-NMR (400 MHz, DMSO-d₆): δ [ppm]=12.43 (br. s, 1H), 8.26 (d, 1H), 8.19 (d, 1H), 8.12-8.05 (m, 1H), 7.97-7.89 (m, 1H), 7.86 (s, 1H), 7.68 (d, 1H), 7.28 (s, 1H), 6.96 (dd, 1H), 4.58-4.44 (m, 2H), 4.05 (t, 2H), 3.52-3.50 (m, 1H), 3.39 (dd, 1H), 3.17 (dd, 1H), 3.02-2.91 (m, 1H), 2.28-2.18 (m, 1H), 2.12-2.01 (m, 1H), 1.79-1.70 (m, 2H), 1.48-1.22 (m, 6H), 1.04 (d, 1H), 0.84 (t, 3H).

LC/MS (Method 1, ESIpos): Rₜ=1.36 min, m/z=506 [M+H]⁺.

Separation of the Enantiomers:

47 mg of the compound from Example 67 were dissolved in 5 ml of acetonitrile and separated into the enantiomers by preparative HPLC on a chiral phase (see Examples 68 and 69) [column: Daicel Chiralpak AD-H, 5 μm, 250 mm×20 mm; flow rate: 20 ml/min; detection: 220 nm; injection volume: 0.4 ml; temperature: 20° C.; mobile phase: 70% methanol/30% acetonitrile, isocratic, run time 20 min]:

### Example 68

### (−)-4-[6-(Heptyloxy)-1-benzofur-2-yl]-4-oxo-2-[2-(4-oxo-1,2,3-benzotriazin-3(4H)-yl)ethyl]butanoic acid (enantiomer 1)

9 mg (chemical purity 94%) of the title compound were obtained.

ee value=100%; [α]²⁰=−99.70, 436 nm, c=0.32 g/100 ml, chloroform

¹H-NMR (400 MHz, DMSO-d₆): δ [ppm]=12.43 (br. s, 1H), 8.26 (d, 1H), 8.19 (d, 1H), 8.12-8.05 (m, 1H), 7.97-7.89 (m, 1H), 7.86 (s, 1H), 7.68 (d, 1H), 7.28 (s, 1H), 6.96 (dd, 1H), 4.58-4.44 (m, 2H), 4.05 (t, 2H), 3.52-3.50 (m, 1H), 3.39 (dd, 1H), 3.17 (dd, 1H), 3.02-2.91 (m, 1H), 2.28-2.18 (m, 1H), 2.12-2.01 (m, 1H), 1.79-1.70 (m, 2H), 1.48-1.22 (m, 6H), 1.04 (d, 1H), 0.84 (t, 3H).

LC/MS (Method 1, ESIpos): Rₜ=1.36 min, m/z=506 [M+H]⁺.

### Example 69

### (+)-4-[6-(Heptyloxy)-1-benzofur-2-yl]-4-oxo-2-[2-(4-oxo-1,2,3-benzotriazin-3(4H)-yl)ethyl]butanoic acid acid (enantiomer 2)

8 mg (chemical purity 100%) of the title compound were obtained.

ee value=100%; [α]²⁰=+101.2°, 436 nm, c=0.26 g/100 ml, chloroform

¹H-NMR (400 MHz, DMSO-d₆): δ [ppm]=12.43 (br. s, 1H), 8.26 (d, 1H), 8.19 (d, 1H), 8.12-8.05 (m, 1H), 7.97-7.89 (m, 1H), 7.86 (s, 1H), 7.68 (d, 1H), 7.28 (s, 1H), 6.96 (dd, 1H), 4.58-4.44 (m, 2H), 4.05 (t, 2H), 3.52-3.50 (m, 1H), 3.39 (dd, 1H), 3.17 (dd, 1H), 3.02-2.91 (m, 1H), 2.28-2.18 (m, 1H), 2.12-2.01 (m, 1H), 1.79-1.70 (m, 2H), 1.48-1.22 (m, 6H), 1.04 (d, 1H), 0.84 (t, 3H).

LC/MS (Method 1, ESIpos): Rₜ=1.36 min, m/z=506 [M+H]⁺.

### Example 70

### (+/−)-4-{6-[(4-Methylpentyl)oxy]-1-benzofur-2-yl}-4-oxo-2-[2-(4-oxo-1,2,3-benzotriazin-3(4H)-yl)ethyl]butanoic acid

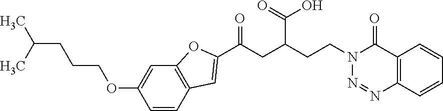 [SMILES: CC(C)CCCOc1ccc2cc(C(=O)CC(CCn3nnc4ccccc4c3=O)C(=O)O)oc2c1]

Analogously to the method described in Example 36, 30 mg (0.18 mmol) of 1-bromo-4-methylpentane were reacted with 85 mg (0.15 mmol) of the compound from Example 43A. Purification of the crude product by preparative HPLC [Method 10] gave 34 mg (46% of theory, purity 100%) of the title compound.

LC/MS (Method 4): Rₜ=1.25 min; MS (ESIpos): m/z=492 [M+H]⁺.

### Example 71

### (+/−)-4-[6-(3-Cyclohexylpropoxy)-1-benzofur-2-yl]-4-oxo-2-[2-(4-oxo-1,2,3-benzotriazin-3(4H)-yl)ethyl]butanoic acid

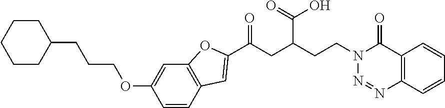 [SMILES: O=C(CC(CCn1nnc2ccccc2c1=O)C(=O)O)c1cc2ccc(OCCCC3CCCCC3)cc2o1]

Analogously to the method described in Example 36, 40 mg (0.18 mmol) of (3-bromopropyl)cyclohexane were reacted with 85 mg (0.15 mmol) of the compound from Example 43A. Purification of the crude product by preparative HPLC [Method 10] gave 31 mg (38% of theory, purity 100%) of the title compound.

LC/MS (Method 4): Rₜ=1.31 min; MS (ESIpos): m/z=532 [M+H]⁺.

### Example 72

### (+/−)-4-{6-[(5-Methylhexyl)oxy]-1-benzofur-2-yl}-4-oxo-2-[2-(4-oxo-1,2,3-benzotriazin-3(4H)-yl)ethyl]butanoic acid

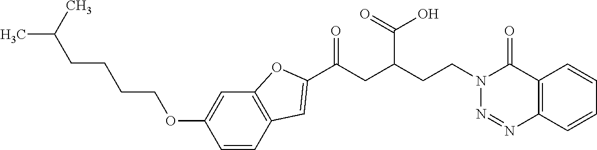 [SMILES: CC(C)CCCCOc1ccc2cc(C(=O)CC(CCn3nnc4ccccc4c3=O)C(=O)O)oc2c1]

Analogously to the method described in Example 36, 32 mg (0.18 mmol) of 1-bromo-5-methylhexane were reacted with 85 mg (0.15 mmol) of the compound from Example 43A. Purification of the crude product by preparative HPLC [Method 10] gave 29 mg (38% of theory, purity 98%) of the title compound.

LC/MS (Method 4): Rₜ=1.28 min; MS (ESIpos): m/z=506 [M+H]⁺.

### Example 73

### (+/−)-4-Oxo-2-[2-(4-oxo-1,2,3-benzotriazin-3(4H)-yl)ethyl]-4-[6-(2-phenylethoxy)-1-benzofur-2-yl]butanoic acid

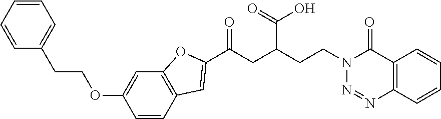 [SMILES: O=C(CC(CCn1nnc2ccccc2c1=O)C(=O)O)c1cc2ccc(OCCc3ccccc3)cc2o1]

Analogously to the method described in Example 36, 33 mg (0.18 mmol) of (2-bromoethyl)benzene were reacted with 85 mg (0.15 mmol) of the compound from Example 43A. Purification of the crude product by preparative HPLC [Method 10] gave 10 mg (12% of theory, purity 93%) of the title compound.

LC/MS (Method 4): Rₜ=1.20 min; MS (ESIpos): m/z=512 [M+H]⁺.

### Example 74

### (+/−)-4-Oxo-2-[2-(4-oxo-1,2,3-benzotriazin-3(4H)-yl)ethyl]-4-{6-[2-(tetrahydro-2H-pyran-4-yl)ethoxy]-1-benzofur-3-yl}butanoic acid

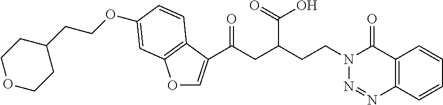 [SMILES: O=C(CC(CCn1nnc2ccccc2c1=O)C(=O)O)c1coc2cc(OCCC3CCOCC3)ccc12]

A solution of 163 mg (0.22 mmol, purity 75%) of the compound from Example 71A in 2 ml of dioxane was stirred under reflux for 1 h. After cooling to RT and concentration, the residue was purified by thick-layer chromatography (PLC silica gel 60 F 254, 1 mm; 20×20 cm with concentration zone; mobile phase dichloromethane/methanol 9:1). 90 mg (76% of theory, purity 95%) of the title compound were obtained.

¹H-NMR (400 MHz, DMSO-d₆): δ [ppm]=12.40 (br. s, 1H), 8.95 (s, 1H), 8.25 (d, 1H), 8.21 (d, 1H), 8.09 (dd, 1H), 7.95 (dd, 1H), 7.88 (s, 1H), 7.33 (d, 1H), 6.99 (dd, 1H), 4.57-4.44 (m, 2H), 4.07 (dd, 2H), 3.83 (dd, 2H), 3.39 (d, 1H), 3.26-3.18 (m, 2H), 3.01-2.95 (m, 1H), 2.29-2.18 (m, 1H), 2.09-2.01 (m, 1H), 1.71-1.62 (m, 5H), 1.28-1.18 (m, 3H).

LC/MS (Method 1, ESIpos): Rₜ=1.06 min, m/z=520 [M+H]⁺.

### Example 75

### (+/−)-4-[6-(2-Cyclopentylethoxy)-1-benzofur-3-yl]-4-oxo-2-[2-(4-oxo-1,2,3-benzotriazin-3(4H)-yl)ethyl]butanoic acid

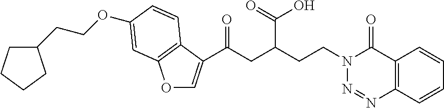 [SMILES: O=C(CC(CCn1nnc2ccccc2c1=O)C(=O)O)c1coc2cc(OCCC3CCCC3)ccc12]

A solution of 142 mg (0.25 mmol, purity 95%) of the compound from Example 72A in 3 ml of dioxane was stirred under reflux for 1 h. After cooling to RT and concentration, the residue was purified by thick-layer chromatography (PLC silica gel 60 F 254, 1 mm; 20×20 cm with concentration zone; mobile phase dichloromethane/methanol 9:1). 91 mg (70% of theory, purity 96%) of the title compound were obtained.

¹H-NMR (400 MHz, DMSO-d₆): δ [ppm]=12.41 (br. s, 1H), 8.95 (s, 1H), 8.25 (d, 1H), 8.21 (d, 1H), 8.09 (dd, 1H), 7.95 (dd, 1H), 7.88 (s, 1H), 7.30 (d, 1H), 6.98 (dd, 1H), 4.57-4.44 (m, 2H), 4.03 (dd, 2H), 3.39 (d, 1H), 3.20 (dd, 1H), 3.01-2.95 (m, 1H), 2.27-2.09 (m, 1H), 2.09-2.00 (m, 1H), 1.99-1.93 (m, 1H), 1.82-1.73 (m, 4H), 1.64-1.46 (m, 4H), 1.23-1.11 (m, 2H).

LC/MS (Method 1, ESIpos): Rₜ=1.31 min, m/z=504 [M+H]⁺.

### Example 76

### (+/−)-4-[6-(2-Cyclohexylethoxy)-1-benzofur-3-yl]-4-oxo-2-[2-(4-oxo-1,2,3-benzotriazin-3(4H)-yl)ethyl]butanoic acid

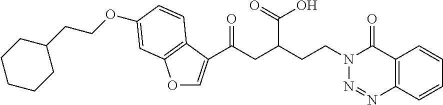 [SMILES: O=C(CC(CCn1nnc2ccccc2c1=O)C(=O)O)c1coc2cc(OCCC3CCCCC3)ccc12]

A solution of 117 mg (0.21 mmol) of the compound from Example 73A in 6.5 ml of dioxane was stirred under reflux for 4 h. After cooling to RT and concentration, the residue was purified by means of preparative HPLC (Method 6). 43 mg (40% of theory, purity 100%) of the title compound were obtained.

¹H-NMR (400 MHz, DMSO-d₆): δ [ppm]=12.38 (br. s, 1H), 8.95 (s, 1H), 8.26 (d, 1H), 8.21 (d, 1H), 8.09 (dd, 1H), 7.94 (dd, 1H), 7.87 (d, 1H), 7.30 (d, 1H), 6.98 (dd, 1H), 4.57-4.44 (m, 2H), 4.05 (dd, 2H), 3.40 (d, 1H), 3.20 (dd, 1H), 3.01-2.95 (m, 1H), 2.27-2.09 (m, 1H), 2.09-2.01 (m, 1H), 1.74 (d, 2H), 1.69-1.61 (m, 5H), 1.50-1.44 (m, 1H), 1.27-1.12 (m, 3H), 0.96 (dd, 2H).

LC/MS (Method 1, ESIpos): Rₜ=1.36 min, m/z=518 [M+H]⁺.

### Example 77

### (+/−)-4-{6-[(3-Chlorobenzyl)oxy]-1-benzofur-3-yl}-4-oxo-2-[2-(4-oxo-1,2,3-benzotriazin-3(4H)-yl)ethyl]butanoic acid

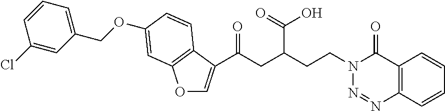 [SMILES: O=C(CC(CCn1nnc2ccccc2c1=O)C(=O)O)c1coc2cc(OCc3cccc(Cl)c3)ccc12]

A solution of 115 mg (0.20 mmol) of the compound from Example 74A in 6 ml of dioxane was stirred under reflux for 16 h. After cooling to RT and concentration, the residue was purified by means of preparative HPLC (Method 6). 26 mg (25% of theory, purity 100%) of the title compound were obtained.

¹H-NMR (400 MHz, DMSO-d₆): δ [ppm]=12.40 (br. s, 1H), 8.97 (s, 1H), 8.26 (d, 1H), 8.20 (d, 1H), 8.09 (dd, 1H), 7.95-7.88 (m, 2H), 7.55 (br. s, 1H), 7.45-7.40 (m, 4H), 7.10 (dd, 1H), 5.20 (s, 2H), 4.57-4.44 (m, 2H), 3.37 (dd, 1H), 3.21 (dd, 1H), 3.01-2.94 (m, 1H), 2.29-2.18 (m, 1H), 2.09-2.01 (m, 1H).

LC/MS (Method 1, ESIpos): Rₜ=1.23 min, m/z=532 [M+H]⁺.

### Example 78

### (+/−)-4-[6-(Cyclopentylmethoxy)-1-benzofur-3-yl]-4-oxo-2-[2-(4-oxo-1,2,3-benzotriazin-3(4H)-yl)ethyl]butanoic acid

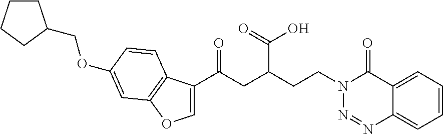 [SMILES: O=C(CC(CCn1nnc2ccccc2c1=O)C(=O)O)c1coc2cc(OCC3CCCC3)ccc12]

A solution of 448 mg (0.76 mmol, purity 90%) of the compound from Example 75A in 21 ml of dioxane was stirred under reflux for 16 h. After cooling to RT and concentration, the residue was purified by column chromatography (80 g silica gel, mobile phase dichloromethane/methanol 97:3). The product thus obtained was then after-purified by means of preparative HPLC (Method 6). 157 mg (42% of theory, purity 100%) of the title compound were obtained.

¹H-NMR (400 MHz, DMSO-d₆): δ [ppm]=12.40 (br. s, 1H), 8.95 (s, 1H), 8.25 (d, 1H), 8.21 (d, 1H), 8.12-8.06 (m, 1H), 7.96-7.90 (m, 1H), 7.87 (d, 1H), 7.29 (d, 1H), 6.98 (dd, 1H), 4.60-4.42 (m, 2H), 3.90 (d, 2H), 3.41-3.34 (m, 1H), 3.25-3.17 (m, 1H), 3.02-2.93 (m, 1H), 2.40-2.16 (m, 2H), 2.11-1.98 (m, 1H), 1.85-1.72 (m, 2H), 1.67-1.48 (m, 4H), 1.39-1.29 (m, 2H).

LC/MS (Method 1, ESIpos): Rₜ=1.28 min, m/z=490 [M+H]⁺.

Separation of the Enantiomers:

110 mg of the compound from Example 78 were dissolved in 10 ml of ethanol and 1 ml of acetonitrile and separated into the enantiomers by preparative HPLC on a chiral phase (see Examples 79 and 80) [column: Daicel Chiralcel OZ-H, 5 μm, 250 mm×20 mm; flow rate: 15 ml/min; detection: 220 nm; injection volume: 0.6 ml; temperature: 40° C.; mobile phase: 50% isohexane/50% ethanol+0.2% acetic acid, isocratic, run time 15 min]:

### Example 79

### (−)-4-[6-(Cyclopentylmethoxy)-1-benzofur-3-yl]-4-oxo-2-[2-(4-oxo-1,2,3-benzotriazin-3(4H)-yl)ethyl]butanoic acid (enantiomer 1)

41 mg (chemical purity 100%) of the title compound were obtained.

ee value=99%; [α]_{D}²⁰=−6.5°, 589 nm, c=0.25 g/100 ml, chloroform

¹H-NMR (400 MHz, DMSO-d₆): δ [ppm]=12.39 (br. s, 1H), 8.95 (s, 1H), 8.25 (d, 1H), 8.21 (d, 1H), 8.13-8.05 (m, 1H), 7.97-7.91 (m, 1H), 7.87 (d, 1H), 7.29 (d, 1H), 6.98 (dd, 1H), 4.58-4.42 (m, 2H), 3.90 (d, 2H), 3.41-3.34 (m, 1H), 3.25-3.16 (m, 1H), 3.03-2.92 (m, 1H), 2.39-2.16 (m, 2H), 2.11-1.99 (m, 1H), 1.84-1.72 (m, 2H), 1.68-1.48 (m, 4H), 1.42-1.28 (m, 2H).

LC/MS (Method 1, ESIpos): Rₜ=1.25 min, m/z=490 [M+H]⁺.

### Example 80

### (+)-4-[6-(Cyclopentylmethoxy)-1-benzofur-3-yl]-4-oxo-2-[2-(4-oxo-1,2,3-benzotriazin-3(4H)-yl)ethyl]butanoic acid (enantiomer 2)

48 mg (chemical purity 100%) of the title compound were obtained.

ee value=99%; [α]_{D}²⁰=+2.0°, 589 nm, c=0.28 g/100 ml, chloroform

¹H-NMR (400 MHz, DMSO-d₆): δ [ppm]=12.40 (br. s, 1H), 8.95 (s, 1H), 8.25 (dd, 1H), 8.21 (d, 1H), 8.09 (td, 1H), 7.97-7.91 (m, 1H), 7.88 (d, 1H), 7.29 (d, 1H), 6.98 (dd, 1H), 4.58-4.43 (m, 2H), 3.90 (d, 2H), 3.41-3.34 (m, 1H), 3.25-3.16 (m, 1H), 3.03-2.92 (m, 1H), 2.39-2.16 (m, 2H), 2.11-2.00 (m, 1H), 1.84-1.73 (m, 2H), 1.68-1.48 (m, 4H), 1.40-1.29 (m, 2H).

LC/MS (Method 1, ESIpos): Rₜ=1.25 min, m/z=490 [M+H]⁺.

### Example 81

### (+/−)-4-[6-(Cyclohexylmethoxy)-1-benzofur-3-yl]-4-oxo-2-[2-(4-oxo-1,2,3-benzotriazin-3(4H)-yl)ethyl]butanoic acid

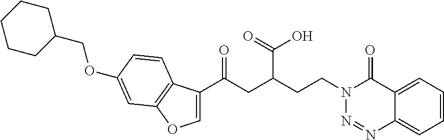 [SMILES: O=C(CC(CCn1nnc2ccccc2c1=O)C(=O)O)c1coc2cc(OCC3CCCCC3)ccc12]

A solution of 360 mg (0.57 mmol, purity 87%) of the compound from Example 76A in 3 ml of dioxane was stirred under reflux for 16 h. After cooling to RT and concentration, the residue was dried in vacuo. 294 mg (97% of theory, purity 95%) of the title compound were obtained.

LC/MS (Method 1, ESIpos): Rₜ=1.34 min, m/z=504 [M+H]⁺.

Separation of the Enantiomers:

294 mg of the compound from Example 81 were dissolved in 60 ml of a mixture of acetonitrile and methanol and separated into the enantiomers by preparative HPLC on a chiral phase (see Examples 82 and 83) [column: Daicel Chiralpak AD-H, 5 μm, 250 mm×20 mm; flow rate: 20 ml/min; detection: 220 nm; injection volume: 0.5 ml; temperature: 20° C.; mobile phase: 30% acetonitrile/70% ethanol, isocratic, run time 11 min]:

### Example 82

### (+)-4-[6-(Cyclohexylmethoxy)-1-benzofur-3-yl]-4-oxo-2-[2-(4-oxo-1,2,3-benzotriazin-3(4H)-yl)ethyl]butanoic acid (enantiomer 1)

28 mg (chemical purity 100%) of the title compound were obtained.

ee value=99.9%; [α]²⁰=+41.10, 365 nm, c=0.16 g/100 ml, chloroform

¹H-NMR (400 MHz, DMSO-d₆): δ [ppm]=12.41 (br. s, 1H), 8.95 (s, 1H), 8.25 (d, 1H), 8.21 (d, 1H), 8.09 (dd, 1H), 7.93 (dd, 1H), 7.87 (d, 1H), 7.29 (d, 1H), 6.98 (dd, 1H), 4.57-4.44 (m, 2H), 3.84 (d, 2H), 3.40 (d, 1H), 3.20 (dd, 1H), 3.01-2.95 (m, 1H), 2.26-2.18 (m, 1H), 2.09-2.01 (m, 1H), 1.84-1.64 (m, 6H), 1.30-1.16 (m, 3H), 1.10-1.01 (dd, 2H).

LC/MS (Method 1, ESIpos): Rₜ=1.31 min, m/z=504 [M+H]⁺.

### Example 83

### (−)-4-[6-(Cyclohexylmethoxy)-1-benzofur-3-yl]-4-oxo-2-[2-(4-oxo-1,2,3-benzotriazin-3(4H)-yl)ethyl]butanoic acid (enantiomer 2)

25 mg (chemical purity 100%) of the title compound were obtained.

ee value=83%; [α]²⁰=−49.90, 365 nm, c=0.25 g/100 ml, chloroform

¹H-NMR (400 MHz, DMSO-d₆): δ [ppm]=12.41 (br. s, 1H), 8.95 (s, 1H), 8.25 (d, 1H), 8.21 (d, 1H), 8.09 (dd, 1H), 7.94 (dd, 1H), 7.87 (d, 1H), 7.28 (d, 1H), 6.98 (dd, 1H), 4.57-4.44 (m, 2H), 3.84 (d, 2H), 3.40 (d, 1H), 3.20 (dd, 1H), 3.01-2.95 (m, 1H), 2.26-2.18 (m, 1H), 2.09-2.01 (m, 1H), 1.84-1.64 (m, 6H), 1.31-1.16 (m, 3H), 1.10-1.01 (dd, 2H).

LC/MS (Method 1, ESIpos): Rₜ=1.31 min, m/z=504 [M+H]⁺.

### Example 84

### (+/−)-4-[6-(Benzyloxy)-1-benzofur-3-yl]-4-oxo-2-[2-(4-oxo-1,2,3-benzotriazin-3(4H)-yl)ethyl]butanoic acid

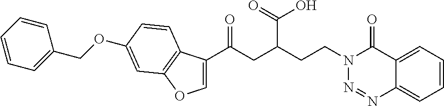 [SMILES: O=C(CC(CCn1nnc2ccccc2c1=O)C(=O)O)c1coc2cc(OCc3ccccc3)ccc12]

A solution of 80 mg (0.12 mmol) of the compound from Example 54A in 1.5 ml of a 4 N solution of hydrogen chloride in dioxane was stirred firstly for 16 h at RT and then for 2 h under reflux. After cooling to RT and concentration, the residue was admixed with dioxane, the solution was again concentrated and the residue was finally dried in vacuo. 72 mg of a crude product were obtained, 30 mg of which were stirred into acetonitrile. The solid present was filtered off, washed with acetonitrile and dried in vacuo. 25 mg (41% of theory, purity 99%) of the title compound were obtained.

¹H-NMR (400 MHz, DMSO-d₆): δ [ppm]=12.36 (br. s, 1H), 8.95 (s, 1H), 8.25 (dd, 1H), 8.20 (d, 1H), 8.09 (td, 1H), 7.97-7.87 (m, 2H), 7.50-7.45 (m, 2H), 7.43-7.30 (m, 4H), 7.07 (dd, 1H), 5.17 (s, 2H), 4.58-4.43 (m, 2H), 3.37 (dd, 1H), 3.21 (dd, 1H), 3.03-2.92 (m, 1H), 2.29-2.17 (m, 1H), 2.11-1.98 (m, 1H).

LC/MS (Method 1, ESIpos): Rₜ=1.16 min, m/z=498 [M+H]⁺.

Separation of the Enantiomers:

40 mg of the compound from Example 84 were dissolved in 5 ml of acetonitrile with the addition of 3 ml of methanol and separated into the enantiomers by preparative HPLC on a chiral phase (see Example 85) [column: Daicel Chiralpak AD-H, 5 μm, 250 mm×20 mm; flow rate: 20 ml/min; detection: 220 nm; injection volume: 0.5 ml; temperature: 20° C.; mobile phase: 29% acetonitrile/67% ethanol/4% acetonitrile with 5% glacial acetic acid, isocratic, run time 12 min]:

### Example 85

### (+)-4-[6-(Benzyloxy)-1-benzofur-3-yl]-4-oxo-2-[2-(4-oxo-1,2,3-benzotriazin-3(4H)-yl)ethyl]butanoic acid (enantiomer 1)

8 mg (chemical purity 93%) of the title compound were obtained.

ee value=100%; [α]²⁰=+1.30, 546 nm, c=0.31 g/100 ml, chloroform

¹H-NMR (400 MHz, DMSO-d₆): δ [ppm]=12.39 (br. s, 1H), 8.96 (s, 1H), 8.25 (dd, 1H), 8.21 (d, 1H), 8.09 (td, 1H), 7.97-7.87 (m, 2H), 7.51-7.45 (m, 2H), 7.44-7.30 (m, 4H), 7.07 (dd, 1H), 5.17 (s, 2H), 4.59-4.42 (m, 2H), 3.41-3.34 (m, 1H), 3.25-3.15 (m, 1H), 3.04-2.92 (m, 1H), 2.29-2.17 (m, 1H), 2.11-1.99 (m, 1H).

LC/MS (Method 1, ESIpos): Rₜ=1.16 min, m/z=498 [M+H]⁺.

The corresponding enantiomer 2 was obtained in a yield of 10 mg with a chemical purity of 60% according to LC/MS and an ee value of 98%:

LC/MS (Method 1, ESIpos): Rₜ=1.16 min, m/z=498 [M+H]⁺.

### Example 86

### (+/−)-4-Oxo-2-[2-(4-oxo-1,2,3-benzotriazin-3(4H)-yl)ethyl]-4-(6-propoxy-1-benzofur-3-yl)butanoic acid

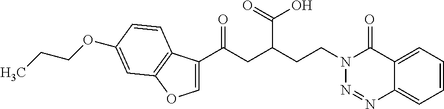 [SMILES: CCCOc1ccc2c(C(=O)CC(CCn3nnc4ccccc4c3=O)C(=O)O)coc2c1]

A solution of 109 mg (0.18 mmol) of the compound from Example 62A in 2 ml of a 4 N solution of hydrogen chloride in dioxane was stirred firstly for 16 h at RT and then for 2.5 h under reflux. After cooling to RT and concentration, the residue was admixed with dichloromethane, the solution was again concentrated and the residue was finally dried in vacuo. 80 mg (96% of theory, purity 97%) of the title compound were obtained.

¹H-NMR (400 MHz, DMSO-d₆): δ [ppm]=12.37 (br. s, 1H), 8.94 (s, 1H), 8.25 (d, 1H), 8.20 (d, 1H), 8.11-8.07 (m, 1H), 7.96-7.90 (m, 1H), 7.88 (d, 1H), 7.28 (d, 1H), 6.98 (dd, 1H), 4.57-4.43 (m, 2H), 3.98 (t, 2H), 3.57 (s, 1H), 3.36 (dd, 1H), 3.20 (dd, 1H), 3.03-2.93 (m, 1H), 2.27-2.18 (m, 1H), 2.11-2.00 (m, 1H), 1.80-1.71 (m, 2H), 0.99 (t, 2H).

LC/MS (Method 1, ESIpos): Rₜ=1.12 min, m/z=450 [M+H]⁺.

Separation of the Enantiomers:

65 mg of the compound from Example 86 were dissolved in 2 ml of methanol and separated into the enantiomers by preparative HPLC on a chiral phase (see Examples 87 and 88) [column: Daicel Chiralpak AD-H, 5 μm, 250 mm×20 mm; flow rate: 30 ml/min; detection: 270 nm; injection volume: 0.1 ml; temperature: 25° C.; mobile phase: 70% methanol/30% acetonitrile, isocratic, run time 13 min]:

### Example 87

### (−)-4-Oxo-2-[2-(4-oxo-1,2,3-benzotriazin-3(4H)-yl)ethyl]-4-(6-propoxy-1-benzofur-3-yl)butanoic acid (enantiomer 1)

23 mg (chemical purity 99%) of the title compound were obtained.

ee value=100%; [α]_{D}²⁰=−7.2°, 589 nm, c=0.32 g/100 ml, chloroform

¹H-NMR (400 MHz, DMSO-d₆): δ [ppm]=12.37 (br. s, 1H), 8.94 (s, 1H), 8.25 (d, 1H), 8.20 (d, 1H), 8.11-8.07 (m, 1H), 7.96-7.90 (m, 1H), 7.88 (d, 1H), 7.28 (d, 1H), 6.98 (dd, 1H), 4.57-4.43 (m, 2H), 3.98 (t, 2H), 3.57 (s, 1H), 3.36 (dd, 1H), 3.20 (dd, 1H), 3.03-2.93 (m, 1H), 2.27-2.18 (m, 1H), 2.11-2.00 (m, 1H), 1.80-1.71 (m, 2H), 0.99 (t, 2H).

LC/MS (Method 1, ESIpos): Rₜ=1.11 min, m/z=450 [M+H]⁺.

### Example 88

### (+)-4-Oxo-2-[2-(4-oxo-1,2,3-benzotriazin-3(4H)-yl)ethyl]-4-(6-propoxy-1-benzofur-3-yl)butanoic acid (enantiomer 2)

22 mg (chemical purity 99%) of the title compound were obtained.

ee value=97%; [α]_{D}²⁰=+4.1°, 589 nm, c=0.36 g/100 ml, chloroform

¹H-NMR (400 MHz, DMSO-d₆): δ [ppm]=12.37 (br. s, 1H), 8.94 (s, 1H), 8.25 (d, 1H), 8.20 (d, 1H), 8.11-8.07 (m, 1H), 7.96-7.90 (m, 1H), 7.88 (d, 1H), 7.28 (d, 1H), 6.98 (dd, 1H), 4.57-4.43 (m, 2H), 3.98 (t, 2H), 3.57 (s, 1H), 3.36 (dd, 1H), 3.20 (dd, 1H), 3.03-2.93 (m, 1H), 2.27-2.18 (m, 1H), 2.11-2.00 (m, 1H), 1.80-1.71 (m, 2H), 0.99 (t, 2H).

LC/MS (Method 1, ESIpos): Rₜ=1.11 min, m/z=450 [M+H]⁺.

### Example 89

### (+/−)-4-Oxo-2-[2-(4-oxo-1,2,3-benzotriazin-3(4H)-yl)ethyl]-4-[6-(pentyloxy)-1-benzofur-3-yl]butanoic acid

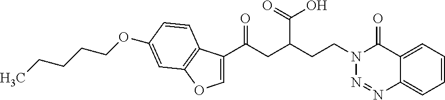 [SMILES: CCCCCOc1ccc2c(C(=O)CC(CCn3nnc4ccccc4c3=O)C(=O)O)coc2c1]

A solution of 117 mg (0.19 mmol) of the compound from Example 63A in 2 ml of a 4 N solution of hydrogen chloride in dioxane was stirred firstly for 16 h at RT and then for 2.5 h under reflux. After cooling to RT and concentration, the residue was admixed with dichloromethane, the solution was again concentrated and the residue was finally dried in vacuo. 87 mg (97% of theory, purity 98%) of the title compound were obtained.

¹H-NMR (400 MHz, DMSO-d₆): δ [ppm]=12.37 (br. s, 1H), 8.94 (s, 1H), 8.25 (d, 1H), 8.20 (d, 1H), 8.12-8.06 (m, 1H), 7.95-7.90 (m, 1H), 7.88 (d, 1H), 7.28 (d, 1H), 6.98 (dd, 1H), 4.51-4.44 (m, 2H), 4.02 (t, 2H), 3.36 (dd, 1H), 3.20 (dd, 1H), 3.02-2.93 (m, 1H), 2.28-2.17 (m, 1H), 2.12-1.98 (m, 1H), 1.77-1.70 (m, 2H), 1.45-1.31 (m, 4H), 0.9 (t, 3H).

LC/MS (Method 1, ESIpos): Rₜ=1.26 min, m/z=478 [M+H]⁺.

Separation of the Enantiomers:

74 mg of the compound from Example 89 were dissolved in 5 ml of methanol and separated into the enantiomers by preparative HPLC on a chiral phase (see Examples 90 and 91) [column: Daicel Chiralpak AD-H, 5 μm, 250 mm×20 mm; flow rate: 20 ml/min; detection: 220 nm; injection volume: 0.2 ml; temperature: 25° C.; mobile phase: 30% acetonitrile/70% methanol+0.2% acetic acid, isocratic, run time 5.5 min]:

### Example 90

### 4-Oxo-2-[2-(4-oxo-1,2,3-benzotriazin-3(4H)-yl)ethyl]-4-[6-(pentyloxy)-1-benzofur-3-yl]butanoic acid (enantiomer 1)

18 mg (chemical purity 99%) of the title compound were obtained.

Rₜ=6.90 min, ee value=100% [column: Daicel Chiralpak AD-H, 250 mm×4.6 mm, 5 μm; flow rate: 1.0 ml/min; detection: 270 nm; temperature: 30° C.; mobile phase: 50% methanol+0.2% glacial acetic acid/50% acetonitrile+0.2% glacial acetic acid, isocratic].

¹H-NMR (400 MHz, DMSO-d₆): δ [ppm]=12.38 (br. s, 1H), 8.94 (s, 1H), 8.25 (d, 1H), 8.20 (d, 1H), 8.12-8.06 (m, 1H), 7.95-7.90 (m, 1H), 7.88 (d, 1H), 7.28 (d, 1H), 6.98 (dd, 1H), 4.51-4.44 (m, 2H), 4.02 (t, 2H), 3.36 (dd, 1H), 3.20 (dd, 1H), 3.02-2.93 (m, 1H), 2.28-2.17 (m, 1H), 2.12-1.98 (m, 1H), 1.77-1.70 (m, 2H), 1.45-1.31 (m, 4H), 0.9 (t, 3H).

LC/MS (Method 1, ESIpos): Rₜ=1.26 min, m/z=478 [M+H]⁺.

### Example 91

### 4-Oxo-2-[2-(4-oxo-1,2,3-benzotriazin-3(4H)-yl)ethyl]-4-[6-(pentyloxy)-1-benzofur-3-yl]butanoic acid (enantiomer 2)

15 mg (chemical purity 99%) of the title compound were obtained.

Rₜ=8.03 min, ee value=100% [column: Daicel Chiralpak AD-H, 250 mm×4.6 mm, 5 μm; flow rate: 1.0 ml/min; detection: 270 nm; temperature: 30° C.; mobile phase: 50% methanol+0.2% glacial acetic acid/50% acetonitrile+0.2% glacial acetic acid, isocratic].

¹H-NMR (400 MHz, DMSO-d₆): δ [ppm]=12.38 (br. s, 1H), 8.94 (s, 1H), 8.25 (d, 1H), 8.20 (d, 1H), 8.12-8.06 (m, 1H), 7.95-7.90 (m, 1H), 7.88 (d, 1H), 7.28 (d, 1H), 6.98 (dd, 1H), 4.51-4.44 (m, 2H), 4.02 (t, 2H), 3.36 (dd, 1H), 3.20 (dd, 1H), 3.02-2.93 (m, 1H), 2.28-2.17 (m, 1H), 2.12-1.98 (m, 1H), 1.77-1.70 (m, 2H), 1.45-1.31 (m, 4H), 0.9 (t, 3H).

LC/MS (Method 1, ESIpos): Rₜ=1.26 min, m/z=478 [M+H]⁺.

### Example 92

### (+/−)-4-{6-[(3,4-Dichlorobenzyl)oxy]-1-benzofur-3-yl}-4-oxo-2-[2-(4-oxo-1,2,3-benzotriazin-3(4H)-yl)ethyl]butanoic acid

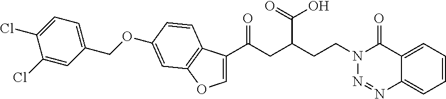 [SMILES: O=C(CC(CCn1nnc2ccccc2c1=O)C(=O)O)c1coc2cc(OCc3ccc(Cl)c(Cl)c3)ccc12]

A solution of 100 mg (0.14 mmol) of the compound from Example 64A in 1.5 ml of a 4 N solution of hydrogen chloride in dioxane was stirred firstly for 16 h at RT and then for 2 h under reflux. After cooling to RT and concentration, the residue was admixed with dichloromethane, the solution was again concentrated and the residue was finally dried in vacuo. 71 mg (78% of theory, purity 96%) of the title compound were obtained.

¹H-NMR (400 MHz, DMSO-d₆): δ [ppm]=12.37 (br. s, 1H), 8.97 (s, 1H), 8.25 (d, 1H), 8.20 (d, 1H), 8.12-8.05 (m, 1H), 7.97-7.87 (m, 2H), 7.76 (d, 1H), 7.67 (d, 1H), 7.48 (dd, 1H), 7.40 (d, 1H), 7.09 (dd, 1H), 5.20 (s, 2H), 4.58-4.42 (m, 2H), 3.33 (dd, 1H), 3.21 (m, 1H), 3.01-2.95 (m, 1H), 2.29-2.17 (m, 1H), 2.11-1.99 (m, 1H).

LC/MS (Method 1, ESIpos): Rₜ=1.30 min, m/z=568 [M+H]⁺.

Separation of the Enantiomers:

51 mg of the compound from Example 92 were partially dissolved in 10 ml of acetonitrile/ethanol, filtered and then separated into the enantiomers by preparative HPLC on a chiral phase (see Examples 93 and 94) [column: Daicel Chiralpak AD-H, 5 μm, 250 mm×20 mm; flow rate: 20 ml/min; detection: 220 nm; injection volume: 1 ml; temperature: 20° C.; mobile phase: 30% acetonitrile/70% ethanol, isocratic, run time 20 min]:

### Example 93

### 4-{6-[(3,4-Dichlorobenzyl)oxy]-1-benzofur-3-yl}-4-oxo-2-[2-(4-oxo-1,2,3-benzotriazin-3(4H)-yl)ethyl]butanoic acid (enantiomer 1)

23 mg (chemical purity 90%) of the title compound were obtained.

Rₜ=8.91 min, ee value=100% [column: Daicel Chiralpak AD-H, 250 mm×4.6 mm, 5 μm; flow rate: 1.0 ml/min; detection: 220 nm; temperature: 40° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.37 (br. s, 1H), 8.96 (s, 1H), 8.25 (d, 1H), 8.20 (d, 1H), 8.12-8.05 (m, 1H), 7.97-7.87 (m, 2H), 7.76 (d, 1H), 7.67 (d, 1H), 7.48 (dd, 1H), 7.40 (d, 1H), 7.09 (dd, 1H), 5.20 (s, 2H), 4.58-4.42 (m, 2H), 3.33 (dd, 1H), 3.21 (m, 1H), 3.01-2.95 (m, 1H), 2.29-2.17 (m, 1H), 2.11-1.99 (m, 1H).

LC/MS (Method 1, ESIpos): Rₜ=1.29 min, m/z=568 [M+H]⁺.

### Example 94

### 4-{6-[(3,4-Dichlorobenzyl)oxy]-1-benzofur-3-yl}-4-oxo-2-[2-(4-oxo-1,2,3-benzotriazin-3(4H)-yl)ethyl]butanoic acid (enantiomer 2)

22 mg (chemical purity 98%) of the title compound were obtained.

Rₜ=13.23 min, ee value=94% [column: Daicel Chiralpak AD-H, 250 mm×4.6 mm, 5 μm; flow rate: 1.0 ml/min; detection: 220 nm; temperature: 40° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.37 (br. s, 1H), 8.96 (s, 1H), 8.25 (d, 1H), 8.20 (d, 1H), 8.12-8.05 (m, 1H), 7.97-7.87 (m, 2H), 7.76 (d, 1H), 7.67 (d, 1H), 7.48 (dd, 1H), 7.40 (d, 1H), 7.09 (dd, 1H), 5.20 (s, 2H), 4.58-4.42 (m, 2H), 3.33 (dd, 1H), 3.21 (m, 1H), 3.01-2.95 (m, 1H), 2.29-2.17 (m, 1H), 2.11-1.99 (m, 1H).

LC/MS (Method 1, ESIpos): Rₜ=1.29 min, m/z=568 [M+H]⁺.

### Example 95

### (+/−)-4-Oxo-2-[2-(4-oxo-1,2,3-benzotriazin-3(4H)-yl)ethyl]-4-[6-(tetrahydro-2H-pyran-4-ylmethoxy)-1-benzofur-3-yl]butanoic acid

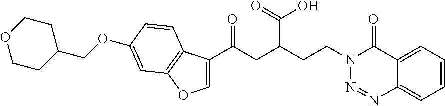 [SMILES: O=C(CC(CCn1nnc2ccccc2c1=O)C(=O)O)c1coc2cc(OCC3CCOCC3)ccc12]

Method A:

A solution of 88 mg (0.13 mmol) of the compound from Example 65A in 1.5 ml of a 4 N solution of hydrogen chloride in dioxane was stirred firstly for 16 h at RT and then for 2 h under reflux. After cooling to RT and concentration, the residue was admixed with dichloromethane, the solution was again concentrated and the residue was finally dried in vacuo. This residue was then admixed with dioxane, the solution was again concentrated and the residue was dried once again in vacuo. 40 mg (55% of theory, purity 94%) of the title compound were obtained.

¹H-NMR (400 MHz, DMSO-d₆): δ [ppm]=12.37 (br. s, 1H), 8.95 (s, 1H), 8.25 (dd, 1H), 8.21 (d, 1H), 8.09 (td, 1H), 7.96-7.91 (m, 1H), 7.88 (d, 1H), 7.30 (d, 1H), 6.99 (dd, 1H), 4.58-4.43 (m, 2H), 3.94-3.82 (m, 4H), 3.42-3.33 (m, 1H), 3.32-3.29 (m, 2H, hidden), 3.25-3.14 (m, 1H), 3.04-2.91 (m, 1H), 2.29-2.15 (m, 1H), 2.11-1.95 (m, 2H), 1.75-1.63 (m, 2H), 1.44-1.21 (m, 2H).

LC/MS (Method 1, ESIpos): Rₜ=1.03 min, m/z=506 [M+H]⁺.

Method B:

A solution of 408 mg (0.66 mmol, purity 89%) of the compound from Example 146A in 3.5 ml of dioxane was stirred under reflux for 1 h. After cooling to RT and concentration, the residue was dried in vacuo. 223 mg (80% of theory, purity 83%) of the title compound were obtained.

Separation of the Enantiomers:

322 mg of the compound from Example 95 were dissolved in 24 ml of of an ethanol/methanol mixture and separated into the enantiomers by preparative HPLC on a chiral phase (see Examples 96 and 97) [column: Daicel Chiralpak AD-H, 5 μm, 250 mm×20 mm; flow rate: 20 ml/min; detection: 230 nm; injection volume: 0.12 ml; temperature: 25° C.; mobile phase 52% acetonitrile/40% ethanol/8% acetonitrile-glacial acetic acid mixture (95:5), isocratic, run time 7.5 min]:

### Example 96

### (+)-4-Oxo-2-[2-(4-oxo-1,2,3-benzotriazin-3(4H)-yl)ethyl]-4-[6-(tetrahydro-2H-pyran-4-ylmethoxy)-1-benzofur-3-yl]butanoic acid (enantiomer 1)

112 mg (chemical purity 100%) of the title compound were obtained.

ee value=100%; [α]²⁰=+65.7°, 365 nm, c=0.25 g/100 ml, chloroform

¹H-NMR (400 MHz, DMSO-d₆): δ [ppm]=12.34 (br. s, 1H), 8.94 (s, 1H), 8.23 (dd, 2H), 8.09 (t, 1H), 7.96-7.84 (m, 2H), 7.30 (d, 1H), 6.99 (dd, 1H), 4.62-4.39 (m, 2H), 3.94-3.81 (m, 4H), 3.42-3.28 (m, 3H, partially hidden), 3.26-3.12 (m, 1H), 3.03-2.91 (m, 1H), 2.29-2.16 (m, 1H), 2.10-1.96 (m, 2H), 1.73-1.63 (m, 2H), 1.42-1.26 (m, 2H).

LC/MS (Method 1, ESIpos): Rₜ=1.03 min, m/z=506 [M+H]⁺.

### Example 97

### (−)-4-Oxo-2-[2-(4-oxo-1,2,3-benzotriazin-3(4H)-yl)ethyl]-4-[6-(tetrahydro-2H-pyran-4-ylmethoxy)-1-benzofur-3-yl]butanoic acid (enantiomer 2)

110 mg (chemical purity 100%) of the title compound were obtained.

ee value=98%; [α]²⁰=−62.8°, 365 nm, c=0.27 g/100 ml, chloroform

¹H-NMR (400 MHz, DMSO-d₆): δ [ppm]=12.32 (br. s, 1H), 8.94 (s, 1H), 8.25 (d, 1H), 8.20 (d, 1H), 8.09 (t, 1H), 7.93 (t, 1H), 7.88 (d, 1H), 7.30 (d, 1H), 6.99 (dd, 1H), 4.60-4.42 (m, 2H), 3.93-3.83 (m, 4H), 3.42-3.31 (m, 3H), 3.20 (dd, 1H), 3.03-2.91 (m, 1H), 2.29-2.16 (m, 1H), 2.10-1.98 (m, 2H), 1.74-1.64 (m, 2H), 1.41-1.26 (m, 2H).

LC/MS (Method 1, ESIpos): Rₜ=1.03 min, m/z=506 [M+H]⁺.

### Example 98

### (+/−)-4-[6-(Hexyloxy)-1-benzofur-3-yl]-4-oxo-2-[2-(4-oxo-1,2,3-benzotriazin-3(4H)-yl)ethyl]butanoic acid

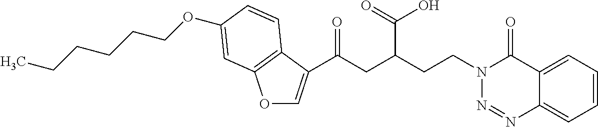 [SMILES: CCCCCCOc1ccc2c(C(=O)CC(CCn3nnc4ccccc4c3=O)C(=O)O)coc2c1]

A solution of 120 mg (0.19 mmol) of the compound from Example 66A in 2 ml of a 4 N solution of hydrogen chloride in dioxane was stirred firstly for 16 h at RT and then for 2 h under reflux. After cooling to RT and concentration, the residue was admixed with dioxane, the solution was again concentrated and the residue was finally dried in vacuo. 83 mg (85% of theory, purity 93%) of the title compound were obtained.

¹H-NMR (400 MHz, DMSO-d₆): δ [ppm]=12.36 (br. s, 1H), 8.94 (s, 1H), 8.25 (dd, 1H), 8.20 (d, 1H), 8.12-8.06 (m, 1H), 7.96-7.90 (m, 1H), 7.87 (d, 1H), 7.28 (d, 1H), 6.98 (dd, 1H), 4.58-4.42 (m, 2H), 4.02 (t, 2H), 3.40-3.32 (m, 1H), 3.24-3.16 (m, 1H), 3.03-2.92 (m, 1H), 2.29-2.17 (m, 1H), 2.11-1.98 (m, 1H), 1.78-1.68 (m, 2H), 1.48-1.37 (m, 2H), 1.35-1.26 (m, 4H), 0.91-0.83 (m, 3H).

LC/MS (Method 1, ESIpos): Rₜ=1.33 min, m/z=492 [M+H]⁺.

Separation of the Enantiomers:

69 mg of the compound from Example 98 were dissolved in 2 ml of methanol and separated into the enantiomers by preparative HPLC on a chiral phase (see Examples 99 and 100) [column: Daicel Chiralpak AD-H, 5 μm, 250 mm×20 mm; flow rate: 20 ml/min; detection: 230 nm; injection volume: 0.3 ml; temperature: 25° C.; mobile phase: 45% acetonitrile/50% methanol/5% acetonitrile/glacial acetic acid mixture (95:5), isocratic, run time 10 min]:

### Example 99

### (−)-4-[6-(Hexyloxy)-1-benzofur-3-yl]-4-oxo-2-[2-(4-oxo-1,2,3-benzotriazin-3(4H)-yl)ethyl]butanoic acid (enantiomer 1)

24 mg (chemical purity 99%) of the title compound were obtained.

ee value=100%; [α]_{D}²⁰=−3.8°, 589 nm, c=0.32 g/100 ml, chloroform

¹H-NMR (400 MHz, DMSO-d₆): δ [ppm]=12.37 (br. s, 1H), 8.94 (s, 1H), 8.25 (dd, 1H), 8.20 (d, 1H), 8.12-8.04 (m, 1H), 7.96-7.90 (m, 1H), 7.87 (d, 1H), 7.28 (d, 1H), 6.98 (dd, 1H), 4.59-4.42 (m, 2H), 4.02 (t, 2H), 3.41-3.32 (m, 1H), 3.23-3.15 (m, 1H), 3.02-2.91 (m, 1H), 2.29-2.16 (m, 1H), 2.13-1.99 (m, 1H), 1.78-1.67 (m, 2H), 1.48-1.38 (m, 2H), 1.36-1.26 (m, 4H), 0.91-0.83 (m, 3H).

LC/MS (Method 1, ESIpos): Rₜ=1.33 min, m/z=492 [M+H]⁺.

### Example 100

### (+)-4-[6-(Hexyloxy)-1-benzofur-3-yl]-4-oxo-2-[2-(4-oxo-1,2,3-benzotriazin-3(4H)-yl)ethyl]butanoic acid (enantiomer 2)

22 mg (chemical purity 95%) of the title compound were obtained.

ee value=100%; [α]_{D}²⁰=+2.3°, 589 nm, c=0.33 g/100 ml, chloroform

¹H-NMR (400 MHz, DMSO-d₆): δ [ppm]=12.36 (br. s, 1H), 8.93 (s, 1H), 8.25 (dd, 1H), 8.20 (d, 1H), 8.12-8.06 (m, 1H), 7.96-7.90 (m, 1H), 7.87 (d, 1H), 7.28 (d, 1H), 6.97 (dd, 1H), 4.59-4.43 (m, 2H), 4.02 (t, 2H), 3.40-3.32 (m, 1H), 3.24-3.14 (m, 1H), 3.02-2.92 (m, 1H), 2.28-2.17 (m, 1H), 2.11-1.98 (m, 1H), 1.78-1.66 (m, 2H), 1.49-1.38 (m, 2H), 1.37-1.27 (m, 4H), 0.91-0.84 (m, 3H).

LC/MS (Method 1, ESIpos): Rₜ=1.33 min, m/z=492 [M+H]⁺.

### Example 101

### (+/−)-4-[6-(Heptyloxy)-1-benzofur-3-yl]-4-oxo-2-[2-(4-oxo-1,2,3-benzotriazin-3(4H)-yl)ethyl]butanoic acid

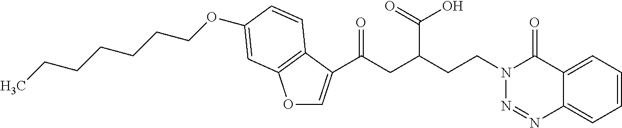 [SMILES: CCCCCCCOc1ccc2c(C(=O)CC(CCn3nnc4ccccc4c3=O)C(=O)O)coc2c1]

A solution of 175 mg (0.26 mmol) of the compound from Example 67A in 3 ml of a 4 N solution of hydrogen chloride in dioxane was stirred firstly for 16 h at RT and then for 2.5 h under reflux. After cooling to RT and concentration, the residue was admixed with dioxane, the solution was again concentrated and the residue was dried in vacuo. Then, the residue was taken up in THF and purified by preparative HPLC (column: Reprosil C18; mobile phase: acetonitrile/water with 0.05% formic acid). 46 mg (34% of theory, purity 99%) of the title compound were obtained.

¹H-NMR (400 MHz, DMSO-d₆): δ [ppm]=12.40 (br. s, 1H), 8.96 (s, 1H), 8.27 (d, 1H), 8.22 (d, 1H), 8.13-8.09 (m, 1H), 7.98-7.92 (m, 1H), 7.89 (d, 1H), 7.30 (d, 1H), 6.99 (dd, 1H), 4.59-4.45 (m, 2H), 4.03 (t, 2H), 3.39 (dd, 1H), 3.22 (dd, 1H), 3.05-2.98 (m, 1H), 2.28-2.20 (m, 1H), 2.10-2.04 (m, 1H), 1.78-1.71 (m, 2H), 1.46-1.30 (m, 8H), 0.88 (t, 3H).

LC/MS (Method 1, ESIpos): Rₜ=1.39 min, m/z=506 [M+H]⁺.

Separation of the Enantiomers:

37 mg of the compound from Example 101 were dissolved in 5 ml of methanol/acetonitrile and separated into the enantiomers by preparative HPLC on a chiral phase (see Examples 102 and 103) [column: Daicel Chiralpak AD-H, 5 μm, 250 mm×20 mm; flow rate: 30 ml/min; detection: 220 nm; injection volume: 0.5 ml; temperature: 20° C.; mobile phase: 70% acetonitrile/30% methanol, isocratic, run time 12 min]:

### Example 102

### (−)-4-[6-(Heptyloxy)-1-benzofur-3-yl]-4-oxo-2-[2-(4-oxo-1,2,3-benzotriazin-3(4H)-yl)ethyl]butanoic acid (enantiomer 1)

16 mg (chemical purity 99%) of the title compound were obtained.

ee value=100%; [α]_{D}²⁰=−7.4°, 589 nm, c=0.30 g/100 ml, chloroform

¹H-NMR (400 MHz, DMSO-d₆): δ [ppm]=12.40 (br. s, 1H), 8.96 (s, 1H), 8.27 (d, 1H), 8.22 (d, 1H), 8.13-8.09 (m, 1H), 7.98-7.92 (m, 1H), 7.89 (d, 1H), 7.30 (d, 1H), 6.99 (dd, 1H), 4.59-4.45 (m, 2H), 4.03 (t, 2H), 3.39 (dd, 1H), 3.22 (dd, 1H), 3.05-2.98 (m, 1H), 2.28-2.20 (m, 1H), 2.10-2.04 (m, 1H), 1.78-1.71 (m, 2H), 1.46-1.30 (m, 8H), 0.88 (t, 3H).

LC/MS (Method 1, ESIpos): Rₜ=1.39 min, m/z=506 [M+H]⁺.

### Example 103

### (+)-4-[6-(Heptyloxy)-1-benzofur-3-yl]-4-oxo-2-[2-(4-oxo-1,2,3-benzotriazin-3(4H)-yl)ethyl]butanoic acid acid (enantiomer 2)

16 mg (chemical purity 99%) of the title compound were obtained.

ee value=100%; [α]_{D}²⁰=+1.7°, 589 nm, c=0.33 g/100 ml, chloroform

¹H-NMR (400 MHz, DMSO-d₆): δ [ppm]=12.40 (br. s, 1H), 8.96 (s, 1H), 8.27 (d, 1H), 8.22 (d, 1H), 8.13-8.09 (m, 1H), 7.98-7.92 (m, 1H), 7.89 (d, 1H), 7.30 (d, 1H), 6.99 (dd, 1H), 4.59-4.45 (m, 2H), 4.03 (t, 2H), 3.39 (dd, 1H), 3.22 (dd, 1H), 3.05-2.98 (m, 1H), 2.28-2.20 (m, 1H), 2.10-2.04 (m, 1H), 1.78-1.71 (m, 2H), 1.46-1.30 (m, 8H), 0.88 (t, 3H).

LC/MS (Method 1, ESIpos): Rₜ=1.39 min, m/z=506 [M+H]⁺.

### Example 104

### (+/−)-4-(6-Isobutoxy-1-benzofur-3-yl)-4-oxo-2-[2-(4-oxo-1,2,3-benzotriazin-3(4H)-yl)ethyl]butanoic acid

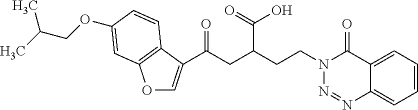 [SMILES: CC(C)COc1ccc2c(C(=O)CC(CCn3nnc4ccccc4c3=O)C(=O)O)coc2c1]

Method A:

A solution of 120 mg (0.19 mmol) of the compound from Example 68A in 2 ml of a 4 N solution of hydrogen chloride in dioxane was stirred firstly for 16 h at RT and then for 3 h under reflux. After cooling to RT and concentration, the residue was admixed with dichloromethane, the solution was again concentrated and the residue was dried in vacuo. 56 mg (60% of theory, purity 97%) of the title compound were obtained.

¹H-NMR (400 MHz, DMSO-d₆): δ [ppm]=12.40 (br. s, 1H), 8.95 (s, 1H), 8.26 (d, 1H), 8.21 (d, 1H), 8.11-8.09 (m, 1H), 7.95-7.92 (m, 1H), 7.88 (d, 1H), 7.29 (d, 1H), 6.69 (dd, 1H), 4.56-4.45 (m, 2H), 3.80 (d, 2H), 3.37 (dd, 1H), 3.21 (dd, 1H), 3.00-2.95 (m, 1H), 2.26-2.19 (m, 1H), 2.08-2.01 (m, 2H), 1.00 (d, 6H).

LC/MS (Method 1, ESIpos): Rₜ=1.18 min, m/z=464 [M+H]⁺.

Method B:

A solution of 277 mg (0.49 mmol, purity 89%) of the compound from Example 147A in 3 ml of dioxane was stirred under reflux for 2 h. After cooling to RT and concentration, 255 mg of the title compound were obtained (>100% of theory, purity approx. 93%, still comprising solvent).

Separation of the Enantiomers:

255 mg of the compound from Example 104 were dissolved in 8 ml of an ethanol/acetonitrile mixture (1:1) and separated into the enantiomers by preparative HPLC on a chiral phase (see Examples 105 and 106) [column: Daicel Chiralpak AD-H, 5 μm, 250 mm×20 mm; flow rate: 15 ml/min; detection: 270 nm; injection volume: 0.3 ml; temperature: 25° C.; mobile phase: 95% ethanol/5% acetonitrile+0.2% acetic acid, isocratic, run time 10 min]:

### Example 105

### (+)-4-(6-Isobutoxy-1-benzofur-3-yl)-4-oxo-2-[2-(4-oxo-1,2,3-benzotriazin-3(4H)-yl)ethyl]butanoic acid (enantiomer 1)

69 mg (chemical purity 100%) of the title compound were obtained.

ee value=100%; [α]²⁰=+82.00, 365 nm, c=0.27 g/100 ml, chloroform

¹H-NMR (400 MHz, DMSO-d₆): δ [ppm]=12.40 (br. s, 1H), 8.95 (s, 1H), 8.25 (d, 1H), 8.21 (d, 1H), 8.11-8.08 (m, 1H), 7.95-7.92 (m, 1H), 7.88 (d, 1H), 7.29 (d, 1H), 6.99 (dd, 1H), 4.56-4.45 (m, 2H), 3.80 (d, 2H), 3.36 (dd, 1H), 3.21 (dd, 1H), 3.00-2.95 (m, 1H), 2.26-2.19 (m, 1H), 2.08-2.01 (m, 2H), 1.00 (d, 6H).

LC/MS (Method 1, ESIpos): Rₜ=1.18 min, m/z=464 [M+H]⁺.

### Example 106

### (−)-4-(6-Isobutoxy-1-benzofur-3-yl)-4-oxo-2-[2-(4-oxo-1,2,3-benzotriazin-3(4H)-yl)ethyl]butanoic acid (enantiomer 2)

78 mg (chemical purity 95%) of the title compound were obtained.

ee value=95%; [α]²⁰=−78.3°, 365 nm, c=0.29 g/100 ml, chloroform

¹H-NMR (400 MHz, DMSO-d₆): δ [ppm]=12.40 (br. s, 1H), 8.95 (s, 1H), 8.25 (d, 1H), 8.21 (d, 1H), 8.11-8.08 (m, 1H), 7.95-7.92 (m, 1H), 7.88 (d, 1H), 7.29 (d, 1H), 6.99 (dd, 1H), 4.56-4.45 (m, 2H), 3.80 (d, 2H), 3.36 (dd, 1H), 3.21 (dd, 1H), 3.00-2.95 (m, 1H), 2.26-2.19 (m, 1H), 2.08-2.01 (m, 2H), 1.00 (d, 6H).

LC/MS (Method 1, ESIpos): Rₜ=1.18 min, m/z=464 [M+H]⁺.

### Example 107

### (+/−)-4-(6-Butoxy-1-benzofur-3-yl)-4-oxo-2-[2-(4-oxo-1,2,3-benzotriazin-3(4H)-yl)ethyl]butanoic acid

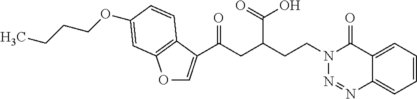 [SMILES: CCCCOc1ccc2c(C(=O)CC(CCn3nnc4ccccc4c3=O)C(=O)O)coc2c1]

A solution of 38 mg (0.06 mmol) of the compound from Example 69A in 1 ml of a 4 N solution of hydrogen chloride in dioxane was stirred firstly for 2 h at RT and then for 5.5 h under reflux. After cooling to RT and concentration, the residue was admixed with dichloromethane, the solution was again concentrated and the residue was dried in vacuo. 29 mg (92% of theory, purity 90%) of the title compound were obtained.

¹H-NMR (400 MHz, DMSO-d₆): δ [ppm]=12.33 (br. s, 1H), 8.94 (s, 1H), 8.25 (d, 1H), 8.20 (d, 1H), 8.09 (t, 1H), 7.93 (t, 1H), 7.88 (d, 1H), 7.29 (d, 1H), 6.98 (dd, 1H), 4.61-4.42 (m, 2H), 4.03 (t, 2H), 3.42-3.32 (m, 1H), 3.20 (dd, 1H), 3.02-2.90 (m, 1H), 2.29-2.16 (m, 1H), 2.11-1.98 (m, 1H), 1.78-1.66 (m, 2H), 1.51-1.37 (m, 2H), 0.94 (t, 3H).

LC/MS (Method 1, ESIpos): Rₜ=1.21 min, m/z=464 [M+H]⁺.

Separation of the Enantiomers:

21 mg of the compound from Example 107 were dissolved in 5 ml of ethanol and separated into the enantiomers by preparative HPLC on a chiral phase (see Examples 108 and 109) [column: Daicel Chiralpak AD-H, 5 μm, 250 mm×20 mm; flow rate: 20 ml/min; detection: 220 nm; injection volume: 0.2 ml; temperature: 20° C.; mobile phase: 70% ethanol/30% acetonitrile, isocratic, run time 10 min]:

### Example 108

### (+)-4-(6-Butoxy-1-benzofur-3-yl)-4-oxo-2-[2-(4-oxo-1,2,3-benzotriazin-3(4H)-yl)ethyl]butanoic acid (enantiomer 1)

5 mg (chemical purity 95%) of the title compound were obtained.

ee value=100%; [α]²⁰=+46.20, 365 nm, c=0.15 g/100 ml, chloroform

¹H-NMR (500 MHz, DMSO-d₆): δ [ppm]=12.35 (br. s, 1H), 8.95 (s, 1H), 8.25 (dd, 1H), 8.21 (d, 1H), 8.09 (td, 1H), 7.96-7.92 (m, 1H), 7.88 (d, 1H), 7.29 (d, 1H), 6.98 (dd, 1H), 4.60-4.41 (m, 2H), 4.02 (t, 2H), 3.40-3.34 (m, 1H), 3.21 (dd, 1H), 3.02-2.93 (m, 1H), 2.27-2.17 (m, 1H), 2.09-1.99 (m, 1H), 1.76-1.67 (m, 2H), 1.50-1.40 (m, 2H), 0.94 (t, 3H).

LC/MS (Method 1, ESIpos): Rₜ=1.17 min, m/z=464 [M+H]⁺.

### Example 109

### (−)-4-(6-Butoxy-1-benzofur-3-yl)-4-oxo-2-[2-(4-oxo-1,2,3-benzotriazin-3(4H)-yl)ethyl]butanoic acid (enantiomer 2)

4 mg (chemical purity 100%) of the title compound were obtained.

ee value=94%; [α]²⁰=−40.5°, 365 nm, c=0.35 g/100 ml, chloroform

¹H-NMR (500 MHz, DMSO-d₆): δ [ppm]=12.39 (br. s, 1H), 8.95 (s, 1H), 8.25 (d, 1H), 8.21 (d, 1H), 8.12-8.07 (m, 1H), 7.96-7.91 (m, 1H), 7.88 (d, 1H), 7.29 (d, 1H), 6.98 (dd, 1H), 4.58-4.42 (m, 2H), 4.02 (t, 2H), 3.41-3.33 (m, 1H), 3.25-3.17 (m, 1H), 3.02-2.92 (m, 1H), 2.29-2.16 (m, 1H), 2.10-1.98 (m, 1H), 1.77-1.66 (m, 2H), 1.51-1.40 (m, 2H), 0.94 (t, 3H).

LC/MS (Method 1, ESIpos): Rₜ=1.21 min, m/z=464 [M+H]⁺.

### Example 110

### (+/−)-4-Oxo-2-[2-(4-oxo-1,2,3-benzotriazin-3(4H)-yl)ethyl]-4-[6-(2-phenylethoxy)-1-benzofur-3-yl]butanoic acid

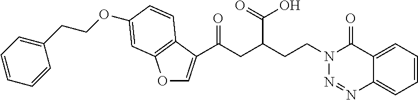 [SMILES: O=C(CC(CCn1nnc2ccccc2c1=O)C(=O)O)c1coc2cc(OCCc3ccccc3)ccc12]

A solution of 39 mg (0.06 mmol) of the compound from Example 70A in 1 ml of a 4 N solution of hydrogen chloride in dioxane was stirred firstly for 16 h at RT and then for 5 h under reflux. After cooling to RT and concentration, the residue was admixed with dichloromethane, the solution was again concentrated and the residue was dried in vacuo. Then, the residue was purified by preparative HPLC (column: Reprosil C18; mobile phase: acetonitrile/water gradient with 0.05% formic acid). 11 mg (35% of theory, purity 95%) of the title compound were obtained.

¹H-NMR (400 MHz, DMSO-d₆): δ [ppm]=12.39 (br. s, 1H), 8.95 (s, 1H), 8.25 (dd, 1H), 8.21 (d, 1H), 8.09 (td, 1H), 7.96-7.90 (m, 1H), 7.87 (d, 1H), 7.37-7.28 (m, 5H), 7.26-7.19 (m, 1H), 6.98 (dd, 1H), 4.59-4.41 (m, 2H), 4.25 (t, 2H), 3.43-3.35 (m, 1H), 3.20 (dd, 1H), 3.07 (t, 2H), 3.01-2.93 (m, 1H), 2.28-2.16 (m, 1H), 2.11-1.97 (m, 1H).

LC/MS (Method 1, ESIpos): Rₜ=1.20 min, m/z=512 [M+H]⁺.

### Example 111

### (+/−)-4-[6-(Cyclopropylmethoxy)-1-benzothiophen-2-yl]-4-oxo-2-[2-(4-oxo-1,2,3-benzotriazin-3(4H)-yl)ethyl]butanoic acid

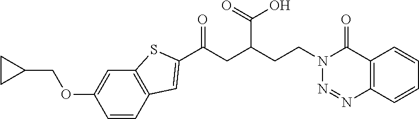 [SMILES: O=C(CC(CCn1nnc2ccccc2c1=O)C(=O)O)c1cc2ccc(OCC3CC3)cc2s1]

A solution of 507 mg (0.80 mmol) of the compound from Example 85A in 10 ml of dioxane was admixed with 10 ml of a 4 N solution of hydrogen chloride in dioxane. The mixture was heated in a microwave apparatus firstly for 10 min at 113° C., then for 10 min at 150° C. After cooling to RT and concentration, the residue was purified by means of preparative HPLC (column: Reprosil C18; mobile phase: acetonitrile/water gradient with 0.2% trifluoroacetic acid in the water). 222 mg (58% of theory, purity 99%) of the title compound were obtained.

¹H-NMR (400 MHz, DMSO-d₆): δ [ppm]=12.41 (br. s, 1H), 8.32 (s, 1H), 8.26 (dd, 1H), 8.20 (d, 1H), 8.09 (td, 1H), 7.96-7.91 (m, 1H), 7.90 (d, 1H), 7.56 (d, 1H), 7.09 (dd, 1H), 4.59-4.43 (m, 2H), 3.92 (d, 2H), 3.56-3.47 (m, 1H), 3.42-3.26 (m, 1H), 3.02-2.90 (m, 1H), 2.30-2.17 (m, 1H), 2.14-2.01 (m, 1H), 1.33-1.20 (m, 1H), 0.63-0.54 (m, 2H), 0.40-0.30 (m, 2H).

LC/MS (Method 1, ESIpos): Rₜ=1.12 min, m/z=478 [M+H]⁺.

Separation of the Enantiomers:

222 mg of the compound from Example 111 were dissolved in 4 ml of acetonitrile/methanol (7:3) and separated into the enantiomers by preparative HPLC on a chiral phase (see Examples 112 and 113) [column: Daicel Chiralpak AD-H, 5 μm, 250 mm×20 mm; flow rate: 20 ml/min; detection: 220 nm; injection volume: 0.5 ml; temperature: 20° C.; mobile phase: 70% acetonitrile+0.2% acetic acid/30% methanol, isocratic, run time 16 min]:

### Example 112

### (+)-4-[6-(Cyclopropylmethoxy)-1-benzothiophen-2-yl]-4-oxo-2-[2-(4-oxo-1,2,3-benzotriazin-3(4H)-yl)ethyl]butanoic acid (enantiomer 1)

52 mg (chemical purity 100%) of the title compound were obtained.

ee value=100%; [α]_{D}²⁰=+33.6°, 589 nm, c=0.31 g/100 ml, chloroform

¹H-NMR (400 MHz, DMSO-d₆): δ [ppm]=12.41 (br. s, 1H), 8.32 (s, 1H), 8.26 (d, 1H), 8.20 (d, 1H), 8.12-8.05 (m, 1H), 7.97-7.86 (m, 2H), 7.56 (d, 1H), 7.09 (dd, 1H), 4.59-4.43 (m, 2H), 3.92 (d, 2H), 3.51 (dd, 1H), 3.32-3.27 (m, 1H), 3.02-2.89 (m, 1H), 2.30-2.17 (m, 1H), 2.13-2.01 (m, 1H), 1.34-1.20 (m, 1H), 0.64-0.53 (m, 2H), 0.38-0.30 (m, 2H).

LC/MS (Method 1, ESIpos): Rₜ=1.12 min, m/z=478 [M+H]⁺.

### Example 113

### (−)-4-[6-(Cyclopropylmethoxy)-1-benzothiophen-2-yl]-4-oxo-2-[2-(4-oxo-1,2,3-benzotriazin-3(4H)-yl)ethyl]butanoic acid (enantiomer 2)

52 mg (chemical purity 100%) of the title compound were obtained.

ee value=100%; [α]_{D}²⁰=−38.0°, 589 nm, c=0.26 g/100 ml, chloroform

¹H-NMR (400 MHz, DMSO-d₆): δ [ppm]=12.40 (br. s, 1H), 8.32 (s, 1H), 8.26 (dd, 1H), 8.20 (d, 1H), 8.09 (td, 1H), 7.96-7.86 (m, 2H), 7.56 (d, 1H), 7.09 (dd, 1H), 4.60-4.42 (m, 2H), 3.92 (d, 2H), 3.51 (dd, 1H), 3.31-3.26 (m, 1H), 3.01-2.89 (m, 1H), 2.30-2.16 (m, 1H), 2.13-2.00 (m, 1H), 1.34-1.21 (m, 1H), 0.63-0.55 (m, 2H), 0.38-0.31 (m, 2H).

LC/MS (Method 1, ESIpos): Rₜ=1.12 min, m/z=478 [M+H]⁺.

### Example 114

### (+/−)-4-Oxo-2-[2-(4-oxo-1,2,3-benzotriazin-3(4H)-yl)ethyl]-4-[6-(2-phenylethoxy)-1-benzothiophen-2-yl]butanoic acid

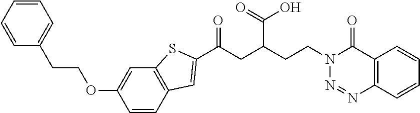 [SMILES: O=C(CC(CCn1nnc2ccccc2c1=O)C(=O)O)c1cc2ccc(OCCc3ccccc3)cc2s1]

A solution of 71 mg (0.10 mmol) of the compound from Example 86A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 30 mg (50% of theory, purity 92%) of the title compound were obtained.

¹H-NMR (400 MHz, DMSO-d₆): δ [ppm]=12.41 (br. s, 1H), 8.32 (s, 1H), 8.26 (d, 1H), 8.20 (d, 1H), 8.09 (dd, 1H), 7.94 (d, 1H), 7.90 (d, 1H), 7.64 (d, 1H), 7.37-7.30 (m, 4H), 7.23 (dd, 1H), 7.07 (dd, 1H), 4.58-4.45 (m, 2H), 4.30 (dd, 2H), 3.51 (dd, 1H), 3.31 (dd, 1H), 3.09 (dd, 2H), 2.99-2.93 (m, 1H), 2.28-2.19 (m, 1H), 2.12-2.03 (m, 1H).

LC/MS (Method 1, ESIpos): Rₜ=1.23 min, m/z=528 [M+H]⁺.

### Example 115

### (+/−)-4-[6-(2-Cyclopropyl-2-oxoethoxy)-1-benzothiophen-2-yl]-4-oxo-2-[2-(4-oxo-1,2,3-benzotriazin-3(4H)-yl)ethyl]butanoic acid

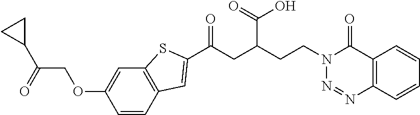 [SMILES: O=C(CC(CCn1nnc2ccccc2c1=O)C(=O)O)c1cc2ccc(OCC(=O)C3CC3)cc2s1]

A solution of 125 mg (0.19 mmol) of the compound from Example 87A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 37 mg (38% of theory, purity 100%) of the title compound were obtained.

¹H-NMR (400 MHz, DMSO-d₆): δ [ppm]=12.41 (br. s, 1H), 8.34 (s, 1H), 8.26 (d, 1H), 8.20 (d, 1H), 8.09 (dd, 1H), 7.94 (d, 1H), 7.92 (d, 1H), 7.54 (d, 1H), 7.12 (dd, 1H), 4.58-4.45 (m, 2H), 3.32 (dd, 1H), 3.13-3.07 (m, 1H), 3.01-2.93 (m, 1H), 2.28-2.18 (m, 2H), 2.12-2.04 (m, 1H), 1.18 (t, 1H), 1.01-0.92 (m, 5H).

LC/MS (Method 1, ESIpos): Rₜ=1.00 min, m/z=506 [M+H]⁺.

### Example 116

### (+/−)-4-Oxo-2-[2-(4-oxo-1,2,3-benzotriazin-3(4H)-yl)ethyl]-4-(6-propoxy-1-benzothiophen-2-yl)butanoic acid

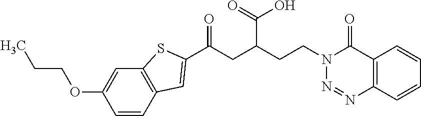 [SMILES: CCCOc1ccc2cc(C(=O)CC(CCn3nnc4ccccc4c3=O)C(=O)O)sc2c1]

A solution of 43 mg (0.07 mmol) of the compound from Example 88A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 14 mg (39% of theory, purity 88%) of the title compound were obtained.

¹H-NMR (400 MHz, DMSO-d₆): δ [ppm]=12.39 (br. s, 1H), 8.34-8.30 (m, 1H), 8.26 (d, 1H), 8.20 (d, 1H), 8.09 (t, 1H), 7.97-7.85 (m, 2H), 7.59 (d, 1H), 7.08 (dd, 1H), 4.60-4.43 (m, 2H), 4.03 (t, 2H), 3.56-3.45 (m, 1H), 3.42-3.32 (m, 1H), 3.03-2.91 (m, 1H), 2.30-2.18 (m, 1H), 2.15-2.02 (m, 1H), 1.83-1.69 (m, 2H), 1.00 (t, 3H).

LC/MS (Method 1, ESIpos): Rₜ=1.15 min, m/z=466 [M+H]⁺.

### Example 117

### (+/−)-4-Oxo-2-[2-(4-oxo-1,2,3-benzotriazin-3(4H)-yl)ethyl]-4-{6-[tetrahydrofuran-2-ylmethoxy]-1-benzothiophen-2-yl}butanoic acid

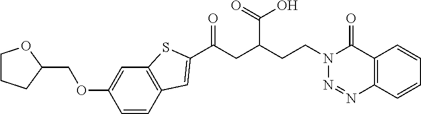 [SMILES: O=C(CC(CCn1nnc2ccccc2c1=O)C(=O)O)c1cc2ccc(OCC3CCCO3)cc2s1]

A solution of 111 mg (0.17 mmol) of the compound from Example 89A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 24 mg (28% of theory, purity 98%) of the title compound were obtained.

¹H-NMR (400 MHz, DMSO-d₆): δ [ppm]=12.38 (br. s, 1H), 8.33 (s, 1H), 8.26 (dd, 1H), 8.20 (d, 1H), 8.09 (td, 1H), 7.96-7.88 (m, 2H), 7.61 (d, 1H), 7.10 (dd, 1H), 4.62-4.39 (m, 2H), 4.24-4.15 (m, 1H), 4.12-3.97 (m, 2H), 3.85-3.75 (m, 1H), 3.74-3.64 (m, 1H), 3.56-3.46 (m, 1H), 3.32-3.27 (m, 1H), 3.02-2.89 (m, 1H), 2.30-2.17 (m, 1H), 2.14-1.97 (m, 2H), 1.96-1.78 (m, 2H), 1.77-1.61 (m, 1H).

LC/MS (Method 1, ESIpos): Rₜ=1.04 min, m/z=508 [M+H]⁺.

### Example 118

### (+/−)-4-Oxo-2-[2-(4-oxo-1,2,3-benzotriazin-3(4H)-yl)ethyl]-4-[6-(pentyloxy)-1-benzothiophen-2-yl]butanoic acid

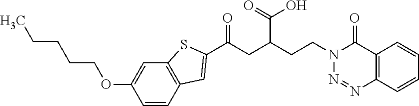 [SMILES: CCCCCOc1ccc2cc(C(=O)CC(CCn3nnc4ccccc4c3=O)C(=O)O)sc2c1]

A solution of 56 mg (0.09 mmol) of the compound from Example 90A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 15 mg (34% of theory, purity 96%) of the title compound were obtained.

¹H-NMR (400 MHz, DMSO-d₆): δ [ppm]=12.42 (br. s, 1H), 8.33 (s, 1H), 8.26 (d, 1H), 8.20 (d, 1H), 8.09 (dd, 1H), 7.94 (d, 1H), 7.90 (d, 1H), 7.60 (d, 1H), 7.09 (dd, 1H), 4.58-4.45 (m, 2H), 4.06 (dd, 2H), 3.51 (dd, 1H), 3.00-2.93 (m, 1H), 2.28-2.19 (m, 1H), 2.12-2.01 (m, 2H), 1.79-1.72 (m, 2H), 1.45-1.33 (m, 4H), 0.91 (t, 3H).

LC/MS (Method 1, ESIpos): Rₜ=1.29 min, m/z=494 [M+H]⁺.

### Example 119

### (+/−)-4-{6-[Oxetan-2-ylmethoxy]-1-benzothiophen-2-yl}-4-oxo-2-[2-(4-oxo-1-benzotriazin-3(4H)-yl)ethyl]butanoic acid

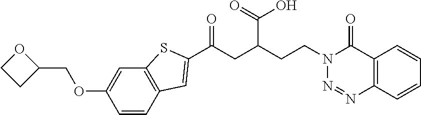 [SMILES: O=C(CC(CCn1nnc2ccccc2c1=O)C(=O)O)c1cc2ccc(OCC3CCO3)cc2s1]

18.1 mg (0.12 mmol) of 2-(bromomethyl)oxetane were initially introduced onto a metal mutititre plate and admixed with 58 mg (0.10 mmol) of the compound from Example 84A, dissolved in 0.6 ml of DMF. Then, 69 mg (0.50 mmol) of potassium carbonate were added, and the mixture was shaken for 18 h at 70° C. After cooling to RT, the mixture was filtered and the filtrate was concentrated. The residue was taken up in 0.4 ml of dioxane and transferred to a plastic multititer plate. After adding 0.4 ml of a 4 N solution of hydrogen chloride in dioxane, the mixture was heated in a microwave apparatus for 7 min at 150° C. Then, a further 0.15 ml of the 4 N solution of hydrogen chloride in dioxane were added, and the mixture was heated once again for 10 min in the microwave apparatus at 150° C. After cooling to RT, the mixture was concentrated, and the residue was taken up in 0.6 ml of DMF and purified by preparative HPLC (Method 10). 5 mg (10% of theory, purity 95%) of the title compound were obtained.

LC/MS (Method 4): Rₜ=1.08 min; MS (ESIpos): m/z=494 [M+H]⁺.

### Example 120

### (+/−)-4-Oxo-2-[2-(4-oxo-1,2,3-benzotriazin-3(4H)-yl)ethyl]-4-[6-(4,4,4-trifluorobutoxy)-1-benzothiophen-2-yl]butanoic acid

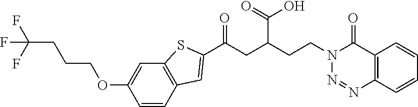 [SMILES: O=C(CC(CCn1nnc2ccccc2c1=O)C(=O)O)c1cc2ccc(OCCCC(F)(F)F)cc2s1]

Analogously to the method described in Example 119, 23 mg (0.12 mmol) of 4-bromo-1,1,1triflurorobutane were reacted with 58 mg (0.10 mmol) of the compound from Example 84A. Purification of the crude product by preparative HPLC [Method 10] gave 5 mg (10% of theory, purity 100%) of the title compound.

LC/MS (Method 4): Rₜ=1.21 min; MS (ESIpos): m/z=534 [M+H]⁺.

### Example 121

### (+/−)-4-[6-(Cyclobutylmethoxy)-1-benzothiophen-2-yl]-4-oxo-2-[2-(4-oxo-1,2,3-benzotriazin-3(4H)-yl)ethyl]butanoic acid

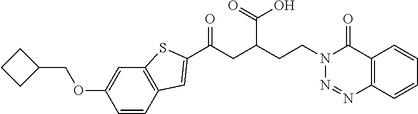 [SMILES: O=C(CC(CCn1nnc2ccccc2c1=O)C(=O)O)c1cc2ccc(OCC3CCC3)cc2s1]

Analogously to the method described in Example 119, 18 mg (0.12 mmol) of (bromomethyl)cyclobutane were reacted with 58 mg (0.10 mmol) of the compound from Example 84A. Purification of the crude product by preparative HPLC [Method 10] gave 13 mg (26% of theory, purity 100%) of the title compound.

LC/MS (Method 4): Rₜ=1.23 min; MS (ESIpos): m/z=492 [M+H]⁺.

### Example 122

### (+/−)-4-[6-(3-Methylbutoxy)-1-benzothiophen-2-yl]-4-oxo-2-[2-(4-oxo-1,2,3-benzotriazin-3(4H)-yl)ethyl]butanoic acid

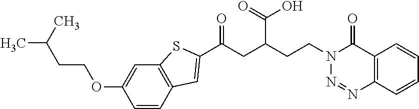 [SMILES: CC(C)CCOc1ccc2cc(C(=O)CC(CCn3nnc4ccccc4c3=O)C(=O)O)sc2c1]

Analogously to the method described in Example 119, 18 mg (0.12 mmol) of 1-bromo-3-methylbutane were reacted with 58 mg (0.10 mmol) of the compound from Example 84A. Purification of the crude product by preparative HPLC [Method 10] gave 15 mg (29% of theory, purity 97%) of the title compound.

LC/MS (Method 4): Rₜ=1.25 min; MS (ESIpos): m/z=494 [M+H]⁺.

### Example 123

### (+/−)-4-Oxo-2-[2-(4-oxo-1,2,3-benzotriazin-3(4H)-yl)ethyl]-4-[6-(pyridin-4-ylmethoxy)-1-benzothiophen-2-yl]butanoic acid

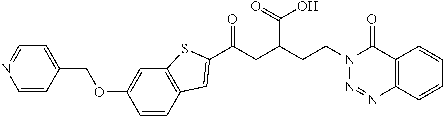 [SMILES: O=C(CC(CCn1nnc2ccccc2c1=O)C(=O)O)c1cc2ccc(OCc3ccncc3)cc2s1]

Analogously to the method described in Example 119, 21 mg (0.12 mmol) of 4-(bromomethyl)pyridine were reacted with 58 mg (0.10 mmol) of the compound from Example 84A. Purification of the crude product by preparative HPLC [Method 10] gave 1 mg (1.8% of theory, purity 92%) of the title compound.

LC/MS (Method 4): Rₜ=0.97 min; MS (ESIpos): m/z=515 [M+H]⁺.

### Example 124

### (+/−)-4-{6-[2-(3-Methoxyphenyl)-2-oxoethoxy]-1-benzothiophen-2-yl}-4-oxo-2-[2-(4-oxo-1,2,3-benzotriazin-3(4H)-yl)ethyl]butanoic acid

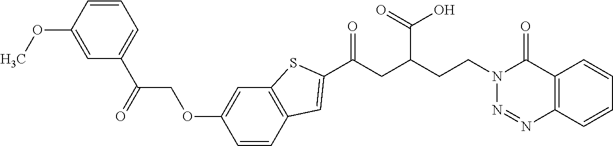 [SMILES: COc1cccc(C(=O)COc2ccc3cc(C(=O)CC(CCn4nnc5ccccc5c4=O)C(=O)O)sc3c2)c1]

Analogously to the method described in Example 119, 27 mg (0.12 mmol) of 2-bromo-1-(3-methoxyphenyl)ethanone were reacted with 58 mg (0.10 mmol) of the compound from Example 84A. Purification of the crude product by preparative HPLC [Method 10] gave 5 mg (9% of theory, purity 94%) of the title compound.

LC/MS (Method 4): Rₜ=1.16 min; MS (ESIpos): m/z=572 [M+H]⁺.

### Example 125

### (+/−)-4-{6-[(4-Fluorobenzyl)oxy]-1-benzothiophen-2-yl}-4-oxo-2-[2-(4-oxo-1,2,3-benzotriazin-3(4H)-yl)ethyl]butanoic acid

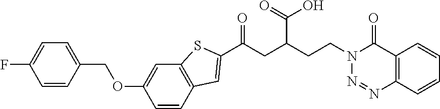 [SMILES: O=C(CC(CCn1nnc2ccccc2c1=O)C(=O)O)c1cc2ccc(OCc3ccc(F)cc3)cc2s1]

Analogously to the method described in Example 119, 23 mg (0.12 mmol) of 1-(bromomethyl)-4-fluorobenzene were reacted with 58 mg (0.10 mmol) of the compound from Example 84A. Purification of the crude product by preparative HPLC [Method 10] gave 4 mg (8% of theory, purity 100%) of the title compound.

LC/MS (Method 4): Rₜ=1.21 min; MS (ESIpos): m/z=532 [M+H]⁺.

### Example 126

### (+/−)-4-(6-Butoxy-1-benzothiophen-2-yl)-4-oxo-2-[2-(4-oxo-1,2,3-benzotriazin-3(4H)-yl)ethyl]butanoic acid

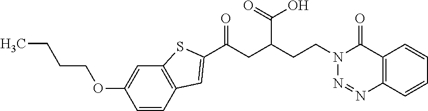 [SMILES: CCCCOc1ccc2cc(C(=O)CC(CCn3nnc4ccccc4c3=O)C(=O)O)sc2c1]

Analogously to the method described in Example 119, 16 mg (0.12 mmol) of 1-bromobutane were reacted with 58 mg (0.10 mmol) of the compound from Example 84A. Purification of the crude product by preparative HPLC [Method 10] gave 9 mg (18% of theory, purity 100%) of the title compound.

LC/MS (Method 4): Rₜ=1.22 min; MS (ESIpos): m/z=480 [M+H]⁺.

### Example 127

### (+/−)-4-(6-Ethoxy-1-benzothiophen-2-yl)-4-oxo-2-[2-(4-oxo-1,2,3-benzotriazin-3(4H)-yl)ethyl]butanoic acid

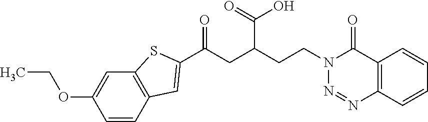 [SMILES: CCOc1ccc2cc(C(=O)CC(CCn3nnc4ccccc4c3=O)C(=O)O)sc2c1]

Analogously to the method described in Example 119, 13 mg (0.12 mmol) of bromoethane were reacted with 58 mg (0.10 mmol) of the compound from Example 84A. Purification of the crude product by preparative HPLC [Method 10] gave 6 mg (13% of theory, purity 97%) of the title compound.

LC/MS (Method 4): Rₜ=1.13 min; MS (ESIpos): m/z=452 [M+H]⁺.

### Example 128

### (+/−)-4-[6-(Cyclopropylmethoxy)-1-benzothiophen-2-yl]-4-oxo-2-{2-[4-oxo-6-(trifluoromethyl)-1,2,3-benzotriazin-3(4H)-yl]ethyl}butanoic acid

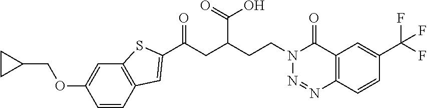 [SMILES: O=C(CC(CCn1nnc2ccc(C(F)(F)F)cc2c1=O)C(=O)O)c1cc2ccc(OCC3CC3)cc2s1]

A solution of 471 mg (0.54 mmol, purity 81%) of the compound from Example 91A in 10 ml of dioxane was admixed with 10 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 138 mg (47% of theory, purity 100%) of the title compound were obtained.

¹H-NMR (400 MHz, DMSO-d₆): δ [ppm]=12.41 (br. s, 1H), 8.49 (s, 1H), 8.45-8.35 (m, 2H), 8.32 (s, 1H), 7.89 (d, 1H), 7.56 (d, 1H), 7.09 (dd, 1H), 4.62-4.46 (m, 2H), 3.92 (d, 2H), 3.51 (dd, 1H), 3.33-3.26 (m, 1H), 3.03-2.92 (m, 1H), 2.30-2.19 (m, 1H), 2.15-2.00 (m, 1H), 1.32-1.19 (m, 1H), 0.63-0.55 (m, 2H), 0.39-0.30 (m, 2H).

LC/MS (Method 1, ESIpos): Rₜ=1.23 min, m/z=546 [M+H]⁺.

Separation of the Enantiomers:

128 mg of the compound from Example 128 were dissolved in 2.5 ml of acetonitrile, admixed with 2 ml of ethanol and then separated into the enantiomers by preparative HPLC on a chiral phase (see Examples 129 and 130) [column: Daicel Chiralpak AD-H, 5 μm, 250 mm×20 mm; flow rate: 20 ml/min; detection: 320 nm; injection volume: 0.4 ml; temperature: 25° C.; mobile phase: 50% ethanol/50% acetonitrile+0.2% acetic acid, isocratic, run time 28 min]:

### Example 129

### 4-[6-(Cyclopropylmethoxy)-1-benzothiophen-2-yl]-4-oxo-2-{2-[4-oxo-6-(trifluoromethyl)-1,2,3-benzotriazin-3(4H)-yl]ethyl}butanoic acid (enantiomer 1)

34 mg (chemical purity 96%) of the title compound were obtained.

Rₜ=8.39 min, ee value=100% [column: Daicel Chiralpak AD-H, 250 mm×4.6 mm, 5 μm; flow rate: 1.0 ml/min; detection: 270 nm; temperature: 40° C.; mobile phase: 50% ethanol+0.2% glacial acetic acid/50% acetonitrile+0.2% glacial acetic acid, isocratic].

¹H-NMR (400 MHz, DMSO-d₆): δ [ppm]=12.33 (br. s, 1H), 8.48 (s, 1H), 8.44-8.35 (m, 2H), 8.31 (s, 1H), 7.89 (d, 1H), 7.55 (d, 1H), 7.09 (dd, 1H), 4.62-4.46 (m, 2H), 3.92 (d, 2H), 3.51 (dd, 1H), 3.32-3.27 (m, 1H), 3.04-2.93 (m, 1H), 2.32-2.19 (m, 1H), 2.17-2.03 (m, 1H), 1.32-1.20 (m, 2H), 0.63-0.55 (m, 2H), 0.39-0.31 (m, 2H).

LC/MS (Method 1, ESIpos): Rₜ=1.23 min, m/z=546 [M+H]⁺.

### Example 130

### 4-[6-(Cyclopropylmethoxy)-1-benzothiophen-2-yl]-4-oxo-2-{2-[4-oxo-6-(trifluoromethyl)-1,2,3-benzotriazin-3(4H)-yl]ethyl}butanoic acid (enantiomer 2)

29 mg (chemical purity 100%) of the title compound were obtained.

Rₜ=16.40 min, ee value=97% [column: Daicel Chiralpak AD-H, 250 mm×4.6 mm, 5 μm; flow rate: 1.0 ml/min; detection: 270 nm; temperature: 40° C.; mobile phase: 50% ethanol+0.2% glacial acetic acid/50% acetonitrile+0.2% glacial acetic acid, isocratic].

¹H-NMR (400 MHz, DMSO-d₆): δ [ppm]=12.36 (br. s, 1H), 8.48 (s, 1H), 8.44-8.35 (m, 2H), 8.32-8.27 (m, 1H), 7.89 (d, 1H), 7.55 (d, 1H), 7.09 (dd, 1H), 4.63-4.44 (m, 2H), 3.92 (d, 2H), 3.56-3.43 (m, 2H), 3.30-3.24 (m, 1H), 3.02-2.91 (m, 1H), 2.31-2.20 (m, 1H), 2.15-2.02 (m, 1H), 1.33-1.20 (m, 2H), 0.63-0.54 (m, 2H), 0.39-0.30 (m, 2H).

LC/MS (Method 1, ESIpos): Rₜ=1.23 min, m/z=546 [M+H]⁺.

### Example 131

### (+/−)-4-(6-Methoxy-1-benzothiophen-2-yl)-4-oxo-2-{2-[4-oxo-6-(trifluoromethyl)-1,2,3-benzotriazin-3(4H)-yl]ethyl}butanoic acid

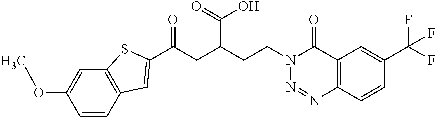 [SMILES: COc1ccc2cc(C(=O)CC(CCn3nnc4ccc(C(F)(F)F)cc4c3=O)C(=O)O)sc2c1]

A solution of 93 mg (0.13 mmol, purity 95%) of the compound from Example 91A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 36 mg (53% of theory, purity 100%) of the title compound were obtained.

¹H-NMR (400 MHz, DMSO-d₆): δ [ppm]=12.42 (br. s, 1H), 8.49 (br. s, 1H), 8.43-8.37 (m, 2H), 8.33 (br. s, 1H), 7.90 (d, 1H), 7.60 (d, 1H), 7.09 (dd, 1H), 4.61-4.49 (m, 2H), 3.86 (s, 3H), 3.51 (dd, 1H), 3.30 (d, 1H), 3.02-2.95 (m, 1H), 2.30-2.21 (m, 1H), 2.14-2.06 (m, 1H).

LC/MS (Method 1, ESIpos): Rₜ=1.12 min, m/z=506 [M+H]⁺.

### Example 132

### (+/−)-4-Oxo-2-{2-[4-oxo-6-(trifluoromethyl)-1,2,3-benzotriazin-3(4H)-yl]ethyl}-4-(6-propoxy-1-benzothiophen-2-yl)butanoic acid

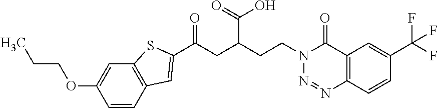 [SMILES: CCCOc1ccc2cc(C(=O)CC(CCn3nnc4ccc(C(F)(F)F)cc4c3=O)C(=O)O)sc2c1]

A solution of 104 mg (0.12 mmol, purity 76%) of the compound from Example 91A in 2 ml of dioxane was admixed with 2 ml of a 4 N solution of hydrogen chloride in dioxane. The mixture was heated at 150° C. for 10 min in a microwave apparatus. After cooling to RT and concentration, the residue was purified by means of preparative HPLC (column: Reprosil C18; mobile phase: acetonitrile/water gradient with 0.2% trifluoroacetic acid in the water). 35 mg (56% of theory, purity 100%) of the title compound were obtained.

¹H-NMR (400 MHz, DMSO-d₆): δ [ppm]=12.44 (br. s, 1H), 8.49 (s, 1H), 8.45-8.35 (m, 2H), 8.32 (s, 1H), 7.89 (d, 1H), 7.59 (d, 1H), 7.08 (dd, 1H), 4.62-4.46 (m, 2H), 4.03 (t, 2H), 3.51 (dd, 1H), 3.33-3.26 (m, 1H), 3.05-2.92 (m, 1H), 2.31-2.20 (m, 1H), 2.15-2.04 (m, 1H), 1.83-1.70 (m, 2H), 1.00 (t, 3H).

LC/MS (Method 1, ESIpos): Rₜ=1.24 min, m/z=534 [M+H]⁺.

### Example 133

### (+/−)-4-Oxo-2-[2-(4-oxo-1,2,3-benzotriazin-3(4H)-yl)ethyl]-4-[6-(tetrahydro-2H-pyran-4-ylmethoxy)-1,2-benzoxazol-3-yl]butanoic acid

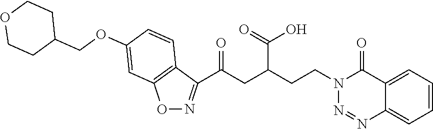 [SMILES: O=C(CC(CCn1nnc2ccccc2c1=O)C(=O)O)c1noc2cc(OCC3CCOCC3)ccc12]

A solution of 356 mg (0.65 mmol) of the compound from Example 118A in 10 ml of dioxane was stirred under reflux for 10 h. After cooling to RT and concentration, the residue was purified by means of preparative HPLC (Method 6). 243 mg (74% of theory, purity 100%) of the title compound were obtained.

¹H-NMR (400 MHz, DMSO-d₆): δ [ppm]=12.53 (br. s, 1H), 8.26 (dd, 1H), 8.21 (d, 1H), 8.09 (td, 1H), 7.96-7.90 (m, 2H), 7.47 (d, 1H), 7.12 (dd, 1H), 4.60-4.43 (m, 2H), 3.98 (d, 2H), 3.89 (dd, 2H), 3.61 (dd, 1H), 3.45-3.29 (m, 3H), 3.12-3.00 (m, 1H), 2.34-2.21 (m, 1H), 2.20-1.98 (m, 2H), 1.74-1.64 (m, 2H), 1.43-1.27 (m, 2H).

LC/MS (Method 1, ESIpos): Rₜ=1.05 min, m/z=507 [M+H]⁺.

Separation of the Enantiomers:

233 mg of the compound from Example 133 were dissolved in 10 ml of DMSO and separated into the enantiomers by preparative HPLC on a chiral phase (see Examples 134 and 135) [column: Daicel Chiralpak IB, 5 μm 250 mm×20 mm; flow rate: 20 ml/min; detection: 285 nm; injection volume: 0.08 ml; temperature: 25° C.; mobile phase: 5% acetonitrile/95% ethanol+0.2% acetic acid, isocratic, run time 3.5 min]:

### Example 134

### (2R)-4-Oxo-2-[2-(4-oxo-1,2,3-benzotriazin-3(4H)-yl)ethyl]-4-[6-(tetrahydro-2H-pyran-4-ylmethoxy)-1,2-benzoxazol-3-yl]butanoic acid (enantiomer 1)

68 mg (chemical purity 100%) of the title compound were obtained.

ee value=100%; [α]_{D}²⁰=−2.3°, 589 nm, c=0.34 g/100 ml, chloroform

¹H-NMR (400 MHz, DMSO-d₆): δ [ppm]=12.54 (br. s, 1H), 8.26 (d, 1H), 8.21 (d, 1H), 8.14-8.05 (m, 1H), 7.98-7.88 (m, 2H), 7.47 (d, 1H), 7.11 (dd, 1H), 4.60-4.43 (m, 2H), 3.98 (d, 2H), 3.89 (dd, 2H), 3.61 (dd, 1H), 3.45-3.27 (m, 3H), 3.11-3.00 (m, 1H), 2.34-2.21 (m, 1H), 2.20-1.97 (m, 2H), 1.70 (d, 2H), 1.36 (qd, 2H).

LC/MS (Method 1, ESIpos): Rₜ=1.05 min, m/z=507 [M+H]⁺.

Single-crystal X-ray analysis carried out with this enantiomer revealed an R configuration for the chiral carbon atom of the compound. The crystal data obtained is given in the following table:

Crystal Data from X-Ray Structural Analysis in Respect of Example 134:

| Space group | P2(1) 2(1) 2(1) |
|---|---|
| Cell parameters: | |
| a (Å) | 9.44853(14) |
| b (Å) | 9.95248(13) |
| c (Å) | 25.1782(4) |
| α (°) | 90 |
| β (°) | 90 |
| γ (°) | 90 |
| Volume (Å³) | 2367.66(6) |
| Molecules per unit cell | 4 |
| calculated density (Mg/m³) | 1.421 |

[Single crystal obtained by crystallization from acetonitrile/water (1:1) at RT; diffractometer: Oxford Diffraction Xcalibur series, CCD area detector (model Ruby); radiation: CuKα radiation 1.54178 Å; temperature 108 K; resolution: 0.89 Å; θ range: 3.511-65.735°; scan type: fullsphere data collection omega and phi scans; index ranges: −11≦h≦10, −11≦k≦6, −29≦1≦29; collected reflections: 14201; independent reflections: 4003 [R(int)=0.0420]; completeness in respect of theta=67.679°: 94.4%].

### Example 135

### (2S)-4-Oxo-2-[2-(4-oxo-1,2,3-benzotriazin-3(4H)-yl)ethyl]-4-[6-(tetrahydro-2H-pyran-4-ylmethoxy)-1,2-benzoxazol-3-yl]butanoic acid (enantiomer 2)

62 mg (chemical purity 100%) of the title compound were obtained.

ee value=98%; [α]_{D}²⁰=+1.3°, 589 nm, c=0.31 g/100 ml, chloroform

¹H-NMR (400 MHz, DMSO-d₆): δ [ppm]=12.54 (br. s, 1H), 8.26 (d, 1H), 8.21 (d, 1H), 8.13-8.06 (m, 1H), 7.98-7.88 (m, 2H), 7.47 (d, 1H), 7.11 (dd, 1H), 4.60-4.44 (m, 2H), 3.98 (d, 2H), 3.89 (dd, 2H), 3.61 (dd, 1H), 3.45-3.27 (m, 3H), 3.12-2.99 (m, 1H), 2.35-2.21 (m, 1H), 2.19-1.98 (m, 2H), 1.70 (d, 2H), 1.36 (qd, 2H).

LC/MS (Method 1, ESIpos): Rₜ=1.05 min, m/z=507 [M+H]⁺.

### Example 136

### (+/−)-4-{6-[(4-Fluorobenzyl)oxy]-1,2-benzoxazol-3-yl}-4-oxo-2-[2-(4-oxo-1,2,3-benzotriazin-3(4H)-yl)ethyl]butanoic acid

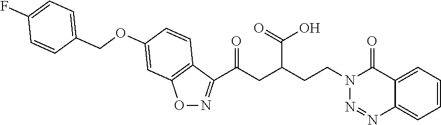 [SMILES: O=C(CC(CCn1nnc2ccccc2c1=O)C(=O)O)c1noc2cc(OCc3ccc(F)cc3)ccc12]

A solution of 116 mg (0.21 mmol) of the compound from Example 119A in 5 ml of dioxane was stirred under reflux for 20 h. After cooling to RT and concentration, the residue was stirred in acetonitrile. The solid was filtered off, washed with pentane and dried in vacuo. 75 mg (70% of theory, purity 100%) of the title compound were obtained.

¹H-NMR (400 MHz, DMSO-d₆): δ [ppm]=12.54 (br. s, 1H), 8.26 (d, 1H), 8.21 (d, 1H), 8.09 (dd, 1H), 7.96-7.92 (m, 2H), 7.58-7.55 (m, 3H), 7.27 (dd, 2H), 7.19 (dd, 1H), 5.23 (d, 2H), 4.58-4.46 (m, 2H), 3.65-3.57 (m, 1H), 3.42 (d, 1H), 3.09-3.03 (m, 1H), 2.31-2.22 (m, 1H), 2.17-2.12 (m, 1H).

LC/MS (Method 1, ESIpos): Rₜ=1.16 min, m/z=517 [M+H]⁺.

Separation of the Enantiomers:

51 mg of the compound from Example 136 were dissolved in 15 ml of of an acetonitrile/methanol mixture at elevated temperature and separated into the enantiomers by preparative HPLC on a chiral phase (see Examples 137 and 138) [column: Daicel Chiralpak AD-H, 5 μm, 250 mm×20 mm; flow rate: 25 ml/min; detection: 210 nm; injection volume: 1 ml; temperature: 40° C.; mobile phase: 30% acetonitrile/70% methanol+0.2% acetic acid, isocratic, run time 6 min]:

### Example 137

### 4-{6-[(4-Fluorobenzyl)oxy]-1,2-benzoxazol-3-yl}-4-oxo-2-[2-(4-oxo-1,2,3-benzotriazin-3(4H)-yl)ethyl]butanoic acid (enantiomer 1)

15 mg (chemical purity 90%) of the title compound were obtained.

Rₜ=7.23 min, ee value=100% [column: Daicel Chiralpak AD-H, 250 mm×4.6 mm, 5 μm; flow rate: 1.0 ml/min; detection: 270 nm; temperature: 40° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.55 (br. s, 1H), 8.25 (d, 1H), 8.20 (d, 1H), 8.09 (dd, 1H), 7.97-7.92 (m, 2H), 7.60-7.53 (m, 3H), 7.25 (dd, 2H), 7.20 (dd, 1H), 5.23 (d, 2H), 4.58-4.46 (m, 2H), 3.68-3.56 (m, 1H), 3.39 (d, 1H), 3.11-3.02 (m, 1H), 2.32-2.23 (m, 1H), 2.17-2.10 (m, 1H).

LC/MS (Method 1, ESIpos): Rₜ=1.15 min, m/z=517 [M+H]⁺.

### Example 138

### 4-{6-[(4-Fluorobenzyl)oxy]-1,2-benzoxazol-3-yl}-4-oxo-2-[2-(4-oxo-1,2,3-benzotriazin-3(4H)-yl)ethyl]butanoic acid (enantiomer 2)

20 mg (chemical purity 100%) of the title compound were obtained.

Rₜ=8.21 min, ee value=96% [column: Daicel Chiralpak AD-H, 250 mm×4.6 mm, 5 μm; flow rate: 1.0 ml/min; detection: 270 nm; temperature: 40° C.; mobile phase: 70% methanol+0.2% glacial acetic acid/30% acetonitrile+0.2% glacial acetic acid, isocratic].

¹H-NMR (400 MHz, DMSO-d₆): δ [ppm]=12.54 (br. s, 1H), 8.25 (d, 1H), 8.21 (d, 1H), 8.09 (dd, 1H), 7.97-7.92 (m, 2H), 7.60-7.53 (m, 3H), 7.25 (dd, 2H), 7.18 (dd, 1H), 5.23 (d, 2H), 4.58-4.46 (m, 2H), 3.65-3.58 (m, 1H), 3.39 (d, 1H), 3.11-3.02 (m, 1H), 2.32-2.23 (m, 1H), 2.17-2.10 (m, 1H).

LC/MS (Method 1, ESIpos): Rₜ=1.15 min, m/z=517 [M+H]⁺.

### Example 139

### (+/−)-4-{6-[(3-Carbamoylbenzyl)oxy]-1,2-benzoxazol-3-yl}-4-oxo-2-[2-(4-oxo-1,2,3-benzotriazin-3(4H)-yl)ethyl]butanoic acid

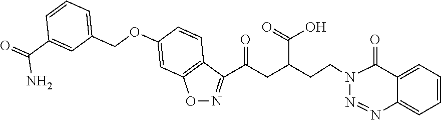 [SMILES: NC(=O)c1cccc(COc2ccc3c(C(=O)CC(CCn4nnc5ccccc5c4=O)C(=O)O)noc3c2)c1]

A solution of 112 mg (0.19 mmol) of the compound from Example 120A in 5 ml of dioxane was stirred under reflux for 10 h. After cooling to RT and concentration, the residue was taken up in DMSO and purified by means of preparative HPLC (Method 6). 83 mg (81% of theory, purity 100%) of the title compound were obtained.

¹H-NMR (400 MHz, DMSO-d₆): δ [ppm]=12.53 (br. s, 1H), 8.25 (d, 1H), 8.20 (d, 1H), 8.09 (dd, 1H), 8.05-7.99 (m, 2H), 7.96-7.91 (m, 2H), 7.86 (m, 1H), 7.65 (d, 1H), 7.59 (d, 1H), 7.50 (dd, 1H), 7.42 (br. s, 1H), 7.20 (dd, 1H), 5.30 (s, 2H), 4.58-4.46 (m, 2H), 3.61 (dd, 1H), 3.41 (dd, 1H), 3.10-3.03 (m, 1H), 2.32-2.23 (m, 1H), 2.17-2.09 (m, 1H).

LC/MS (Method 1, ESIpos): Rₜ=0.94 min, m/z=542 [M+H]⁺.

### Example 140

### (+/−)-4-Oxo-2-[2-(4-oxo-1,2,3-benzotriazin-3(4H)-yl)ethyl]-4-[6-(pentyloxy)-1,2-benzoxazol-3-yl]butanoic acid

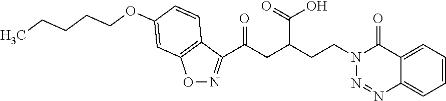 [SMILES: CCCCCOc1ccc2c(C(=O)CC(CCn3nnc4ccccc4c3=O)C(=O)O)noc2c1]

A solution of 77 mg (0.15 mmol) of the compound from Example 121A in 4 ml of dioxane was stirred under reflux for 8 h. After cooling to RT and concentration, the residue was stirred in 1.5 ml of acetonitrile. The solid was filtered off, washed with pentane and dried in vacuo. 47 mg (64% of theory, purity 97%) of the title compound were obtained.

¹H-NMR (400 MHz, DMSO-d₆): δ [ppm]=12.53 (br. s, 1H), 8.26 (d, 1H), 8.21 (d, 1H), 8.09 (dd, 1H), 7.95 (d, 1H), 7.91 (d, 1H), 7.45 (d, 1H), 7.10 (dd, 1H), 4.58-4.46 (m, 2H), 4.10 (dd, 2H), 3.61 (dd, 1H), 3.40 (dd, 1H), 3.10-3.03 (m, 1H), 2.32-2.23 (m, 1H), 2.17-2.09 (m, 1H), 1.80-1.13 (m, 2H), 1.46-1.33 (m, 4H), 0.91 (t, 3H).

LC/MS (Method 1, ESIpos): Rₜ=1.26 min, m/z=479 [M+H]⁺.

### Example 141

### (+/−)-4-[6-(2-Cyclohexylethoxy)-1,2-benzoxazol-3-yl]-4-oxo-2-[2-(4-oxo-1,2,3-benzotriazin-3(4H)-yl)ethyl]butanoic acid

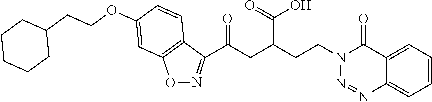 [SMILES: O=C(CC(CCn1nnc2ccccc2c1=O)C(=O)O)c1noc2cc(OCCC3CCCCC3)ccc12]

A solution of 109 mg (0.19 mmol) of the compound from Example 122A in 6 ml of dioxane was stirred under reflux for 8 h. After cooling to RT and concentration, the residue was stirred in 1.5 ml of acetonitrile. The solid was filtered off, washed with pentane and dried in vacuo. 81 mg (79% of theory, purity 97%) of the title compound were obtained.

¹H-NMR (400 MHz, DMSO-d₆): δ [ppm]=12.53 (br. s, 1H), 8.26 (d, 1H), 8.21 (d, 1H), 8.09 (dd, 1H), 7.95 (d, 1H), 7.90 (d, 1H), 7.45 (d, 1H), 7.10 (dd, 1H), 4.58-4.46 (m, 2H), 4.13 (dd, 2H), 3.61 (dd, 1H), 3.40 (dd, 1H), 3.09-3.03 (m, 1H), 2.32-2.23 (m, 1H), 2.17-2.09 (m, 1H), 1.75 (d, 2H), 1.69-1.61 (m, 5H), 1.53-1.45 (m, 1H), 1.27-1.13 (m, 3H), 0.96 (dd, 2H).

LC/MS (Method 1, ESIpos): Rₜ=1.39 min, m/z=519 [M+H]⁺.

Separation of the Enantiomers:

60 mg of the compound from Example 141 were dissolved in 8 ml of ethanol/acetonitrile and separated into the enantiomers by preparative HPLC on a chiral phase (see Examples 142 and 143) [column: Daicel Chiralpak AD-H, 5 μm, 250 mm×20 mm; flow rate: 20 ml/min; detection: 230 nm; injection volume: 0.08 ml; temperature: 25° C.; mobile phase: 22% acetonitrile/70% ethanol/8% acetonitrile+5% glacial acetic acid, isocratic, run time 15 min]:

### Example 142

### (−)-4-[6-(2-Cyclohexylethoxy)-1,2-benzoxazol-3-yl]-4-oxo-2-[2-(4-oxo-1,2,3-benzotriazin-3(4H)-yl)ethyl]butanoic acid (enantiomer 1)

12 mg (chemical purity 100%) of the title compound were obtained.

ee value=100%; [α]²⁰=−32.9°, 365 nm, c=0.28 g/100 ml, chloroform

¹H-NMR (400 MHz, DMSO-d₆): δ [ppm]=12.55 (br. s, 1H), 8.25 (d, 1H), 8.21 (d, 1H), 8.09 (dd, 1H), 7.95 (d, 1H), 7.90 (d, 1H), 7.45 (d, 1H), 7.10 (dd, 1H), 4.58-4.46 (m, 2H), 4.13 (dd, 2H), 3.60 (dd, 1H), 3.40 (dd, 1H), 3.09-3.03 (m, 1H), 2.32-2.23 (m, 1H), 2.17-2.09 (m, 1H), 1.75 (d, 2H), 1.69-1.61 (m, 5H), 1.53-1.45 (m, 1H), 1.27-1.13 (m, 3H), 0.96 (dd, 2H).

LC/MS (Method 1, ESIpos): Rₜ=1.38 min, m/z=519 [M+H]⁺.

### Example 143

### (+)-4-[6-(2-Cyclohexylethoxy)-1,2-benzoxazol-3-yl]-4-oxo-2-[2-(4-oxo-1,2,3-benzotriazin-3(4H)-yl)ethyl]butanoic acid (enantiomer 2)

13 mg (chemical purity 100%) of the title compound were obtained.

ee value=95%; [α]²⁰=+26.1°, 365 nm, c=0.30 g/100 ml, chloroform

¹H-NMR (400 MHz, DMSO-d₆): δ [ppm]=12.55 (br. s, 1H), 8.25 (d, 1H), 8.21 (d, 1H), 8.09 (dd, 1H), 7.95 (d, 1H), 7.90 (d, 1H), 7.45 (d, 1H), 7.10 (dd, 1H), 4.58-4.46 (m, 2H), 4.13 (dd, 2H), 3.60 (dd, 1H), 3.40 (dd, 1H), 3.09-3.03 (m, 1H), 2.32-2.23 (m, 1H), 2.17-2.09 (m, 1H), 1.75 (d, 2H), 1.69-1.61 (m, 5H), 1.53-1.45 (m, 1H), 1.27-1.13 (m, 3H), 0.96 (dd, 2H).

LC/MS (Method 1, ESIpos): Rₜ=1.38 min, m/z=519 [M+H]⁺.

### Example 144

### (+/−)-4-Oxo-2-[2-(4-oxo-1,2,3-benzotriazin-3(4H)-yl)ethyl]-4-[6-(2-phenylethoxy)-1,2-benzoxazol-3-yl]butanoic acid

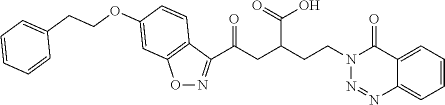 [SMILES: O=C(CC(CCn1nnc2ccccc2c1=O)C(=O)O)c1noc2cc(OCCc3ccccc3)ccc12]

A solution of 104 mg (0.19 mmol) of the compound from Example 123A in 6 ml of dioxane was stirred under reflux for 12 h. After cooling to RT and concentration, the residue was purified by means of preparative HPLC (Method 6). 78 mg (81% of theory, purity 100%) of the title compound were obtained.

¹H-NMR (400 MHz, DMSO-d₆): δ [ppm]=12.53 (br. s, 1H), 8.25 (d, 1H), 8.21 (d, 1H), 8.09 (dd, 1H), 7.95 (d, 1H), 7.90 (d, 1H), 7.49 (s, 1H), 7.37-7.31 (m, 4H), 7.25-7.22 (m, 1H), 7.11 (dd, 1H), 4.57-4.45 (m, 2H), 4.34 (dd, 2H), 3.61 (dd, 1H), 3.40 (dd, 1H), 3.11-3.03 (m, 3H), 2.32-2.22 (m, 1H), 2.17-2.09 (m, 1H).

LC/MS (Method 1, ESIpos): Rₜ=1.22 min, m/z=513 [M+H]⁺.

### Example 145

### (+/−)-4-[6-(Benzyloxy)-1,2-benzoxazol-3-yl]-4-oxo-2-[2-(4-oxo-1,2,3-benzotriazin-3(4H)-yl)ethyl]butanoic acid

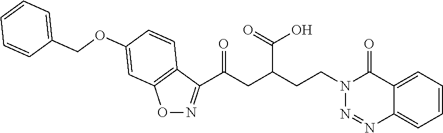 [SMILES: O=C(CC(CCn1nnc2ccccc2c1=O)C(=O)O)c1noc2cc(OCc3ccccc3)ccc12]

A solution of 55 mg (0.09 mmol, purity 90%) of the compound from Example 116A in 2 ml of dioxane was stirred under reflux for 8 h. After cooling to RT and concentration, the residue was purified by means of preparative HPLC (Method 6). 39 mg (84% of theory, purity 100%) of the title compound were obtained.

¹H-NMR (400 MHz, DMSO-d₆): δ [ppm]=12.53 (br. s, 1H), 8.25 (d, 1H), 8.21 (d, 1H), 8.09 (dd, 1H), 7.95-7.91 (m, 2H), 7.57 (d, 1H), 7.50 (d, 2H), 7.42 (dd, 2H), 7.38-7.35 (m, 1H), 7.19 (dd, 1H), 5.25 (s, 2H), 4.57-4.45 (m, 2H), 3.62 (dd, 1H), 3.40 (dd, 1H), 3.11-3.03 (m, 1H), 2.32-2.22 (m, 1H), 2.17-2.09 (m, 1H).

LC/MS (Method 1, ESIpos): Rₜ=1.17 min, m/z=499 [M+H]⁺.

### Example 146

### (+/−)-4-{6-[(3-Chlorobenzyl)oxy]-1,2-benzoxazol-3-yl}-4-oxo-2-[2-(4-oxo-1,2,3-benzotriazin-3(4H)-yl)ethyl]butanoic acid

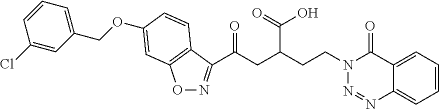 [SMILES: O=C(CC(CCn1nnc2ccccc2c1=O)C(=O)O)c1noc2cc(OCc3cccc(Cl)c3)ccc12]

A solution of 126 mg (0.22 mmol) of the compound from Example 124A in 7 ml of dioxane was stirred under reflux for 18 h. After cooling to RT and concentration, the residue was stirred in acetonitrile. The solid was filtered off, washed with pentane and dried in vacuo. 94 mg (81% of theory, purity 100%) of the title compound were obtained.

¹H-NMR (400 MHz, DMSO-d₆): δ [ppm]=12.53 (br. s, 1H), 8.25 (d, 1H), 8.21 (d, 1H), 8.09 (t, 1H), 7.98-7.91 (m, 2H), 7.58 (d, 2H), 7.48-7.38 (m, 3H), 7.22 (d, 1H), 5.27 (s, 2H), 4.61-4.39 (m, 2H), 3.66-3.55 (m, 1H), 3.40 (dd, 1H), 3.12-3.00 (m, 1H), 2.35-2.20 (m, 1H), 2.19-2.07 (m, 1H).

LC/MS (Method 1, ESIpos): Rₜ=1.22 min, m/z=533 [M+H]⁺.

### Example 147

### (+/−)-4-{6-[(4-Chlorobenzyl)oxy]-1,2-benzoxazol-3-yl}-4-oxo-2-[2-(4-oxo-1,2,3-benzotriazin-3(4H)-yl)ethyl]butanoic acid

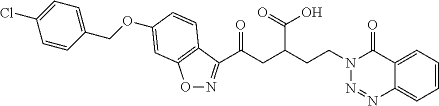 [SMILES: O=C(CC(CCn1nnc2ccccc2c1=O)C(=O)O)c1noc2cc(OCc3ccc(Cl)cc3)ccc12]

A solution of 151 mg (0.26 mmol) of the compound from Example 125A in 9 ml of dioxane was stirred under reflux for 18 h. After cooling to RT and concentration, the residue was stirred in acetonitrile. The solid was filtered off, washed with pentane and dried in vacuo. 102 mg (74% of theory, purity 100%) of the title compound were obtained.

¹H-NMR (400 MHz, DMSO-d₆): δ [ppm]=12.53 (br. s, 1H), 8.25 (d, 1H), 8.20 (d, 1H), 8.12-8.06 (m, 1H), 7.97-7.90 (m, 2H), 7.58-7.44 (m, 5H), 7.19 (dd, 1H), 5.25 (s, 2H), 4.52 (tq, 2H), 3.68-3.54 (m, 1H), 3.40 (dd, 1H), 3.12-3.00 (m, 1H), 2.34-2.20 (m, 1H), 2.19-2.06 (m, 1H).

LC/MS (Method 1, ESIpos): Rₜ=1.23 min, m/z=533 [M+H]⁺.

### Example 148

### (+/−)-4-Oxo-2-[2-(4-oxo-1,2,3-benzotriazin-3(4H)-yl)ethyl]-4-[6-(pyridin-3-ylmethoxy)-1,2-benzoxazol-3-yl]butanoic acid

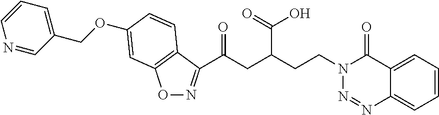 [SMILES: O=C(CC(CCn1nnc2ccccc2c1=O)C(=O)O)c1noc2cc(OCc3cccnc3)ccc12]

A solution of 36 mg (0.07 mmol) of the compound from Example 126A in 2.5 ml of dioxane was stirred under reflux for 5 h. After cooling to RT and concentration, the residue was purified by thick-layer chromatography (PLC silica gel 60 F 254, 1 mm; 20×20 cm with concentration zone; mobile phase dichloromethane/methanol 95:5). 27 mg (81% of theory, purity 100%) of the title compound were obtained.

¹H-NMR (400 MHz, DMSO-d₆): δ [ppm]=12.55 (br. s, 1H), 8.73 (s, 1H), 8.59 (d, 1H), 8.25 (d, 1H), 8.21 (d, 1H), 8.09 (dd, 1H), 7.95-7.92 (m, 3H), 7.62 (s, 1H), 7.46 (d, 1H), 7.20 (dd, 1H), 5.30 (s, 2H), 4.58-4.45 (m, 2H), 3.61 (dd, 1H), 3.40 (dd, 1H), 3.11-3.03 (m, 1H), 2.32-2.22 (m, 1H), 2.17-2.09 (m, 1H).

LC/MS (Method 1, ESIpos): Rₜ=0.88 min, m/z=450 [M+H]⁺.

### Example 149

### (+/−)-4-Oxo-2-[2-(4-oxo-1,2,3-benzotriazin-3(4H)-yl)ethyl]-4-{6-[2-(tetrahydro-2H-pyran-4-yl)ethoxy]-1,2-benzoxazol-3-yl}butanoic acid

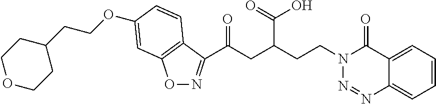 [SMILES: O=C(CC(CCn1nnc2ccccc2c1=O)C(=O)O)c1noc2cc(OCCC3CCOCC3)ccc12]

A solution of 97 mg (0.17 mmol) of the compound from Example 127A in 5 ml of dioxane was stirred under reflux for 4 h. After cooling to RT and concentration, the residue was purified by means of preparative HPLC (Method 6). 60 mg (67% of theory, purity 100%) of the title compound were obtained.

¹H-NMR (400 MHz, DMSO-d₆): δ [ppm]=12.53 (br. s, 1H), 8.25 (dd, 1H), 8.21 (d, 1H), 8.09 (td, 1H), 7.98-7.85 (m, 2H), 7.48 (d, 1H), 7.10 (dd, 1H), 4.60-4.42 (m, 2H), 4.16 (t, 2H), 3.84 (dd, 2H), 3.67-3.54 (m, 1H), 3.44-3.34 (m, 1H, partially hidden), 3.29 (td, 2H), 3.11-2.99 (m, 1H), 2.34-2.21 (m, 1H), 2.20-2.05 (m, 1H), 1.76-1.68 (m, 3H), 1.64 (d, 2H), 1.35-1.14 (m, 2H).

LC/MS (Method 1, ESIpos): Rₜ=0.88 min, m/z=521 [M+H]⁺.

### Example 150

### (+/−)-4-[6-(Cyclopropylmethoxy)-1,2-benzoxazol-3-yl]-4-oxo-2-[2-(4-oxo-1,2,3-benzotriazin-3(4H)-yl)ethyl]butanoic acid

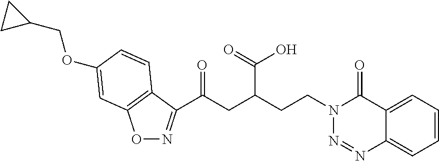 [SMILES: O=C(CC(CCn1nnc2ccccc2c1=O)C(=O)O)c1noc2cc(OCC3CC3)ccc12]

A solution of 85 mg (0.17 mmol) of the compound from Example 128A in 5 ml of dioxane was stirred under reflux for 5 h. After cooling to RT and concentration, the residue was taken up in DMSO and purified by means of preparative HPLC (Method 6). 72 mg (92% of theory, purity 100%) of the title compound were obtained.

¹H-NMR (400 MHz, DMSO-d₆): δ [ppm]=12.53 (br. s, 1H), 8.25 (d, 1H), 8.21 (d, 1H), 8.09 (dd, 1H), 7.95 (d, 1H), 7.91 (s, 1H), 7.42 (s, 1H), 7.12 (dd, 1H), 4.58-4.45 (m, 2H), 3.96 (d, 2H), 3.61 (dd, 1H), 3.40 (dd, 1H), 3.09-3.03 (m, 1H), 2.32-2.22 (m, 1H), 2.17-2.09 (m, 1H), 1.34-1.23 (m, 1H), 0.61 (d, 2H), 0.36 (d, 2H).

LC/MS (Method 1, ESIpos): Rₜ=1.12 min, m/z=463 [M+H]⁺.

### Example 151

### (+/−)-4-[6-(2-Cyclopentylethoxy)-1,2-benzoxazol-3-yl]-4-oxo-2-[2-(4-oxo-1,2,3-benzotriazin-3(4H)-yl)ethyl]butanoic acid

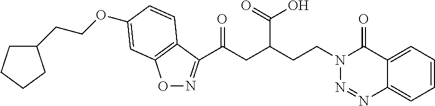 [SMILES: O=C(CC(CCn1nnc2ccccc2c1=O)C(=O)O)c1noc2cc(OCCC3CCCC3)ccc12]

A solution of 98 mg (0.18 mmol) of the compound from Example 129A in 5 ml of dioxane was stirred under reflux for 16 h. After cooling to RT and concentration, the residue was purified by means of preparative HPLC (Method 6). 52 mg (58% of theory, purity 100%) of the title compound were obtained.

¹H-NMR (400 MHz, DMSO-d₆): δ [ppm]=12.53 (br. s, 1H), 8.26 (d, 1H), 8.21 (d, 1H), 8.09 (dd, 1H), 7.95 (d, 1H), 7.91 (s, 1H), 7.45 (dd, 1H), 7.10 (dd, 1H), 4.58-4.45 (m, 2H), 4.12 (dd, 2H), 3.61 (dd, 1H), 3.40 (dd, 1H), 3.09-3.03 (m, 1H), 2.32-2.22 (m, 1H), 2.17-2.09 (m, 1H), 1.99-1.92 (m, 1H), 1.82-1.75 (m, 4H), 1.64-1.49 (m, 4H), 1.21-1.12 (m, 2H).

LC/MS (Method 2, ESIpos): Rₜ=2.86 min, m/z=505 [M+H]⁺.

### Example 152

### (+/−)-4-[6-(Cyclopentylmethoxy)-1,2-benzoxazol-3-yl]-4-oxo-2-[2-(4-oxo-1,2,3-benzotriazin-3(4H)-yl)ethyl]butanoic acid

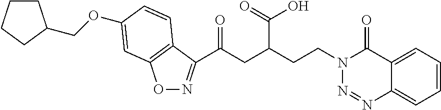 [SMILES: O=C(CC(CCn1nnc2ccccc2c1=O)C(=O)O)c1noc2cc(OCC3CCCC3)ccc12]

A solution of 88 mg (0.17 mmol) of the compound from Example 130A in 5 ml of dioxane was stirred under reflux for 16 h. After cooling to RT and concentration, the residue was purified by means of preparative HPLC (Method 6). 42 mg (52% of theory, purity 100%) of the title compound were obtained.

¹H-NMR (400 MHz, DMSO-d₆): δ [ppm]=12.53 (br. s, 1H), 8.26 (d, 1H), 8.21 (d, 1H), 8.09 (dd, 1H), 7.95 (d, 1H), 7.91 (s, 1H), 7.45 (d, 1H), 7.12 (d, 1H), 4.58-4.45 (m, 2H), 3.98 (d, 2H), 3.61 (dd, 1H), 3.40 (dd, 1H), 3.09-3.03 (m, 1H), 2.39-2.32 (m, 1H), 2.30-2.23 (m, 1H), 2.17-2.09 (m, 1H), 1.87-1.74 (m, 2H), 1.69-1.49 (m, 4H), 1.40-1.29 (m, 2H).

LC/MS (Method 1, ESIpos): Rₜ=1.30 min, m/z=491 [M+H]⁺.

### Example 153

### (+/−)-4-[6-(Cyclohexylmethoxy)-1,2-benzoxazol-3-yl]-4-oxo-2-[2-(4-oxo-1,2,3-benzotriazin-3(4H)-yl)ethyl]butanoic acid

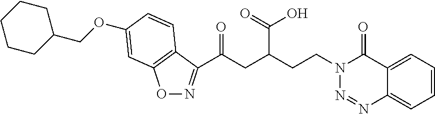 [SMILES: O=C(CC(CCn1nnc2ccccc2c1=O)C(=O)O)c1noc2cc(OCC3CCCCC3)ccc12]

A solution of 73 mg (0.13 mmol) of the compound from Example 131A in 5 ml of dioxane was stirred under reflux for 16 h. After cooling to RT and concentration, the residue was purified by means of preparative HPLC (Method 6). 40 mg (58% of theory, purity 100%) of the title compound were obtained.

¹H-NMR (400 MHz, DMSO-d₆): δ [ppm]=12.53 (br. s, 1H), 8.26 (d, 1H), 8.21 (d, 1H), 8.09 (dd, 1H), 7.95 (d, 1H), 7.91 (s, 1H), 7.45 (s, 1H), 7.10 (d, 1H), 4.58-4.45 (m, 2H), 3.92 (d, 2H), 3.61 (dd, 1H), 3.40 (dd, 1H), 3.09-3.03 (m, 1H), 2.32-2.23 (m, 1H), 2.17-2.10 (m, 1H), 1.88-1.59 (m, 6H), 1.31-1.16 (m, 3H), 1.11-1.03 (m, 2H).

LC/MS (Method 1, ESIpos): Rₜ=1.36 min, m/z=505 [M+H]⁺.

### Example 154

### (+/−)-4-[6-(Benzyloxy)-1,2-benzoxazol-3-yl]-4-oxo-2-{2-[4-oxo-6-(trifluoromethyl)-1,2,3-benzotriazin-3(4H)-yl]ethyl}butanoic acid

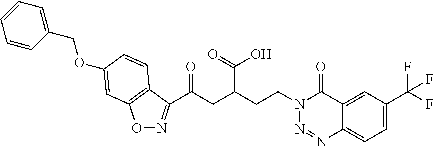 [SMILES: O=C(CC(CCn1nnc2ccc(C(F)(F)F)cc2c1=O)C(=O)O)c1noc2cc(OCc3ccccc3)ccc12]

A solution of 62 mg (0.10 mmol) of the compound from Example 117A in 2.5 ml of dioxane was stirred under reflux for 24 h. After cooling to RT and concentration, the residue was purified by means of preparative HPLC (Method 6). 43 mg (74% of theory, purity 100%) of the title compound were obtained.

¹H-NMR (400 MHz, DMSO-d₆): δ [ppm]=12.54 (br. s, 1H), 8.49 (s, 1H), 8.45-8.38 (m, 2H), 7.93 (d, 1H), 7.57 (d, 1H), 7.52-7.47 (m, 2H), 7.45-7.32 (m, 3H), 7.19 (dd, 1H), 5.25 (s, 2H), 4.64-4.44 (m, 2H), 3.61 (dd, 1H), 3.40 (dd, 1H), 3.14-3.00 (m, 1H), 2.35-2.23 (m, 1H), 2.20-2.08 (m, 1H).

LC/MS (Method 1, ESIpos): Rₜ=1.29 min, m/z=567 [M+H]⁺.

### Example 155

### (+/−)-4-Oxo-2-{2-[4-oxo-6-(trifluoromethyl)-1,2,3-benzotriazin-3(4H)-yl]ethyl}-4-[6-(tetrahydro-2H-pyran-4-ylmethoxy)-1,2-benzoxazol-3-yl]butanoic acid

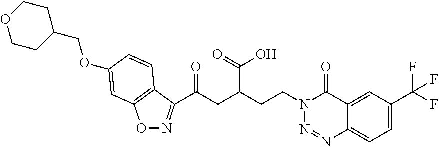 [SMILES: O=C(CC(CCn1nnc2ccc(C(F)(F)F)cc2c1=O)C(=O)O)c1noc2cc(OCC3CCOCC3)ccc12]

A solution of 48 mg (0.08 mmol) of the compound from Example 132A in 2.5 ml of dioxane was stirred under reflux for 16 h. After cooling to RT and concentration, the residue was purified by means of preparative HPLC (Method 6). 30 mg (60% of theory, purity 90%) of the title compound were obtained.

¹H-NMR (400 MHz, DMSO-d₆): δ [ppm]=12.53 (br. s, 1H), 8.48 (s, 1H), 8.46-8.35 (m, 2H), 7.90 (d, 1H), 7.46 (d, 1H), 7.11 (dd, 1H), 4.63-4.46 (m, 2H), 3.98 (d, 2H), 3.89 (dd, 2H), 3.61 (dd, 1H), 3.43-3.31 (m, 3H), 3.14-3.02 (m, 1H), 2.36-2.23 (m, 1H), 2.22-1.98 (m, 2H), 1.70 (d, 2H), 1.36 (qd, 2H).

LC/MS (Method 1, ESIpos): Rₜ=1.17 min, m/z=575 [M+H]⁺.

### Example 156

### (+/−)-4-[6-(Benzyloxy)-2,1-benzoxazol-3-yl]-4-oxo-2-[2-(4-oxo-1,2,3-benzotriazin-3(4H)-yl)ethyl]butanoic acid

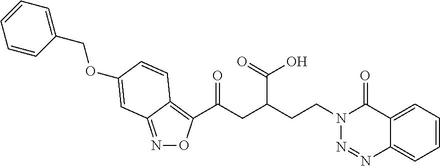 [SMILES: O=C(CC(CCn1nnc2ccccc2c1=O)C(=O)O)c1onc2cc(OCc3ccccc3)ccc12]

A solution of 23 mg (0.04 mmol) of the compound from Example 137A in 2 ml of dioxane was stirred under reflux for 3.5 h. After cooling to RT and concentration, the residue was taken up in DMSO and purified by means of preparative HPLC (Method 6). The product thus obtained was stirred in pentane, and the solvent was filtered off. This procedure was repeated twice more and the solid was finally dried in vacuo. 8 mg (36% of theory, purity 100%) of the title compound were obtained.

¹H-NMR (400 MHz, DMSO-d₆): δ [ppm]=12.53 (br. s, 1H), 8.43-7.69 (m, 5H), 7.60-6.96 (m, 7H), 5.24 (s, 2H), 4.70-4.29 (m, 2H), 3.75-3.50 (m, 1H), 3.48-3.37 (m, 1H), 3.10-2.93 (m, 1H), 2.33-1.99 (m, 2H).

LC/MS (Method 1, ESIpos): Rₜ=1.13 min, m/z=499 [M+H]⁺.

### Example 157

### (+/−)-(1RS,2SR,5RS)-2-{[4-Oxo-6-(trifluoromethyl)-1,2,3-benzotriazin-3(4H)-yl]methyl}-5-{[5-(2-phenylethoxy)-1-benzofur-2-yl]carbonyl}cyclopentanecarboxylic acid (racemate)

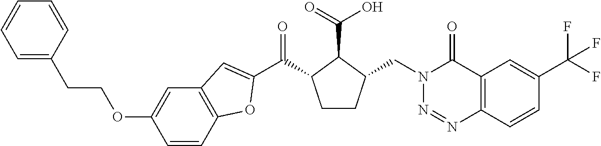 [SMILES: O=C(O)[C@H]1[C@H](Cn2nnc3ccc(C(F)(F)F)cc3c2=O)CC[C@@H]1C(=O)c1cc2cc(OCCc3ccccc3)ccc2o1]

A solution of 695 mg (0.68 mmol, purity 69%) of the compound from Example 145A in 16 ml of dichloromethane was admixed with ice cooling with 8 ml (0.10 mmol) of trifluoroacetic acid and the mixture was stirred for 15 min at 0° C. Then, the cooling bath was removed and stirring was continued for 1 h at RT. Concentration was then carried out, and the residue was purified by preparative HPLC (Method 6). 289 mg (70% of theory, purity 100%) of the title compound were obtained.

¹H-NMR (400 MHz, DMSO-d₆): δ [ppm]=12.23 (br. s, 1H), 8.51 (s, 1H), 8.46-8.36 (m, 2H), 7.86 (s, 1H), 7.61 (d, 1H), 7.38-7.28 (m, 5H), 7.26-7.20 (m, 1H), 7.14 (dd, 1H), 4.58 (d, 2H), 4.23 (t, 2H), 4.08-3.97 (m, 1H), 3.20 (t, 1H), 3.06 (t, 2H), 2.96-2.82 (m, 1H), 2.19-2.07 (m, 1H), 2.03-1.90 (m, 1H), 1.88-1.75 (m, 1H), 1.65-1.50 (m, 1H).

LC/MS (Method 1, ESIpos): Rₜ=1.31 min, m/z=606 [M+H]⁺.

Separation of the Enantiomers:

410 mg of the compound from Example 157 were dissolved in 6 ml of ethanol and separated into the enantiomers by preparative SFC on a chiral phase (see Examples 158 and 159) [column: Daicel Chiralpak IC, 5 μm 250 mm×4.6 mm; flow rate: 80 ml/min; detection: 220 nm; injection volume: 0.75 ml; temperature: 40° C.; mobile phase: 70% CO₂/30% ethanol, isocratic, run time 13 min]:

### Example 158

### (−)-(1RS,2SR,5RS)-2-{[4-Oxo-6-(trifluoromethyl)-1,2,3-benzotriazin-3(4H)-yl]methyl}-5-{[5-(2-phenylethoxy)-1-benzofur-2-yl]carbonyl}cyclopentanecarboxylic acid (enantiomer 1)

187 mg (chemical purity 97%) of the title compound were obtained.

ee value=99.9%; [α]_{D}²⁰=−71.4°, 589 nm, c=0.35 g/100 ml, chloroform

¹H-NMR (400 MHz, DMSO-d₆): δ [ppm]=12.23 (br. s, 1H), 8.51 (s, 1H), 8.46-8.38 (m, 2H), 7.86 (d, 1H), 7.61 (d, 1H), 7.38-7.27 (m, 5H), 7.26-7.19 (m, 1H), 7.14 (dd, 1H), 4.58 (d, 2H), 4.23 (t, 2H), 4.07-3.98 (m, 1H), 3.20 (t, 1H), 3.07 (t, 2H), 2.95-2.84 (m, 1H), 2.20-2.08 (m, 1H), 2.02-1.90 (m, 1H), 1.88-1.76 (m, 1H), 1.65-1.51 (m, 1H).

LC/MS (Method 1, ESIpos): Rₜ=1.33 min, m/z=606 [M+H]⁺.

### Example 159

### (+)-(1RS,2SR,5RS)-2-{[4-Oxo-6-(trifluoromethyl)-1,2,3-benzotriazin-3(4H)-yl]methyl}-5-{[5-(2-phenylethoxy)-1-benzofur-2-yl]carbonyl}cyclopentanecarboxylic acid (enantiomer 2)

177 mg (chemical purity 100%) of the title compound were obtained.

ee value=99.9%; [α]_{D}²⁰=+70.5°, 589 nm, c=0.33 g/100 ml, chloroform

¹H-NMR (400 MHz, DMSO-d₆): δ [ppm]=12.23 (br. s, 1H), 8.51 (s, 1H), 8.47-8.36 (m, 2H), 7.86 (s, 1H), 7.61 (d, 1H), 7.37-7.28 (m, 5H), 7.26-7.19 (m, 1H), 7.14 (dd, 1H), 4.58 (d, 2H), 4.24 (t, 2H), 4.03 (q, 1H), 3.20 (t, 1H), 3.07 (t, 2H), 2.96-2.82 (m, 1H), 2.21-2.08 (m, 1H), 2.04-1.90 (m, 1H), 1.88-1.75 (m, 1H), 1.65-1.50 (m, 1H).

LC/MS (Method 1, ESIpos): Rₜ=1.33 min, m/z=606 [M+H]⁺.

### B. ASSESSMENT OF PHARMACOLOGICAL EFFICACY

The pharmacological activity of the compounds according to the invention can be demonstrated by in vitro and in vivo studies as known to the person skilled in the art. The application examples which follow describe the biological action of the compounds according to the invention, without restricting the invention to these examples.

Abbreviations and Acronyms:
APMA 4-aminophenylmercuric acetate
Brij®-35 polyoxyethylene lauryl ether
BSA bovine serum albumin
CYP cytochrome P450
Dap (or Dpa) L-2,3-diaminopropionic acid (β-amino-L-alanine)
DMSO dimethyl sulphoxide
Dnp 2,4-dinitrophenyl
EDTA ethylenediaminetetraacetic acid
HEPES 2-[4-(2-hydroxyethyl)piperazin-1-yl]ethanesulphonic acid
HME human macrophage elastase
IC inhibition concentration
Mca (7-methoxycoumarin-4-yl)acetyl
MMP matrix metallopeptidase
MTP microtitre plate
NADP⁺ nicotinamide adenine dinucleotide phosphate (oxidized form)
NADPH nicotinamide adenine dinucleotide phosphate (reduced form)
Nval norvaline
PEG polyethylene glycol
Tris tris(hydroxymethyl)aminomethane
v/v volume to volume ratio (of a solution)
w/w weight to weight ratio (of a solution)

B-1. In Vitro HME Inhibition Test:

The potency of the compounds according to the invention with respect to HME (MMP-12) is determined in an in vitro inhibition test. The HME-mediated amidolytic cleavage of a suitable peptide substrate leads to an increase in fluorescent light therein. The signal intensity of the fluorescent light is directly proportional to the enzyme activity. The active concentration of a test compound at which half the enzyme is inhibited (50% signal intensity of the fluorescent light) is reported as the IC₅₀ value.

Standard In Vitro HME Inhibition Test:

In a 384 hole microtiter plate, in a total test volume of 41 μl, the test buffer (0.1 M HEPES pH 7.4, 0.15 M NaCl, 0.03 M CaCl₂, 0.004 mM ZnCl₂, 0.02 M EDTA, 0.005% Brij®), the enzyme (0.5 nM HME; R&D Systems, 917-MP, autocatalytic activation according to the manufacturer's instructions) and the intramolecularly quenched substrate [5 μM Mca-Pro-Leu-Gly-Leu-Glu-Glu-Ala-Dap(Dnp)-NH₂; Bachem, M-2670] are incubated in the absence and presence of the test substance (as a solution in DMSO) at 37° C. for two hours. The fluorescence intensity of the test mixtures is measured (excitation 323 mm, emission 393 nm). The IC₅₀ values are ascertained by plotting the fluorescent light intensity against the active ingredient concentration.

High-Sensitivity In Vitro HME Inhibition Test:

If sub-nanomolar IC values are found for highly potent test substances in the standard HME inhibition test described above, a modified test is used to determine them more accurately. In this case, an enzyme concentration ten times lower is used (final concentration, e.g. 0.05 nM) in order to achieve an increased test sensitivity. The incubation period chosen for the test is correspondingly longer (e.g. 16 hours).

In Vitro HME Inhibition Test in the Presence of Serum Albumin in the Reaction Buffer:

This test corresponds to the standard HME inhibition test described above, except using a modified reaction buffer. This reaction buffer additionally contains bovine serum albumin (BSA, fatty acid-free, A6003, Sigma-Aldrich) of final concentration 2% (w/w), which corresponds to about half the physiological serum albumin content. The enzyme concentration in this modified test is slightly increased (e.g. 0.75 nM), as is the incubation time (e.g. three hours).

Table 1 below shows, for individual working examples of the invention, the IC₅₀ values from the standard or high-sensitivity HME inhibition test (in some cases as mean values from two or more independent individual determinations and rounded to two significant figures):

TABLE 1
Inhibition of human macrophage elastase (HME/hMMP-12)

| Example | HME/hMMP-12 |
|---|---|
| No. | IC₅₀ [nM] |
| 1 | 2.6 |
| 2 | 2.6 |
| 3 | 4.1 |
| 4 | 4.3 |
| 5 | 2.1 |
| 6 | 4.0 |
| 7 | 2.8 |
| 8 | 2.0 |
| 9 | 370 |
| 10 | 0.62 |
| 11 | 4.3 |
| 12 | 900 |
| 13 | 6.1 |
| 14 | 0.65 |
| 15 | 4.8 |
| 16 | 24 |
| 17 | 94 |
| 18 | 6.0 |
| 19 | 4.7 |
| 20 | 860 |
| 21 | 2.2 |
| 22 | 2.5 |
| 23 | 0.81 |
| 24 | 210 |
| 25 | 0.2 |
| 26 | 160 |
| 27 | 0.19 |
| 28 | 3.1 |
| 29 | 770 |
| 30 | 0.99 |
| 31 | 4.8 |
| 32 | 0.68 |
| 33 | 350 |
| 34 | 1.1 |
| 35 | 2.0 |
| 36 | 0.33 |
| 37 | 4.6 |
| 38 | 0.72 |
| 40 | 4.6 |
| 41 | 3.9 |
| 42 | 3.2 |
| 43 | 1.3 |
| 44 | 0.62 |
| 45 | 2.3 |
| 46 | 1.9 |
| 47 | 0.94 |
| 48 | 4.0 |
| 49 | 0.55 |
| 50 | 1.6 |
| 51 | 4.1 |
| 52 | 2.1 |
| 53 | 2.9 |
| 54 | 5.6 |
| 55 | 3.7 |
| 56 | 1400 |
| 57 | 2.1 |
| 58 | 5.0 |
| 59 | 980 |
| 60 | 1.8 |
| 61 | 4.6 |
| 62 | 570 |
| 63 | 2.2 |
| 64 | 2.9 |
| 65 | 340 |
| 66 | 2.4 |
| 67 | 3.8 |
| 68 | 890 |
| 69 | 1.0 |
| 70 | 4.1 |
| 71 | 2.2 |
| 72 | 3.1 |
| 73 | 2.8 |
| 74 | 0.18 |
| 75 | 0.22 |
| 76 | 0.66 |
| 77 | 2.9 |
| 78 | 2.0 |
| 79 | 1900 |
| 80 | 0.46 |
| 82 | 0.089 |
| 83 | 1.4 |
| 84 | 4.0 |
| 85 | 0.38 |
| 86 | 1.4 |
| 87 | 470 |
| 88 | 1.1 |
| 89 | 0.35 |
| 90 | 81 |
| 91 | 0.23 |
| 92 | 1.3 |
| 93 | 97 |
| 94 | 0.82 |
| 95 | 0.6 |
| 96 | 0.81 |
| 97 | 25 |
| 98 | 0.4 |
| 99 | 100 |
| 100 | 0.18 |
| 101 | 0.53 |
| 102 | 2100 |
| 103 | 0.23 |
| 104 | 1.1 |
| 105 | 0.21 |
| 106 | 31 |
| 107 | 0.61 |
| 108 | 0.23 |
| 109 | 10 |
| 110 | 0.54 |
| 111 | 3.3 |
| 112 | 2.2 |
| 113 | 4300 |
| 114 | 0.3 |
| 115 | 3.1 |
| 116 | 4.2 |
| 117 | 5.9 |
| 118 | 2.7 |
| 119 | 3.7 |
| 120 | 3.6 |
| 121 | 3.1 |
| 122 | 4.5 |
| 123 | 2.7 |
| 124 | 0.95 |
| 125 | 4.0 |
| 126 | 2.9 |
| 127 | 2.8 |
| 128 | 1.1 |
| 129 | 13 |
| 130 | 0.12 |
| 131 | 1.2 |
| 132 | 0.51 |
| 133 | 0.24 |
| 134 | 24 |
| 135 | 0.16 |
| 136 | 0.63 |
| 137 | 35 |
| 138 | 0.33 |
| 139 | 0.58 |
| 140 | 0.28 |
| 141 | 0.12 |
| 142 | 15 |
| 143 | 0.019 |
| 144 | 1.4 |
| 145 | 0.71 |
| 146 | 1.1 |
| 147 | 1.7 |
| 148 | 1.8 |
| 149 | 0.58 |
| 150 | 3.8 |
| 151 | 0.27 |
| 152 | 1.4 |
| 153 | 1.3 |
| 154 | 0.75 |
| 155 | 0.39 |
| 156 | 2.4 |
| 157 | 0.055 |
| 158 | 260 |
| 159 | 0.063 |

B-2. In Vitro MMP Inhibition Tests

The potency of the compounds according to the invention with respect to other MMPs (and hence their selectivity) is likewise determined in in vitro inhibition tests. The MMP-mediated amidolytic cleavage of a suitable peptide substrate leads to an increase in fluorescent light here too. The signal intensity of the fluorescent light is directly proportional to the enzyme activity. The active concentration of a test compound at which half the enzyme is inhibited (50% signal intensity of the fluorescent light) is reported as the IC₅₀ value.

a) Human MMPs:

In Vitro MMP-1 Inhibition Test:

Recombinant MMP-1 (R&D Systems, 901-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-1 reaction is measured by measuring the fluorescence intensity (excitation 320 nm, emission 410 nm) over a suitable period of time (e.g. over 120 min at a temperature of 32° C.).

In Vitro MMP-2 Inhibition Test:

Recombinant MMP-2 (R&D Systems, 902-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-2 reaction is measured by measuring the fluorescence intensity (excitation 320 nm, emission 410 nm) over a suitable period of time (e.g. over 120 min at a temperature of 32° C.).

In Vitro MMP-3 Inhibition Test:

Recombinant MMP-3 (R&D Systems, 513-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration e.g. 10 μM; R&D Systems, ES-002), so as to result in a total test volume of 50 μl. The course of the MMP-3 reaction is measured by measuring the fluorescence intensity (excitation 320 nm, emission 410 nm) over a suitable period of time (e.g. over 120 min at a temperature of 32° C.).

In Vitro MMP-7 Inhibition Test:

Recombinant MMP-7 (from R&D Systems, 907-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-7 reaction is measured by measuring the fluorescence intensity (excitation 320 nm, emission 410 nm) over a suitable period of time (e.g. over 120 min at a temperature of 32° C.).

In Vitro MMP-8 Inhibition Test:

Recombinant MMP-8 (R&D Systems, 908-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-8 reaction is measured by measuring the fluorescence intensity (excitation 320 nm, emission 410 nm) over a suitable period of time (e.g. over 120 min at a temperature of 32° C.).

In Vitro MMP-9 Inhibition Test:

Recombinant MMP-9 (R&D Systems, 911-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-9 reaction is measured by measuring the fluorescence intensity (excitation 320 nm, emission 410 nm) over a suitable period of time (e.g. over 120 min at a temperature of 32° C.).

In Vitro MMP-10 Inhibition Test:

Recombinant MMP-10 (R&D Systems, 910-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration e.g. 10 μM; R&D Systems, ES-002), so as to result in a total test volume of 50 μl. The course of the MMP-10 reaction is measured by measuring the fluorescence intensity (excitation 320 nm, emission 410 nm) over a suitable period of time (e.g. over 120 min at a temperature of 32° C.).

In Vitro MMP-13 Inhibition Test:

Recombinant MMP-13 (R&D Systems, 511-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-13 reaction is measured by measuring the fluorescence intensity (excitation 320 nm, emission 410 nm) over a suitable period of time (e.g. over 120 min at a temperature of 32° C.).

In Vitro MMP-14 Inhibition Test:

Recombinant MMP-14 (R&D Systems, 918-MP) is enzymatically activated in accordance with the manufacturer's instructions using recombinant furin (R&D Systems, 1503-SE). 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-14 reaction is measured by measuring the fluorescence intensity (excitation 320 nm, emission 410 nm) over a suitable period of time (e.g. over 120 min at a temperature of 32° C.).

In Vitro MMP-16 Inhibition Test:

Recombinant MMP-16 (R&D Systems, 1785-MP) is enzymatically activated in accordance with the manufacturer's instructions using recombinant furin (R&D Systems, 1503-SE). 1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-16 reaction is measured by measuring the fluorescence intensity (excitation 320 nm, emission 410 nm) over a suitable period of time (e.g. over 120 min at a temperature of 32° C.).

Tables 2A and 2B below show, for representative working examples of the invention, the IC₅₀ values from these tests relating to the inhibition of human MMPs (in some cases as mean values from two or more independent individual determinations and rounded to two significant figures):

TABLE 2A
Inhibition of human MMPs

| Example | MMP-1 | MMP-2 | MMP-3 | MMP-7 | MMP-8 |
|---|---|---|---|---|---|
| No. | IC₅₀ [nM] | IC₅₀ [nM] | IC₅₀ [nM] | IC₅₀ [nM] | IC₅₀ [nM] |
| 1 | >40000 | >40000 | >40000 | >40000 | >40000 |
| 2 | >40000 | 4600 | 25000 | >40000 | 14000 |
| 5 | >40000 | 5000 | >40000 | >40000 | 7700 |
| 6 | >40000 | 430 | 26000 | >40000 | 3900 |
| 7 | >40000 | 920 | 27000 | >40000 | 22000 |
| 8 | >40000 | >40000 | >40000 | >40000 | 30000 |
| 13 | >40000 | 1300 | >40000 | >40000 | 5000 |
| 21 | >40000 | 1900 | 22000 | >40000 | 14000 |
| 23 | >40000 | 1900 | >40000 | >40000 | 6300 |
| 30 | >40000 | 6100 | >40000 | >40000 | 32000 |
| 31 | >40000 | 4400 | >40000 | >40000 | 35000 |
| 34 | >40000 | 490 | 34000 | 25000 | 4900 |
| 37 | >40000 | 3300 | 39000 | >40000 | 12000 |
| 44 | >40000 | >40000 | 23000 | >40000 | 28000 |
| 50 | >40000 | 3800 | 34000 | >40000 | 36000 |
| 51 | >40000 | 19000 | >40000 | 35000 | 34000 |
| 58 | >40000 | 10000 | >40000 | >40000 | 10000 |
| 69 | >40000 | 2900 | 36000 | >40000 | >40000 |
| 71 | >40000 | >40000 | >40000 | >40000 | >40000 |
| 73 | >40000 | 1300 | 29000 | >40000 | >40000 |
| 74 | >40000 | 20 | 16000 | >40000 | 370 |
| 75 | >40000 | 390 | >40000 | >40000 | 3300 |
| 80 | >40000 | 490 | 5700 | >40000 | 440 |
| 82 | >40000 | 170 | 16000 | >40000 | 580 |
| 89 | >40000 | 290 | >40000 | >40000 | 6700 |
| 91 | >40000 | 96 | 17000 | >40000 | 940 |
| 100 | >40000 | 140 | >40000 | >40000 | 2800 |
| 101 | >40000 | 660 | >40000 | >40000 | 12000 |
| 111 | >40000 | 350 | 9700 | 4100 | 460 |
| 112 | >40000 | 220 | 6400 | 3200 | 240 |
| 114 | >40000 | 83 | 3200 | 5300 | 3200 |
| 118 | >40000 | 350 | 16000 | 30000 | 9300 |
| 135 | >40000 | 270 | 12000 | >40000 | 170 |
| 136 | >40000 | 320 | 15000 | >40000 | 1200 |
| 140 | >40000 | 95 | 14000 | >40000 | 570 |
| 144 | >40000 | 730 | 18000 | >40000 | 2500 |
| 145 | >40000 | 430 | 8900 | >40000 | 2800 |
| 148 | >40000 | 270 | 9100 | >40000 | 270 |
| 150 | >40000 | 510 | 33000 | 29000 | 400 |
| 153 | >40000 | 420 | 12000 | >40000 | 1700 |
| 154 | >40000 | 5600 | 10000 | 18000 | 970 |
| 155 | >40000 | 4500 | 19000 | 24000 | 82 |
| 156 | >40000 | 1400 | 13000 | >40000 | 2800 |
| 157 | >40000 | 880 | 1100 | 160 | 150 |
| 159 | >40000 | 1700 | 2200 | 300 | 310 |

TABLE 2B
Inhibition of human MMPs

| Example | MMP-9 | MMP-10 | MMP-13 | MMP-14 | MMP-16 |
|---|---|---|---|---|---|
| No. | IC₅₀ [nM] | IC₅₀ [nM] | IC₅₀ [nM] | IC₅₀ [nM] | IC₅₀ [nM] |
| 1 | >40000 | 28000 | >40000 | >40000 | >40000 |
| 2 | 24000 | 16000 | 2200 | >40000 | >40000 |
| 5 | 9100 | 34000 | 13000 | 34000 | >40000 |
| 6 | 3400 | 7000 | 730 | 37000 | >40000 |
| 7 | 5000 | 9300 | 5000 | >40000 | >40000 |
| 8 | 13000 | >40000 | 22000 | >40000 | >40000 |
| 13 | 3800 | 15000 | 4600 | 17000 | >40000 |
| 21 | 5600 | 13000 | 1800 | 30000 | >40000 |
| 23 | 13000 | 11000 | 5300 | 15000 | 30000 |
| 30 | 27000 | 30000 | 10000 | 31000 | >40000 |
| 31 | 9700 | >40000 | 5100 | >40000 | >40000 |
| 34 | 38000 | 14000 | 1600 | 7900 | >40000 |
| 37 | 17000 | 29000 | 5200 | >40000 | >40000 |
| 44 | 9900 | 29000 | 2100 | 13000 | >40000 |
| 50 | 26000 | 20000 | 4000 | >40000 | >40000 |
| 51 | 30000 | 36000 | 11000 | >40000 | >40000 |
| 58 | 10000 | >40000 | >40000 | 2200 | >40000 |
| 69 | 13000 | 28000 | 3600 | >40000 | >40000 |
| 71 | >40000 | >40000 | 30000 | >40000 | >40000 |
| 73 | 2200 | >40000 | 4700 | >40000 | >40000 |
| 74 | 180 | 2100 | 250 | 1400 | 6400 |
| 75 | 450 | 7700 | 3800 | 6500 | >40000 |
| 80 | 280 | 12000 | 3600 | 1900 | 21000 |
| 82 | 94 | 6800 | 1300 | 2500 | >40000 |
| 89 | 230 | 22000 | 670 | 13000 | >40000 |
| 91 | 41 | 4700 | 370 | 4000 | 13000 |
| 100 | 74 | 11000 | 140 | 4000 | >40000 |
| 101 | 420 | >40000 | 1100 | >40000 | >40000 |
| 111 | 1100 | 4700 | 1900 | 6000 | >40000 |
| 112 | 590 | 2500 | 1300 | 2800 | 37000 |
| 114 | 240 | 3400 | 250 | 2100 | 28000 |
| 118 | 1200 | 10000 | 1100 | 19000 | >40000 |
| 135 | 1700 | 9000 | 1500 | 3500 | 36000 |
| 136 | 190 | 12000 | 970 | 4900 | 30000 |
| 140 | 43 | 6300 | 330 | 3400 | 24000 |
| 144 | 760 | 29000 | 1600 | 2500 | 15000 |
| 145 | 170 | 10000 | 1500 | 4000 | 25000 |
| 148 | 910 | 200 | 500 | 8500 | >40000 |
| 150 | 650 | >40000 | 2400 | 900 | 11000 |
| 153 | 450 | 14000 | 2800 | 5500 | >40000 |
| 154 | 250 | 3300 | 2300 | 26000 | 13000 |
| 155 | 2800 | 5100 | 2700 | >40000 | 30000 |
| 156 | 170 | 28000 | 2200 | 9300 | 38000 |
| 157 | 190 | 190 | 290 | 700 | 4900 |
| 159 | 680 | 340 | 660 | 2600 | 3000 |

On comparison of the inhibition data shown in Tables 1 and 2A/2B, it is found that the compounds according to the invention in general and the more active stereoisomers thereof in particular have very high inhibitory potency (frequently in the nano- or sub-nanomolar range) with respect to HME, and simultaneously a very high selectivity (generally two to four orders of magnitude or even higher) with respect to related human MMPs.

b) Rodent MMPs:

In Vitro Mouse MMP-2 Inhibition Test:

Recombinant mouse MMP-2 (R&D Systems, 924-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-2 reaction is measured by measuring the fluorescence intensity (excitation 320 nm, emission 410 nm) over a suitable period of time (e.g. over 120 min at a temperature of 32° C.).

In Vitro Mouse MMP-3 Inhibition Test:

Recombinant mouse MMP-3 (R&D Systems, 548-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Arg-Pro-Lys-Pro-Val-Glu-Nval-Trp-Arg-Lys(Dnp)-NH₂ substrate (final concentration e.g. 5 μM; R&D Systems, ES-002), so as to result in a total test volume of 50 μl. The course of the MMP-3 reaction is measured by measuring the fluorescence intensity (excitation 320 nm, emission 410 nm) over a suitable period of time (e.g. over 120 min at a temperature of 32° C.).

In Vitro Mouse MMP-7 Inhibition Test:

Recombinant mouse MMP-7 (R&D Systems, 2967-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.5 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-7 reaction is measured by measuring the fluorescence intensity (excitation 320 nm, emission 410 nm) over a suitable period of time (e.g. over 120 min at a temperature of 32° C.).

In Vitro Mouse MMP-8 Inhibition Test:

Recombinant mouse MMP-8 (R&D Systems, 2904-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-8 reaction is measured by measuring the fluorescence intensity (excitation 320 nm, emission 410 nm) over a suitable period of time (e.g. over 120 min at a temperature of 32° C.).

In Vitro Mouse MMP-9 Inhibition Test:

Recombinant mouse MMP-9 (R&D Systems, 909-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 5 μM; R&D Systems, ES-001), so as to result in a total test volume of 50 μl. The course of the MMP-9 reaction is measured by measuring the fluorescence intensity (excitation 320 nm, emission 410 nm) over a suitable period of time (e.g. over 120 min at a temperature of 32° C.).

In Vitro Mouse MMP-12 Inhibition Test:

Recombinant mouse MMP-12 (R&D Systems, 3467-MP) is autocatalytically activated in accordance with the manufacturer's instructions. 1 μl of the test compound to be analysed (as a solution in DMSO, suitable concentrations e.g. 1 nM to 30 μM) is pipetted into 24 μl of activated enzyme (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-12 reaction is measured by measuring the fluorescence intensity (excitation 320 nm, emission 410 nm) over a suitable period of time (e.g. over 120 min at a temperature of 32° C.).

High-Sensitivity In Vitro Mouse MMP-12 Inhibition Test:

If sub-nanomolar IC values are found for highly potent test substances in the mouse MMP-12 inhibition test described above, a modified test is used to determine them more accurately. In this case, an enzyme concentration ten times lower is used (final concentration, e.g. 0.1 nM) in order to achieve an increased test sensitivity. The incubation period chosen for the test is correspondingly longer (e.g. 16 hours).

In Vitro Rat MMP-2 Inhibition Test:

Recombinant rat MMP-2 (R&D Systems, 924-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 10 μM; R&D Systems, ES-001), so as to result in a total test volume of 50 μl. The course of the MMP-2 reaction is measured by measuring the fluorescence intensity (excitation 320 nm, emission 410 nm) over a suitable period of time (e.g. over 120 min at a temperature of 32° C.).

In Vitro Rat MMP-8 Inhibition Test:

Recombinant rat MMP-8 (R&D Systems, 3245-MP) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 2 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Lys-Pro-Leu-Gly-Leu-Dpa(Dnp)-Ala-Arg-NH₂ substrate (final concentration e.g. 5 μM; R&D Systems, ES-010), so as to result in a total test volume of 50 μl. The course of the MMP-8 reaction is measured by measuring the fluorescence intensity (excitation 320 nm, emission 410 nm) over a suitable period of time (e.g. over 120 min at a temperature of 32° C.).

In Vitro Rat MMP-9 Inhibition Test:

Recombinant mouse MMP-9 (R&D Systems, 5427-MM) is chemically activated in accordance with the manufacturer's instructions using APMA. 1 μl of the test compound to be analysed (as a solution in DMSO, suitable concentrations e.g. 1 nM to 30 μM) is pipetted into 24 μl of activated enzyme (final concentration e.g. 0.1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 5 μM; R&D Systems, ES-001), so as to result in a total test volume of 50 μl. The course of the MMP-9 reaction is measured by measuring the fluorescence intensity (excitation 320 nm, emission 410 nm) over a suitable period of time (e.g. over 120 min at a temperature of 32° C.).

In Vitro Rat MMP-12 Inhibition Test:

Rat MMP-12 (Uniprot NP_446415.1; construct L96-V277) is expressed with an additional N-terminal His tag and a consecutive TEV cleavage sequence by means of a pDEco7 vector in E. coli (BL21). The protein thus expressed in recombinant form forms an intracellular insoluble protein compartment (called an inclusion body). This is solubilized after separation and intensive washing under denaturing conditions. For this purpose, the inclusion body pellet fragment from a 250 ml E. coli culture is taken up in a volume of 120 ml of buffer A (50 mM Tris pH 7.4, 100 mM NaCl, 0.03 mM ZnCl₂, 10 mM CaCl₂, 8 M urea). The soluble protein is renatured by dialysing 60 ml batches of the sample repeatedly at 4-8° C. against buffer B (50 mM Tris pH 7.4, 100 mM NaCl, 0.03 mM ZnCl₂, 10 mM CaCl₂). After the dialysis, the sample is centrifuged (25 000×g). The refolded protein is obtained in the supernatant with a yield of 3.7 mg per 250 ml of E. coli culture. The protein thus obtained is enzymatically active without further purifying operations or protease-mediated cleavage processes.

1 μl of the test compound to be analysed (as a solution in DMSO, suitable concentrations e.g. 1 nM to 30 μM) is pipetted into 24 μl of MMP-12 protein (final concentration e.g. 1 nM) in reaction buffer (50 mM Tris/HCl pH 7.5, 10 mM CaCl₂, 150 mM NaCl, 0.05% Brij®-35) in a white 384-hole microtiter plate (MTP). The enzymatic reaction is started by adding the intramolecularly quenched Mca-Pro-Leu-Gly-Leu-Dpa(Dnp)-Ala-Arg-NH₂ substrate (final concentration e.g. 5 μM; R&D Systems, ES-001), so as to result in a total test volume of 50 μl. The course of the MMP-12 reaction is measured by measuring the fluorescence intensity (excitation 320 nm, emission 410 nm) over a suitable period of time (e.g. over 120 min at a temperature of 32° C.).

B-3. Animal Model of Pulmonary Emphysema

Elastase-induced pulmonary emphysema in mice, rats or hamsters is a widely used animal model for pulmonary emphysema [The Fas/Fas-ligand pathway does not mediate the apoptosis in elastase-induced emphysema in mice, Sawada et al., Exp. Lung Res. 33, 277-288 (2007)]. The animals receive an orotracheal instillation of porcine pancreas elastase. The treatment of the animals with the test substance starts on the day of the instillation of the porcine pancreas elastase and extends over a period of 3 weeks. At the end of the study, lung compliance is determined and alveolar morphometry is conducted.

B-4. Animal Model of Silica-Induced Pulmonary Inflammation

Orotracheal administration of silica in mice, rats or hamsters leads to inflammation in the lung [Involvement of leukotrienes in the pathogenesis of silica-induced pulmonary fibrosis in mice, Shimbori et al., Exp. Lung Res. 36, 292-301 (2010)]. The animals are treated with the test substance on the day of instillation of the silica. After 24 hours, a bronchio-alveolar lavage is carried out to determine the cell content and the biomarkers.

B-5. Animal Model of Silica-Induced Pulmonary Fibrosis

Silica-induced pulmonary fibrosis in mice, rats or hamsters is a widely used animal model for pulmonary fibrosis [Involvement of leukotrienes in the pathogenesis of silica-induced pulmonary fibrosis in mice, Shimbori et al., Exp. Lung Res. 36, 292-301 (2010)]. The animals receive an orotracheal instillation of silica. The treatment of the animals with the test substance starts on the day of the instillation of the silica or therapeutically a week later and extends over a period of 6 weeks. At the end of the study, a bronchio-alveolar lavage to determine the cell content and the biomarkers and a histological assessment of pulmonary fibrosis are carried out.

B-6. Animal Model of ATP-Induced Pulmonary Inflammation

Intratracheal administration of ATP (adenosine triphosphate) in mice leads to inflammation in the lung [Acute lung inflammation and ventilator-induced lung injury caused by ATP via the P2Y receptors: An experimental study, Matsuyama et al., Respir. Res. 9:79 (2008)]. On the day of the instillation of ATP, the animals are treated with the test substance for a duration of 24 h (by gavage, by addition to the feed or drinking water, using an osmotic minipump, by subcutaneous or intraperitoneal injection or by inhalation). At the end of the experiment, a bronchio-alveolar lavage is conducted to determine the cell content and the pro-inflammatory markers.

B-7. CYP Inhibition Test

The ability of substances to inhibit the CYP enzymes CYP1A2, CYP2C9, CYP2D6 and CYP3A4 in humans is examined using pooled human liver microsomes as enzyme source in the presence of standard substrates (see below) which form CYP-specific metabolites. The inhibition effects are studied at six different concentrations of the test compounds [2.8, 5.6, 8.3, 16.7, 20 (or 25) and 50 μM) and compared with the extent of the CYP-specific metabolite formation of the standard substrates in the absence of the test compounds, and the corresponding IC₅₀ are calculated. A standard inhibitor that specifically inhibits an individual CYP isoform is always included in the incubation, in order to make results comparable between different series.

The incubation of phenacetin, diclofenac, tolbutamide, dextromethorphan or midazolam with human liver microsomes in the presence of six different concentrations of each test compound (as potential inhibitor) is carried out on a workstation (Tecan, Genesis, Crailsheim, Germany). Standard incubation mixtures contain 1.3 mM NADP⁺, 3.3 mM MgCl₂×6 H₂O, 3.3 mM glucose 6-phosphate, glucose 6-phosphate dehydrogenase (0.4 U/ml) and 100 mM phosphate buffer (pH 7.4) in a total volume of 200 μl. Test compounds are preferably dissolved in acetonitrile. 96-Well plates are incubated for a defined period of time at 37° C. with pooled human liver microsomes. The reactions are stopped by addition of 100 μl of acetonitrile with a suitable internal standard present therein. Precipitated proteins are removed by centrifugation, and the supernatants are combined and analysed by LC-MS/MS.

B-8. Hepatocyte Assay for Determination of Metabolic Stability

The metabolic stability of test compounds towards hepatocytes is determined by incubating the compounds at low concentrations (preferably below or around 1 μM) and at low cell counts (preferably at 1*10⁶ cells/ml) in order to ensure maximum linearity of kinetic conditions in the experiment. Seven samples from the incubation solution are taken for the LC-MS analysis within a fixed time pattern, in order to determine the half-life (i.e. the degradation) of the particular compound. This half-life is used to calculate various “Clearance” parameters (CL) and “Fₘₐₓ” values (see below).

The CL and Fₘₐₓ values are a measure of the phase 1 and phase 2 metabolism of the compounds in the hepatocytes. In order to minimize the influence of the organic solvent on the enzymes in the incubation batches, the concentration thereof is generally limited to 1% (acetonitrile) or 0.1% (DMSO).

For all species and breeds, a hepatocyte cell count in the liver of 1.1*10⁸ cells/g of liver is expected. CL parameters calculated on the basis of half-lives which extend considerably beyond the incubation time (typically 90 minutes) can only be regarded as rough guide values.

The parameters calculated and the meanings thereof are:

| | |
|---|---|
| Fₘₐₓ well-stirred [%] | maximum possible bioavailability after oral |
| | administration |
| Calculation: | (1 − CL_{blood} well-stirred/QH) * 100 |
| CL_{blood} well-stirred | calculated blood clearance (well-stirred model) |
| [L/(h*kg)] | |
| Calculation: | (QH * CL′_{intrinsic})/(QH + CL′_{intrinsic}) |
| CL′_{intrinsic} | maximum ability of the liver (of the hepatocytes) |
| [ml/(min*kg)] | to metabolize a compound (assuming that the |
| | liver blood flow is not rate-limiting) |
| Calculation: | CL′_{intrinsic, apparent} * species-specific hepatocyte |
| | count [1.1 * 10⁸/g liver] * species-specific |
| | liver weight [g/kg] |
| CL′_{intrinsic, apparent} | normalizes the elimination constant by dividing it |
| [ml/(min*mg)] | by the hepatocyte cell count x used (x * 10⁶/ml) |
| Calculation: | kₑₗ [1/min]/(cell count [x * 10⁶]/ |
| | incubation volume [ml]) |

(QH = species-specific liver blood flow).

Table 3 below shows, for representative working examples of the invention, the CL and Fₘₐₓ values from this assay after incubation of the compounds with rat hepatocytes (some as mean values from two or more independent individual determinations):

TABLE 3
Calculated blood clearance and bioavailability
after incubation with rat hepatocytes

| Example | CL_{blood} | Fₘₐₓ |
|---|---|---|
| No. | [L/(h*kg)] | [%] |
| 5 | 1.62 | 61.4 |
| 32 | 1.32 | 68.5 |
| 38 | 0.75 | 82.1 |
| 57 | 2.14 | 48.9 |
| 73 | 1.39 | 66.9 |
| 94 | 1.55 | 63.2 |
| 112 | 1.99 | 52.5 |
| 130 | 2.09 | 50.3 |
| 133 | 0.80 | 81.1 |
| 134 | 1.18 | 71.8 |
| 135 | 1.36 | 67.5 |
| 136 | 0.73 | 82.7 |
| 140 | 1.49 | 64.5 |
| 144 | 1.74 | 58.6 |
| 145 | 1.53 | 63.7 |
| 154 | 1.22 | 70.9 |
| 155 | 1.21 | 71.1 |
| 159 | 0.65 | 84.5 |

B-9. Metabolic Study

To determine the metabolic profile of the compounds according to the invention, they are incubated with liver microsomes or with primary fresh hepatocytes from various animal species (e.g. rats, dogs), and also of human origin, in order to obtain and to compare information about a very substantially complete hepatic phase I and phase II metabolism, and about the enzymes involved in the metabolism.

The compounds according to the invention are incubated with a concentration of about 1-10 μM. To this end, stock solutions of the compounds having a concentration of 0.1-1 mM in acetonitrile are prepared, and then pipetted with a 1:100 dilution into the incubation mixture. The liver microsomes are incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes are incubated in suspension in William's E medium, likewise at 37° C. After an incubation time of 0-4 h, the incubation mixtures are stopped with acetonitrile (final concentration about 30%) and the protein is centrifuged off at about 15 000×g. The samples thus stopped are either analysed directly or stored at −20° C. until analysis.

The analysis is carried out by high-performance liquid chromatography with ultraviolet and mass spectrometry detection (HPLC-UV-MS/MS). To this end, the supernatants of the incubation samples are chromatographed with suitable C18 reverse-phase columns and variable mobile phase mixtures of acetonitrile and 10 mM aqueous ammonium formate solution or 0.05% aqueous formic acid. The UV chromatograms in conjunction with the mass spectrometry data serve for identification, structural elucidation and quantitative estimation of the metabolites, and for quantitative determination of the metabolic decrease in the compounds according to the invention in the incubation mixtures.

B-10. Pharmacokinetic Studies In Vivo

The substance to be examined is administered to rats or mice intravenously as a solution (e.g. in corresponding plasma with a small addition of DMSO or in a PEG/ethanol/water mixture), and peroral administration is effected as a solution (e.g. in Solutol/ethanol/water or PEG/ethanol/water mixtures) or as a suspension (e.g. in tylose), in each case via a gavage. After administration of the substance, blood is obtained from the animals at fixed time points. It is heparinized, then plasma is obtained from it by centrifugation. The test substance is quantified analytically in the plasma by LC-MS/MS. The plasma concentration/time plots determined in this way are used to calculate, using an internal standard and with the aid of a validated computer program, the pharmacokinetic parameters, such as AUC (area under the concentration/time curve), Cₘₐₓ (maximum plasma concentration), t_{1/2} (half-life), V_{SS} (distribution volume) and CL (clearance), and the absolute and relative bioavailability F and Fᵣₑₗ (i.v./p.o. comparison or comparison of suspension to solution after p.o. administration).

### C. WORKING EXAMPLES OF PHARMACEUTICAL COMPOSITIONS

The compounds according to the invention can be converted to pharmaceutical preparations as follows:

Tablet:

Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

Production:

The mixture of compound according to the invention, lactose and starch is granulated with a 5% solution (w/w) of the PVP in water. The granules are dried and then mixed with the magnesium stearate for 5 minutes. This mixture is compressed using a conventional tabletting press (see above for format of the tablet). The guide value used for the pressing is a pressing force of 15 kN.

Suspension for Oral Administration:

Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

Production:

The Rhodigel is suspended in ethanol; the compound according to the invention is added to the suspension. The water is added while stirring. The mixture is stirred for about 6 h until the swelling of the Rhodigel is complete.

Solution for Oral Administration:

Composition:

500 mg of the compound according to the invention, 2.5 g of polysorbate and 97 g of polyethylene glycol 400. 20 g of oral solution correspond to a single dose of 100 mg of the compound according to the invention.

Production:

The compound according to the invention is suspended in the mixture of polyethylene glycol and polysorbate with stirring. The stirring operation is continued until dissolution of the compound according to the invention is complete.

I.V. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. Compound of the formula (I) 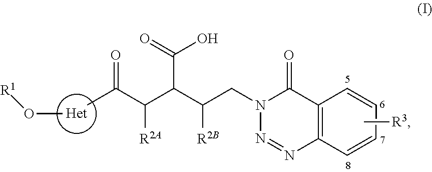
in which
Het represents bicyclic heteroaryl of the formula 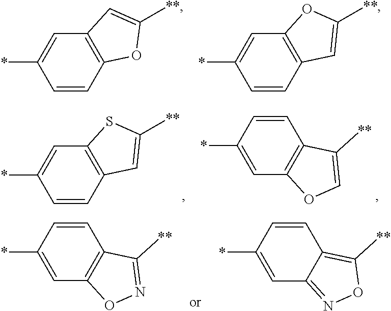
in which * labels the linkage to the R¹O group and ** labels the linkage to the carbonyl group,
R¹ represents (C₁-C₈)-alkyl which can be substituted with cyano or up to six times with fluorine,
or
represents a group of the formula 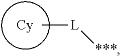
in which *** labels the linkage to the O atom,
L represents —CH₂—, —CH₂—CH₂—, —C(═O)—CH₂—*** or —CH₂—CH₂—CH₂—
and
Cy represents (C₃-C₆)-cycloalkyl, oxetanyl, tetrahydrofuranyl, tetrahydropyranyl, phenyl, thienyl, pyridyl or 2,1,3-benzoxadiazolyl,
where (C₃-C₆)-cycloalkyl, oxetanyl, tetrahydrofuranyl and tetrahydropyranyl can be mono- or disubstituted, identically or differently, with a radical selected from the group fluorine and methyl
and
where phenyl, thienyl and pyridyl can be mono- or disubstituted, identically or differently, with a radical selected from the group fluorine, chlorine, cyano, methyl, difluoromethyl, trifluoromethyl, methoxy, ethoxy, trifluoromethoxy, (trifluoromethyl)sulphanyl, methylsulphonyl, aminocarbonyl, methoxycarbonylamino and 2-oxo-1,3-oxazolidin-3-yl,
R^{2A} and R^{2B} both represent hydrogen or are linked with one another and together form a —CH₂CH₂ bridge
and
R³ represents hydrogen or a substituent selected from the group fluorine, methyl, fluoromethyl, difluoromethyl and trifluoromethyl, where such a substituent can be bonded in the designated position 5, 6, 7 or 8,
and the salts, solvates and solvates of the salts thereof.

2. Compound of the formula (I) according to claim 1 in which
Het represents bicyclic heteroaryl of the formula 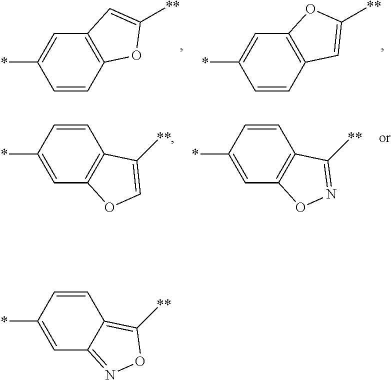
in which * labels the linkage to the R¹O group and ** labels the linkage to the carbonyl group,
R¹ represents (C₄-C₇)-alkyl or a group of the formula 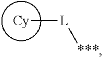
in which *** labels the linkage to the O atom,
L represents —CH₂— or —CH₂—CH₂—
and
Cy represents (C₃-C₆)-cycloalkyl, tetrahydropyranyl or phenyl,
where phenyl can be mono- or disubstituted, identically or differently, with a radical selected from the group fluorine, chlorine, methyl, trifluoromethyl, methoxy and aminocarbonyl,
R^{2A} and R^{2B} both represent hydrogen or are linked with one another and together form a —CH₂CH₂ bridge
and
R³ represents hydrogen or a substituent selected from the group methyl and trifluoromethyl, where such a substituent is bonded in the designated position 6 or 7,
and the salts, solvates and solvates of the salts thereof.

3. Compound of the formula (I) according to claim 1 in which
Het represents bicyclic heteroaryl of the formula 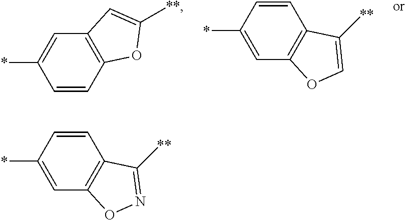
in which * labels the linkage to the R¹O group and ** labels the linkage to the carbonyl group,
R¹ represents (C₄-C₇)-alkyl or a group of the formula 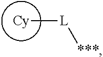
in which *** labels the linkage to the O atom,
L represents —CH₂— or —CH₂—CH₂—
and
Cy represents (C₃-C₆)-cycloalkyl, tetrahydropyran-4-yl or phenyl,
where phenyl can be mono- or disubstituted, identically or differently, with a radical selected from the group fluorine, chlorine, methyl, trifluoromethyl, methoxy and aminocarbonyl,
R^{2A} and R^{2B} both represent hydrogen
and
R³ represents hydrogen or trifluoromethyl which is bonded in the designated position 6,
and the salts, solvates and solvates of the salts thereof.

4. Compound according to claim 1 having the formula (I-A*) 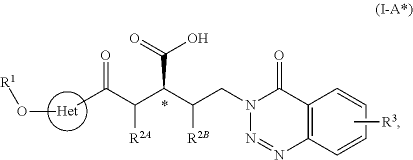
in which Het, R¹ and R³ have the meanings given in claim 1, R^{2A} and R^{2B} in each case represent hydrogen and the chiral carbon atom labelled with * in enantiomerically pure form has the depicted S configuration,
and the salts, solvates and solvates of the salts thereof.

5. Process for preparing a compound of the formula (I) according to claim 1, in which R^{2A} and R^{2B} in each case represent hydrogen, characterized in that di-tert-butyl (2-hydroxyethyl)malonate of the formula (II) 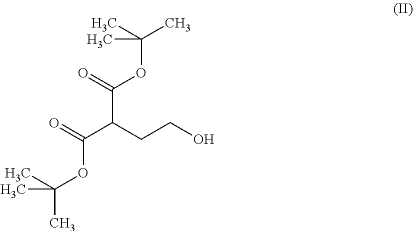 [SMILES: CC(C)(C)OC(=O)C(CCO)C(=O)OC(C)(C)C]
is reacted in the presence of a phosphine and an azodicarboxylate with a benzotriazin-4(3H)-one derivative of the formula (III) 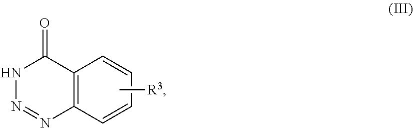
in which R³ has the meaning given in claim 1
to give a compound of the formula (IV) 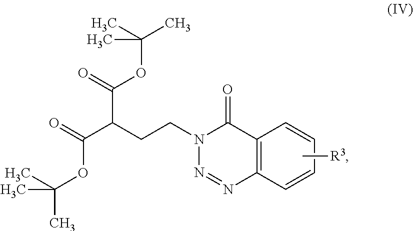
in which R³ has the meaning given in claim 1
and the compound of the formula (IV) is then either
[A] alkylated in the presence of a base with a compound of the formula (V) 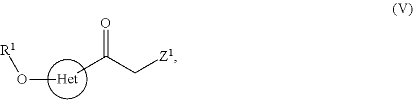
in which Het and R¹ have the meanings given in claim 1
and
Z¹ represents a leaving group such as, for example, chlorine, bromine or iodine,
to give a compound of the formula (VI) 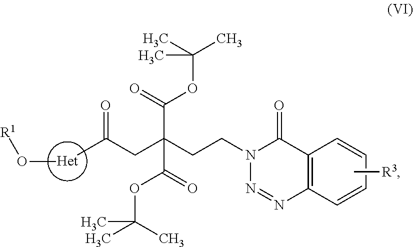
in which Het, R¹ and R³ have the meanings given in claim 1,
or
[B] alkylated firstly with a compound of the formula (VII) 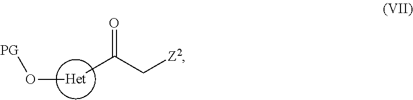
in which Het has the meaning given in claim 1,
PG represents a suitable temporary protective group such as, for example, benzyl
and
Z² represents a leaving group such as, for example, chlorine, bromine or iodine,
in the presence of a base to give a compound of the formula (VIII) 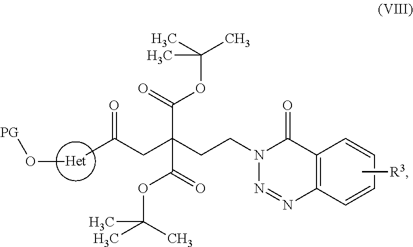
in which Het, PG and R³ have the meanings given above,
then the protective group PG is cleaved off and the resulting compound of the formula (IX) 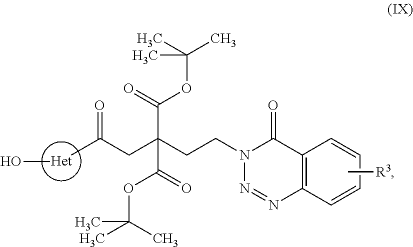
in which Het and R³ have the meanings given in claim 1,
is then in the presence of a base alkylated with a compound of the formula (X)
R¹—Z³ (X),
in which R¹ has the meaning given in claims 1 to 4
and
Z³ represents a leaving group such as, for example, chlorine, bromine, iodine, mesylate, triflate or tosylate,
to give the compound of the formula (VI) 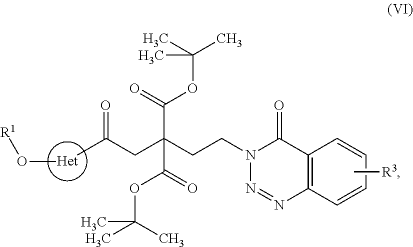
in which Het, R¹ and R³ have the meanings given in claim 1,
and the compound of the formula (VI) thus obtained by method [A] or [B] is finally converted by treatment with an acid and subsequent heating to the carboxylic acid of the formula (I-A) 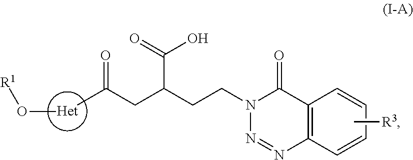
in which Het, R¹ and R³ have the meanings given in claim 1,
and the compounds of the formula (I-A) are optionally separated into their enantiomers and/or diastereomers and/or optionally reacted with the corresponding (i) solvents and/or (ii) bases to give solvates, salts and/or solvates of the salts thereof.

6. Compound as defined in claim 1 for the treatment and/or prevention of diseases.

7. Compound as defined in claim 1 for use in a method for the treatment and/or prevention of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome.

8. Use of a compound as defined in claim 1 for producing a medicament for the treatment and/or prevention of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome.

9. Medicament comprising a compound as defined in claim 1 in combination with one or more inert, nontoxic, pharmaceutically suitable excipients.

10. Medicament comprising a compound as defined in claim 1 in combination with one or more further active ingredients selected from the group consisting of corticosteroids, beta-adrenergic receptor agonists, antimuscarinic substances, PDE 4 inhibitors, PDE 5 inhibitors, sGC activators, sGC stimulators, HNE inhibitors, prostacyclin analogues, endothelin antagonists, statins, antifibrotic agents, antiinflammatory agents, immunomodulating agents, immunosuppressive agents and cytotoxic agents.

11. Medicament according to claim 9 for the treatment and/or prevention of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome.

12. Method for the treatment and/or prevention of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome in humans and animals by administering an effective amount of at least one compound as defined in claim 1.

13. Method for the treatment and/or prevention of chronic obstructive pulmonary disease (COPD), pulmonary emphysema, chronic bronchitis, pulmonary hypertension in COPD (PH-COPD), bronchiectasis, asthma, interstitial pulmonary disorders, idiopathic pulmonary fibrosis (IPF) and pulmonary sarcoidosis, of arteriosclerosis, carotid arteriosclerosis, viral myocarditis, cardiomyopathy and aneurysms, including the sequelae thereof such as stroke, myocardial infarction and peripheral arterial occlusive disease, and also of chronic kidney diseases and Alport's syndrome in humans and animals by administering an effective amount of at least one compound as defined in claim 9.
